(19) United States
(12) Patent Application Publication
(10) Pub. No.: US 2023/0120677 A1
(43) Pub. Date: Apr. 20, 2023

(54) IMMUNOLOGICALLY OPTIMIZED BOTULINUM TOXIN LIGHT CHAIN VARIANTS

(71) Applicants: TRUSTEES OF DARTMOUTH COLLEGE, Hanover, NH (US); CHILDREN'S MEDICAL CENTER CORPORATION, Boston, MA (US)

(72) Inventors: Karl E. GRISWOLD, Lyme, NH (US); Chris BAILEY-KELLOGG, STRAFFORD, VT (US); Yoonjoo CHOI, GYEONGGI-DO (KR); Yongliang FANG, JIANGSU (CN); Deeptak VERMA, FEASTERVILLE-TREVOSE, PA (US); Susan ESZTERHAS, PLAINFIELD, NH (US); Min DONG, WEATOGUE, CT (US); Shin-Ichiro MIYASHITA, BROOKLINE, MA (US)

(21) Appl. No.: 17/759,045

(22) PCT Filed: Jan. 20, 2021
(86) PCT No.: PCT/US21/14145

§ 371 Date: Jul. 19, 2022

Related U.S. Application Data
(60) Provisional application No. 62/963,774, filed on Jan. 21, 2020.

(51) Int. Cl. C07K 14/33; C07K 16/00; C12N 15/70; C12N 15/79; A61P 21/00

(52) U.S. Cl. CPC C07K 14/33; C07K 16/00; C12N 15/70; C12N 15/79; A61P 21/00; C07K 2317/52; C07K 2317/55; C07K 2317/622

(57) ABSTRACT

Deimmunized botulinum toxin light chain (e.g., botulinum toxin serotype A light chains (BoNT/A-LC)) or fragments thereof are provided. Methods for treating or preventing diseases or disorders comprising administering to a subject a deimmunized botulinum toxin light chain (e.g., BoNT/A-LC) are provided.

### RELATED APPLICATIONS

This application claims the benefit of U.S. Provisional Application No. 62/963,774, filed Jan. 21, 2020, the entire disclosure of which is hereby incorporated by reference.

### STATEMENT OF FEDERALLY SPONSORED RESEARCH

This invention was made with government support under Grant No. R01 GM098977 awarded by the National Institutes of Health. The Government has certain rights in this invention.

### FIELD OF THE INVENTION

This disclosure relates to compositions and methods of making immunologically optimized botulinum toxin light chain variants.

### BACKGROUND

Botulinum neurotoxin serotype A (BoNT/A) is a well-known biotherapeutic due to its cosmetic application to treat brow line wrinkles and glabellar frown lines and medical application to treat various diseases in the clinic. However, BoNT/A is also immunogenic and has the potential to induce an adverse immune response, including production of anti-drug antibodies (ADA) in humans. The formation of ADAs can lead to the loss of therapeutic efficacy, altered pharmacokinetics, deposition of toxic immune complexes, and various allergic type reactions. Since some approved indications of BoNT/A are chronic disorders, long-term treatment with repeated dosing is required, and such a treatment regime further increases immunogenicity risk. Indeed, repeated injections of BoNT/A have led to the development of neutralizing antibodies (Nabs) against the toxin, which have been reported to cause treatment failure (Jankovic et al. Toxicon: official journal of the International Society on Toxinology. 54, 614-623. 2009; Troung et al. Parkinsonism & related disorders. 16, 316-323. 2010; Jankovic et al. Neurology. 67, 2233-2235. 2006; Troung et al. Movement disorders: official journal of the Movement Disorder Society. 20, 783-791. 2005; Mejia et al. Movement disorders: official journal of the Movement Disorder Society. 20, 592-597. 2005; Jankovic et al. Neurology. 60, 1186-1188. 2003; Jankovic et al. The Lancet. Neurology. 5, 864-872. 2006; Brin et al. Movement disorders: official journal of the Movement Disorder Society. 23, 1353-1360. 2008).

Therefore, the immunogenicity of BoNT/A, and other BoNT serotypes (i.e., BoNT/B, BoNT/C, BoNT/D, BoNT/E, BoNT/F, and BoNT/G) represents a problem for broader clinical applications beyond cosmetics, where the toxin requires repeated administration at higher doses.

### SUMMARY

Disclosed herein are immunologically optimized botulinum toxin light chain variants and methods of producing the same.

In one aspect, the disclosure provides a deimmunized botulinum toxin light chain or fragment thereof comprising at least one mutation in a botulinum toxin light chain amino acid sequence selected from the group consisting of: a) a botulinum toxin serotype A light chain (BoNT/A-LC) or fragment thereof of SEQ ID NO: 1; b) a botulinum toxin serotype B light chain (BoNT/B-LC) or fragment thereof of SEQ ID NO: 2; c) a botulinum toxin serotype C light chain (BoNT/C-LC) or fragment thereof of SEQ ID NO: 3; d) a botulinum toxin serotype D light chain (BoNT/D-LC) or fragment thereof of SEQ ID NO: 4; e) a botulinum toxin serotype E light chain (BoNT/E-LC) or fragment thereof of SEQ ID NO: 5; f) a botulinum toxin serotype F light chain (BoNT/F-LC) or fragment thereof of SEQ ID NO: 6; or g) a botulinum toxin serotype G light chain (BoNT/G-LC) or fragment thereof of SEQ ID NO: 7, wherein the at least one mutation reduces the immunogenicity of the botulinum toxin light chain or fragment thereof.

In certain embodiments, the deimmunized BoNT/A-LC or fragment thereof comprises a mutation at one or more of V16, Q30, I41, V43, D80, N81, S99, G119, I137, L150, S156, Y184, F193, L199, F212, I225, I234, I236, R240, F242, M252, S258, L276, E278, N279, L283, Y284, Y285, F289, S294, K298, I302, Q310, L321, S323, F330, L335, V354, K358, L360, K363, T364, N367, F368, A371, F373, V381, I385, Y386, T413, K416, F418, L421, F422, I433, I434, T435, and T438 of SEQ ID NO: 1.

In certain embodiments of the deimmunized BoNT/A-LC or fragment thereof, the mutation comprises V16R or V16L; Q30E or Q30T; I41V; V43I; D80N; N81A; S99E; G119S; I137K; L150V; S156G; Y184I; F193S or F193N; L199T or L199Q; F212Y; I225T; I234T; I236G; R240E; F242T or F242S; M252Q; S258K; L276A; E278K; N279K; L283D, L283N, L283E, or L283T; Y284K; Y285A; F289Y; S294K or S249D; K298E; I302T; Q310D; L321K, L321G, or L321N; S323D; F330Y; L335D, L335E, or L335N; V354S or V354A; K358N; L360Q, L360I, or L360K; K363Q; T364S; N367G; F368Q or F368D; A371G; F373K; V381D or V381E; I385V; Y386K, Y386S, or Y386H; T413D or T413E; K416S; F418G, F418K, or F418E; L421V; F422V; I433T; I434K; T435N; T438D; or a combination thereof, of SEQ ID NO: 1.

In certain embodiments, the deimmunized BoNT/B-LC or fragment thereof comprises a mutation at one or more of N16, R31, D82, S100, L140, L157, Q191, S200, I232, I241, P247, Q264, C308, N317, E342, A361, K367, P379, E389, E394, and E421 of SEQ ID NO: 2.

In certain embodiments of the deimmunized BoNT/B-LC or fragment thereof, the mutation comprises N16R; R31E; D82A; S100E; L140K; L157V; Q191I; S200N; I232T; I241T; P247E; Q264K; C308T; N317D; E342N; A361S; K367Q; P379G; E389D; E394K; E421D; or a combination thereof, of SEQ ID NO: 2.

In certain embodiments, the deimmunized BoNT/C-LC or fragment thereof comprises a mutation at one or more of K16, S80, S98, R161, L199, F218, N231, I240, T247, E265, E290, A309, G319, R33K, A363, P381, N390, Q395, and R421 of SEQ ID NO: 3.

In certain embodiments of the deimmunized BoNT/C-LC or fragment thereof, the mutation comprises K16R; S80A; S98E; R161G; L199N; F218Y; N231T; I240T; T247E; E265K; E290D; A309T; G319D; R330K; A363S; P381G; N390D; Q395K; R421D; or a combination thereof, of SEQ ID NO: 3.

In certain embodiments, the deimmunized BoNT/D-LC or fragment thereof comprises a mutation at one or more of N16, T31, E80, E138, L161, L199, F218, I240, R247, Q265, E290, N330, D344, K369, P381, N390, R395, and Q421 of SEQ ID NO: 4.

In certain embodiments of the deimmunized BoNT/D-LC or fragment thereof, the mutation comprises N16R; T31E; E80A; E138K; L161G; L199N; F218Y; I240T; R247E; Q265K; E290D; N330K; D344N; K369Q; P381G; N390D; R395K; Q421D; or a combination thereof, of SEQ ID NO: 4.

In certain embodiments, the deimmunized BoNT/E-LC or fragment thereof comprises a mutation at one or more of E77, N95, E153, F191, F201, I214, A223, Y230, N247, T272, N273, S291, N296, G307, R339, K345, Y356, S366, S371, and T396 of SEQ ID NO: 5.

In certain embodiments of the deimmunized BoNT/E-LC or fragment thereof, the mutation comprises E77A; N95E; E153G; F191N; F201Y; 1214T; A223T; Y230E; N247K; T272D; N273K; S291T; N296D; G307K; R339S; K345Q; Y356G; S366D; S371K; T396D; or a combination thereof, of SEQ ID NO: 5.

In certain embodiments, the deimmunized BoNT/F-LC or fragment thereof comprises a mutation at one or more of D16, K31, S99, L152, Y200, F216, I229, A238, R262, N287, N288, A306, N313, G324, A356, K362, F373, and S388 of SEQ ID NO: 6.

In certain embodiments of the deimmunized BoNT/F-LC or fragment thereof, the mutation comprises D16R; K31E; S99E; L152V; Y200N; F216Y; 1229T; A238T; R262K; N287D; N288K; A306T; N313D; G324K; A356S; K362Q; F373G; S388K; or a combination thereof, of SEQ ID NO: 6.

In certain embodiments, the deimmunized BoNT/G-LC or fragment thereof comprises a mutation at one or more of D16, T31, S100, L157, M191, I232, I241, P247, Q264, N289, A308, S316, D327, D341, A360, K366, P378, T388, N393, and E420 of SEQ ID NO: 7.

In certain embodiments of the deimmunized BoNT/G-LC or fragment thereof, the mutation comprises D16R; T31E; S100E; L157V; M191I; I232T; I241T; P247E; Q264K; N289D; A308T; S316D; D327K; D341N; A360S; K366Q; P378G; T388D; N393K; E420D; or a combination thereof, of SEQ ID NO: 7.

In certain embodiments, the deimmunized botulinum toxin light chain or fragment thereof comprises 2, 3, 4, 5, 6, 7, 8, 9, 10, 11, 12, 13, 14, 15, 16, 17, 18, 19, 20, or more mutations.

In certain embodiments, the deimmunized botulinum toxin light chain or fragment thereof comprises about 0.10% activity or greater relative to a wildtype botulinum toxin light chain or fragment thereof. In certain embodiments, the deimmunized botulinum toxin light chain or fragment thereof comprises about 0.1%, about 1%, about 5%, about 10%, about 20%, about 30%, about 40%, about 50%, about 60%, about 70%, about 80%, about 90%, or about 100% activity relative to a wildtype botulinum toxin light chain or fragment thereof.

In certain embodiments, the deimmunized botulinum toxin light chain or fragment thereof comprises thermostability within about 10° C. of a wildtype botulinum toxin light chain or fragment thereof. In certain embodiments, the deimmunized botulinum toxin light chain or fragment thereof comprises thermostability within about 3° C. to about 8° C. of a wildtype botulinum toxin light chain or fragment thereof. In certain embodiments, the deimmunized botulinum toxin light chain or fragment thereof comprises thermostability about equal to a wildtype botulinum toxin light chain or fragment thereof.

In certain embodiments, the deimmunized botulinum toxin light chain or fragment thereof is fused to a functional moiety.

In certain embodiments, the functional moiety comprises a targeting activity and/or binding activity.

In certain embodiments, the functional moiety is selected from the group consisting of an antigen binding protein or fragment thereof, an imaging molecule, an oligonucleotide, a targeting peptide, and polyethylene glycol (PEG). In certain embodiments, the antigen binding protein fragment comprises an Fc domain, a Fab domain, an scFv, or a single domain antibody.

In certain embodiments, the deimmunized botulinum toxin light chain or fragment thereof further comprises a botulinum toxin heavy chain (BoNT-HC) or fragment thereof.

In certain embodiments, the deimmunized botulinum toxin light chain or fragment thereof further comprises a botulinum toxin serotype A heavy chain (BoNT/A-HC) or fragment thereof.

In certain embodiments, the BoNT-HC or fragment thereof is a serotype other than serotype A.

In certain embodiments, the BoNT-HC serotype is selected from the group consisting of serotype B, serotype C, serotype D, serotype E, serotype F, and serotype G.

In another aspect, the disclosure provides a pharmaceutical composition comprising the deimmunized botulinum toxin light chain or fragment thereof as recited above and a pharmaceutically acceptable carrier.

In another aspect, the disclosure provides a vector encoding the deimmunized botulinum toxin light chain or fragment thereof as recited above.

In another aspect, the disclosure provides a host cell comprising the vector recited above.

In certain embodiments, the host cell comprises prokaryotic host cell or a eukaryotic host cell. In certain embodiments, the host cell comprises an E. coli host cell, a Clostridium genus host cell, a yeast host cell, an insect host cell, or a mammalian host cell. In certain embodiments, the clostridium genus host cell comprises C. botulinum, C. baratii, C. butyricum, or C. argentinense.

In one aspect, the disclosure provides a method of treating or preventing a disease or disorder in a subject that would benefit from a therapeutically effective amount of a botulinum toxin, comprising administering a therapeutically effective amount of the deimmunized botulinum toxin light chain or fragment thereof as recited above to the subject.

In one aspect, the disclosure provides a method of treating or preventing a disease or disorder of inappropriate muscle contraction in a subject, comprising administering a therapeutically effective amount of the deimmunized botulinum toxin light chain or fragment thereof as recited above to the subject.

In one aspect, the disclosure provides a method of treating or preventing a disease or disorder of inappropriate neuron signaling in a subject, comprising administering a therapeutically effective amount of the deimmunized botulinum toxin light chain or fragment thereof as recited above to the subject.

In certain embodiments, the disease or disorder is selected from the group consisting of acute pain, alopecia, aquagenic keratoderma, atrial fibrillation, blepharospasm, bromhidrosis, cerebral palsy, cervical dystonia, chromhidrosis, chronic anal fissures, chronic pain, constipation, depression, dermatosis, eccrine nevus, eczema, esophageal spasms, essential tremor, facial erythema and flushing, genodermatoses, Hailey-Hailey disease, hand dystonia, hemifacial spasm, hidradenitis suppurativa, hyperhydrosis, hypersialorrhoea, hypertrophic scars, keloids, linear IgA bullous dermatosis, migraine headache, notalgia paresthetica, oily skin, postherpetic neuralgia, psoriasis, overactive bladder, premature ejaculation, Raynaud's Disease, spastic paresis, strabismus, tension headache, voice abnormalities, whiplash.

In one aspect, the disclosure provides a method of treating a subject for a cosmetic purpose, comprising administering a therapeutically effective amount of the deimmunized botulinum toxin light chain or fragment thereof as recited above to the subject.

In certain embodiments, the cosmetic purpose is the reduction of facial wrinkles. In certain embodiments, the facial wrinkles comprise brow line wrinkles and glabellar frown lines.

In one aspect, the disclosure provides a method for reducing an antibody response against botulinum toxin light chain or fragment thereof in a subject, comprising administering a therapeutically effective amount of the deimmunized BoNT/A-LC or fragment thereof as recited above to the subject.

In one aspect, the disclosure provides a method for producing a deimmunized botulinum toxin light chain or fragment thereof in a host cell, comprising: a) introducing a vector encoding the deimmunized botulinum toxin light chain or fragment thereof as recited above, into a host cell to produce a deimmunized botulinum toxin light chain-expressing host cell; b) culturing the host cell in a culture system; and c) isolating the deimmunized botulinum toxin light chain or fragment thereof from the culture system.

In one aspect, the disclosure provides a deimmunized botulinum toxin serotype A light chain (BoNT/A-LC) or fragment thereof comprising a mutation at one or more of V16, Q30, I41, V43, D80, N81, S99, G119, I137, L150, S156, Y184, F193, L199, F212, I225, I234, I236, R240, F242, M252, S258, L276, E278, N279, L283, Y284, Y285, F289, S294, K298, I302, Q310, L321, S323, F330, L335, V354, K358, L360, K363, T364, N367, F368, A371, F373, V381, I385, Y386, T413, K416, F418, L421, F422, I433, I434, T435, and T438 of SEQ ID NO: 1.

In one aspect, the disclosure provides a deimmunized botulinum toxin serotype A light chain (BoNT/A-LC) or fragment thereof comprising a mutation at one or more of V16, Q30, N81, S99, G119, I137, L150, S156, Y184, F193, F212, I225, I234, R240, S258, L283, Y284, I302, Q310, L321, L335, V354, L360, A371, V381, Y386, T413, and F418 of SEQ ID NO: 1.

In one aspect, the disclosure provides a deimmunized botulinum toxin serotype A light chain (BoNT/A-LC) or fragment thereof comprising a mutation at one or more of Q30, I41, V43, D80, S99, F193, L199, I236, F242, M252, L276, E278, N279, L283, Y284, Y285, F289, S294, K298, Q310, L321, S323, F330, L335, V354, K358, L360, K363, T364, N367, F368, F373, V381, I385, Y386, T413, K416, F418, L421, F422, I433, I434, T435, and T438 of SEQ ID NO: 1.

In certain embodiments, the mutation comprises V16R or V16L; Q30E or Q30T; I41V; V43I; D80N; N81A; S99E; G119S; I137K; L150V; S156G; Y184I; F193S or F193N; L199T or L199Q; F212Y; I225T; I234T; I236G; R240E; F242T or F242S; M252Q; S258K; L276A; E278K; N279K; L283D, L283N, L283E, or L283T; Y284K; Y285A; F289Y; S294K or S249D; K298E; I302T; Q310D; L321K, L321G, or L321N; S323D; F330Y; L335D, L335E, or L335N; V354S or V354A; K358N; L360Q, L360I, or L360K; K363Q; T364S; N367G; F368Q or F368D; A371G; F373K; V381D or V381E; I385V; Y386K, Y386S, or Y386H; T413D or T413E; K416S; F418G, F418K, or F418E; L421V; F422V; I433T; I434K; T435N; T438D; or a combination thereof.

In certain embodiments, the mutation comprises V16R or V16L; Q30E or Q30T; N81A; S99E; G119S; I137K; L150V; S156G; Y184I; F193S or F193N; F212Y; I225T; I234T; R240E; S258K; L283D, L283N, L283E, or L283T; Y284K; I302T; Q310D; L321K, L321G, or L321N; L335D, L335E, or L335N; V354S or V354A; L360Q, L360I, or L360K; A371G; V381D or V381E; Y386K, Y386S, or Y386H; T413D or T413E; F418G, F418K, or F418E; or a combination thereof.

In certain embodiments, the mutation comprises Q30E or Q30T; I41V; V43I; D80N; S99E; F193S or F193N; L199T or L199Q; I236G; F242T or F242S; M252Q; L276A; E278K; N279K; L283D, L283N, L283E, or L283T; Y284K; Y285A; F289Y; S294K or S249D; K298E; Q310D; L321K, L321G, or L321N; S323D; F330Y; L335D, L335E, or L335N; V354S or V354A; K358N; L360Q, L360I, or L360K; K363Q; T364S; N367G; F368Q or F368D; F373K; V381D or V381E; I385V; Y386K, Y386S, or Y386H; T413D or T413E; K416S; F418G, F418K, or F418E; L421V; F422V; I433T; I434K; T435N; T438D; or a combination thereof.

In certain embodiments, the deimmunized BoNT/A-LC or fragment thereof comprises a mutation at one or more of V16, Q30, N81, S99, I137, L150, S156, F212, R240, 5258, L283, Y284, I302, Q310, L335, V354, L360, A371, V381, T413, and F418 of SEQ ID NO: 1.

In certain embodiments, the mutation comprises V16R; Q30E; N81A; S99E; I137K; L150V; S156G; F212Y; R240E; S258K; L283D, L283N, L283E, or L283T; Y284K; I302T; Q310D; L335D, L335E, or L335N; V354S or V354A; L360Q, L360I, or L360K; A371G; V381D or V381E; T413D or T413E; F418G, F418K, or F418E; or a combination thereof.

In certain embodiments, the deimmunized BoNT/A-LC or fragment thereof comprises a mutation at one or more of Q30, I41, S99, F193, L199, I236, F242, L276, E278, N279, L283, Y284, Y285, S294, K298, Q310, L321, S323, F330, L335, V354, L360, K363, T364, N367, F368, V381, I385, Y386, T413, K416, F418, L421, F422, I433, T435, and T438 of SEQ ID NO: 1.

In certain embodiments, the mutation comprises Q30E or Q30T; I41V; S99E; F193S or F193N; L199T or L199Q; I236G; F242T or F242S; L276A; E278K; N279K; L283D, L283N, L283E, or L283T; Y284K; Y285A; S294K or S249D; K298E; Q310D; L321K, L321G, or L321N; S323D; F330Y; L335D, L335E, or L335N; V354S or V354A; L360Q, L360I, or L360K; K363Q; T364S; N367G; F368Q or F368D; V381D or V381E; I385V; Y386K, Y386S, or Y386H; T413D; K416S; F418G or F418E; L421V; F422V; I434K; T435N; T438D; or a combination thereof.

In certain embodiments, the deimmunized BoNT/A-LC or fragment thereof comprises a mutation at one or more of Q30, S99, F193, L199, F242, L276, N279, L283, Y285, L321, S323, L335, V354, L360, V381, I385, Y386, K416, L421, I433, and T438 of SEQ ID NO: 1.

In certain embodiments, the mutation comprises Q30E or Q30T; S99E; F193S or F193N; L199T or L199Q; F242T or F242S; L276A; N279K; L283D, L283N, L283E, or L283T; Y285A; L321K, L321G, or L321N; S323D; L335D, L335E, or L335N; V354S or V354A; L360Q, L360I, or L360K; V381D or V381E; I385V; Y386K, Y386S, or Y386H; K416S; L421V; T438D; or a combination thereof.

In certain embodiments, the deimmunized BoNT/A-LC or fragment thereof comprises a mutation at one or more of Q30, S99, Y184, F212, L283, I302, A371, V381, T413, and F418 of SEQ ID NO: 1. In certain embodiments, the mutation comprises Q30E; S99E; Y184I; F212Y; L283D; I302T; A371G; V381D; T413D; F418G; or a combination thereof, of SEQ ID NO: 1. In certain embodiments, the deimmunized BoNT/A-LC or fragment thereof comprises an amino acid sequence set forth in SEQ ID NO: 8, or an amino acid sequence having at least 90% identity to the amino acid sequence set forth in SEQ ID NO: 8.

In certain embodiments, the deimmunized BoNT/A-LC or fragment thereof comprises a mutation at one or more of V16, Q30, N81, F212, S258, I302, V354, L360, V381, T413, and F418 of SEQ ID NO: 1. In certain embodiments, the mutation comprises V16R; Q30E; N81A; F212Y; S258K; I302T; V354S; L360Q; V381D; T413D; F418G; or a combination thereof, of SEQ ID NO: 1. In certain embodiments, the deimmunized BoNT/A-LC or fragment thereof comprises an amino acid sequence set forth in SEQ ID NO: 9, or an amino acid sequence having at least 90% identity to the amino acid sequence set forth in SEQ ID NO: 9.

In certain embodiments, the deimmunized BoNT/A-LC or fragment thereof comprises a mutation at one or more of Q30, N81, S156, L283, I302, V354, A371, T413, and F418 of SEQ ID NO: 1. In certain embodiments, the mutation comprises Q30E; N81A; S156G; L283D; I302T; V354S; A371G; T413D; F418G; or a combination thereof, of SEQ ID NO: 1. In certain embodiments, the deimmunized BoNT/A-LC or fragment thereof comprises an amino acid sequence set forth in SEQ ID NO: 10, or an amino acid sequence having at least 90% identity to the amino acid sequence set forth in SEQ ID NO: 10.

In certain embodiments, the deimmunized BoNT/A-LC or fragment thereof comprises a mutation at one or more of V16, Q30, S99, L150, L283, Y284, Q310, V354, and T413 of SEQ ID NO: 1. In certain embodiments, the mutation comprises V16R; Q30E; S99E; L150V; L283D; Y284K; Q310D; V354S; T413D; or a combination thereof, of SEQ ID NO: 1. In certain embodiments, the deimmunized BoNT/A-LC or fragment thereof comprises an amino acid sequence set forth in SEQ ID NO: 13, or an amino acid sequence having at least 90% identity to the amino acid sequence set forth in SEQ ID NO: 13.

In certain embodiments, the deimmunized BoNT/A-LC or fragment thereof comprises a mutation at one or more of Q30, N81, S156, S258, L283, I302, V354, and T413 of SEQ ID NO: 1. In certain embodiments, the mutation comprises Q30E; N81A; S156G; S258K; L283D; I302T; V354S; T413D; or a combination thereof, of SEQ ID NO: 1. In certain embodiments, the deimmunized BoNT/A-LC or fragment thereof comprises an amino acid sequence set forth in SEQ ID NO: 15, or an amino acid sequence having at least 90% identity to the amino acid sequence set forth in SEQ ID NO: 15.

In certain embodiments, the deimmunized BoNT/A-LC or fragment thereof comprises a mutation at one or more of Q30, N81, S99, L150, S258, L283, Y284, Q310, V354, A371, T413, and F418 of SEQ ID NO: 1. In certain embodiments, the mutation comprises Q30E; N81A; S99E; L150V; S258K; L283D; Y284K; Q310D; V354S; A371G; T413D; F418G; or a combination thereof, of SEQ ID NO: 1. In certain embodiments, the deimmunized BoNT/A-LC or fragment thereof comprises an amino acid sequence set forth in SEQ ID NO: 18, or an amino acid sequence having at least 90% identity to the amino acid sequence set forth in SEQ ID NO: 18.

In certain embodiments, the deimmunized BoNT/A-LC or fragment thereof comprises a mutation at one or more of V16, Q30, N81, S156, L283, Q310, V354, A371, T413, and F418 of SEQ ID NO: 1. In certain embodiments, the mutation comprises V16R; Q30E; N81A; S156G; L283D; Q310D; V354S; A371G; T413D; F418G; or a combination thereof, of SEQ ID NO: 1. In certain embodiments, the deimmunized BoNT/A-LC or fragment thereof comprises an amino acid sequence set forth in SEQ ID NO: 24, or an amino acid sequence having at least 90% identity to the amino acid sequence set forth in SEQ ID NO: 24.

In certain embodiments, the deimmunized BoNT/A-LC or fragment thereof comprises a mutation at one or more of Q30, N81, I137, L150, F212, L283, Q310, V354, A371, and V381 of SEQ ID NO: 1. In certain embodiments, the mutation comprises Q30E; N81A; I137K; L150V; F212Y; L283D; Q310D; V354S; A371G; V381D; or a combination thereof, of SEQ ID NO: 1. In certain embodiments, the deimmunized BoNT/A-LC or fragment thereof comprises an amino acid sequence set forth in SEQ ID NO: 27, or an amino acid sequence having at least 90% identity to the amino acid sequence set forth in SEQ ID NO: 27.

In certain embodiments, the deimmunized BoNT/A-LC or fragment thereof comprises a mutation at one or more of V16, N81, I137, S156, F212, R240, L283, I302, Q310, L335, V354, L360, A371, and V381 of SEQ ID NO: 1. In certain embodiments, the mutation comprises V16R; N81A; I137K; S156G; F212Y; R240E; L283D; I302T; Q310D; L335N; V354S; L360Q; A371G; V381D; or a combination thereof, of SEQ ID NO: 1. In certain embodiments, the deimmunized BoNT/A-LC or fragment thereof comprises an amino acid sequence set forth in SEQ ID NO: 30, or an amino acid sequence having at least 90% identity to the amino acid sequence set forth in SEQ ID NO: 30. In certain embodiments, the deimmunized BoNT/A-LC or fragment thereof further comprises a F418G mutation.

In certain embodiments, the deimmunized BoNT/A-LC or fragment thereof comprises a mutation at one or more of V16, N81, I137, S156, F212, R240, L283, Q310, V354, A371, T413, and F418 of SEQ ID NO: 1. In certain embodiments, the mutation comprises V16R; N81A; I137K; S156G; F212Y; R240E; L283D; Q310D; V354S; A371G; T413D; F418G; or a combination thereof, of SEQ ID NO: 1. In certain embodiments, the deimmunized BoNT/A-LC or fragment thereof comprises an amino acid sequence set forth in SEQ ID NO: 41, or an amino acid sequence having at least 90% identity to the amino acid sequence set forth in SEQ ID NO: 41.

In certain embodiments, the deimmunized BoNT/A-LC or fragment thereof comprises a mutation at one or more of V16, S99, I137, S156, F212, R240, L283, Q310, V354, and A371 of SEQ ID NO: 1. In certain embodiments, the mutation comprises V16R; S99E; I137K; S156G; F212Y; R240E; L283D; Q310D; V354S; A371G; or a combination thereof, of SEQ ID NO: 1. In certain embodiments, the deimmunized BoNT/A-LC or fragment thereof comprises an amino acid sequence set forth in SEQ ID NO: 42, or an amino acid sequence having at least 90% identity to the amino acid sequence set forth in SEQ ID NO: 42.

In certain embodiments, the deimmunized BoNT/A-LC or fragment thereof comprises a mutation at one or more of Q30, N81, I137, L150, R240, L283, I302, L360, V381, T413, and F418 of SEQ ID NO: 1. In certain embodiments, the mutation comprises Q30E; N81A; I137K; L150V; R240E; L283D; I302T; L360Q; V381D; T413D; F418G; or a combination thereof, of SEQ ID NO: 1. In certain embodiments, the deimmunized BoNT/A-LC or fragment thereof comprises an amino acid sequence set forth in SEQ ID NO: 46, or an amino acid sequence having at least 90% identity to the amino acid sequence set forth in SEQ ID NO: 46.

In certain embodiments, the deimmunized BoNT/A-LC or fragment thereof comprises a mutation at one or more of S99, F193, L283, V354, V381, and I433 of SEQ ID NO: 1. In certain embodiments, the mutation comprises S99E; F193S; L283E; V354A; V381D; I433T; or a combination thereof, of SEQ ID NO: 1. In certain embodiments, the deimmunized BoNT/A-LC or fragment thereof comprises an amino acid sequence set forth in SEQ ID NO: 94, or an amino acid sequence having at least 90% identity to the amino acid sequence set forth in SEQ ID NO: 94.

In certain embodiments, the deimmunized BoNT/A-LC or fragment thereof comprises a mutation at one or more of S99, F193, L283, L335, V354, V381, and I433 of SEQ ID NO: 1. In certain embodiments, the mutation comprises S99E; F193S; L283E; L335D; V354A; V381D; I433T; or a combination thereof, of SEQ ID NO: 1. In certain embodiments, the deimmunized BoNT/A-LC or fragment thereof comprises an amino acid sequence set forth in SEQ ID NO: 95, or an amino acid sequence having at least 90% identity to the amino acid sequence set forth in SEQ ID NO: 95.

In certain embodiments, the deimmunized BoNT/A-LC or fragment thereof comprises a mutation at one or more of Q30, S99, F193, L283, L335, V354, V381, and T438 of SEQ ID NO: 1. In certain embodiments, the mutation comprises Q30E; S99E; F193S; L283E; L335D; V354A; V381D; T438D; or a combination thereof, of SEQ ID NO: 1. In certain embodiments, the deimmunized BoNT/A-LC or fragment thereof comprises an amino acid sequence set forth in SEQ ID NO: 96, or an amino acid sequence having at least 90% identity to the amino acid sequence set forth in SEQ ID NO: 96.

In certain embodiments, the deimmunized BoNT/A-LC or fragment thereof comprises a mutation at one or more of Q30, S99, L283, L335, V354, V381, K416, I433, and T438 of SEQ ID NO: 1. In certain embodiments, the mutation comprises Q30E; S99E; L283E; L335D; V354A; V381D; K416S; I433T; T438D; or a combination thereof, of SEQ ID NO: 1. In certain embodiments, the deimmunized BoNT/A-LC or fragment thereof comprises an amino acid sequence set forth in SEQ ID NO: 97, or an amino acid sequence having at least 90% identity to the amino acid sequence set forth in SEQ ID NO: 97.

In certain embodiments, the deimmunized BoNT/A-LC or fragment thereof comprises a mutation at one or more of S99, L199, N279, L283, L321, S323, L335, V381, K416, and T438 of SEQ ID NO: 1. In certain embodiments, the mutation comprises S99E; L199T; N279K; L283E; L321K; S323D; L335E; V381D; K416S; T438D; or a combination thereof, of SEQ ID NO: 1. In certain embodiments, the deimmunized BoNT/A-LC or fragment thereof comprises an amino acid sequence set forth in SEQ ID NO: 98, or an amino acid sequence having at least 90% identity to the amino acid sequence set forth in SEQ ID NO: 98.

In certain embodiments, the deimmunized BoNT/A-LC or fragment thereof comprises a mutation at one or more of S99, L199, N279, L283, L321, S323, L335, L360, V381, L421, and T438 of SEQ ID NO: 1. In certain embodiments, the mutation comprises S99E; L199T; N279K; L283E; L321K; S323D; L335E; L360Q; V381D; L421V; T438D; or a combination thereof, of SEQ ID NO: 1. In certain embodiments, the deimmunized BoNT/A-LC or fragment thereof comprises an amino acid sequence set forth in SEQ ID NO: 100, or an amino acid sequence having at least 90% identity to the amino acid sequence set forth in SEQ ID NO: 100.

In certain embodiments, the deimmunized BoNT/A-LC or fragment thereof comprises or consists of an amino acid sequence set forth in any one of SEQ ID NOs: 8-109 (i.e., SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109), or comprises an amino acid sequence having at least 90% identity to the amino acid sequence set forth in any one of SEQ ID NO: NOs: 8-109 (i.e., SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109).

In certain embodiments, the deimmunized BoNT/A-LC or fragment thereof comprises 2, 3, 4, 5, 6, 7, 8, 9, 10, 11, 12, 13, 14, 15, 16, 17, 18, 19, 20, or more mutations.

In certain embodiments, the deimmunized BoNT/A-LC or fragment thereof comprises about 0.1% activity or greater relative to a wildtype BoNT/A-LC or fragment thereof.

In certain embodiments, the deimmunized BoNT/A-LC or fragment thereof comprises about 0.1%, about 1%, about 5%, about 10%, about 20%, about 30%, about 40%, about 50%, about 60%, about 70%, about 80%, about 90%, or about 100% activity relative to a wildtype BoNT/A-LC or fragment thereof.

In certain embodiments, the deimmunized BoNT/A-LC or fragment thereof comprises thermostability within about 10° C. of a wildtype BoNT/A-LC or fragment thereof. In certain embodiments, the deimmunized BoNT/A-LC or fragment thereof comprises thermostability within about 3° C. to about 8° C. of a wildtype BoNT/A-LC or fragment thereof. In certain embodiments, the deimmunized BoNT/A-LC or fragment thereof comprises thermostability about equal to a wildtype BoNT/A-LC or fragment thereof.

In certain embodiments, the deimmunized BoNT/A-LC or fragment thereof is fused to a functional moiety.

In certain embodiments, the functional moiety comprises a targeting activity and/or binding activity.

In certain embodiments, the functional moiety is selected from the group consisting of an antigen binding protein or fragment thereof, an imaging molecule, an oligonucleotide, a targeting peptide, and polyethylene glycol (PEG).

In certain embodiments, the antigen binding protein fragment comprises an Fc domain, a Fab domain, an scFv, or a single domain antibody.

In certain embodiments, the deimmunized BoNT/A-LC or fragment thereof further comprises one or both of an L427A mutation and an L428A mutation. In certain embodiments, the deimmunized BoNT/A-LC or fragment thereof further comprises an L427A mutation and an L428A mutation.

In certain embodiments, the deimmunized BoNT/A-LC or fragment thereof further comprises a P1A mutation.

In certain embodiments, the deimmunized BoNT/A-LC or fragment thereof further comprises a A26V mutation.

In certain embodiments, the deimmunized BoNT/A-LC or fragment thereof further comprises an N terminal methionine.

In certain embodiments, the deimmunized BoNT/A-LC or fragment thereof further comprises a botulinum toxin heavy chain (BoNT-HC) or fragment thereof. In certain embodiments, the deimmunized BoNT/A-LC or fragment thereof further comprises a botulinum toxin serotype A heavy chain (BoNT/A-HC) or fragment thereof. In certain embodiments, the BoNT-HC or fragment thereof is a serotype other than serotype A. In certain embodiments, the BoNT-HC serotype is selected from the group consisting of serotype B, serotype C, serotype D, serotype E, serotype F, and serotype G.

In one aspect, the disclosure provides a pharmaceutical composition comprising the deimmunized BoNT/A-LC or fragment thereof recited above and a pharmaceutically acceptable carrier.

In one aspect, the disclosure provides a vector encoding the deimmunized BoNT/A-LC or fragment thereof recited above.

In one aspect, the disclosure provides a host cell comprising the vector recited above. In certain embodiments, the host cell comprises a prokaryotic host cell or a eukaryotic host cell. In certain embodiments, the host cell comprises an E. coli host cell, a Clostridium genus host cell, a yeast host cell, an insect host cell, or a mammalian host cell. In certain embodiments, the clostridium genus host cell comprises C. botulinum, C. baratii, C. butyricum, or C. argentinense.

In one aspect, the disclosure provides a method of treating or preventing a disease or disorder in a subject that would benefit from a therapeutically effective amount of a botulinum toxin, comprising administering a therapeutically effective amount of the deimmunized BoNT/A-LC or fragment thereof recited above to the subject.

In one aspect, the disclosure provides a method of treating or preventing a disease or disorder of inappropriate muscle contraction in a subject, comprising administering a therapeutically effective amount of the deimmunized BoNT/A-LC or fragment thereof recited above to the subject.

In one aspect, the disclosure provides a method of treating or preventing a disease or disorder of inappropriate neuron signaling in a subject, comprising administering a therapeutically effective amount of the deimmunized BoNT/A-LC or fragment thereof recited above to the subject.

In certain embodiments, the disease or disorder is selected from the group consisting of acute pain, alopecia, aquagenic keratoderma, atrial fibrillation, blepharospasm, bromhidrosis, cerebral palsy, cervical dystonia, chromhidrosis, chronic anal fissures, chronic pain, constipation, depression, dermatosis, eccrine nevus, eczema, esophageal spasms, essential tremor, facial erythema and flushing, genodermatoses, Hailey-Hailey disease, hand dystonia, hemifacial spasm, hidradenitis suppurativa, hyperhydrosis, hypersialorrhoea, hypertrophic scars, keloids, linear IgA bullous dermatosis, migraine headache, notalgia paresthetica, oily skin, postherpetic neuralgia, psoriasis, overactive bladder, premature ejaculation, Raynaud's Disease, spastic paresis, strabismus, tension headache, voice abnormalities, whiplash.

In one aspect, the disclosure provides a method of treating a subject for a cosmetic purpose, comprising administering a therapeutically effective amount of the deimmunized BoNT/A-LC or fragment thereof recited above to the subject.

In certain embodiments, the cosmetic purpose is the reduction of facial wrinkles. In certain embodiments, the facial wrinkles comprise brow line wrinkles and glabellar frown lines.

In one aspect, the disclosure provides a method for reducing an antibody response against BoNT/A-LC or fragment thereof in a subject, comprising administering a therapeutically effective amount of the deimmunized BoNT/A-LC or fragment thereof recited above to the subject.

In one aspect, the disclosure provides a method for producing a deimmunized BoNT/A-LC or fragment thereof in a host cell, comprising: a) introducing a vector encoding the deimmunized BoNT/A-LC or fragment thereof recited above, into a host cell to produce a deimmunized BoNT/A-LC-expressing host cell; b) culturing the host cell in a culture system; and c) isolating the deimmunized BoNT/A-LC or fragment thereof from the culture system.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts the PDB crystal structure 3 BTA illustrating different domains in BoNT/A. The light chain (LC) domain is shown in light grey on the left, whereas the heavy chain (HC) domain is shown in dark and light grey on the right.

FIG. 2 depicts the pareto-curve of BoNT/A-LC deimmunized libraries versus the native BoNT/A-LC. Library plan 444 highlighted was chosen for experimental evaluation.

FIG. 3A-FIG. 3F depict Clover-mRuby2 based FRET sensor for detecting the BoNT/A-LC catalytic activity in vitro. FIG. 3A depicts a schematic representation of the FRET sensor. Clover was connected with mRuby2 via SNAP25 (amino acids 146-206). When the sensor is cleaved by BoNT/A-LC at Q¹⁹⁷R¹⁹⁸, Clover is separated from mRuby2, thus eliminating the FRET signal (FIG. 3B). FIG. 3C depicts the FRET signal (emission2, 600 nm) change over time for sensor incubated with truncated BoNT/A-LC (tALC) (lower curve) or truncated inactive BoNT/A-LC (tIALC) (upper curve). FIG. 3D depicts the Clover signal (emission 1, 525 nm) change over time for the sensor incubated with tALC (upper curve) or tIALC (lower curve). FIG. 3E depicts the Clover:mRuby2 emission ratio (Em 525:Em 600) change over time for the sensor incubated with tALC (upper curve), tIALC (lower curve), or no enzyme (black curve). FIG. 3F depicts different concentrations (0 nM, 0.2 nM, 0.4 nM, 0.8 nM, 1.6 nM, 4 nM, 8 nM, 12 nM, and 16 nM) of tALC that were incubated with 333 nM FRET sensor in vitro respectively and the cleavage kinetics were monitored by using a fluorescence plate reader (excitation, 488 nm; emission 1, 525 nm; and emission 2, 600 nm). The rate of Clover:mRuby2 emission ratio (Em 525:Em 600) change per minute for each concentration of tALC was determined, revealing a linear relationship between them.

FIG. 4A-FIG. 4D depict a schematic representation of the in vivo FRET system. FIG. 4A depicts that when E. coli bearing the pALC-sensor is induced, BoNT/A-LC (ALC) and the FRET sensor are expressed in the E. coli cytoplasm. ALC recognizes and cleaves the SNAP-25 linker, resulting in the mRuby2 fragment with an N-terminal arginine. FIG. 4B depicts that R¹⁹⁸-mRuby2 is recognized by the E. coli enzyme Aat, which appends an N-terminal leucine. Subsequently, the N-terminally modified mRuby2 is degraded into short peptides by ClpS-mediated proteolysis by the E. coli ClpAP proteasome. FIG. 4C depicts that after induction, E. coli bearing pALC-sensor appear green as a result of remaining Clover fluorescent proteins in the cytoplasm. FIG. 4D depicts that when E. coli bearing pIALC-sensor is induced, IALC and the FRET sensor are expressed in the E. coli cytoplasm. Since the IALC cannot degrade the FRET sensor, E. coli appear reddish as a result of FRET.

FIG. 5A-FIG. 5E depict the characterization of the in vivo FRET system for the detection and quantitative analysis of ALC catalytic activity. FIG. 5A depicts cells bearing the pALC-sensor (ALC-sensor) appear to be green under ambient light. FIG. 5B depicts cells bearing the pIALC-sensor (IALC-sensor) appeared to be reddish under ambient light. FIG. 5C depicts that after incubation on agar medium containing IPTG inducer, ALC-sensor (left) colonies appear green and IALC-sensor (right) colonies appear reddish under ambient light.

FIG. 5D depicts ALC-sensor and IALC-sensor from 8 different days that were analyzed on a 96-well fluorescence plate reader. Fluorescence intensity ratios were calculated by dividing the fluorescence intensity of Clover (excitation, 488 nm; emission, 525 nm) over the fluorescence intensity of mRuby2 (excitation, 561 nm; emission, 585 nm). Significance was determined by T test using Graphpad Prism, **** p<0.0001. FIG. 5E depicts ALC-sensor (light grey dots) and IALC-sensor (dark grey dots dots) analyzed by flow cytometry, triggering with forward scatter. Each dot represents a single event (cell). X axis: Clover fluorescence intensity (excitation, 488 nm, emission filter, 525/50 nm). Y axis: mRuby2 fluorescence intensity (excitation, 561 nm, emission filter, 585/40 nm).

FIG. 6 depicts a schematic representation of FACS based high-throughput screening of the combinatorial deimmunized ALC library. (1) The deimmunized ALC variants library (ST1250-2) is synthesized, cloned into the pRSF-Duet vector co-expressing the FRET sensor, and transformed into E. coli BL21 (DE3). (2) Cells bearing the library are grown in LB-Kana and induced with 0.1 mM IPTG. (3) Library population was analyzed by FACS. (4) Cells falling into the sorting gate, which is defined to include events with higher clover signal and lower mRuby2 signal, are sorted and re-cultured to repeat the screening process. (5) Sorted cells are plated on indicating agar plates containing 0.1 mM IPTG. To isolate active ALC variants, cell colonies that exhibit green fluorescence under blue LED light are picked and cultured.

FIG. 7A-FIG. 7C depict the isolation of active deimmunized variants through four rounds of FACS sorting. FIG. 7A depicts (1) FACS analysis of the naive ST1250-2 lib3.0 library. Sorting gate was drawn to include cells with higher Clover signal and lower mRuby2 signal. A total of 5.1×10⁸ cells were detected and 4×10⁵ cells were sorted during this round of screening. Sorted cells (designated as lib3.1) were grown and induced for the next round of screening). (2) FACS analysis of lib3.1 library where a total of 9.3×10⁷ cells were detected and 1.2×10⁶ cells were sorted using the same gate as before. Plasmids from the sorted population lib3.2 were isolated and re-transformed into fresh E. coli BL21 (DE3). The new library population (designated as lib3.2-Re) was grown and induced for the next round of screening. (3) FACS analysis of lib3.2-Re library where a total of 1.2×10⁸ cells were detected and 6.8×10⁶ cells were sorted using the same gate as before. Genes encoding ALC variants from the sorted population lib3.3 were amplified, cloned into pRSF-sensor vector, and transformed into E. coli BL21 (DE3). The new library population (designated as lib3.3-PCR) were grown and induced for the next round of screening. (4) FACS analysis of lib3.3-PCR library where 1.9×10⁷ cells were detected and 1.4×10⁶ cells were sorted using the same gate as before. Sorted cells were washed with LB medium twice and then spread on indicating agar plates containing 0.1 mM IPTG. Eight colonies, which exhibited green fluorescence under blue LED light, were isolated. FIG. 7B depicts FACS analysis of lib3.2 library. FIG. 7C depicts FACS analysis of lib3.3-Re library.

FIG. 8A-FIG. 8B depict the initial enzymatic activity analysis of deimmunized ALC variants. FIG. 8A depicts fluorescence intensity ratio Clover:mRuby2 (Ex1, 488 nm; Em1, 525 nm; Ex2, 561 nm; Em2, 585 nm) was used to indicate the enzymatic activity of ALC variants. G2, G3, G4, G5, 1A4, 1A7, 3C11, ALC-sensor, and IALC-sensor were grown in LB-Kan, induced with 0.1 mM IPTG, and measured by fluorescence microplate reader. Experiments were performed in biological triplicates and error bars represent standard deviation from triplicate measurements. FIG. 8B depicts the Clover:FRET fluorescence ratio (Ex=488 nm; Em1=525 nm:Em2=600 nm) was used to determine the enzymatic activity of ALC variants. A selection of 43 full-length ALC variants and WT ALC (positive control) were grown and induced in deep-well microplate. Soluble whole cell lysates were incubated with 67 nM purified FRET sensor molecule for 2 hours at 37° C. and measured by fluorescence microplate reader. Experiments were performed in biological triplicates and error bars represent standard deviation from triplicate measurements.

FIG. 9A-FIG. 9G depict the activities of full-length BoNT/A (FL/A) containing WT or deimmunized light chains on cultured neurons. FIG. 9A depicts purification of WT and deimmunized LCH_{N}-sort protein. Proteins were produced in E. coli BL21 (DE3) using an autoinduction medium. LCH_{N}-sort were purified by Ni-agarose beads. FIG. 9B depicts a schematic model of the production of the full-length BoNT/A (FL/A) using sortase ligation method. The ligation mixture containing LCH_{N}-sort (5 μM), He (40 μM), Sortase (0.5 μM) and CaCl₂) (10 mM) was incubated for 40 min at room temperature. FIG. 9C depicts ligated FL/A toxins were analyzed by SDS-PAGE. FIG. 9D depicts ligated toxins that were activated using thrombin and analyzed by SDS-PAGE, with or without DTT. FIG. 9E depicts cultured rat cortical neurons that were exposed to different concentrations of FL/A toxins for 12 hours at 37° C. Cell lysates were harvested and cleavages of SNAP-25, Syntaxin, and VAMP were assessed by Western blot. The upper band of SNAP-25 represents uncleaved SNAP-25 and the lower band is cleaved SNAP-25. FIG. 9F depicts cultured rat cortical neurons that were exposed to 5 nM of FL/A toxins for 12 hours at 37° C. Cell lysates were harvested and cleavage of SNAP-25 was assessed by Western blot (upper). The upper band of SNAP-25 represents uncleaved SNAP-25 and the lower band is cleaved SNAP-25. The ratio of cleaved SNAP-25 was calculated by ImageJ software (lower). FIG. 9G depicts cultured rat cortical neurons that were exposed to different concentrations of FL/A toxins for 12 hours at 37° C. Cell lysates were harvested and cleavage of SNAP-25 was assessed by Western blot. The upper band of SNAP-25 represents uncleaved SNAP-25 and the lower band is cleaved SNAP-25.

FIG. 10A-FIG. 10F depict full-length BoNT/A containing G4-5 as being able to induce flaccid paralysis in mice with a shorter half-life in vivo. FIG. 10A depicts the catalytic activity of G4-5 variant on cultured cortical neurons. Neurons were exposed to FL/A for 12 h at 37° C. Cell lysates were harvested and cleavage of SNAP-25 was assessed by Western blot.

FIG. 10B depicts the half-life of G4-5 LC in cultured cortical neurons. Neurons were exposed to 50 pM of FL/A for 0.5, 3 or 6 days at 37° C. Cell lysates were harvested and cleavage of SNAP-25 was assessed by Western blot (upper) and the ratio of cleaved SNAP-25 was calculated by ImageJ software (lower). FIG. 10C depicts mouse hind limb muscles injected with ligated FL/A. The injected limb developed typical flaccid paralysis. The paralysis was scored by the spread of toes. FIG. 10D depicts the quantitatively measured DAS scores of FIG. 10C. FIG. 10E depicts that G4-5 variant FL/A is less potent than WT FL/A and has a shorter high-life in vivo in mice. Ligated FL/A (11 pg of WT, 12 ng of G4-5) were injected to mouse right hind limb muscle and paralysis was scored. FIG. 10F depicts the quantitatively measured DAS scores of additional variants.

FIG. 11A-FIG. 11B depict the immunogenicity of BoNT/A light chain variants in humanized HLA transgenic mice. FIG. 11A depicts immunogenicity of BoNT/A light chain variants in DR4 mice encoding the functional variant of human HLA DRB1*0401. DR4 mice were immunized once per a week for 4 weeks with 50 μg of purified light chain variant in PBS. Serum was collected 1 week after the final immunization and anti-drug IgG antibodies were quantified by direct ELISA against the protein immunogen. FIG. 11B depicts immunogenicity of BoNT/A light chain variants in DR2 mice encoding the functional variant of human HLA DRB1*1501. DR2 mice were immunized with 50 μg, 5 μg, or 0.2 μg of purified light chain variant in PBS.

FIG. 12 depicts an electrophoresis gel of sortase ligated full-length BoNT/A optimized light chain variants, with and without 2-mercaptoethanol.

FIG. 13 depicts the results of a SNAP-25 cleavage assay of sortase ligated full-length BoNT/A optimized light chain variants in cultured rat cortical neurons. Neurons were exposed to full-length BoNT/A for 12 h at 37° C. Cell lysates were harvested and cleavage of SNAP-25 was assessed by Western blot.

FIG. 14 depicts the average DAS score of sortase ligated full-length BoNT/A optimized light chain variants.

FIG. 15A-FIG. 15D depict the immunogenicity of BoNT/A light chain variants in DR4 mice encoding the functional variant of human HLA DRB1*0401. DR4 mice were immunized once per a week for 4 weeks with 50 μg of purified light chain variant in PBS. Serum was collected 1 week after the final immunization and anti-drug IgG antibodies were quantified by direct ELISA against the protein immunogen. FIG. 15A depicts immunogenicity for WT and BoNT/A light chain variants #2 (G4-5-F418G) and #8 (G3-15). FIG. 15B depicts immunogenicity for BoNT/A light chain variants #9 (G3-6), #11 (G3-1), #12 (G2), and #13 (3C11). FIG. 15C depicts immunogenicity for BoNT/A light chain variants #4 (G3-9), #6 (G4-22), #7 (G3-4), and #10 (G4-2). FIG. 15D depicts mean anti-BoNT/A antibody titers, quantified as serum dilution for 50% reduction in response—normalized to wild-type, in DR4 mice 6-weeks after receiving a BoNT/A light chain variant relative to WT BoNT/A light chain. Note that the 50% response titer neglects the peak antibody binding signal as a measure of immunogenicity, such that, for example, variant #2 appears to be more immunogenic than variant #8, whereas FIG. 15A clearly shows that variant #2 is less immunogenic than variant #8.

FIG. 16A-FIG. 16B depict the immunogenicity of BoNT/A light chain variants in in DR2 mice encoding the functional variant of human HLA DRB1*1501. Serum was collected 1 week after the final immunization and anti-drug IgG antibodies were quantified by direct ELISA against the protein immunogen. FIG. 16A depicts immunogenicity for BoNT/A light chain variants #2 (G4-5-F418G) and #10 (G4-2). FIG. 16B depicts immunogenicity for BoNT/A light chain variants #5 (G4-21) and #13 (3C11).

FIG. 17A-FIG. 17L depict the immunogenicity of BoNT/A light chain variants in in DR2 mice encoding the functional variant of human HLA DRB1*1501. Serum was collected 1 week after the final immunization and anti-drug IgG antibodies were quantified by direct ELISA against the protein immunogen. FIG. 17A depicts immunogenicity for WT and BoNT/A light chain variant N1. FIG. 17B depicts immunogenicity for WT and BoNT/A light chain variant N2. FIG. 17C depicts immunogenicity for WT and BoNT/A light chain variant N3. FIG. 17D depicts immunogenicity for WT and BoNT/A light chain variant N4. FIG. 17E depicts immunogenicity for WT and BoNT/A light chain variant N5. FIG. 17F depicts immunogenicity for WT and BoNT/A light chain variant N7. FIG. 17G depicts immunogenicity for WT and BoNT/A light chain variant N9. FIG. 17H depicts immunogenicity for WT and BoNT/A light chain variant N11. FIG. 17I depicts immunogenicity for WT and BoNT/A light chain variant N12. FIG. 17J depicts immunogenicity for WT and BoNT/A light chain variant N13. FIG. 17K depicts immunogenicity for WT and BoNT/A light chain variant N14. FIG. 17L depicts immunogenicity for WT and BoNT/A light chain variant N15.

FIG. 18A-FIG. 18B depict the average DAS score of sortase ligated full-length BoNT/A toxin containing optimized light chain variants N1, N3, and N7, and WT BoNT/A. FIG. 18A depicts WT and the variants administered at 100 pg. FIG. 18B depicts WT administered at 75 pg and the variants administered at 150 pg. Toxin was administered once on day 0, and DAS score was tracked as a function of time in order to quantify duration of toxin action.

### DETAILED DESCRIPTION

Immunologically optimized botulinum toxin light chain variants are provided.

Generally, nomenclature used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

### Botulinum Toxin Serotype a Light Chain (BoNT/A-LC) and Deimmunized Variants Thereof

Produced by Clostridium botulinum, botulinum neurotoxins (BoNTs) are generally categorized into seven serotypes (BoNT/A-G), defined by antigenicity, with each serotype further divided into subtypes based on their distinct amino acid sequences (Rossetto et al. Nature reviews. Microbiology. 12, 535-549. 2014). In nature, BoNTs are initially synthesized as an approximately 150 kDa single polypeptide chain that has low intrinsic bioactivity. This precursor protein is subsequently processed by proteases at a flexible loop region to generate the active form, consisting of a 100 kDa heavy chain (HC) and a 50 kDa light chain (LC). Non-covalent interactions and a single inter-chain disulfide bond hold the heavy chain and light chain together (Rossetto, supra). The heavy chain contains two functional domains: the N terminal (Hn) domain is responsible for translocation of the light chain across endosomal membranes into the neuronal cytosol; the C terminal (Hc) domain recognizes and binds to the receptors on the neuronal cell surface (Yao et al. Nature structural & molecular biology. 23, 656-662. 2016). The light chain is a Zn²⁺-dependent metalloprotease that specifically cleaves and inactivates SNARE (soluble N-ethylmaleimide-sensitive factor attachment protein receptor) proteins that are responsible for neurotransmitter release (Singh et al. Neurotoxicity research. 9, 73-92. 2006).

Among all BoNTs, BoNT serotype A (BoNT/A) is the best known due to its cosmetic application to treat brow line wrinkles and glabellar frown lines, which are normally formed by dermal atrophy and repetitive muscle contraction. Local injection of small amounts of BoNT/A into overactive muscles will cause the cleavage of synaptosomal-associated protein 25 (SNAP-25), resulting in the inhibition of SNAP-25-mediated fusion of neurotransmitter-carrying vesicles with the plasma membrane of peripheral neurons. Subsequently, nerve impulses for muscle contraction are temporarily blocked, resulting in muscle relaxation and thus reducing wrinkles (Lorenc et al. Aesthetic surgery journal. 33, 18S-22S. 2013). In the context of serotype A, “Hn/A” refers to the N terminal domain of the serotype A heavy chain and “Hc/A” refers to the C terminal domain of serotype A heavy chain.

As used herein, the term “botulinum neurotoxin serotype A light chain” or “BoNT/A-LC” or “ALC” refers to the wild type amino acid sequence represented by SEQ ID NO: 1, reproduced below. Specific mutation positions in the BoNT/A-LC amino acid sequence are in reference to SEQ ID NO: 1.

(SEQ ID NO: 1)
PFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIY
STDLGRMLLTSIVRGIPFWGGSTIDTELKVIDTNCINVIQPDGSYRSEE
LNLVIIGPSADIIQFECKSFGHEVLNLTRNGYGSTQYIRFSPDFTFGFE
ESLEVDTNPLLGAGKFATDPAVTLAHELIHAGHRLYGIAINPNRVFKVN
TNAYYEMSGLEVSFEELRTFGGHDAKFIDSLQENEFRLYYYNKFKDIAS
TLNKAKSIVGTTASLQYMKNVFKEKYLLSEDTSGKFSVDKLKFDKLYKM
LTEIYTEDNFVKFFKVLNRKTYLNFDKAVFKINIVPKVNYTIYDGFNLR
NTNLAANFNGQNTEINNMNFTKLKNFTGLFEFYKLLCVRGIITSKTKSL
DKGYNK

In certain embodiments, an N-terminal initiator methionine is present in the BoNT/A-LC amino acid sequence. The N-terminal methionine is encoded in the nucleic acids encoding the wild-type and deimmunized ALC variants of the disclosure, however, in some circumstances the N-terminal methionine is removed by the host cell expressing the protein. In certain embodiments, the N-terminal methionine may remain after expression and purification of the deimmunized ALC variants of the disclosure.

In certain embodiments, the amino acid A26 of SEQ ID NO: 1 is replaced with a V (i.e., a A26V substitution). The A26V substitution is a natural variant BoNT/A-LC. Accordingly, any of the BoNT/A-LC deimmunizing mutations recited herein with respect to SEQ ID NO: 1, may also be applied to the natural variant BoNT/A-LC with a A26V substitution.

As used herein, the term “fragment” in reference to BoNT/A-LC (i.e., BoNT/A-LC or fragment thereof) refers to a BoNT/A-LC amino acid sequence that comprises fewer amino acids than the amino acid sequence of SEQ ID NO: 1. For example, but in no way limiting, a fragment of BoNT/A-LC may have one or more amino acids removed from the N terminus, the C terminus, or internally of SEQ ID NO: 1. The fragment may have 1, 2, 3, 4, 5, 6, 7, 8, 9, 10, 11, 12, 13, 14, 15, 16, 17, 18, 19, 20, 25, 30, 35, 40, 45, 50, or more amino acids removed from the N terminus, the C terminus, or internally of SEQ ID NO: 1. The fragment should retain some level of activity of BoNT/A-LC, such as the ability to cleave SNAP-25 and the fragment should contain one or more deimmunizing mutations described herein.

As used herein, the term “truncated BoNT/A-LC” or “tALC” refers to a shortened version of SEQ ID NO: 1 and deimmunized variants thereof, wherein the cysteine at position 429 (C429) is not present. In certain embodiments, truncated BoNT/A-LC comprises amino acids 1-422, amino acids 1-423, amino acids 1-424, amino acids 1-425, or amino acids 1-426 of SEQ ID NO: 1. The C429 residue may be subject to oxidation, leading to gradual dimerization, destabilization, and aggregation of purified ALC. Truncated BoNT/A-LC is described further in Feltrup et al. Scientific reports. 8, 8884. 2018; Gul et al. PloS one. 5, e12872. 2010; Silvaggi et al. Chemistry & Biology. 14, 533-542. 2007; and Roxas-Duncan et al. Antimicrobial agents and chemotherapy. 53, 3478-3486. 2009.

As used herein, the term “deimmunized” when used in reference BoNT/A-LC, relates to BoNT/A-LC (e.g., BoNT/A-LC variants, derivatives and/or homologues thereof), wherein the specific removal and/or modification of highly immunogenic regions or residues has occurred. The term “deimmunized” is well-known in the art and, among other things, has been employed for the removal of T-cell epitopes from other therapeutic molecules including antibodies (See, e.g., WO 98/52976 or WO 00/34317).

Humoral antibody formation requires the cooperation of helper T-cells with antigen-specific B-cells. To reduce immunogenicity of a molecule, one approach is to reduce the ability of the antigen to interact with and stimulate B-cells and/or reduce their ability to stimulate helper T-cells. The identification of B-cell epitopes is problematic, however, given the fact that they are of indeterminate length, and often dependent on the tertiary structure of the target antigen. In contrast, T-cell epitopes are short (9-15 amino acids), linear peptides (See, e.g., Doytchinova & Flower. Mol. Immunol. 43 (13): 2037-44. 2006). In addition, evidence suggests that reduction of T-cell activation is easier to achieve and has the ability to greatly impact antibody production (see, e.g., Tangri et al. J. Immunol. 174:3187-3196. 2005). The amino acid sequences that include the antigenic determinants that stimulate T-cells are referred to as T-cell epitopes and are displayed in the context of major histocompatibility complex (MHC) molecules on antigen presenting cells. Altering the ability of T-cell epitopes to bind MHC molecules (e.g., by inhibiting the binding of the epitope to the MHC molecule, altering the affinity between the epitope and the MHC molecule, altering the epitope in a manner such that the epitope's orientation is altered while within the binding region of the MHC molecule, or altering the epitope in such a way that its presentation by the MHC molecule is altered) has the potential to render the altered epitopes unable to or less able to stimulate an immunogenic response (e.g., stimulate helper T-cells and B cell responses). Accordingly, using the methods described herein, epitopes of BoNT/A-LC were identified and subsequently altered in an effort to reduce the immunogenicity of BoNT/A-LC and its ability to induce humoral antibody responses. Using the same methods described herein, epitopes of BoNT/B-LC, BoNT/C-LC, BoNT/D-LC, BoNT/E-LC, BoNT/F-LC, and BoNT/G-LC were identified in an effort to reduce the immunogenicity of BoNT/B-LC, BoNT/C-LC, BoNT/D-LC, BoNT/E-LC, BoNT/F-LC, and BoNT/G-LC and their ability to induce humoral antibody responses

Thus, deimmunization involves the identification, modification and/or removal of T-cell epitopes, preferably helper T-cell epitopes. In this context, the term T-cell epitope relates to T-cell epitopes (i.e., small peptides) that are recognized by T-cells in the context of MHC class I and/or class II molecules. Methods for the identification of T-cell epitopes are known in the art (see, e.g., WO 98/52976, WO 00/34317, and US 2004/0180386). Various methods of identification include, but are not limited to, peptide threading, peptide-MHC binding, human T-cell assays, analysis of cytokine expression patterns, ELISPOT assays, class II tetramer epitope mapping, search of MHC-binding motif databases and the additional removal/modification of T-cell epitopes.

Identified T-cell epitopes can be eliminated, substituted and/or modified from BoNT/A-LC or fragments thereof by one or more amino acid substitutions within an identified MHC binding peptide as further described herein. In some embodiments, one or more amino acid substitutions are generated that eliminate or greatly reduce binding to MHC class I and/or class II molecules, or alternatively, altering the MHC binding peptide to a sequence that retains its ability to bind MHC class I or class II molecules but fails to trigger T-cell activation and/or proliferation.

Accordingly, the present disclosure provides a variety of BoNT/A-LC variants, including modification (e.g., mutations such as amino acid substitutions) of immunogenic epitopes, which retain activity while concurrently displaying reduced immunogenicity.

The present disclosure is not limited to any particular BoNT/A-LC variant. Indeed, a variety of variants are provided by the present disclosure including, but not limited to, those described in the Examples and Tables 1-3 and 8. In some embodiments, a BoNT/A-LC variant has a single amino acid substitution (e.g., any one of the amino acid substitutions described herein) when compared with the wild type sequence.

In some embodiments, a BoNT/A-LC variant has two amino acid substitutions when compared with the wild type sequence. In other embodiments, a BoNT/A-LC variant has three amino acid substitutions when compared with the wild-type sequence. In further embodiments, a BoNT/A-LC variant has four or more amino acid substitutions when compared with the wild-type sequence.

Similarly, the present invention is not limited to any particular type of mutation. Mutations of this invention include, but not limited to, amino acid exchange(s), insertion (s), deletion(s), addition(s), substitution(s), inversion(s) and/or duplication(s). These mutations/modification(s) also include conservative and/or homologous amino acid exchange(s). Guidance concerning how to make phenotypically/functionally silent amino acid substitution has been described (see, e.g., Bowie. Science. 247:1306-1310. 1990).

The present invention also provides BoNT/A-LC variants having an amino acid sequence that is at least 60%, at least 70%, at least 80%, at least 90%, at least 95%, or at least 99% identical or homologous to the polypeptide sequences shown in Tables 1-3 and 8.

In certain embodiments, the deimmunized botulinum toxin serotype A light chain (BoNT/A-LC) or fragment thereof comprises a mutation at one or more (i.e., one, two, three, four, five, six, seven, eight, nine, ten, eleven, twelve, thirteen, fourteen, fifteen, sixteen, seventeen, eighteen, nineteen, twenty, or more) of V16, Q30, I41, V43, D80, N81, S99, G119, I137, L150, S156, Y184, F193, L199, F212, I225, I234, I236, R240, F242, M252, S258, L276, E278, N279, L283, Y284, Y285, F289, S294, K298, I302, Q310, L321, S323, F330, L335, V354, K358, L360, K363, T364, N367, F368, A371, F373, V381, I385, Y386, T413, K416, F418, L421, F422, I433, I434, T435, or T438 of SEQ ID NO: 1.

In certain embodiments, the deimmunized botulinum toxin serotype A light chain (BoNT/A-LC) or fragment thereof comprises a mutation at one or more (i.e., one, two, three, four, five, six, seven, eight, nine, ten, eleven, twelve, thirteen, fourteen, fifteen, sixteen, seventeen, eighteen, nineteen, twenty, or more) of V16, Q30, N81, S99, G119, I137, L150, S156, Y184, F193, F212, I225, I234, R240, S258, L283, Y284, I302, Q310, L321, L335, V354, L360, A371, V381, Y386, T413, or F418 of SEQ ID NO: 1.

In certain embodiments, the deimmunized botulinum toxin serotype A light chain (BoNT/A-LC) or fragment thereof comprises a mutation at one or more (i.e., one, two, three, four, five, six, seven, eight, nine, ten, eleven, twelve, thirteen, fourteen, fifteen, sixteen, seventeen, eighteen, nineteen, twenty, or more) of Q30, I41, V43, D80, S99, F193, L199, I236, F242, M252, L276, E278, N279, L283, Y284, Y285, F289, S294, K298, Q310, L321, S323, F330, L335, V354, K358, L360, K363, T364, N367, F368, F373, V381, I385, Y386, T413, K416, F418, L421, F422, I433, I434, T435, or T438 of SEQ ID NO: 1.

In certain embodiments, the deimmunized botulinum toxin serotype A light chain (BoNT/A-LC) or fragment thereof comprises a mutation at one or more (i.e., one, two, three, four, five, six, seven, eight, nine, ten, eleven, twelve, thirteen, fourteen, fifteen, sixteen, seventeen, eighteen, nineteen, twenty, or more) of V16, Q30, N81, S99, I137, L150, S156, F212, R240, S258, L283, Y284, I302, Q310, L335, V354, L360, A371, V381, T413, or F418 of SEQ ID NO: 1.

In certain embodiments, the deimmunized botulinum toxin serotype A light chain (BoNT/A-LC) or fragment thereof comprises a mutation at one or more (i.e., one, two, three, four, five, six, seven, eight, nine, ten, eleven, twelve, thirteen, fourteen, fifteen, sixteen, seventeen, eighteen, nineteen, twenty, or more) of Q30, I41, S99, F193, L199, I236, F242, L276, E278, N279, L283, Y284, Y285, S294, K298, Q310, L321, S323, F330, L335, V354, L360, K363, T364, N367, F368, V381, I385, Y386, T413, K416, F418, L421, F422, I433, T435, or T438 of SEQ ID NO: 1.

In certain embodiments, the deimmunized botulinum toxin serotype A light chain (BoNT/A-LC) or fragment thereof comprises a mutation at one or more (i.e., one, two, three, four, five, six, seven, eight, nine, ten, eleven, twelve, thirteen, fourteen, fifteen, sixteen, seventeen, eighteen, nineteen, twenty, or more) of Q30, S99, F193, L199, F242, L276, N279, L283, Y285, L321, S323, L335, V354, L360, V381, I385, Y386, K416, L421, I433, or T438 of SEQ ID NO: 1.

In certain embodiments, the mutation is a substitution of the wild type amino acid for a different amino acid that confers reduced immunogenicity to BoNT/A-LC. In certain embodiments, the mutation comprises V16R or V16L; Q30E or Q30T; I41V; V43I; D80N; N81A; S99E; G119S; I137K; L150V; S156G; Y184I; F193S or F193N; L199T or L199Q; F212Y; I225T; I234T; I236G; R240E; F242T or F242S; M252Q; S258K; L276A; E278K; N279K; L283D, L283N, L283E, or L283T; Y284K; Y285A; F289Y; S294K or S249D; K298E; I302T; Q310D; L321K, L321G, or L321N; S323D; F330Y; L335D, L335E, or L335N; V354S or V354A; K358N; L360Q, L360I, or L360K; K363Q; T364S; N367G; F368Q or F368D; A371G; F373K; V381D or V381E; I385V; Y386K, Y386S, or Y386H; T413D or T413E; K416S; F418G, F418K, or F418E; L421V; F422V; I433T; I434K; T435N; T438D; or a combination thereof.

In certain embodiments, the deimmunized BoNT/A-LC or fragment thereof comprises 1, 2, 3, 4, 5, 6, 7, 8, 9, 10, 11, 12, 13, 14, 15, 16, 17, 18, 19, 20, or more mutations.

In certain embodiments, the deimmunized BoNT/A-LC or fragment thereof may comprise additional mutations that alter the properties of BoNT/A-LC in a manner separate from deimmunization. For example, but in no way limiting, additional mutations may increase or decrease the catalytic activity of BoNT/A-LC, or increase or decrease the in vivo half-life of BoNT/A-LC. In certain embodiments, the deimmunized BoNT/A-LC or fragment thereof may further comprise one or both of an L427A mutation and an L428A mutation. In certain embodiments, the deimmunized BoNT/A-LC or fragment thereof may further comprise an L427A mutation and an L428A mutation.

In certain embodiments, the deimmunized BoNT/A-LC or fragment thereof may further comprise an L427A mutation and an L428A mutation a P1A mutation.

In certain embodiments, the deimmunized BoNT/A-LC or fragment thereof may be associated with a botulinum toxin heavy chain (BoNT-HC) or fragment thereof. The association may be through the non-covalent interactions and the single inter-chain disulfide bond of wild type BoNT/A known in the art (Rossetto, supra). The association may be through an engineered linker, such as a peptide linker or other polymer.

In certain embodiments, the deimmunized BoNT/A-LC may be part of a full-length botulinum toxin (light chain and heavy chain). The full-length botulinum toxin may comprise the deimmunized BoNT/A-LC or fragment thereof of the disclosure and a BoNT-HC of any serotype. In cases where the deimmunized BoNT/A-LC or fragment thereof is paired with a non-serotype A heavy chain, the full-length botulinum toxin is a chimeric full-length botulinum toxin. In certain embodiments, deimmunized BoNT/A-LC or fragment thereof may further comprise a BoNT-HC serotype selected from serotype B, serotype C, serotype D, serotype E, serotype F, and serotype G.

In certain embodiments, the deimmunized BoNT/A-LC or fragment thereof of the disclosure comprises about 0.1% activity or greater relative to a wildtype BoNT/A-LC or fragment thereof. As used herein, the term “activity” in connection with BoNT/A-LC refers to any known activity of BoNT/A-LC, including, but not limited to, the catalytic activity mediating cleavage of synaptosomal-associated protein 25 (SNAP-25). In certain embodiments, the deimmunized BoNT/A-LC or fragment thereof of the disclosure comprises about 0.1%, about 1%, about 5%, about 10%, about 20%, about 30%, about 40%, about 50%, about 60%, about 70%, about 80%, about 90%, or about 100% activity relative to a wildtype BoNT/A-LC or fragment thereof.

In certain embodiments, the deimmunized BoNT/A-LC or fragment thereof of the disclosure is thermostable relative to a wildtype BoNT/A-LC or fragment thereof. In certain embodiments, the deimmunized BoNT/A-LC or fragment thereof comprises thermostability within about 10° C. of a wildtype BoNT/A-LC or fragment thereof. In certain embodiments, the deimmunized BoNT/A-LC or fragment thereof thermostability within about 3° C. to about 8° C. of a wildtype BoNT/A-LC or fragment thereof. In certain embodiments, the deimmunized BoNT/A-LC or fragment thereof thermostability about equal to a wildtype BoNT/A-LC or fragment thereof.

Protein stability can be determined using several different methods. Three well-established methods for measuring thermostability include, e.g., differential scanning calorimetry (DSC), differential scanning light scattering (DSLS), and differential scanning fluorimetry (DSF). All methods are based on determining the rate of protein unfolding with increasing temperature, which is a measure of protein stability. For instance, if a small increase in temperature results in protein unfolding, the protein is not considered to be stable. DSC directly measures the heat absorption associated with thermal denaturation and has been shown to be sufficiently quantitative for evaluation of stability of protein therapeutics (Wen et al. J. Pharmaceut. Sci. 101:955-964. 2011). The DSLS method measures protein stability based on the assumption that proteins denature irreversibly as they are exposed to increasing temperatures. Using light-scattering, this method monitors the aggregation that occurs as a consequence of denaturation. In DSF, a fluorescent dye is used that fluoresces upon binding hydrophobic residues. As temperature increases, the protein starts to unfold and exposes the hydrophobic residues found in its core, causing an increase in the fluorescent signal. This increase in signal is monitored over a range of temperatures and is used to determine the Tm value.

In certain embodiments, the deimmunized BoNT/A-LC or fragment thereof of the present disclosure elicits less than 90%, less than 80%, less than 70%, less than 60%, less than 50%, less than 40%, less than 30%, less than 20%, or less than 10% of the immune response (e.g., as measured by anti-BoNT/A-LC antibody titers) elicited by non-deimmunized BoNT/A-LC, such as wild type BoNT/A-LC.

In certain embodiments, the deimmunized BoNT/A-LC or fragment thereof of the present disclosure may be fused to one or more functional moieties. As used herein, the term “functional moiety” refers to a non-BoNT/A-LC moiety that confers an additional function to the fusion molecule. The functional moiety may include a peptide, polypeptide, carbohydrate, lipid, or nucleic acid. The functional moiety may comprise a targeting activity, such as a cell or tissue specific targeting activity. The functional moiety may comprise a binding activity, such as an antibody or a non-antibody based binding protein. The functional moiety may facilitate purification of the deimmunized BoNT/A-LC or fragment thereof. Non-limiting examples of functional moieties include binding proteins such as antigen binding proteins or fragments thereof, imaging molecules such as fluorescent dyes and fluorescent proteins, oligonucleotides such as aptamers, siRNAs, antisense oligonucleotides, miRNAs or mRNAs, anthrax toxin protective antigen (PA) or fragments thereof, a targeting peptide, and polyethylene glycol (PEG).

In certain embodiments, the antigen binding protein comprises a traditional Y-shaped antibody, a multispecific antibody such as a bispecific or trispecific antibody, a nanobody, a VHH or heavy chain-only antibody, a diabody. In certain embodiments, the antigen binding protein fragment comprises an Fc domain, a Fab domain, an scFv, or a single domain antibody.

In certain embodiments, the non-antibody based binding protein comprises an affibody, an afflin, affimer, alphabody, lipocalin, avimer, ankyrin repeat motif, fynomer, kunitz domain, and fibronectin and derivatives thereof.

In some embodiments, the present invention provides a plasmid harboring a nucleic acid sequence encoding a deimmunized BoNT/A-LC or fragment thereof. In certain embodiments, the plasmid is an expression vector harboring a nucleic acid sequence encoding a BoNT/A-LC variant (e.g., that displays BoNT/A-LC activity and reduced immunogenicity). The nucleic acid sequence encoding a BoNT/A-LC variant may further comprise a start codon encoding the N-terminal initiator methionine. As described above, this N-terminal initiator methionine may be removed by the host cell expressing the protein.

In some embodiments, the BoNT/A-LC variant is expressed as a fusion protein, e.g., fused to sequences that facilitate purification (e.g., histidine stretches). In some embodiments, an expression vector of the present invention harbors a nucleic acid sequence encoding a deimmunized BoNT/A-LC variant having an amino acid sequence as set forth in Tables 1-3 and 8.

In addition to BoNT/A-LC variant nucleic acids, a plasmid of this invention may also include regulatory sequences, e.g., promoters, transcriptional enhancers and/or sequences that allow for induced expression of lysostaphin variants. For example, one suitable inducible system is a tetracycline-regulated gene expression system (see, e.g., Gossen & Bujard (1992) Proc. Natl. Acad. Sci. USA 89:5547-5551; Gossen et al. (1994) Trends Biotech. 12:58-62). In some embodiments, the inducible system is an isopropyl-b-D-thiogalactoside (IPTG)-inducible promoter.

In certain embodiments, the deimmunized BoNT/A-LC or fragment thereof of the disclosure is expressed in a host cell. In certain embodiments the host cell is a prokaryotic host cell or a eukaryotic host cell. In certain embodiments, the host cell is an E. coli host cell, a Clostridium genus host cell, a yeast host cell, an insect host cell, or a mammalian host cell. In certain embodiments, the clostridium genus host cell comprises C. botulinum, C. baratii, C. butyricum, or C. argentinense.

In one aspect, the disclosure provides a method for producing a deimmunized BoNT/A-LC or fragment thereof in a host cell, comprising: a) introducing a vector encoding the deimmunized BoNT/A-LC or fragment thereof of the disclosure, into a host cell to produce a deimmunized BoNT/A-LC-expressing host cell; b) culturing the host cell in a culture system; and c) isolating the deimmunized BoNT/A-LC or fragment thereof from the culture system. Methods of expressing and purifying proteins, including BoNT, are known in the art. For example, but in no way limiting, an expression vector encoding deimmunized BoNT/A-LC or fragment thereof of the disclosure may be transformed into an E. coli expression host cell, such as a BL21 host strain. Expression of deimmunized BoNT/A-LC may then be induced with the chemical inducer IPTG for a period of time to obtain sufficient amounts of the protein. Following expression of deimmunized BoNT/A-LC, the host cell may be lysed and deimmunized BoNT/A-LC may be purified from host cell contaminants using standard chromatography techniques, including histidine tag affinity chromatography.

The BoNT/A-LC variants of the disclosure are provided below in Tables 1-3. The amino acid sequences of Table 1 and Table 2 comprise a P1A substitution, which was made to facilitate cloning into a screening vector. The library-based BoNT/A-LC variants were analyzed for enzymatic activity based on the P1A substitution. However, when these variants were integrated into full length toxins for neuron assays and mouse testing, the alanine was reverted back to the native proline.

TABLE 1
Amino Acid Sequences of Deimmunized BoNT/A-LC Variants:
Library Design

| Sequence | | |
|---|---|---|
| ID | Sequence | Mutations |
| G1 | AFVNKQFNYKDPVNGVDIAYIKIPNAGQMEPVKA | P1AA \| Q30E \| S99E \| |
| (SEQ ID | FKIHNKIWVIPERDTFTNPEEGDLNPPPEAKQVPVS | Y184I \| F212Y \| |
| NO: 8) | YYDSTYLSTDNEKDNYLKGVTKLFERIYETDLGR | L283D \| I302T \| |
| | MLLTSIVRGIPFWGGSTIDTELKVIDTNCINVIQPDG | A371G \| V381D \| |
| | SYRSEELNLVIIGPSADIIQFECKSFGHEVLNLTRNG | T413D \| F418G \| |
| | YGSTQIIRFSPDFTFGFEESLEVDTNPLLGAGKYAT | |
| | DPAVTLAHELIHAGHRLYGIAINPNRVFKVNTNAY | |
| | YEMSGLEVSFEELRTFGGHDAKFIDSLQENEFRDY | |
| | YYNKFKDIASTLNKAKSTVGTTASLQYMKNVFKE | |
| | KYLLSEDTSGKFSVDKLKFDKLYKMLTEIYTEDNF | |
| | VKFFKVLNRKTYLNFDKGVFKINIVPKDNYTIYDG | |
| | FNLRNTNLAANFNGQNTEINNMNFDKLKNGTGLF | |
| | EFYKLLCVRGIITSKTKSLDKGYNK | |
| G2 | AFVNKQFNYKDPVNGRDIAYIKIPNAGQMEPVKA | P1A \| V16R \| Q30E \| |
| (SEQ ID | FKIHNKIWVIPERDTFTNPEEGDLNPPPEAKQVPVS | N81A \| F212Y \| |
| NO: 9) | YYDSTYLSTDAEKDNYLKGVTKLFERIYSTDLGR | S258K \| I302T \| |
| | MLLTSIVRGIPFWGGSTIDTELKVIDTNCINVIQPDG | V354S \| L360Q \| |
| | SYRSEELNLVIIGPSADIIQFECKSFGHEVLNLTRNG | V381D \| T413D \| |
| | YGSTQYIRF SPDFTFGFEESLEVDTNPLLGAGKYAT | F418G |
| | DPAVTLAHELIHAGHRLYGIAINPNRVFKVNTNAY | |
| | YEMSGLEVKFEELRTFGGHDAKFIDSLQENEFRLY | |
| | YYNKFKDIASTLNKAKSTVGTTASLQYMKNVFKE | |
| | KYLLSEDTSGKFSVDKLKFDKLYKMLTEIYTEDNF | |
| | SKFFKVQNRKTYLNFDKAVFKINIVPKDNYTIYDG | |
| | FNLRNTNLAANFNGQNTEINNMNFDKLKNGTGLF | |
| | EFYKLLCVRGIITSKTKSLDKGYNK | |
| G3-1 | AFVNKQFNYKDPVNGVDIAYIKIPNAGQMEPVKA | P1A \| Q3OE \| N81A \| |
| (SEQ ID | FKIHNKIWVIPERDTFTNPEEGDLNPPPEAKQVPVS | S156G \| L283D \| |
| NO: 10) | YYDSTYLSTDAEKDNYLKGVTKLFERIYSTDLGR | I302T \| V3545 \| |
| | MLLTSIVRGIPFWGGSTIDTELKVIDTNCINVIQPDG | A371G \| T413D \| |
| | SYRSEELNLVIIGPGADIIQFECKSFGHEVLNLTRNG | F418G |
| | YGSTQYIRFSPDFTFGFEESLEVDTNPLLGAGKFAT | |
| | DPAVTLAHELIHAGHRLYGIAINPNRVFKVNTNAY | |
| | YEMSGLEVSFEELRTFGGHDAKFIDSLQENEFRDY | |
| | YYNKFKDIASTLNKAKSTVGTTASLQYMKNVFKE | |
| | KYLLSEDTSGKFSVDKLKFDKLYKMLTEIYTEDNF | |
| | SKFFKVLNRKTYLNFDKGVFKINIVPKVNYTIYDG | |
| | FNLRNTNLAANFNGQNTEINNMNFDKLKNGTGLF | |
| | EFYKLLCVRGIITSKTKSLDKGYNK | |
| G3-2 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 11) | YYDSTYLSTDAEKDNYLKGVTKLFERIYSTDLGR | |
| | MLLTSIVRGIPFWGGSTIDTELKVIDTNCINVIQPDG | |
| | SYRSEELNVVIIGPGADIIQFECKSFGHEVLNLTRN | |
| | GYGSTQYIRFSPDFTFGFEESLEVDTNPLLGAGKYA | |
| | TDPAVTLAHELIHAGHRLYGTAINPNEVFKVNTNA | |
| | YYEMSGLEVKFEELRTFGGHDAKFIDSLQENEFRD | |
| | YYYNKFKDIASTLNKAKSTVGTTASLQYMKNVFK | |
| | EKYLLSEDTSGKFSVDKNKFDKLYKMLTEIYTEDN | |
| | FSKFFKVLNRKTYLNFDKAVFKINIVPKVNYTIYD | |
| | GFNLRNTNLAANFNGQNTEINNMNFDKLKNFTGL | |
| | FEFYKLLCVRGIITSKTKSLDKGYNK | |
| G3-3 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 12) | YYDSTYLSTDAEKDNYLKGVTKLFERIYSTDLGR | |
| | MLLTSIVRGIPFWGGSTIDTELKVIDTNCINVIQPDG | |
| | SYRSEELNLVIIGPGADIIQFECKSFGHEVLNLTRNG | |
| | YGSTQYIRFSPDFTFGFEESLEVDTNPLLGAGKYAT | |
| | DPAVTLAHELIHAGHRLYGTAINPNEVFKVNTNAY | |
| | YEMSGLEVKFEELRTFGGHDAKFIDSLQENEFRDY | |
| | YYNKFKDIASTLNKAKSTVGTTASLQYMKNVFKE | |
| | KYLLSEDTSGKFSVDKNKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| G3-4 | AFVNKQFNYKDPVNGRDIAYIKIPNAGQMEPVKA | P1A \| V16R \| Q30E \| |
| (SEQ ID | FKIHNKIWVIPERDTFTNPEEGDLNPPPEAKQVPVS | S99E \| L150V \| |
| NO: 13) | YYDSTYLSTDNEKDNYLKGVTKLFERIYETDLGR | L283D Y284K |
| | MLLTSIVRGIPFWGGSTIDTELKVIDTNCINVIQPDG | Q310D \| V354S \| |
| | SYRSEELNVVIIGPSADIIQFECKSFGHEVLNLTRNG | T413D |
| | YGSTQYIRFSPDFTFGFEESLEVDTNPLLGAGKFAT | |
| | DPAVTLAHELIHAGHRLYGIAINPNRVFKVNTNAY | |
| | YEMSGLEVSFEELRTFGGHDAKFIDSLQENEFRDK | |
| | YYNKFKDIASTLNKAKSIVGTTASLDYMKNVFKE | |
| | KYLLSEDTSGKFSVDKLKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| G3-5 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 14) | YYDSTYLSTDAEKDNYLKGVTKLFERIYSTDLGR | |
| | MLLTSIVRGIPFWGGSTIDTELKVIDTNCINVIQPDG | |
| | SYRSEELNLVIIGPGADIIQFECKSFGHEVLNLTRNG | |
| | YGSTQYIRFSPDFTFGFEESLEVDTNPLLGAGKYAT | |
| | DPAVTLAHELIHAGHRLYGTAINPNEVFKVNTNAY | |
| | YEMSGLEVKFEELRTFGGHDAKFIDSLQENEFRDY | |
| | YYNKFKDIASTLNKAKSTVGTTASLQYMKNVFKE | |
| | KYLLSEDTSGKFSVDKNKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| G3-6 | AFVNKQFNYKDPVNGVDIAYIKIPNAGQMEPVKA | P1A \| Q30E \| N81A \| |
| (SEQ ID | FKIHNKIWVIPERDTFTNPEEGDLNPPPEAKQVPVS | S156G \| S258K \| |
| NO: 15) | YYDSTYLSTDAEKDNYLKGVTKLFERIYSTDLGR | L283D \| I302T \| |
| | MLLTSIVRGIPFWGGSTIDTELKVIDTNCINVIQPDG | V354S \| T413D |
| | SYRSEELNLVIIGPGADIIQFECKSFGHEVLNLTRNG | |
| | YGSTQYIRFSPDFTFGFEESLEVDTNPLLGAGKFAT | |
| | DPAVTLAHELIHAGHRLYGIAINPNRVFKVNTNAY | |
| | YEMSGLEVKFEELRTFGGHDAKFIDSLQENEFRDY | |
| | YYNKFKDIASTLNKAKSTVGTTASLQYMKNVFKE | |
| | KYLLSEDTSGKFSVDKLKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| G3-7 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 16) | YYDSTYLSTDNEKDNYLKGVTKLFERIYETDLGR | |
| | MLLTSIVRGIPFWGGSTIDTELKVIDTNCINVKQPD | |
| | GSYRSEELNLVIIGPGADIIQFECKSFGHEVLNLTRN | |
| | GYGSTQYIRFSPDFTFGFEESLEVDTNPLLGAGKYA | |
| | TDPAVTLAHELTHAGHRLYGIAINPNEVFKVNTNA | |
| | YYEMSGLEVSFEELRTFGGHDAKFIDSLQENEFRD | |
| | YYYNKFKDIASTLNKAKSIVGTTASLDYMKNVFK | |
| | EKYLLSEDTSGKFSVDKLKFDKLYKMLTEIYTEDN | |
| | FSKFFKVLNRKTYLNFDKGVFKINIVPKVNYTIYD | |
| | GFNLRNTNLAANFNGQNTEINNMNFDKLKNGTGL | |
| | FEFYKLLCVRGIITSKTKSLDKGYNK | |
| G3-8 | AFVNKQFNYKDPVNGRDIAYIKIPNAGQMEPVKA | |
| (SEQ ID | FKIHNKIWVIPERDTFTNPEEGDLNPPPEAKQVPVS | |
| NO: 17) | YYDSTYLSTDNEKDNYLKGVTKLFERIYETDLGR | |
| | MLLTSIVRGIPFWGGSTIDTELKVIDTNCINVIQPDG | |
| | SYRSEELNVVIIGPSADIIQFECKSFGHEVLNLTRNG | |
| | YGSTQYIRFSPDFTFGFEESLEVDTNPLLGAGKYAT | |
| | DPAVTLAHELIHAGHRLYGTAINPNEVFKVNTNAY | |
| | YEMSGLEVKFEELRTFGGHDAKFIDSLQENEFRDY | |
| | YYNKFKDIASTLNKAKSTVGTTASLQYMKNVFKE | |
| | KYLLSEDTSGKFSVDKNKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| G3-9 | AFVNKQFNYKDPVNGVDIAYIKIPNAGQMEPVKA | P1A \| Q30E \| N81A \| |
| (SEQ ID | FKIHNKIWVIPERDTFTNPEEGDLNPPPEAKQVPVS | S99E \| L150V \| |
| NO: 18) | YYDSTYLSTDAEKDNYLKGVTKLFERIYETDLGR | S258K \| L283D \| |
| | MLLTSIVRGIPFWGGSTIDTELKVIDTNCINVIQPDG | Y284K \| Q310D \| |
| | SYRSEELNVVIIGPSADIIQFECKSFGHEVLNLTRNG | V355S \| A371G \| |
| | YGSTQYIRFSPDFTFGFEESLEVDTNPLLGAGKFAT | T413D \| F418G \| |
| | DPAVTLAHELIHAGHRLYGIAINPNRVFKVNTNAY | |
| | YEMSGLEVSFEELRTFGGHDAKFIDSLQENEFRDK | |
| | YYNKFKDIASTLNKAKSIVGTTASLDYMKNVFKE | |
| | KYLLSEDTSGKFSVDKLKFDKLYKMLTEIYTEDNF | |
| | SKFFKVLNRKTYLNFDKGVFKINIVPKVNYTIYDG | |
| | FNLRNTNLAANFNGQNTEINNMNFDKLKNGTGLF | |
| | EFYKLLCVRGIITSKTKSLDKGYNK | |
| G3-10 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 19) | YYDSTYLSTDAEKDNYLKGVTKLFERIYSTDLGR | |
| | MLLTSIVRGIPFWGGSTIDTELKVIDTNCINVIQPDG | |
| | SYRSEELNLVIIGPGADIIQFECKSFGHEVLNLTRNG | |
| | YGSTQYIRFSPDFTFGFEESLEVDTNPLLGAGKYAT | |
| | DPAVTLAHELIHAGHRLYGTAINPNEVFKVNTNAY | |
| | YEMSGLEVKFEELRTFGGHDAKFIDSLQENEFRDK | |
| | YYNKFKDIASTLNKAKSTVGTTASLQYMKNVFKE | |
| | KYLLSEDTSGKFSVDKNKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| G3-11 | AFVNKQFNYKDPVNGRDIAYIKIPNAGQMEPVKA | |
| (SEQ ID | FKIHNKIWVIPERDTFTNPEEGDLNPPPEAKQVPVS | |
| NO: 20) | YYDSTYLSTDAEKDNYLKGVTKLFERIYSTDLGR | |
| | MLLTSIVRGIPFWGGSTIDTELKVIDTNCINVIQPDG | |
| | SYRSEELNLVIIGPGADIIQFECKSFGHEVLNLTRNG | |
| | YGSTQYIRFSPDFTFGFEESLEVDTNPLLGAGKYAT | |
| | DPAVTLAHELIHAGHRLYGTAINPNEVFKVNTNAY | |
| | YEMSGLEVKFEELRTFGGHDAKFIDSLQENEFRDY | |
| | YYNKFKDIASTLNKAKSTVGTTASLQYMKNVFKE | |
| | KYLLSEDTSGKFSVDKNKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| G3-12 | AFVNKQFNYKDPVNGRDIAYIKIPNAGQMEPVKA | |
| (SEQ ID | FKIHNKIWVIPERDTFTNPEEGDLNPPPEAKQVPVS | |
| NO: 21) | YYDSTYLSTDAEKDNYLKGVTKLFERIYSTDLGR | |
| | MLLTSIVRGIPFWGGSTIDTELKVIDTNCINVIQPDG | |
| | SYRSEELNLVIIGPGADIIQFECKSFGHEVLNLTRNG | |
| | YGSTQYIRFSPDFTFGFEESLEVDTNPLLGAGKYAT | |
| | DPAVTLAHELIHAGHRLYGTAINPNEVFKVNTNAY | |
| | YEMSGLEVKFEELRTFGGHDAKFIDSLQENEFRDY | |
| | YYNKFKDIASTLNKAKSTVGTTASLQYMKNVFKE | |
| | KYLLSEDTSGKFSVDKNKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| G3-13 | AFVNKQFNYKDPVNGRDIAYIKIPNAGQMEPVKA | |
| (SEQ ID | FKIHNKIWVIPERDTFTNPEEGDLNPPPEAKQVPVS | |
| NO: 22) | YYDSTYLSTDAEKDNYLKGVTKLFERIYSTDLGR | |
| | MLLTSIVRGIPFWGGSTIDTELKVIDTNCINVIQPDG | |
| | SYRSEELNLVIIGPGADIIQFECKSFGHEVLNLTRNG | |
| | YGSTQYIRFSPDFTFGFEESLEVDTNPLLGAGKYAT | |
| | DPAVTLAHELIHAGHRLYGTAINPNEVFKVNTNAY | |
| | YEMSGLEVKFEELRTFGGHDAKFIDSLQENEFRDY | |
| | YYNKFKDIASTLNKAKSTVGTTASLQYMKNVFKE | |
| | KYLLSEDTSGKFSVDKNKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| G3-14 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 23) | YYDSTYLSTDNEKDNYLKGVTKLFERIYETDLGR | |
| | MLLTSIVRGIPFWGGSTIDTELKVIDTNCINVIQPDG | |
| | SYRSEELNLVIIGPGADIIQFECKSFGHEVLNLTRNG | |
| | YGSTQYIRFSPDFTFGFEESLEVDTNPLLGAGKYAT | |
| | DPAVTLAHELIHAGHRLYGTAINPNEVFKVNTNAY | |
| | YEMSGLEVKFEELRTFGGHDAKFIDSLQENEFRDY | |
| | YYNKFKDIASTLNKAKSTVGTTASLQYMKNVFKE | |
| | KYLLSEDTSGKFSVDKNKFDKLYKMLTEIYTEDNF | |
| | SKFFKVLNRKTYLNFDKAVFKINIVPKVNYTIYDG | |
| | FNLRNTNLAANFNGQNTEINNMNFDKLKNGTGLF | |
| | EFYKLLCVRGIITSKTKSLDKGYNK | |
| G3-15 | AFVNKQFNYKDPVNGRDIAYIKIPNAGQMEPVKA | P1A \| V16R \| Q30E \| |
| (SEQ ID | FKIHNKIWVIPERDTFTNPEEGDLNPPPEAKQVPVS | N81A \| S156G \| |
| NO: 24) | YYDSTYLSTDAEKDNYLKGVTKLFERIYSTDLGR | L283D \| Q310D \| |
| | MLLTSIVRGIPFWGGSTIDTELKVIDTNCINVIQPDG | V354S \| A371G \| |
| | SYRSEELNLVIIGPGADIIQFECKSFGHEVLNLTRNG | T413D \| F418G |
| | YGSTQYIRFSPDFTFGFEESLEVDTNPLLGAGKFAT | |
| | DPAVTLAHELIHAGHRLYGIAINPNRVFKVNTNAY | |
| | YEMSGLEVSFEELRTFGGHDAKFIDSLQENEFRDY | |
| | YYNKFKDIASTLNKAKSIVGTTASLDYMKNVFKE | |
| | KYLLSEDTSGKFSVDKLKFDKLYKMLTEIYTEDNF | |
| | SKFFKVLNRKTYLNFDKGVFKINIVPKVNYTIYDG | |
| | FNLRNTNLAANFNGQNTEINNMNFDKLKNGTGLF | |
| | EFYKLLCVRGIITSKTKSLDKGYNK | |
| G3-16 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 25) | YYDSTYLSTDAEKDNYLKGVTKLFERIYSTDLGR | |
| | MLLTSIVRGIPFWGGSTIDTELKVIDTNCINVIQPDG | |
| | SYRSEELNLVIIGPSADIIQFECKSFGHEVLNLTRNG | |
| | YGSTQYIRFSPDFTFGFEESLEVDTNPLLGAGKFAT | |
| | DPAVTLAHELIHAGHRLYGTAINPNEVFKVNTNAY | |
| | YEMSGLEVSFEELRTFGGHDAKFIDSLQENEFRDK | |
| | YYNKFKDIASTLNKAKSIVGTTASLDYMKNVFKE | |
| | KYLLSEDTSGKFSVDKLKFDKLYKMLTEIYTEDNF | |
| | SKFFKVLNRKTYLNFDKGVFKINIVPKVNYTIYDG | |
| | FNLRNTNLAANFNGQNTEINNMNFDKLKNFTGLF | |
| | EFYKLLCVRGIITSKTKSLDKGYNK | |
| G4-1 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 26) | YYDSTYLSTDNEKDNYLKGVTKLFERIYSTDLGR | |
| | MLLTSIVRGIPFWGGSTIDTELKVIDTNCINVKQPD | |
| | GSYRSEELNLVIIGPGADIIQFECKSFGHEVLNLTRN | |
| | GYGSTQYIRFSPDFTFGFEESLEVDTNPLLGAGKYA | |
| | TDPAVTLAHELIHAGHRLYGIAINPNEVFKVNTNA | |
| | YYEMSGLEVKFEELRTFGGHDAKFIDSLQENEFRD | |
| | YYYNKFKDIASTLNKAKSTVGTTASLDYMKNVFK | |
| | EKYLLSEDTSGKFSVDKLKFDKLYKMLTEIYTEDN | |
| | FSKFFKVQNRKTYLNFDKGVFKINIVPKVNYTIYD | |
| | GFNLRNTNLAANFNGQNTEINNMNFTKLKNGTGL | |
| | FEFYKLLCVRGIITSKTKSLDKGYNK | |
| G4-2 | AFVNKQFNYKDPVNGVDIAYIKIPNAGQMEPVKA | P1A \| Q30E \| N81A \| |
| (SEQ ID | FKIHNKIWVIPERDTFTNPEEGDLNPPPEAKQVPVS | I137K \| L150V \| |
| NO: 27) | YYDSTYLSTDAEKDNYLKGVTKLFERIYSTDLGR | F212Y \| L283D \| |
| | MLLTSIVRGIPFWGGSTIDTELKVIDTNCINVKQPD | Q310D \| V3545 \| |
| | GSYRSEELNVVIIGPSADIIQFECKSFGHEVLNLTRN | A371G \| V381D |
| | GYGSTQYIRFSPDFTFGFEESLEVDTNPLLGAGKYA | |
| | TDPAVTLAHELIHAGHRLYGIAINPNRVFKVNTNA | |
| | YYEMSGLEVSFEELRTFGGHDAKFIDSLQENEFRD | |
| | YYYNKFKDIASTLNKAKSIVGTTASLDYMKNVFK | |
| | EKYLLSEDTSGKFSVDKLKFDKLYKMLTEIYTEDN | |
| | FSKFFKVLNRKTYLNFDKGVFKINIVPKDNYTIYD | |
| | GFNLRNTNLAANFNGQNTEINNMNFTKLKNFTGL | |
| | FEFYKLLCVRGIITSKTKSLDKGYNK | |
| G4-3 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 28) | YYDSTYLSTDAEKDNYLKGVTKLFERIYETDLGR | |
| | MLLTSIVRGIPFWGGSTIDTELKVIDTNCINVKQPD | |
| | GSYRSEELNVVIIGPSADIIQFECKSFGHEVLNLTRN | |
| | GYGSTQYIRFSPDFTFGFEESLEVDTNPLLGAGKYA | |
| | TDPAVTLAHELIHAGHRLYGIAINPNRVFKVNTNA | |
| | YYEMSGLEVKFEELRTFGGHDAKFIDSLQENEFRD | |
| | YYYNKFKDIASTLNKAKSTVGTTASLQYMKNVFK | |
| | EKYKLSEDTSGKFSVDKNKFDKLYKMLTEIYTED | |
| | NFSKFFKVLNRKTYLNFDKGVFKINIVPKVNYTIY | |
| | DGFNLRNTNLAANFNGQNTEINNMNFDKLKNGTG | |
| | LFEFYKLLCVRGIITSKTKSLDKGYNK | |
| G4-4 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 29) | YYDSTYLSTDAEKDNYLKGVTKLFERIYETDLGR | |
| | MLLTSIVRGIPFWGGSTIDTELKVIDTNCINVKQPD | |
| | GSYRSEELNLVIIGPGADIIQFECKSFGHEVLNLTRN | |
| | GYGSTQYIRFSPDFTFGFEESLEVDTNPLLGAGKYA | |
| | TDPAVTLAHELTHAGHRLYGIAINPNEVFKVNTNA | |
| | YYEMSGLEVSFEELRTFGGHDAKFIDSLQENEFRD | |
| | YYYNKFKDIASTLNKAKSIVGTTASLDYMKNVFK | |
| | EKYLLSEDTSGKFSVDKLKFDKLYKMLTEIYTEDN | |
| | FSKFFKVQNRKTYLNFDKGVFKINIVPKVNYTIYD | |
| | GFNLRNTNLAANFNGQNTEINNMNFTKLKNFTGL | |
| | FEFYKLLCVRGIITSKTKSLDKGYNK | |
| G4-5 | AFVNKQFNYKDPVNGRDIAYIKIPNAGQMQPVKA | P1A \| V16R \| N81A \| |
| (SEQ ID | FKIHNKIWVIPERDTFTNPEEGDLNPPPEAKQVPVS | I137K \| S156G \| |
| NO: 30) | YYDSTYLSTDAEKDNYLKGVTKLFERIYSTDLGR | F212Y \| R240E \| |
| | MLLTSIVRGIPFWGGSTIDTELKVIDTNCINVKQPD | L283D \| I302T \| |
| | GSYRSEELNLVIIGPGADIIQFECKSFGHEVLNLTRN | Q310D \| L33SN \| |
| | GYGSTQYIRFSPDFTFGFEESLEVDTNPLLGAGKYA | V354S \| L360Q \| |
| | TDPAVTLAHELIHAGHRLYGIAINPNEVFKVNTNA | A371G \| V381D |
| | YYEMSGLEVSFEELRTFGGHDAKFIDSLQENEFRD | |
| | YYYNKFKDIASTLNKAKSTVGTTASLDYMKNVFK | |
| | EKYLLSEDTSGKFSVDKNKFDKLYKMLTEIYTEDN | |
| | FSKFFKVQNRKTYLNFDKGVFKINIVPKDNYTIYD | |
| | GFNLRNTNLAANFNGQNTEINNMNFTKLKNFTGL | |
| | FEFYKLLCVRGIITSKTKSLDKGYNK | |
| G4-6 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 31) | YYDSTYLSTDAEKDNYLKGVTKLFERIYSTDLGR | |
| | MLLTSIVRGIPFWGGSTIDTELKVIDTNCINVKQPD | |
| | GSYRSEELNLVIIGPGADIIQFECKSFGHEVLNLTRN | |
| | GYGSTQYIRFSPDFTFGFEESLEVDTNPLLGAGKYA | |
| | TDPAVTLAHELIHAGHRLYGIAINPNRVFKVNTNA | |
| | YYEMSGLEVKFEELRTFGGHDAKFIDSLQENEFRD | |
| | YYYNKFKDIASTLNKAKSTVGTTASLQYMKNVFK | |
| | EKYLLSEDTSGKFSVDKLKFDKLYKMLTEIYTEDN | |
| | FSKFFKVLNRKTYLNFDKGVFKINIVPKVNYTIYD | |
| | GFNLRNTNLAANFNGQNTEINNMNFTKLKNGTGL | |
| | FEFYKLLCVRGIITSKTKSLDKGYNK | |
| G4-7 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 32) | YYDSTYLSTDNEKDNYLKGVTKLFERIYETDLGR | |
| | MLLTSIVRGIPFWGGSTIDTELKVIDTNCINVKQPD | |
| | GSYRSEELNVVIIGPSADIIQFECKSFGHEVLNLTRN | |
| | GYGSTQYIRFSPDFTFGFEESLEVDTNPLLGAGKYA | |
| | TDPAVTLAHELTHAGHRLYGIAINPNRVFKVNTNA | |
| | YYEMSGLEVKFEELRTFGGHDAKFIDSLQENEFRD | |
| | YYYNKFKDIASTLNKAKSTVGTTASLQYMKNVFK | |
| | EKYLLSEDTSGKFSVDKLKFDKLYKMLTEIYTEDN | |
| | FSKFFKVLNRKTYLNFDKGVFKINIVPKVNYTIYD | |
| | GFNLRNTNLAANFNGQNTEINNMNFDKLKNFTGL | |
| | FEFYKLLCVRGIITSKTKSLDKGYNK | |
| G4-8 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 33) | YYDSTYLSTDAEKDNYLKGVTKLFERIYETDLGR | |
| | MLLTSIVRGIPFWGGSTIDTELKVIDTNCINVKQPD | |
| | GSYRSEELNVVIIGPSADIIQFECKSFGHEVLNLTRN | |
| | GYGSTQYIRFSPDFTFGFEESLEVDTNPLLGAGKYA | |
| | TDPAVTLAHELIHAGHRLYGIAINPNEVFKVNTNA | |
| | YYEMSGLEVSFEELRTFGGHDAKFIDSLQENEFRD | |
| | YYYNKFKDIASTLNKAKSTVGTTASLDYMKNVFK | |
| | EKYLLSEDTSGKFSVDKNKFDKLYKMLTEIYTEDN | |
| | FSKFFKVQNRKTYLNFDKGVFKINIVPKVNYTIYD | |
| | GFNLRNTNLAANFNGQNTEINNMNFTKLKNGTGL | |
| | FEFYKLLCVRGIITSKTKSLDKGYNK | |
| G4-9 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 34) | YYDSTYLSTDNEKDNYLKGVTKLFERIYETDLGR | |
| | MLLTSIVRGIPFWGGSTIDTELKVIDTNCINVKQPD | |
| | GSYRSEELNVVIIGPSADIIQFECKSFGHEVLNLTRN | |
| | GYGSTQYIRFSPDFTFGFEESLEVDTNPLLGAGKYA | |
| | TDPAVTLAHELTHAGHRLYGIAINPNRVFKVNTNA | |
| | YYEMSGLEVKFEELRTFGGHDAKFIDSLQENEFRD | |
| | YYYNKFKDIASTLNKAKSTVGTTASLDYMKNVFK | |
| | EKYLLSEDTSGKFSVDKLKFDKLYKMLTEIYTEDN | |
| | FSKFFKVLNRKTYLNFDKGVFKINIVPKDNYTIYD | |
| | GFNLRNTNLAANFNGQNTEINNMNFTKLKNFTGL | |
| | FEFYKLLCVRGIITSKTKSLDKGYNK | |
| G4-10 | AFVNKQFNYKDPVNGVDIAYIKIPNAGQMQPVKA | |
| (SEQ ID | FKIHNKIWVIPERDTFTNPEEGDLNPPPEAKQVPVS | |
| NO: 35) | YYDSTYLSTDNEKDNYLKGVTKLFERIYETDLGR | |
| | MLLTSIVRGIPFWGGSTIDTELKVIDTNCINVKQPD | |
| | GSYRSEELNLVIIGPGADIIQFECKSFGHEVLNLTRN | |
| | GYGSTQYIRFSPDFTFGFEESLEVDTNPLLGAGKYA | |
| | TDPAVTLAHELTHAGHRLYGIAINPNEVFKVNTNA | |
| | YYEMSGLEVSFEELRTFGGHDAKFIDSLQENEFRD | |
| | YYYNKFKDIASTLNKAKSIVGTTASLDYMKNVFK | |
| | EKYLLSEDTSGKFSVDKLKFDKLYKMLTEIYTEDN | |
| | FSKFFKVLNRKTYLNFDKGVFKINIVPKVNYTIYD | |
| | GFNLRNTNLAANFNGQNTEINNMNFDKLKNGTGL | |
| | FEFYKLLCVRGIITSKTKSLDKGYNK | |
| G4-13 | AFVNKQFNYKDPVNGLDIAYIKIPNAGQMQPVKA | |
| (SEQ ID | FKIHNKIWVIPERDTFTNPEEGDLNPPPEAKQVPVS | |
| NO: 36) | YYDSTYLSTDAEKDNYLKGVTKLFERIYSTDLGR | |
| | MLLTSIVRGIPFWGGSTIDTELKVIDTNCINVKQPD | |
| | GSYRSEELNLVIIGPGADIIQFECKSFGHEVLNLTRN | |
| | GYGSTQYIRFSPDFTFGFEESLEVDTNPLLGAGKYA | |
| | TDPAVTLAHELIHAGHRLYGIAINPNEVFKVNTNA | |
| | YYEMSGLEVSFEELRTFGGHDAKFIDSLQENEFRD | |
| | YYYNKFKDIASTLNKAKSTVGTTASLDYMKNVFK | |
| | EKYLLSEDTSGKFSVDKNKFDKLYKMLTEIYTEDN | |
| | FSKFFKVQNRKTYLNFDKGVFKINIVPKDNYTIYD | |
| | GFNLRNTNLAANFNGQNTEINNMNFTKLKNFTGL | |
| | FEFYKLLCVRGIITSKTKSLDKGYNK | |
| G4-16 | AFVNKQFNYKDPVNGVDIAYIKIPNAGQMQPVKA | |
| (SEQ ID | FKIHNKIWVIPERDTFTNPEEGDLNPPPEAKQVPVS | |
| NO: 37) | YYDSTYLSTDNEKDNYLKGVTKLFERIYETDLGR | |
| | MLLTSIVRGIPFWGGSTIDTELKVIDTNCINVKQPD | |
| | GSYRSEELNLVIIGPGADIIQFECKSFGHEVLNLTRN | |
| | GYGSTQYIRFSPDFTFGFEESLEVDTNPLLGAGKYA | |
| | TDPAVTLAHELTHAGHRLYGIAINPNEVFKVNTNA | |
| | YYEMSGLEVSFEELRTFGGHDAKFIDSLQENEFRD | |
| | YYYNKFKDIASTLNKAKSIVGTTASLDYMKNVFK | |
| | EKYLLSEDTSGKFSVDKLKFDKLYKMLTEIYTEDN | |
| | FSKFFKVLNRKTYLNFDKGVFKINIVPKVNYTIYD | |
| | GFNLRNTNLAANFNGQNTEINNMNFDKLKNGTGL | |
| | FEFYKLLCVRGIITSKTKSLDKGYNK | |
| G4-17 | AFVNKQFNYKDPVNGVDIAYIKIPNAGQMQPVKA | |
| (SEQ ID | FKIHNKIWVIPERDTFTNPEEGDLNPPPEAKQVPVS | |
| NO: 38) | YYDSTYLSTDNEKDNYLKGVTKLFERIYETDLGR | |
| | MLLTSIVRGIPFWGGSTIDTELKVIDTNCINVKQPD | |
| | GSYRSEELNLVIIGPGADIIQFECKSFGHEVLNLTRN | |
| | GYGSTQYIRFSPDFTFGFEESLEVDTNPLLGAGKYA | |
| | TDPAVTLAHELTHAGHRLYGIAINPNEVFKVNTNA | |
| | YYEMSGLEVSFEELRTFGGHDAKFIDSLQENEFRD | |
| | YYYNKFKDIASTLNKAKSTVGTTASLDYMKNVFK | |
| | EKYLLSEDTSGKFSVDKLKFDKLYKMLTEIYTEDN | |
| | FSKFFKVLNRKTYLNFDKGVFKINIVPKVNYTIYD | |
| | GFNLRNTNLAANFNGQNTEINNMNFDKLKNGTGL | |
| | FEFYKLLCVRGIITSKTKSLDKGYNK | |
| G4-19 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 39) | YYDSTYLSTDAEKDNYLKGVTKLFERIYSTDLGR | |
| | MLLTSIVRGIPFWGGSTIDTELKVIDTNCINVKQPD | |
| | GSYRSEELNVVIIGPSADIIQFECKSFGHEVLNLTRN | |
| | GYGSTQYIRFSPDFTFGFEESLEVDTNPLLGAGKYA | |
| | TDPAVTLAHELIHAGHRLYGIAINPNRVFKVNTNA | |
| | YYEMSGLEVKFEELRTFGGHDAKFIDSLQENEFRD | |
| | YYYNKFKDIASTLNKAKSIVGTTASLDYMKNVFK | |
| | EKYLLSEDTSGKFSVDKLKFDKLYKMLTEIYTEDN | |
| | FSKFFKVLNRKTYLNFDKGVFKINIVPKVNYTIYD | |
| | GFNLRNTNLAANFNGQNTEINNMNFDKLKNGTGL | |
| | FEFYKLLCVRGIITSKTKSLDKGYNK | |
| G4-20 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 40) | YYDSTYLSTDAEKDNYLKGVTKLFERIYSTDLGR | |
| | MLLTSIVRGIPFWGGSTIDTELKVIDTNCINVKQPD | |
| | GSYRSEELNLVIIGPSADIIQFECKSFGHEVLNLTRN | |
| | GYGSTQYIRFSPDFTNGFEESLEVDTNPLLGAGKY | |
| | ATDPAVTLAHELTHAGHRLYGIAINPNEVFKVNTN | |
| | AYYEMSGLEVSFEELRTFGGHDAKFIDSLQENEFR | |
| | DYYYNKFKDIASTLNKAKSIVGTTASLDYMKNVF | |
| | KEKYLLSEDTSGKFSVDKLKFDKLYKMLTEIYTED | |
| | NFSKFFKVLNRKTYLNFDKGVFKINIVPKVNYTIY | |
| | DGFNLRNTNLAANFNGQNTEINNMNFDKLKNGTG | |
| | LFEFYKLLCVRGIITSKTKSLDKGYNK | |
| G4-21 | AFVNKQFNYKDPVNGRDIAYIKIPNAGQMQPVKA | P1A \| V16R \| N81A \| |
| (SEQ ID | FKIHNKIWVIPERDTFTNPEEGDLNPPPEAKQVPVS | I137K \| S156G \| |
| NO: 41) | YYDSTYLSTDAEKDNYLKGVTKLFERIYSTDLGR | F212Y \| R240E \| |
| | MLLTSIVRGIPFWGGSTIDTELKVIDTNCINVKQPD | L283D \| Q310D \| |
| | GSYRSEELNLVIIGPGADIIQFECKSFGHEVLNLTRN | V354S \| A371G \| |
| | GYGSTQYIRFSPDFTFGFEESLEVDTNPLLGAGKYA | T413D \| F418G |
| | TDPAVTLAHELIHAGHRLYGIAINPNEVFKVNTNA | |
| | YYEMSGLEVSFEELRTFGGHDAKFIDSLQENEFRD | |
| | YYYNKFKDIASTLNKAKSIVGTTASLDYMKNVFK | |
| | EKYLLSEDTSGKFSVDKLKFDKLYKMLTEIYTEDN | |
| | FSKFFKVLNRKTYLNFDKGVFKINIVPKVNYTIYD | |
| | GFNLRNTNLAANFNGQNTEINNMNFDKLKNGTGL | |
| | FEFYKLLCVRGIITSKTKSLDKGYNK | |
| G4-22 | AFVNKQFNYKDPVNGRDIAYIKIPNAGQMQPVKA | P1A \| V16R \| S99E \| |
| (SEQ ID | FKIHNKIWVIPERDTFTNPEEGDLNPPPEAKQVPVS | I137K \| S156G \| |
| NO: 42) | YYDSTYLSTDNEKDNYLKGVTKLFERIYETDLGR | F212Y \| R240E \| |
| | MLLTSIVRGIPFWGGSTIDTELKVIDTNCINVKQPD | L283D \| Q310D \| |
| | GSYRSEELNLVIIGPGADIIQFECKSFGHEVLNLTRN | V354S \| A371G |
| | GYGSTQYIRFSPDFTFGFEESLEVDTNPLLGAGKYA | |
| | TDPAVTLAHELIHAGHRLYGIAINPNEVFKVNTNA | |
| | YYEMSGLEVSFEELRTFGGHDAKFIDSLQENEFRD | |
| | YYYNKFKDIASTLNKAKSIVGTTASLDYMKNVFK | |
| | EKYLLSEDTSGKFSVDKLKFDKLYKMLTEIYTEDN | |
| | FSKFFKVLNRKTYLNFDKGVFKINIVPKVNYTIYD | |
| | GFNLRNTNLAANFNGQNTEINNMNFTKLKNFTGL | |
| | FEFYKLLCVRGIITSKTKSLDKGYNK | |
| G4-23 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 43) | YYDSTYLSTDAEKDNYLKGVTKLFERIYETDLGR | |
| | MLLTSIVRGIPFWGGSTIDTELKVIDTNCINVKQPD | |
| | GSYRSEELNVVIIGPSADIIQFECKSFGHEVLNLTRN | |
| | GYGSTQYIRFSPDFTFGFEESLEVDTNPLLGAGKYA | |
| | TDPAVTLAHELIHAGHRLYGIAINPNRVFKVNTNA | |
| | YYEMSGLEVKFEELRTFGGHDAKFIDSLQENEFRD | |
| | YYYNKFKDIASTLNKAKSTVGTTASLQYMKNVFK | |
| | EKYKLSEDTSGKFSVDKNKFDKLYKMLTEIYTED | |
| | NFSKFFKVLNRKTYLNFDKGVFKINIVPKVNYTIY | |
| | DGFNLRNTNLAANFNGQNTEINNMNFDKLKNGTG | |
| | LFEFYKLLCVRGIITSKTKSLDKGYNK | |
| G4-24 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 44) | YYDSTYLSTDAEKDNYLKGVTKLFERIYSTDLGR | |
| | MLLTSIVRGIPFWGGSTIDTELKVIDTNCINVKQPD | |
| | GSYRSEELNLVIIGPGADIIQFECKSFGHEVLNLTRN | |
| | GYGSTQYIRFSPDFTFGFEESLEVDTNPLLGAGKYA | |
| | TDPAVTLAHELIHAGHRLYGIAINPNEVFKVNTNA | |
| | YYEMSGLEVSFEELRTFGGHDAKFIDSLQENEFRD | |
| | YYYNKFKDIASTLNKAKSTVGTTASLDYMKNVFK | |
| | EKYLLSEDTSGKFSVDKNKFDKLYKMLTEIYTEDN | |
| | FSKFFKVQNRKTYLNFDKGVFKINIVPKDNYTIYD | |
| | GFNLRNTNLAANFNGQNTEINNMNFTKLKNFTGL | |
| | FEFYKLLCVRGIITSKTKSLDKGYNK | |
| G5 | AFVNKQFNYKDPVNGVDIAYIKIPNAGQMEPVKA | |
| (SEQ ID | FKIHNKIWVIPERDTFTNPEEGDLNPPPEAKQVPVS | |
| NO: 45) | YYDSTYLSTDAEKDNYLKGVTKLFERIYSTDLGR | |
| | MLLT SIVRGIPFWGGSTIDTELKVIDTNCINVIQPDG | |
| | SYRSEELNLVIIGPGADIIQFECKSFGHEVLNLTRNG | |
| | YGSTQYIRFSPDFTFGFEESLEVDTNPLLGAGKYAT | |
| | DPAVTLAHELIHAGHRLYGTAINPNEVFKVNTNAY | |
| | YEMSGLEVKFEELRTFGGHDAKFIDSLQENEFRDY | |
| | YYNKFKDIASTLNKAKSTVGTTASLQYMKNVFKE | |
| | KYLLSEDTSGKFSVDKNKFDKLYKMLTEIYTEDNF | |
| | SKFFKVLNRKTYLNFDKAVFKINIVPKVNYTIYDG | |
| | FNLRNTNLAANFNGQNTEINNMNFDKLKNFTGLF | |
| | EFYKLLCVRGIITSKTKSLDKGYNK | |
| 3C11 | AFVNKQFNYKDPVNGVDIAYIKIPNAGQMEPVKA | P1A \| Q3OE \| N81A \| |
| (SEQ ID | FKIHNKIWVIPERDTFTNPEEGDLNPPPEAKQVPVS | I137K \| L150V \| |
| NO: 46) | YYDSTYLSTDAEKDNYLKGVTKLFERIYSTDLGR | R240E \| L283D \| |
| | MLLTSIVRGIPFWGGSTIDTELKVIDTNCINVIQPDG | I302T \| L360Q \| |
| | SYRSEELNVVIIGPSADIIQFECKSFGHEVLNLTRNG | V381D \| T413D \| |
| | YGSTQYIRFSPDFTFGFEESLEVDTNPLLGAGKFAT | F418G |
| | DPAVTLAHELIHAGHRLYGIAINPNEVFKVNTNAY | |
| | YEMSGLEVSFEELRTFGGHDAKFIDSLQENEFRDY | |
| | YYNKFKDIASTLNKAKSTVGTTASLQYMKNVFKE | |
| | KYLLSEDTSGKFSVDKLKFDKLYKMLTEIYTEDNF | |
| | VKFFKVQNRKTYLNFDKAVFKINIVPKDNYTIYDG | |
| | FNLRNTNLAANFNGQNTEINNMNFDKLKNGTGLF | |
| | EFYKLLCVRGIITSKTKSLDKGYNK | |
| 1A4 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 47) | YYDSTYLSTDNEKDNYLKGVTKLFERIYSTDLGR | |
| | MLLTSIVRGIPFWGSSTIDTELKVIDTNCINVKQPD | |
| | GSYRSEELNVVIIGPSADIIQFECKSFGHEVLNLTRN | |
| | GYGSTQIIRFSPDFTFGFEESLEVDTNPLLGAGKYA | |
| | TDPAVTLAHELIHAGHRLYGIAINPNRVFKVNTNA | |
| | YYEMSGLEVKFEELRTFGGHDAKFIDSLQENEFRD | |
| | KYYNKFKDIASTLNKAKSIVGTTASLDYMKNVFK | |
| | EKYKLSEDTSGKFSVDKNKFDKLYKMLTEIYTED | |
| | NFVKFFKVQNRKTYLNFDKAVFKINIVPKVNYTIK | |
| | DGFNLRNTNLAANFNGQNTEINNMNFDKLKNGTG | |
| | LFEFYKLLCVRGIITSKTKSLDKGYNK | |
| 1A7 | AFVNKQFNYKDPVNGRDIAYIKIPNAGQMQPVKA | |
| (SEQ ID | FKIHNKIWVIPERDTFTNPEEGDLNPPPEAKQVPVS | |
| NO: 48) | YYDSTYLSTDNEKDNYLKGVTKLFERIYSTDLGR | |
| | MLLTSIVRGIPFWGGSTIDTELKVIDTNCINVKQPD | |
| | GSYRSEELNVVIIGPSADIIQFECKSFGHEVLNLTRN | |
| | GYGSTQIIRFSPDFTFGFEESLEVDTNPLLGAGKYA | |
| | TDPAVTLAHELIHAGHRLYGIAINPNRVFKVNTNA | |
| | YYEMSGLEVKFEELRTFGGHDAKFIDSLQENEFRD | |
| | KYYNKFKDIASTLNKAKSIVGTTASLDYMKNVFK | |
| | EKYKLSEDTSGKFSVDKNKFDKLYKMLTEIYTED | |
| | NFVKFFKVQNRKTYLNFDKAVFKINIVPKVNYTIK | |
| | DGFNLRNTNLAANFNGQNTEINNMNFDKLKNGTG | |
| | LFEFYKLLCVRGIITSKTKSLDKGYNK | |

The mutation positions and specific mutations of the deimmunized BoNT/A-LC variants of Table 1 are only recited for select variants, as shown in the fight “Mutations” column. None-the-less, the mutation positions and specific mutations of all of the deimmunized BoNT/A-LC variants of Table 1 may be readily determined by aligning each sequence with the WT BoNT/A-LC sequence of SEQ ID NO: 1.

TABLE 2
Amino Acid Sequences of Deimmunized BoNT/A-LC Variants:
Additional Variants From Library Design

| Sequence ID | Sequence |
|---|---|
| S012252-1 | AFVNKQFNYKDPVNGVDIAYIKIPNAGQMQPVKAFKIHNKIW |
| (SEQ ID NO: 49) | VIPERDTFTNPEEGDLNPPPEAKQVPVSYYDSTYLSTDNEKDN |
| | YLKGVTKLFERIYSTDLGRMLLTSIVRGIPFWGGSTIDTELKVID |
| | TNCINVIQPDGSYRSEELNLVIIGPSADIIQFECKSFGHEVLNLTR |
| | NGYGSTQYIRFSPDFTFGFEESLEVDTNPLLGAGKFATDPAVTL |
| | AHQLIHAGHRLYGIAINPNRVFKVNTNAYYEMSGLEVSFEELR |
| | TFGGHDAKFIDSLQENEFRLYYYNKFKDIASTLNKAKSIVGTT |
| | ASLQYMKNVFKEKYLLSEDTSGKFSVDKLKFDKLYKMLTEIY |
| | TEDNFVKFFKVLNRKTFLNFDKAVFKINIVPKVNYTIYDGFNL |
| | RNTNLAANFNGQNTEINNMNFTKLKNFTGLFEFYKLLCVRGII |
| | TSKTKSLDKGYNK |
| S012252-2 | AFVNKQFNYKDPVNGVDIAYIKIPNAGQMEPVKAFKIHNKIW |
| (SEQ ID NO: 50) | VIPERDTFTNPEEGDLNPPPEAKQVPVSYYDSTYLSTDNEKDN |
| | YLKGVTKLFERIYETDLGRMLLTSIVRGIPFWGGSTIDTELKVI |
| | DTNCINVKQPDGSYRSEELNVVIIGPGADIIQFECKSFGHEVLNL |
| | TRNGYGSTQYIRFSPDFTFGFEESLKVDTNPLLGAGKFATDPAV |
| | TLAHELIHAGHRLYGIAINPNEVFKVNTNAYYEMSGLEVSFEE |
| | LRTFGGHDAKFIDSLQENEFRDYYYNKFKDIASTLNKAKSTVG |
| | TTASLQYMKNVFKEKYLLSEDTSGKFSVDKNKFDKLYKMLTE |
| | IYTEDNFSKFFKVLNRKTYLNFDKAVFKINIVPKDNYTIYDGFN |
| | LCNTNLAANFNGQNTEINNMNFDKLKNFTGLFEFYKLLCVRGI |
| | ITSKTKSLDKGYNK |
| S012252-3 | AFVNKQFNYKDPVNGVDIAYIKIPNAGQMQPVKAFKIHNKIW |
| (SEQ ID NO: 51) | VIPERDTFTNPEEGDLNPPPEAKQVPVSYYDSTYLSTDNEKDN |
| | YLKGVTKLFERIYSTDLGRMLLTSIVRGIPFWGGSTIDTELKVID |
| | TNCINVKQPDGSYRSEELNLVIIGPGADIIQFECKSFGHEVLNLT |
| | RNGYGSTQYIRFSPDFTFGFEESLEVDTNPLLGAGKYATDPAV |
| | TLAHELTHAGHRLYGIAINPNRVFKVNTNAYYEMSGLEVSFEE |
| | LRTFGGHDAKFIDSLQENEFRDKYYNKFKDIASTLNKAKSTVG |
| | TTASLQYMKNVFKEKYKLSEDTSGKFSVDKNKFDKLYKMLTE |
| | IYTEDNFSKFFKVLNRKTYLNFDKGVFKINIVPKVNYTIKDGFN |
| | LRNTNLAANFNGQNTEINNMNFDKLKNGTGLFEFYKLLCVRG |
| | IITSKTKSLDKGYNK |
| S012252-4 | AFVNKQFNYKDPVNGRDIAYIKIPNAGQMQPVKAFKIHNKIW |
| (SEQ ID NO: 52) | VIPERDTFTNPEEGDLNPPPEAKQVPVSYYDSTYLSTDAEKDN |
| | YLKGVTKLFERIYETDLGRMLLTSIVRGIPFWGGSTIDTELKVI |
| | DTNCINVIQPDGSYRSEELNLVIIGPGADIIQFECKSFGHEVLNL |
| | TRNGYGSTQYIRFSPDFTFGFEESLEVDTNPLLGAGKYATDPA |
| | VTLAHELTHAGHRLYGIAINPNRVFKVNTNAYYEMSGLEVKF |
| | EELRTFGGHDAKFIDSLQENEFRLYYYNKFKDIASTLNKAKST |
| | VGTTASLQYMKNVFKEKYKLSEDTSGKFSVDKNKFDKLYKM |
| | LTEIYTEDNFVKFFKVLNRKTYLNFDKAVFKINIVPKDNYTIYD |
| | GFNLRNTNLAANFNGQNTEINNMNFDKLKNFTGLFEFYKLLC |
| | VRGIITSKTKSLDKGYNK |
| S012252-5 | AFVNKQFNYKDPVNGRDIAYIKIPNAGQMQPVKAFKIHNKIW |
| (SEQ ID NO: 53) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNVVIIGPSADIIQFECKSFGHEVLNLT |
| | RNGYGSTQYIRFSPDFTNGFEESLEVDTNPLLGAGKYATDPAV |
| | TLAHELIHAGHRLYGIAINPNRVFKVNTNAYYEMSGLEVSFEE |
| | LRTFGGHDAKFIDSLQENEFRDSYYNKFKDIASTLNKAKSTVG |
| | TTASLDYMKNVFKEKYLLSEDTSGKFSVDKLKFDKLYKMLTE |
| | IYTEDNFVKFFKVQNRKTYLNFDKAVFKINIVPKVNYTIYDGF |
| | NLRNTNLAANFNGQNTEINNMNFTKLKNFTGLFEFYKLLCVR |
| | GIITSKTKSLDKGYNK |
| S012252-6 | AFVNKQFNYKDPVNGRDIAYIKIPNAGQMQPVKAFKIHNKIW |
| (SEQ ID NO: 54) | VIPERDTFTNPEEGDLNPPPEAKQVPVSYYDSTYLSTDNEKDN |
| | YLKGVTKLFERIYETDLGRMLLTSIVRGIPFWGGSTIDTELKVI |
| | DTNCINVKQPDGSYRSEELNVVIIGPGADIIQFECKSFGHEVLNL |
| | TRNGYGSTQYIRFSPDFTFGFEESLEVDTNPLLGAGKYATDPA |
| | VTLAHELIHAGHRLYGIAINPNEVFKVNTNAYYEMSGLEVSFE |
| | ELRTFGGHDAKFIDSLQENEFRDYYYNKFKDIASTLNKAKSTV |
| | GTTASLDYMKNVFKEKYLLSEDTSGKFSVDKLKFDKLYKMLT |
| | EIYTEDNFSKFFKVQNRKTYLNFDKGVFKINIVPKDNYTIYDGF |
| | NLRNTNLAANFNGQNTEINNMNFDKLKNGTGLFEFYKLLCVR |
| | GIITSKTKSLDKGYNK |
| S012252-7 | AFVNKQFNYKDPVNGRDIAYIKIPNAGQMQPVKAFKIHNKIW |
| (SEQ ID NO: 55) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNLVIIGPSADIIQFECKSFGHEVLNLT |
| | RNGYGSTQYIRFSPDFTNGFEESLEVDTNPLLGAGKYATDPAV |
| | TLAHELIHAGHRLYGIAINPNRVFKVNTNAYYEMSGLEVSFEE |
| | LRTFGGHDAKFIDSLQENEFRDYYYNKFKDIASTLNKAKSTVG |
| | TTASLQYMKNVFKEKYLLSEDTSGKFSVDKLKFDKLYKMLTE |
| | IYTEDNFVKFFKVLNRKTYLNFDKGVFKINIVPKDNYTIKDGFN |
| | LRNTNLAANFNGQNTEINNMNFDKLKNGTGLFEFYKLLCVRD |
| | IITSKTKSLDKGYNK |
| S012252-8 | AFVNKQFNYKDPVNGRDIAYIKIPNAGQMQPVKAFKIHNKIW |
| (SEQ ID NO: 56) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVIQPDGSYRSEELNLVIIGPGADIIQFECKSFGHEVLNLT |
| | RNGYGSTQYIRFSPDFTFGFEESLEVDTNPLLGAGKYATDPAV |
| | TLAHELIHAGHRLYGIAINPNEVFKVNTNAYYEMSGLEVSFEE |
| | LRTFGGHDAKFIDSLQENEFRDKYYNKFKDIASTLNKAKSTVG |
| | TTASLQYMKNVFKEKYLLSEDTSGKFSVDKNKFDKLYKMLTE |
| | IYTEDNFVKFFKVQNRKTYLNFDKGVFKINIVPKVNYTIKDGF |
| | NLRNTNLAANFNGQNTEINNMNFDKLKNGTGLFEFYKLLCVR |
| | GIITSKTKSLDKGYNK |
| S012252-9 | AFVNKQFNYKDPVNGRDIAYIKIPNAGQMQPVKAFKIHNKIW |
| (SEQ ID NO: 57) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNLVIIGPGADIIQFECKSFGHEVLNLT |
| | RNGYGSTQYIRFSPDFTFGFEESLEVDTNPLLGAGKYATDPAV |
| | TLAHELIHAGHRLYGIAINPNEVFKVNTNAYYEMSGLEVSFEE |
| | LRTFGGHDAKFIDSLQENEFRDYYYNKFKDIASTLNKAKSTVG |
| | TTASLQYMKNVFKEKYLLSEDTSGKFSVDKLKFDKLYKMLTE |
| | IYTEDNFSKFFKVQNRKTYLNFDKAVFKINIVPKVNYTIKDGFN |
| | LRNTNLAANFNGQNTEINNMNFDKLKNGTGLFEFYKLLCVRG |
| | IITSKTKSLDKGYNK |
| S012252-10 | AFVNKQFNYKDPVNGRDIAYIKIPNAGQMQPVKAFKIHNKIW |
| (SEQ ID NO: 58) | VIPERDTFTNPEEGDLNPPPEAKQVPVSYYDSTYLSTDNEKDN |
| | YLKGVTKLFERIYETDLGRMLLTSIVRGIPFWGGSTIDTELKVI |
| | DTNCINVIQPDGSYRSEELNLVIIGPSADIIQFECKSFGHEVLNLT |
| | RNGYGSTQYIRFSPDFTFGFEESLEVDTNPLLGAGKYATDPAV |
| | TLAHELIHAGHRLYGIAINPNRVFKVNTNAYYEMSGLEVSFEE |
| | LRTFGGHDAKFIDSLQENEFRDYYYNKFKDIASTLNKAKSIVG |
| | TTASLDYMKNVFKEKYLLSEDTSGKFSVDKLKFDKLYKMLTE |
| | IYTEDNFVKFFKVLNRKTYLNFDKAVFKINIVPKVNYTIYDGFN |
| | LRNTNLAANFNGQNTEINNMNFDKLKNFTGLFEFYKLLCVRGI |
| | ITSKTKSLDKGYNK |
| S012252-11 | AFVNKQFNYKDPVNGVDIAYIKIPNAGQMQPVKAFKIHNKIW |
| (SEQ ID NO: 59) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNLVIIGPGADIIQFECKSFGHEVLNLT |
| | RNGYGSTQYIRFSPDFTFGFEESLEVDTNPLLGAGKYATDPAV |
| | TLAHELIHAGHRLYGIAINPNRVFKVNTNAYYEMSGLEVSFEE |
| | LRTFGGHDAKFIDSLQENEFRDYYYNKFKDIASTLNKAKSTVG |
| | TTASLDYMKNVFKEKYLLSEDTSGKFSVDKNKFDKLYKMLTE |
| | IYTEDNFSKFFKVQNRKTYLNFDKGVFKINIVPKDNYTIYDGFN |
| | LRNTNLAANFNGQNTEINNMNFTKLKNFTGLFEFYKLLCVRGI |
| | ITSKTKSLDKGYNK |
| S012252-12 | AFVNKQFNYKDPVNGRDIAYIKIPNAGQMQPVKAFKIHNKIW |
| (SEQ ID NO: 60) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNLVIIGPSADIIQFECKSFGHEVLNLT |
| | RNGYGSTQYIRFSPDFTNGFEESLEVDTNPLLGAGKFATDPAV |
| | TLAHELIHAGHRLYGIAINPNRVFKVNTNAYYEMSGLEVSFEE |
| | LRTFGGHDAKFIDSLQENEFRLKYYNKFKDIASTLNKAKSTVG |
| | TTASLDYMKNVFKEKYLLSEDTSGKFSVDKLKFDKLYKMLTE |
| | IYTEDNFSKFFKVLNRKTYLNFDKGVFKINIVPKDNYTIKDGFN |
| | LRNTNLAANFNGQNTEINNMNFDKLKNGTGLFEFYKLLCVRG |
| | IITSKTKSLDKGYNK |
| S012252-13 | AFVNKQFNYKDPVNGRDIAYIKIPNAGQMQPVKAFKIHNKIW |
| (SEQ ID NO: 61) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNVVIIGPSADIIQFECKSFGHEVLNLT |
| | RNGYGSTQYIRFSPDFTFGFEESLEVDTNPLLGAGKYATDPAV |
| | TLAHELIHAGHRLYGIAINPNEVFKVNTNAYYEMSGLEVKFEE |
| | LRTFGGHDAKFIDSLQENEFRDYYYNKFKDIASTLNKAKSTVG |
| | TTASLQYMKNVFKEKYLLSEDTSGKFSVDKNKFDKLYKMLTE |
| | IYTEDNFVKFFKVLNRKTYLNFDKAVFKINIVPKDNYTIKDGFN |
| | LRNTNLAANFNGQNTEINNMNFTKLKNGTGLFEFYKLLCVRGI |
| | ITSKTKSLDKGYNK |
| S012252-14 | AFVNKQFNYKDPVNGVDIAYIKIPNAGQMQPVKAFKIHNKIW |
| (SEQ ID NO: 62) | VIPERDTFTNPEEGDLNPPPEAKQVPVSYYDSTYLSTDNEKDN |
| | YLKGVTKLFERIYSTDLGRMLLTSIVRGIPFWGGSTIDTELKVID |
| | TNCINVIQPDGSYRSEELNLVIIGPSADIIQFECKSFGHEVLNLTR |
| | NGYGSTQYIRFSPDFTFGFEESLEVDTNPLLGAGKFATDPAVTL |
| | AHELIHAGHRLYGIAINPNRVFKVNTNAYYEMSGLEVSFEELR |
| | TFGGHDAKFIDSLQENEFRLYYYNKFKDIASTLNKAKSIVGTT |
| | ASLQYMKNVFKEKYLLSEDTSGKFSVDKLKFDKLYKMLTEIY |
| | TEDNFVKFFKVLNRKTYLNFDKAVFKINIVPKVNYTIYDGFNL |
| | RNTNLAANFNGQNTEINNMNFTKLKNFTGLFEFYKLLCVRGII |
| | TSKTKSLDKGYNK |
| S012252-15 | AFVNKQFNYKDPVNGVDIAYIKIPNAGQMEPVKAFKIHNKIW |
| (SEQ ID NO: 63) | VIPERDTFTNPEEGDLNPPPEAKQVPVSYYDSTYLSTDAEKDN |
| | YLKGVTKLFERIYETDLGRMLLTSIVRGIPFWGGSTIDTELKVI |
| | DTNCINVKQPDGSYRSEELNLVIIGPSADIIQFECKSFGHEVLNL |
| | TRNGYGSTQYIRFSPDFTFGFEESLEVDTNPLLGAGKYATDPA |
| | VTLAHELIHAGHRLYGIAINPNEVFKVNTNAYYEMSGLEVKFE |
| | ELRTFGGHDAKFIDSLQENEFRDKYYNKFKDIASTLNKAKSIV |
| | GTTASLQYMKNVFKEKYLLSEDTSGKFSVDKNKFDKLYKMLT |
| | EIYTEDNFVKFFKVQNRKTYLNFDKAVFKINIVPKVNYTIKDGF |
| | NLRNTNLAANFNGQNTEINNMNFTKLKNGTGLFEFYKLLCVR |
| | GIITSKTKSLDKGYNK |
| S012252-16 | AFVNKQFNYKDPVNGVDIAYIKIPNAGQMQPVKAFKIHNKIW |
| (SEQ ID NO: 64) | VIPERDTFTNPEEGDLNPPPEAKQVPVSYYDSTYLSTDAEKDN |
| | YLKGVTKLFERIYETDLGRMLLTSIVRGIPFWGGSTIDTELKVI |
| | DTNCINVIQPDGSYRSEELNLVIIGPGADIIQFECKSFGHEVLNL |
| | TRNGYGSTQYIRFSPDFNFGFEESLEVDTNPLLGAGKYATDPA |
| | VTLAHELIHAGHRLYGIAINPNEVFKVNTNAYYEMSGLEVSFE |
| | ELRTFGGHDAKFIDSLQENEFRDYYYNKFKDIASTLNKAKSIV |
| | GTTASLDYMKNVFKEKYLLSEDTSGKFSVDKNKFDKLYKMLT |
| | EIYTEDNFSKFFKVLNRKTYLNFDKGVFKINIVPKVNYTIYDGF |
| | NLRNTNLAANFNGQNTEINNMNFDKLKNGTGLFEFYKLLCVR |
| | GIITSKTKSLDKGYNK |
| S012252-17 | AFVNKQFNYKDPVNGVDIAYIKIPNAGQMQPVKAFKIHNKIW |
| (SEQ ID NO: 65) | VIPERDTFTNPEEGDLNPPPEAKQVPVSYYDSTYLSTDAEKDN |
| | YLKGVTKLFERIYETDLGRMLLTSIVRGIPFWGGSTIDTELKVI |
| | DTNCINVKQPDGSYRSEELNLVIIGPGADIIQFECKSFGHEVLNL |
| | TRNGYGSTQYIRFSPDFTFGFEESLEVDTNPLLGAGKYATDPA |
| | VTLAHELIHAGHRLYGIAINPNRVFKVNTNAYYEMSGLEVKFE |
| | ELRTFGGHDAKFIDSLQENEFRDYYYNKFKDIASTLNKAKSIV |
| | GTTASLQYMKNVFKEKYLLSEDTSGKFSVDKNKFDKLYKMLT |
| | EIYTEDNFSKFFKVQNRKTYLNFDKAVFKINIVPKDNYTIYDGF |
| | NLRNTNLAANFNGQNTEINNMNFDKLKNFTGLFEFYKLLCVR |
| | GIITSKTKSLDKGYNK |
| S012252-18 | AFVNKQFNYKDPVNGRDIAYIKIPNAGQMQPVKAFKIHNKIW |
| (SEQ ID NO: 66) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNLVIIGPGADIIQFECKSFGHEVLNLT |
| | RNGYGSTQYIRFSPDFTNGFEESLEVDTNPLLGAGKFATDPAV |
| | TLAHELIHAGHRLYGIAINPNRVFKVNTNAYYEMSGLEVKFEE |
| | LRTFGGHDAKFIDSLQENEFRDYYYNKFKDIASTLNKAKSTVG |
| | TTASLQYMKNVFKEKYLLSEDTSGKFSVDKLKFDKLYKMLTE |
| | IYTEDNFVKFFKVQNRKTYLNFDKAVFKINIVPKVNYTIYDGF |
| | NLRNTNLAANFNGQNTEINNMNFTKLKNFTGLFEFYKLLCVR |
| | GIITSKTKSLDKGYNK |
| S012252-19 | AFVNKQFNYKDPVNGVDIAYIKIPNAGQMEPVKAFKIHNKIW |
| (SEQ ID NO: 67) | VIPERDTFTNPEEGDLNPPPEAKQVPVSYYDSTYLSTDNEKDN |
| | YLKGVTKLFERIYETDLGRMLLTSIVRGIPFWGGSTIDTELKVI |
| | DTNCINVIQPDGSYRSEELNLVIIGPSADIIQFECKSFGHEVLNLT |
| | RNGYGSTQIIRFSPDFTFGFEESLEVDTNPLLGAGKYATDPAVT |
| | LAHELIHAGHRLYGIAINPNRVFKVNTNAYYEMSGLEVSFEEL |
| | RTFGGHDAKFIDSLQENEFRDYYYNKFKDIASTLNKAKSTVGT |
| | TASLQYMKNVFKEKYLLSEDTSGKFSVDKLKFDKLYKMLTEI |
| | YTEDNFVKFFKVLNRKTYLNFDKAVFKINIVPKVNYTIKDGFN |
| | LRNTNLAANFNGQNTEINNMNFDKLKNGTGLFEFYKLLCVRG |
| | IITSKTKSLDKGYNK |
| S012252-20 | AFVNKQFNYKDPVNGRDIAYIKIPNAGQMQPVKAFKIHNKIW |
| (SEQ ID NO: 68) | VIPERDTFTNPEEGDLNPPPEAKQVPVSYYDSTYLSTDNEKDN |
| | YLKGVTKLFERIYETDLGRMLLTSIVRGIPFWGGSTIDTELKVI |
| | DTNCINVIQPDGSYRSEELNLVIIGPSADIIQFECKSFGHEVLNLT |
| | RNGYGSTQYIRFSPDFTFGFEESLEVDTNPLLGAGKYATDPAV |
| | TLAHELIHAGHRLYGIAINPNRVFKVNTNAYYEMSGLEVSFEE |
| | LRTFGGHDAKFIDSLQENEFRDYYYNKFKDIASTLNKAKSIVG |
| | TTASLDYMKNVFKEKYLLSEDTSGKFSVDKLKFDKLYKMLTE |
| | IYTEDNFVKFFKVLNRKTYLNFDKAVFKINIVPKVNYTIYDGFN |
| | LRNTNLAANFNGQNTEINNMNFDKLKNFTGLFEFYKLLCVRGI |
| | ITSKTKSLDKGYNK |
| S012252-21 | AFVNKQFNYKDPVNGRDIAYIKIPNAGQMQPVKAFKIHNKIW |
| (SEQ ID NO: 69) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNLVIIGPSADIIQFECKSFGHEVLNLT |
| | RNGYGSTQYIRFSPDFTFGFEESLEVDTNPLLGAGKYATDPAV |
| | TLAHELIHAGHRLYGIAINPNRVFKVNTNAYYEMSGLEVKFEE |
| | LRTFGGHDAKFIDSLQENEFRDYYYNKFKDIASTLNKAKSTVG |
| | TTASLQYMKNVFKEKYLLSEDTSGKFSVDKLKFDKLYKMLTE |
| | IYTEDNFVKFFKVLNRKTYLNFDKAVFKINIVPKVNYTIYDGFN |
| | LRNTNLAANFNGQNTEINNMNFTKLKNGTGLFEFYKLLCVRGI |
| | ITSKTKSLDKGYNK |
| S012252-22 | AFVNKQFNYKDPVNGVDIAYIKIPNAGQMEPVKAFKIHNKIW |
| (SEQ ID NO: 70) | VIPERDTFTNPEEGDLNPPPEAKQVPVSYYDSTYLSTDNEKDN |
| | YLKGVTKLFERIYETDLGRMLLTSIVRGIPFWGGSTIDTELKVI |
| | DTNCINVIQPDGSYRSEELNLVIIGPGADIIQFECKSFGHEVLNL |
| | TRNGYGSTQIIRFSPDFTFGFEESLEVDTNPLLGAGKYATDPAV |
| | TLAHELTHAGHRLYGIAINPNEVFKVNTNAYYEMSGLEVKFEE |
| | LRTFGGHDAKFIDSLQENEFRLYYYNKFKDIASTLNKAKSIVGT |
| | TASLQYMKNVFKEKYLLSEDTSGKFSVDKNKFDKLYKMLTEI |
| | YTEDNFSKFFKVLNRKTYLNFDKGVFKINIVPKDNYTIYDGFN |
| | LRNTNLAANFNGQNTEINNMNFDKLKNFTGLFEFYKLLCVRGI |
| | ITSKTKSLDKGYNK |
| S012252-23 | AFVNKQFNYKDPVNGRDIAYIKIPNAGQMQPVKAFKIHNKIW |
| (SEQ ID NO: 71) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNLVIIGPGADIIQFECKSFGHEVLNLT |
| | RNGYGSTQYIRFSPDFTFGFEESLEVDTNPLLGAGKYATDPAV |
| | TLAHELIHAGHRLYGIAINPNEVFKVNTNAYYEMSGLEVSFEE |
| | LRTFGGHDAKFIDSLQENEFRDYYYNKFKDIASTLNKAKSTVG |
| | TTASLQYMKNVFKEKYLLSEDTSGKFSVDKLKFDKLYKMLTE |
| | IYTEDNFSKFFKVQNRKTYLNFDKAVFKINIVPKVNYTIKDGFN |
| | LRNTNLAANFNGQNTEINNMNFDKLKNGTGLFEFYKLLCVRG |
| | IITSKTKSLDKGYNK |
| S012252-24 | AFVNKQFNYKDPVNGRDIAYIKIPNAGQMEPVKAFKIHNKIWV |
| (SEQ ID NO: 72) | IPERDTFTNPEEGDLNPPPEAKQVPVSYYDSTYLSTDAEKDNYL |
| | KGVTKLFERIYETDLGRMLLTSIVRGIPFWGGSTIDTELKVIDT |
| | NCINVIQPDGSYRSEELNLVIIGPSADIIQFECKSFGHEVLNLTRN |
| | GYGSTQYIRFSPDFTFGFEESLEVDTNPLLGAGKFATDPAVTLA |
| | HELIHAGHRLYGIAINPNEVFKVNTNAYYEMSGLEVSFEELRTF |
| | GGHDAKFIDSLQENEFRDYYYNKFKDIASTLNKAKSTVGTTAS |
| | LDYMKNVFKEKYLLSEDTSGKFSVDKLKFDKLYKMLTEIYTE |
| | DNFVKFFKVQNRKTYLNFDKAVFKINIVPKVNYTIYDGFNLRN |
| | TNLAANFNGQNTEINNMNFDKLKNFTGLFEFYKLLCVRGIITS |
| | KTKSLDKGYNK |
| S012252-25 | AFVNKQFNYKDPVNGRDIAYIKIPNAGQMEPVKAFKIHNKIWV |
| (SEQ ID NO: 73) | IPERDTFTNPEEGDLNPPPEAKQVPVSYYDSTYLSTDAEKDNYL |
| | KGVTKLFERIYSTDLGRMLLTSIVRGIPFWGGSTIDTELKVIDTN |
| | CINVKQPDGSYRSEELNLVIIGPGADIIQFECKSFGHEVLNLTRN |
| | GYGSTQYIRFSPDFTFGFEESLEVDTNPLLGAGKYATDPAVTLA |
| | HELIHAGHRLYGIAINPNEVFKVNTNAYYEMSGLEVSFEELRTF |
| | GGHDAKFIDSLQENEFRDYYYNKFKDIASTLNKAKSTVGTTAS |
| | LQYMKNVFKEKYLLSEDTSGKFSVDKLKFDKLYKMLTEIYTE |
| | DNFSKFFKVLNRKTYLNFDKGVFKINIVPKVNYTIYDGFNLRN |
| | TNLAANFNGQNTEINNMNFDKLKNGTGLFEFYKLLCVRGIITS |
| | KTKSLDKGYNK |
| S012252-26 | AFVNKQFNYKDPVNGVDIAYIKIPNAGQMEPVKAFKIHNKIW |
| (SEQ ID NO: 74) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVIQPDGSYRSEELNLVIIGPSADIIQFECKSFGHEVLNLTR |
| | NGYGSTQIIRFSPDFTFGFEESLEVDTNPLLGAGKFATDPAVTL |
| | AHELIHAGHRLYGIAINPNRVFKVNTNAYYEMSGLEVSFEELR |
| | TFGGHDAKFIDSLQENEFRDYYYNKFKDIASTLNKAKSTVGTT |
| | ASLDYMKNVFKEKYLLSEDTSGKFSVDKNKFDKLYKMLTEIY |
| | TEDNFSKFFKVQNRKTYLNFDKGVFKINIVPKDNYTIYDGFNL |
| | RNTNLAANFNGQNTEINNMNFTKLKNFTGLFEFYKLLCVRGII |
| | TSKTKSLDKGYNK |
| S012252-27 | AFVNKQFNYKDPVNGRDIAYIKIPNAGQMQPVKAFKIHNKIW |
| (SEQ ID NO: 75) | VIPERDTFTNPEEGDLNPPPEAKQVPVSYYDSTYLSTDAEKDN |
| | YLKGVTKLFERIYETDLGRMLLTSIVRGIPFWGGSTIDTELKVI |
| | DTNCINVIQPDGSYRSEELNLVIIGPSADIIQFECKSFGHEVLNLT |
| | RNGYGSTQIIRFSPDFTFGFEESLEVDTNPLLGAGKYATDPAVT |
| | LAHELIHAGHRLYGIAINPNRVFKVNTNAYYEMSGLEVSFEEL |
| | RTFGGHDAKFIDSLQENEFRDYYYNKFKDIASTLNKAKSIVGT |
| | TASLQYMKNVFKEKYKLSEDTSGKFSVDKNKFDKLYKMLTEI |
| | YTEDNFVKFFKVQNRKTYLNFDKAVFKINIVPKVNYTIYDGFN |
| | LRNTNLAANFNGQNTEINNMNFDKLKNGTGLFEFYKLLCVRG |
| | IITSKTKSLDKGYNK |
| S012252-28 | AFVNKQFNYKDPVNGRDIAYIKIPNAGQMQPVKAFKIHNKIW |
| (SEQ ID NO: 76) | VIPERDTFTNPEEGDLNPPPEAKQVPVSYYDSTYLSTDNEKDN |
| | YLKGVTKLFERIYETDLGRMLLTSIVRGIPFWGGSTIDTELKVI |
| | DTNCINVIQPDGSYRSEELNLVIIGPSADIIQFECKSFGHEVLNLT |
| | RNGYGSTQIIRFSPDFTFGFEESLEVDTNPLLGAGKYATDPAVT |
| | LAHELIHAGHRLYGIAINPNRVFKVNTNAYYEMSGLEVSFEEL |
| | RTFGGHDAKFIDSLQENEFRDYYYNKFKDIASTLNKAKSTVGT |
| | TASLQYMKNVFKEKYLLSEDTSGKFSVDKLKFDKLYKMLTEI |
| | YTEDNFVKFFKVLNRKTYLNFDKAVFKINIVPKVNYTIKDGFN |
| | LRNTNLAANFNGQNTEINNMNFDKLKNGTGLFEFYKLLCVRG |
| | IITSKTKSLDKGYNK |
| S012252-29 | AFVNKQFNYKDPVNGVDIAYIKIPNAGQMQPVKAFKIHNKIW |
| (SEQ ID NO: 77) | VIPERDTFTNPEEGDLNPPPEAKQVPVSYYDSTYLSTDAEKDN |
| | YLKGVTKLFERIYETDLGRMLLTSIVRGIPFWGGSTIDTELKVI |
| | DTNCINVKQPDGSYRSEELNLVIIGPGADIIQFECKSFGHEVLNL |
| | TRNGYGSTQYIRFSPDFTFGFEESLEVDTNPLLGAGKYATDPA |
| | VTLAHELIHAGHRLYGIAINPNRVFKVNTNAYYEMSGLEVKFE |
| | ELRTFGGHDAKFIDSLQENEFRDYYYNKFKDIASTLNKAKSIV |
| | GTTASLQYMKNVFKEKYLLSEDTSGKFSVDKNKFDKLYKMLT |
| | EIYTEDNFSKFFKVQNRKTYLNFDKAVFKINIVPKDNYTIYDGF |
| | NLRNTNLAANFNGQNTEINNMNFDKLKNFTGLFEFYKLLCVR |
| | GIITSKTKSLDKGYNK |
| S012252-30 | AFVNKQFNYKDPVNGVDIAYIKIPNAGQMQPVKAFKIHNKIW |
| (SEQ ID NO: 78) | VIPERDTFTNPEEGDLNPPPEAKQVPVSYYDSTYLSTDNEKDN |
| | YLKGVTKLFERIYSTDLGRMLLTSIVRGIPFWGGSTIDTELKVID |
| | TNCINVIQPDGSYRSEELNLVIIGPSADIIQFECKSFGHEVLNLTR |
| | NGYGSTQYIRFSPDFTFGFEESLEVDTNPLLGAGKFATDAAVTL |
| | AHQLMHAGHRLYGIAINPNRVFKVNTNAYYEMSGLEVSFEEL |
| | RTFGGHDAKFIDSLQENEFRLYYYNKFKDIASTLNKAKSIVGTT |
| | ASLQYMKNVFKEKYLLSEDTSGKFSVDKLKFDKLYKMLTEIY |
| | TEDNFVKFFKVLNRKTFLNFDKAVFKINIVPKVNYTIYDGFNL |
| | RNTNLAANFNGQNTEINNMNFTKLKNFTGLFEFYKLLCVRGII |
| | TSKTKSLDKGYNK |
| S012252-32 | AFVNKQFNYKDPVNGRDIAYIKIPNAGQMQPVKAFKIHNKIW |
| (SEQ ID NO: 79) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVKQPDGSYRSEELNVVIIGPSADIIQFECKSFGHEVLNLT |
| | RNGYGSTQYIRFSPDFTFGFEESLEVDTNPLLGAGKYATDPAV |
| | TLAHELIHAGHRLYGIAINPNEVFKVNTNAYYEMSGLEVKFEE |
| | LRTFGGHDAKFIDSLQENEFRDYYYNKFKDIASTLNKAKSTVG |
| | TTASLQYMKNVFKEKYLLSEDTSGKFSVDKNKFDKLYKMLTE |
| | IYTEDNFVKFFKVLNRKTYLNFDKAVFKINIVPKDNYTIKDGFN |
| | LRNTNLAANFNGQNTEINNMNFTKLKNGTGLFEFYKLLCVRGI |
| | ITSKTKSLDKGYNK |
| A186614 | AFVNKQFNYKDPVNGRDIAYIKIPNAGQMEPVKAFKIHNKIWV |
| (SEQ ID NO: 80) | IPERDTFTNPEEGDLNPPPEAKQVPVSYYDSTYLSTDAEKDNYL |
| | KGVTKLFERIYSTDLGRMLLTSIVRGIPFWGGSTIDTELKVIDTN |
| | CINVKQPDGSYRSEELNVVIIGPSADIIQFECKSFGHEVLNLTRN |
| | GYGSTQYIRFSPDFTFGFEESLEVDTNPLLGAGKYATDPAVTLA |
| | HELTHAGHRLYGIAINPNRVFKVNTNAYYEMSGLEVKFEELRT |
| | FGGHDAKFIDSLQENEFRLYYYNKFKDIASTLNKAKSTVGTTA |
| | SLQYMKNVFKEKYLLSEDTSGKFSVDKLKFDKLYKMLTEIYT |
| | EDNFSKFFKVQNRKTYLNFDKAVFKINIVPKDNYTIKDGFNLR |
| | NTNLAANFNGQNTEINNMNFDKLKNGTGLFEFYKLLCVRGIIT |
| | SKTKSLDKGYNK |
| A186615 | AFVNKQFNYKDPVNGRDIAYIKIPNAGQMQPVKAFKIHNKIW |
| (SEQ ID NO: 81) | VIPERDTFTNPEEGDLNPPPEAKQVPVSYYDSTYLSTDNEKDN |
| | YLKGVTKLFERIYETDLGRMLLTSIVRGIPFWGGSTIDTELKVI |
| | DTNCINVIQPDGSYRSEELNLVIIGPSADIIQFECKSFGHEVLNLT |
| | RHGYGSTQYIRSVRDFTLPSAESLEVDTHPLVGAAKYATDPAA |
| | TLAHELIHAGHRLSGIAINPNRVFKVNTNAYYEMSGLEVSFEEL |
| | RTFGGHDAKFIDSLQENEFRDYYYNKFKDIASTLNKAKSIVGT |
| | TASLDYMKNVFKEKYLLSEDTSGKFSVDKNKFDKLYKMLTEI |
| | YTEDNFVKFFKVLNRKTYLNFDKGVFKINIVPKVNYTIKDGFN |
| | LRNTNLAANFNGQNTEINNMNFDKLKNGTGLFEFYKLLCVRG |
| | IITSKTKSLDKGYNK |
| A186616 | AFVNKQFNYKDPVNGVDIAYIKIPNAGQMQPVKAFKIHNKIW |
| (SEQ ID NO: 82) | VIPERDTFTNPEEGDLNPPPEAKQVPVSYYDSTYLSTDAEKDN |
| | YLKGVTKLFERIYETDLGRMLLTSIVRGIPFWGGSTIDTELKVI |
| | DTNCINVKQPDGSYRSEELNLVIIGPSADIIQFECKSFGHEVLNL |
| | TRNGYGSTQIIRFSPDFTFGFEESLEVDTNPLLGAGKFATDPAV |
| | TLAHELIHAGHRLYGIAINPNEVFKVNTNAYYEMSGLEVSFEE |
| | LRTFGGHDAKFIDSLQENEFRDYYYNKFKDIASTLNKAKSTVG |
| | TTASLDYMKNVFKEKYLLSEDTSGKFSVDKLKFDKLYKMLTE |
| | IYTEDNFVKFFKVQNRKTYLNFDKAVFKINIVPKVNYTIKDGF |
| | NLRNTNLAANFNGQNTEINNMNFDKLKNFTGLFEFYKLLCVR |
| | GIITSKTKSLDKGYNK |
| A187579 | AFVNKQFNYKDPVNGRDIAYIKIPNAGQMQPVKAFKIHNKIW |
| (SEQ ID NO: 83) | VIPERDTFTNPEEGDLNPPPEAKQVPVSYYDSTYLSTDNEKDN |
| | YLKGVTKLFERIYSTDLGRMLLTSIVRGIPFWGGSTIDTELKVID |
| | TNCINVKQPDGSYRSEELNVVIIGPGADIIQFECKSFGHEVLNLT |
| | RNGYGSTQIIRFSPDFTFGFEESLEVDTNPLLGAGKYATDPAVT |
| | LAHELIHAGHRLYGIAINPNRVFKVNTNAYYEMSGLEVKFEEL |
| | RTFGGHDAKFIDSLQENEFRDKYYNKFKDIASTLNKAKSTVGT |
| | TASLDYMKNVFKEKYLLSEDTSGKFSVDKNKFDKLYKMLTEI |
| | YTEDNFSKFFKVLNRKTYLNFDKGVFKINIVPKVNYTIKDGFN |
| | LRNTNLAANFNGQNTEINNMNFTKLKNFTGLFEFYKLLCVRGI |
| | ITIKTKSLDKGYNK |
| A187719 | AFVNKQFNYKDPVNGRDIAYIKIPNAGQMEPVKAFKIHNKIWV |
| (SEQ ID NO: 84) | IPERDTFTNPEEGDLNPPPEAKQVPVSYYDSTYLSTDNEKDNYL |
| | KGVTKLFERIYETDLGRMLLTSIVRGIPFWGGSTIDTELKVIDT |
| | NCINVKQPDGSYRSEELNLVIIGPSADIIQFECKSFGHEVLNLTR |
| | NGYGSTQYIRFSPDFTFGFEESLEVDTNPLLGAGKFATDPAVTL |
| | AHELIHAGHRLYGIAINPNRVFKVNTNAYYEMSGLEVKFEELR |
| | TFGGHDAKFIDSLQENEFRDYYYNKFKDIASTLNKAKSIVGTT |
| | ASLQYMKNVFKEKYLLSEDTSGKFSVDKNKFDKLYKMLTEIY |
| | TEDNFVKFFKVQNRKTYLNFDKAVFKINIVPKDNYTIYDGFNL |
| | RNTNLAANFNGQNTEINNMNFDKLKNFTGLFEFYKLLCVRGII |
| | TSKTKSLDKGYNK |
| A187720 | AFVNKRFNYKDPVSGRDIAYIKIPNAGQMQPVKAFKIHNKIWV |
| (SEQ ID NO: 85) | IPERDTFTNPEEGDLNPPPEAKQVPVSYYDRTYLSTDNEKDNY |
| | LKGVTKLFERIYETDLGRMLLTSIVRGIPFWGGSTIDTELKVIDT |
| | NCINVKQPDGSYRSEELNLVIIGPGADIIQFECRSFGHEVLNLTR |
| | NGYGSTQYIRFSPDFTNGFEESLEVDTNPLLGAGKFATDPAVTL |
| | AHELIHAGHRLYGIAINPNRVFKVNTNAYYEMSGLEVKFEELR |
| | TFGGHDAKFIDSLQENEFRLYYYNKFKDIASTLNKAKSTVGTT |
| | ASLQYMKNVFKEKYLLSEDTSGKFSVDKLKFDKLYKMLTEIY |
| | TEDNFSKFFKVQNRKTYLNFDKAVFKINIVPKDNYTIKDGFNL |
| | RNTNLAANFNGQNTEINNMNFDKLKNGTGLFEFYKLLCVRGII |
| | TSKTKSLDKGYNK |
| A187721 | AFVNKQFNYKDPVNGRDIAYIKIPNAGQMQPVKAFKIHNKIW |
| (SEQ ID NO: 86) | VIPERDTFTNPEEGDLNPPPEAKQVPVSYYDSTYLSTDAEKDN |
| | YLKGVTKLFERIYETDLGRMLLTSIVRGIPFWGGSTIDTELKVI |
| | DTNCINVKQPDGSYRSEELNLVIIGPSADIIQFECKSFGHEVLNL |
| | TRNGYGSTQIIRFSPDFTFGFEESLEVDTNPLLGAGKYATDPAV |
| | TLAHELTHAGHRLYGIAINPNRVFKVNTNAYYEMSGLEVSFEE |
| | LRTFGGHDAKFIDSLQENEFRDYYYNKFKDIASTLNKAKSIVG |
| | TTASLDYMKNVFKEKYLLSEDTSGKFSVDKNKFDKLYKMLTE |
| | IYTEDNFVKFFKVQNRKTYLNFDKGVFKINIVPKDNYTIYDGF |
| | NLRNTNLAANFNGQNTEINNMNFDKLKNFTGLFEFYKLLCVR |
| | GIITSKTKSLDKGYNK |
| A189994 | AFVNKQFNYKDPVNGRDIAYIKIPNAGQMQPVKAFKIHNKIW |
| (SEQ ID NO: 87) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVIQPDGSYRSEELNLVIIGPSADIIQFECKSFGHEVLNLTR |
| | NGYGSTQIIRFSPDFTFGFEESLEVDTNPLLGAGKFATDPAVTL |
| | AHELIHAGHRLYGIAINPNRVFKVNTNAYYEMSGLEVKFEELR |
| | TFGGHDAKFIDSLQENEFRLYYYNKFKDIASTLNKAKSTVGTT |
| | ASLQYMKNVFKEKYLLSEDTSGKFSVDKNKFDKLYKMLTEIY |
| | TEDNFVKFFKVQNRKTYLNFDKAVFKINIVPKVNYTIYDGFNL |
| | RNTNLAANFNGQNTEINNMNFTKLKNFTGLFEFYKLLCVRGII |
| | TSKTKSLDKGYNK |
| A189995 | AFVNKQFNYKDPVNGVDIAYIKIPNAGQMEPVKAFKIHNKIW |
| (SEQ ID NO: 88) | VIPERDTFTNPEEGDLNPPPEAKQVPVSYYDSTYLSTDNEKDN |
| | YLKGVTKLFERIYSTDLGRMLLTSIVRGIPFWGGSTIDTELKVID |
| | TNCINVIQPDGSYRSEELNLVIIGPSADIIQFECKSFGHEVLNLTR |
| | NGYGSTQYIRFSPDFTFGFEESLEVDTNPLLGAGKFATDPAVTL |
| | AHELIHAGHRLYGIAINPNEVFKVNTNAYYEMSGLEVSFEELR |
| | TFGGHDAKFIDSLQENEFRDKYYNKFKDIASTLNKAKSTVGTT |
| | ASLQYMKNVFKEKYLLSEDTSGKFSVDKLKFDKLYKMLTEIY |
| | TEDNFSKFFKVQNRKTYLNFDKAVFKINIVPKDNYTIKDGFNL |
| | RNTNLAANFNGQNTEINNMNFDKLKNGTGLFEFYKLLCVRGII |
| | TSKTKSLDKGYNK |
| A190251 | AFVNKQFNYKDPVNGRDIAYIKIPNAGQMEPVKAFKIHNKIWV |
| (SEQ ID NO: 89) | IPERDTFTNPEEGDLNPPPEAKQVPVSYYDSTYLSTDAEKDNYL |
| | KGVTKLFERIYSTDLGRMLLTSIVRGIPFWGGSTIDTELKVIDTN |
| | CINVIQPDGSYRSEELNLVIIGPSADIIQFECKSFGHEVLNLTRNG |
| | YGSTQIIRFSPDFTFGFEESLEVDTNPLLGAGKFATDPAVTLAH |
| | ELIHAGHRLYGIAINPNRVFKVNTNAYYEMSGLEVKFEELRTF |
| | GGHDAKFIDSLQENEFRDKYYNKFKDIASTLNKAKSIVGTTAS |
| | LQYMKNVFKEKYLLSEDTSGKFSVDKNKFDKLYKMLTEIYTE |
| | DNFSKFFKVLNRKTYLNFDKAVFKINIVPKVNYTIYDGFNLRN |
| | TNLAANFNGQNTEINNMNFTKLKNFTGLFEFYKLLCVRGIITSK |
| | TKSLDKGYNK |
| A190252 | AFVNKQFNYKDPVNGRDIAYIKIPNACQMEPVKAFKIHNKIWV |
| (SEQ ID NO: 90) | IPERDTFTNPEEGDLNPPPEAKQVPVSYYDSTYLSTDNEKDNYL |
| | KGVTKLFERIYSTDLGRMLLTSIVRGIPFWGGSTIDTELKVIDTN |
| | CINVKQPDGSYRSEELNVVIIGPGADIIQFECKSFGHEVLNLTRN |
| | GYGSTQYIRFSPDFTFGFEESLEVDTNPLLGAGKYATDPAVTLA |
| | HELIHAGHRLYGTAINPNRVFKVNTNAYYEMSGLEVSFEELRT |
| | FGGHDAKFIDSLQENEFRDKYYNKFKDIASTLNKANSIVGTTA |
| | SLQYMKNVFKEKYLLSEDTSGKFSVDKLKFDKLYKMLTEIYT |
| | EDNFVKFFKVQNRKTYLNFDKAVFKINIVPKVNYTIYDGFNLR |
| | NTNLAANFNGQNTEINNMNFTKLKNFTGLFEFYKLLCVRGIITS |
| | KTKSLDKGYNK |
| A190253 | AFVNKQFNYKDPVNGRDIAYIKIPNACQMEPVKAFKIHNKIWV |
| (SEQ ID NO: 91) | IPERDTFTNPEEGDLNPPPEAKQVPVSYYDSTYLSTDNEKDNYL |
| | KGVTKLFERIYSTDLGRMLLTSIVRGIPFWGGSTIDTELKVIDTN |
| | CINVKQPDGSYRSEELNVVIIGPGADIIQFECKSFGHEVLNLTRN |
| | GYGSTQYIRFSPDFTFGFEESLEVDTNPLLGAGKYATDPAVTLA |
| | HELIHAGHRLYGTAINPNRVFKVNTNAYYEMSGLEVSFEELRT |
| | FGGHDAKFIDSLQENEFRDKYYNKFKDIASTLNKANSIVGTTA |
| | SLQYMKNVFKEKYLLSEDTSGKFSVDKLKFDKLYKMLTEIYT |
| | EDNFVKFFKVQNRKTYLNFDKAVFKINIVPKVNYTIYDGFNLR |
| | NTNLAANFNGQNTEINNMNFTKLKNFTGLFEFYKLLCVRGIITS |
| | KTKSLDKGYNK |
| A190254 | AFVNKQFNYKDPVNGVDIAYIKIPNAGQMQPVKAFKIHNKIW |
| (SEQ ID NO: 92) | VIPERDTFTNPEEGDLNPPPEAKQVPVSYYDSTYLSTDAEKDN |
| | YLKGVTKLFERIYSTDLGRMLLTSIVRGIPFWGGSTIDTELKVID |
| | TNCINVIQPDGSYRSEELNVVIIGPSADIIQFECKSFGHEVLNLTR |
| | NGYGSTQYIRFSPDFTFGFEESLEVDTNPLLGAGKYATDPAVTL |
| | AHELTHAGHRLYGIAINPNRVFKVNTNAYYEMSGLEVSFEELR |
| | TFGGHDAKFIDSLQENEFRLYYYNKFKDIASTLNKAKSTVGTT |
| | ASLQYMKNVFKEKYLLSEDTSGKFSVDKNKFDKLYKMLTEIY |
| | TEDNFSKFFKVLNRKTYLNFDKAVFKINIVPKDNYTIYDGFNL |
| | RNTNLAANFNGQNTEINNMNFDKLKNFTGLFEFYKLLCVRGII |
| | TSKTKSLDKGYNK |
| A190255 | AFVNKQFNYKDPVNGVDIAYIKIPNAGQMQPVKAFKIHNKIW |
| (SEQ ID NO: 93) | VIPERDTFTNPEEGDLNPPPEAKQVPVSYYDSTYLSTDNEKDN |
| | YLKGVTKLFERIYSTDLGRMLLTSIVRGIPFWGGSTIDTELKVID |
| | TNCINVIQPDGSYRSEELNLVIIGPSADIIQFECKSFGHEVLNLTR |
| | NGYGSTQIIRFSPDFTFGFEESLEVDTNPLLGAGKYATDPAVTL |
| | AHELIHAGHRLYGIAINPNRVFKVNTNAYYEMSGLEVKFEELR |
| | TFGGHDAKFIDSLQENEFRDKYYNKFKDIASTLNKAKSTVGTT |
| | ASLDYMKNVFKEKYLLSEDTSGKFSVDKNKFDKLYKMLTEIY |
| | TEDNFSKFFKVLNRKTYLNFDKGVFKINIVPKVNYTIKDGFNL |
| | RNTNLAANFNGQNTEINNMNFTKLKNFTGLFEFYKLLCVRGII |
| | TSKTKSLDKGYNK |

The mutation positions and specific mutations of the deimmunized BoNT/A-LC variants of Table 2 may be readily determined by aligning each sequence with the WT BoNT/A-LC sequence of SEQ ID NO: 1.

TABLE 3
Amino Acid Sequences of Deimmunized BoNT/A-LC Variants:
Optimized Design

| Sequence | | |
|---|---|---|
| ID | Sequence | Mutations |
| yj_6_5 | PFVNKQFNYKDPVNGVDIAYIKIPNAGQMQPVKA | S99E \| F193S \| |
| (SEQ ID | FKIHNKIWVIPERDTFTNPEEGDLNPPPEAKQVPVS | L283E \| V354A \| |
| NO: 94) | YYDSTYLSTDNEKDNYLKGVTKLFERIYETDLGR | V381D \| I433T |
| | MLLTSIVRGIPFWGGSTIDTELKVIDTNCINVIQPDG | |
| | SYRSEELNLVIIGPSADIIQFECKSFGHEVLNLTRNG | |
| | YGSTQYIRFSPDFTSGFEESLEVDTNPLLGAGKFAT | |
| | DPAVTLAHELIHAGHRLYGIAINPNRVFKVNTNAY | |
| | YEMSGLEVSFEELRTFGGHDAKFIDSLQENEFREY | |
| | YYNKFKDIASTLNKAKSIVGTTASLQYMKNVFKE | |
| | KYLLSEDTSGKFSVDKLKFDKLYKMLTEIYTEDNF | |
| | AKFFKVLNRKTYLNFDKAVFKINIVPKDNYTIYDG | |
| | FNLRNTNLAANFNGQNTEINNMNFTKLKNFTGLFE | |
| | FYKLLCVRGTITSKTKSLDKGYNK | |
| Yj_7_6 | PFVNKQFNYKDPVNGVDIAYIKIPNAGQMQPVKA | S99E \| F193S \| |
| (SEQ ID | FKIHNKIWVIPERDTFTNPEEGDLNPPPEAKQVPVS | L283E \| L335D \| |
| NO: 95) | YYDSTYLSTDNEKDNYLKGVTKLFERIYETDLGR | V354A \| V381D \| |
| | MLLTSIVRGIPFWGGSTIDTELKVIDTNCINVIQPDG | I433T |
| | SYRSEELNLVIIGPSADIIQFECKSFGHEVLNLTRNG | |
| | YGSTQYIRFSPDFTSGFEESLEVDTNPLLGAGKFAT | |
| | DPAVTLAHELIHAGHRLYGIAINPNRVFKVNTNAY | |
| | YEMSGLEVSFEELRTFGGHDAKFIDSLQENEFREY | |
| | YYNKFKDIASTLNKAKSIVGTTASLQYMKNVFKE | |
| | KYLLSEDTSGKFSVDKDKFDKLYKMLTEIYTEDNF | |
| | AKFFKVLNRKTYLNFDKAVFKINIVPKDNYTIYDG | |
| | FNLRNTNLAANFNGQNTEINNMNFTKLKNFTGLFE | |
| | FYKLLCVRGTITSKTKSLDKGYNK | |
| Yj_8_14 | PFVNKQFNYKDPVNGVDIAYIKIPNAGQMEPVKAF | Q30E \| S99E \| F193S \| |
| (SEQ ID | KIHNKIWVIPERDTFTNPEEGDLNPPPEAKQVPVSY | L283E \| L335D \| |
| NO: 96) | YDSTYLSTDNEKDNYLKGVTKLFERIYETDLGRM | V354A \| V381D \| |
| | LLTSIVRGIPFWGGSTIDTELKVIDTNCINVIQPDGS | T438D |
| | YRSEELNLVIIGPSADIIQFECKSFGHEVLNLTRNGY | |
| | GSTQYIRFSPDFTSGFEESLEVDTNPLLGAGKFATD | |
| | PAVTLAHELIHAGHRLYGIAINPNRVFKVNTNAYY | |
| | EMSGLEVSFEELRTFGGHDAKFIDSLQENEFREYY | |
| | YNKFKDIASTLNKAKSIVGTTASLQYMKNVFKEK | |
| | YLLSEDTSGKFSVDKDKFDKLYKMLTEIYTEDNFA | |
| | KFFKVLNRKTYLNFDKAVFKINIVPKDNYTIYDGF | |
| | NLRNTNLAANFNGQNTEINNMNFTKLKNFTGLFEF | |
| | YKLLCVRGIITSKDKSLDKGYNK | |
| Yj _9_1 | PFVNKQFNYKDPVNGVDIAYIKIPNAGQMEPVKAF | Q30E \| S99E \| L283E \| |
| (SEQ ID | KIHNKIWVIPERDTFTNPEEGDLNPPPEAKQVPVSY | L335D \| V354A \| |
| NO: 97) | YDSTYLSTDNEKDNYLKGVTKLFERIYETDLGRM | V381D \| K416S \| |
| | LLTSIVRGIPFWGGSTIDTELKVIDTNCINVIQPDGS | I433T \| T438D |
| | YRSEELNLVIIGPSADIIQFECKSFGHEVLNLTRNGY | |
| | GSTQYIRFSPDFTFGFEESLEVDTNPLLGAGKFATD | |
| | PAVTLAHELIHAGHRLYGIAINPNRVFKVNTNAYY | |
| | EMSGLEVSFEELRTFGGHDAKFIDSLQENEFREYY | |
| | YNKFKDIASTLNKAKSIVGTTASLQYMKNVFKEK | |
| | YLLSEDTSGKFSVDKDKFDKLYKMLTEIYTEDNFA | |
| | KFFKVLNRKTYLNFDKAVFKINIVPKDNYTIYDGF | |
| | NLRNTNLAANFNGQNTEINNMNFTKLSNFTGLFEF | |
| | YKLLCVRGTITSKDKSLDKGYNK | |
| GG_1_10 | PFVNKQFNYKDPVNGVDIAYIKIPNAGQMQPVKA | S99E \| L199T \| |
| (SEQ ID | FKIHNKIWVIPERDTFTNPEEGDLNPPPEAKQVPVS | N279K \| L283E \| |
| NO: 98) | YYDSTYLSTDNEKDNYLKGVTKLFERIYETDLGR | L321K \| S323D \| |
| | MLLTSIVRGIPFWGGSTIDTELKVIDTNCINVIQPDG | L335E \| V381D \| |
| | SYRSEELNLVIIGPSADIIQFECKSFGHEVLNLTRNG | K416S \| T438D |
| | YGSTQYIRFSPDFTFGFEESTEVDTNPLLGAGKFAT | |
| | DPAVTLAHELIHAGHRLYGIAINPNRVFKVNTNAY | |
| | YEMSGLEVSFEELRTFGGHDAKFIDSLQEKEFREY | |
| | YYNKFKDIASTLNKAKSIVGTTASLQYMKNVFKE | |
| | KYKLDEDTSGKFSVDKEKFDKLYKMLTEIYTEDN | |
| | FVKFFKVLNRKTYLNFDKAVFKINIVPKDNYTIYD | |
| | GFNLRNTNLAANFNGQNTEINNMNFTKLSNFTGLF | |
| | EFYKLLCVRGIITSKDKSLDKGYNK | |
| GG_1_10r | PFVNKQFNYKDPVNGVDIAYIKIPNAGQMQPVKA | S99E \| N279K \| |
| 2 | FKIHNKIWVIPERDTFTNPEEGDLNPPPEAKQVPVS | L283E \| L321K \| |
| (SEQ ID | YYDSTYLSTDNEKDNYLKGVTKLFERIYETDLGR | S323D \| V381D \| |
| NO: 99) | MLLTSIVRGIPFWGGSTIDTELKVIDTNCINVIQPDG | K416S \| T438D |
| | SYRSEELNLVIIGPSADIIQFECKSFGHEVLNLTRNG | |
| | YGSTQYIRFSPDFTFGFEESLEVDTNPLLGAGKFAT | |
| | DPAVTLAHELIHAGHRLYGIAINPNRVFKVNTNAY | |
| | YEMSGLEVSFEELRTFGGHDAKFIDSLQEKEFREY | |
| | YYNKFKDIASTLNKAKSIVGTTASLQYMKNVFKE | |
| | KYKLDEDTSGKFSVDKLKFDKLYKMLTEIYTEDN | |
| | FVKFFKVLNRKTYLNFDKAVFKINIVPKDNYTIYD | |
| | GFNLRNTNLAANFNGQNTEINNMNFTKLSNFTGLF | |
| | EFYKLLCVRGIITSKDKSLDKGYNK | |
| gg_8_11 | PFVNKQFNYKDPVNGVDIAYIKIPNAGQMQPVKA | S99E \| L199T \| |
| (SEQ ID | FKIHNKIWVIPERDTFTNPEEGDLNPPPEAKQVPVS | N279K \| L283E \| |
| NO: 100) | YYDSTYLSTDNEKDNYLKGVTKLFERIYETDLGR | L321K \| 5323D \| |
| | MLLTSIVRGIPFWGGSTIDTELKVIDTNCINVIQPDG | L335E \| L360Q \| |
| | SYRSEELNLVIIGPSADIIQFECKSFGHEVLNLTRNG | V381D \| L421V \| |
| | YGSTQYIRFSPDFTFGFEESTEVDTNPLLGAGKFAT | T438D |
| | DPAVTLAHELIHAGHRLYGIAINPNRVFKVNTNAY | |
| | YEMSGLEVSFEELRTFGGHDAKFIDSLQEKEFREY | |
| | YYNKFKDIASTLNKAKSIVGTTASLQYMKNVFKE | |
| | KYKLDEDTSGKFSVDKEKFDKLYKMLTEIYTEDN | |
| | FVKFFKVQNRKTYLNFDKAVFKINIVPKDNYTIYD | |
| | GFNLRNTNLAANFNGQNTEINNMNFTKLKNFTGV | |
| | FEFYKLLCVRGIITSKDKSLDKGYNK | |
| yj_11_3 | PFVNKQFNYKDPVNGVDIAYIKIPNAGQMEPVKAF | Q30E \| S99E \| L283E \| |
| (SEQ ID | KIHNKIWVIPERDTFTNPEEGDLNPPPEAKQVPVSY | Y285K \| L335D \| |
| NO: 101) | YDSTYLSTDNEKDNYLKGVTKLFERIYETDLGRM | V354A \| V381D \| |
| | LLTSIVRGIPFWGGSTIDTELKVIDTNCINVIQPDGS | Y386S \| K416S \| |
| | YRSEELNLVIIGPSADIIQFECKSFGHEVLNLTRNGY | I433T \| T438D |
| | GSTQYIRFSPDFTFGFEESLEVDTNPLLGAGKFATD | |
| | PAVTLAHELIHAGHRLYGIAINPNRVFKVNTNAYY | |
| | EMSGLEVSFEELRTFGGHDAKFIDSLQENEFREYK | |
| | YNKFKDIASTLNKAKSIVGTTASLQYMKNVFKEK | |
| | YLLSEDTSGKFSVDKDKFDKLYKMLTEIYTEDNFA | |
| | KFFKVLNRKTYLNFDKAVFKINIVPKDNYTISDGF | |
| | NLRNTNLAANFNGQNTEINNMNFTKLSNFTGLFEF | |
| | YKLLCVRGTITSKDKSLDKGYNK | |
| gg_6_12 | PFVNKQFNYKDPVNGVDIAYIKIPNAGQMEPVKAF | Q30E \| S99E \| L199T \| |
| (SEQ ID | KIHNKIWVIPERDTFTNPEEGDLNPPPEAKQVPVSY | N279K \| L283E \| |
| NO: 102) | YDSTYLSTDNEKDNYLKGVTKLFERIYETDLGRM | L321K \| S323D \| |
| | LLTSIVRGIPFWGGSTIDTELKVIDTNCINVIQPDGS | L335D \| V381D \| |
| | YRSEELNLVIIGPSADIIQFECKSFGHEVLNLTRNGY | L421V \| I433T \| |
| | GSTQYIRFSPDFTFGFEESTEVDTNPLLGAGKFATD | T438D |
| | PAVTLAHELIHAGHRLYGIAINPNRVFKVNTNAYY | |
| | EMSGLEVSFEELRTFGGHDAKFIDSLQEKEFREYY | |
| | YNKFKDIASTLNKAKSIVGTTASLQYMKNVFKEK | |
| | YKLDEDTSGKFSVDKDKFDKLYKMLTEIYTEDNF | |
| | VKFFKVLNRKTYLNFDKAVFKINIVPKDNYTIYDG | |
| | FNLRNTNLAANFNGQNTEINNMNFTKLKNFTGVF | |
| | EFYKLLCVRGTITSKDKSLDKGYNK | |
| Yj_12_1 | PFVNKQFNYKDPVNGVDIAYIKIPNAGQMEPVKAF | Q30E \| S99E \| F1935 \| |
| (SEQ ID | KIHNKIWVIPERDTFTNPEEGDLNPPPEAKQVPVSY | L283E \| Y285K \| |
| NO: 103) | YDSTYLSTDNEKDNYLKGVTKLFERIYETDLGRM | L335D \| V354A \| |
| | LLTSIVRGIPFWGGSTIDTELKVIDTNCINVIQPDGS | V381D \| Y386S \| |
| | YRSEELNLVIIGPSADIIQFECKSFGHEVLNLTRNGY | K416S \| I433T \| |
| | GSTQYIRFSPDFTSGFEESLEVDTNPLLGAGKFATD | T438D |
| | PAVTLAHELIHAGHRLYGIAINPNRVFKVNTNAYY | |
| | EMSGLEVSFEELRTFGGHDAKFIDSLQENEF REYK | |
| | YNKFKDIASTLNKAKSIVGTTASLQYMKNVFKEK | |
| | YLLSEDTSGKFSVDKDKFDKLYKMLTEIYTEDNFA | |
| | KFFKVLNRKTYLNFDKAVFKINIVPKDNYTISDGF | |
| | NLRNTNLAANFNGQNTEINNMNFTKLSNFTGLFEF | |
| | YKLLCVRGTITSKDKSLDKGYNK | |
| gg_7_13 | PFVNKQFNYKDPVNGVDIAYIKIPNAGQMEPVKAF | Q30E \| S99E \| L199T \| |
| (SEQ ID | KIHNKIWVIPERDTFTNPEEGDLNPPPEAKQVPVSY | L276A \| N279K \| |
| NO: 104) | YDSTYLSTDNEKDNYLKGVTKLFERIYETDLGRM | L283E \| S323D \| |
| | LLTSIVRGIPFWGGSTIDTELKVIDTNCINVIQPDGS | L335D \| V381D \| |
| | YRSEELNLVIIGPSADIIQFECKSFGHEVLNLTRNGY | K416S \| L421V \| |
| | GSTQYIRFSPDFTFGFEESTEVDTNPLLGAGKFATD | I433T \| T438D |
| | PAVTLAHELIHAGHRLYGIAINPNRVFKVNTNAYY | |
| | EMSGLEVSFEELRTFGGHDAKFIDSAQEKEFREYY | |
| | YNKFKDIASTLNKAKSIVGTTASLQYMKNVFKEK | |
| | YLLDEDTSGKFSVDKDKFDKLYKMLTEIYTEDNF | |
| | VKFFKVLNRKTYLNFDKAVFKINIVPKDNYTIYDG | |
| | FNLRNTNLAANFNGQNTEINNMNFTKLSNFTGVFE | |
| | FYKLLCVRGTITSKDKSLDKGYNK | |
| gg_7_13r | PFVNKQFNYKDPVNGVDIAYIKIPNAGQMEPVKAF | Q30E \| S99E \| L276A \| |
| 2 | KIHNKIWVIPERDTFTNPEEGDLNPPPEAKQVPVSY | N279K \| L283E \| |
| (SEQ ID | YDSTYLSTDNEKDNYLKGVTKLFERIYETDLGRM | S323D \| V381D \| |
| NO: 105) | LLTSIVRGIPFWGGSTIDTELKVIDTNCINVIQPDGS | K416S \| L421V \| |
| | YRSEELNLVIIGPSADIIQFECKSFGHEVLNLTRNGY | I433T \| T438D |
| | GSTQYIRFSPDFTFGFEESLEVDTNPLLGAGKFATD | |
| | PAVTLAHELIHAGHRLYGIAINPNRVFKVNTNAYY | |
| | EMSGLEVSFEELRTFGGHDAKFIDSAQEKEFREYY | |
| | YNKFKDIASTLNKAKSIVGTTASLQYMKNVFKEK | |
| | YLLDEDTSGKFSVDKLKFDKLYKMLTEIYTEDNFV | |
| | KFFKVLNRKTYLNFDKAVFKINIVPKDNYTIYDGF | |
| | NLRNTNLAANFNGQNTEINNMNFTKLSNFTGVFEF | |
| | YKLLCVRGTITSKDKSLDKGYNK | |
| gg_25_13 | PFVNKQFNYKDPVNGVDIAYIKIPNAGQMQPVKA | S99E \| L199T \| |
| (SEQ ID | FKIHNKIWVIPERDTFTNPEEGDLNPPPEAKQVPVS | F242T \| N279K \| |
| NO: 106) | YYDSTYLSTDNEKDNYLKGVTKLFERIYETDLGR | L283E \| S323D \| |
| | MLLTSIVRGIPFWGGSTIDTELKVIDTNCINVIQPDG | L335D \| L360Q \| |
| | SYRSEELNLVIIGPSADIIQFECKSFGHEVLNLTRNG | V381D \| K416S \| |
| | YGSTQYIRFSPDFTFGFEESTEVDTNPLLGAGKFAT | L421V \| I433T \| |
| | DPAVTLAHELIHAGHRLYGIAINPNRVTKVNTNAY | T438D |
| | YEMSGLEVSFEELRTFGGHDAKFIDSLQEKEFREY | |
| | YYNKFKDIASTLNKAKSIVGTTASLQYMKNVFKE | |
| | KYLLDEDTSGKFSVDKDKFDKLYKMLTEIYTEDN | |
| | FVKFFKVQNRKTYLNFDKAVFKINIVPKDNYTIYD | |
| | GFNLRNTNLAANFNGQNTEINNMNFTKLSNFTGV | |
| | FEFYKLLCVRGTITSKDKSLDKGYNK | |
| gg_37_14 | PFVNKQFNYKDPVNGVDIAYIKIPNAGQMQPVKA | S99E \| L199T \| |
| (SEQ ID | FKIHNKIWVIPERDTFTNPEEGDLNPPPEAKQVPVS | F242T \| N279K \| |
| NO: 107) | YYDSTYLSTDNEKDNYLKGVTKLFERIYETDLGR | L283E \| L321K \| |
| | MLLTSIVRGIPFWGGSTIDTELKVIDTNCINVIQPDG | S323D \| L335D \| |
| | SYRSEELNLVIIGPSADIIQFECKSFGHEVLNLTRNG | V354A \| L360Q \| |
| | YGSTQYIRFSPDFTFGFEESTEVDTNPLLGAGKFAT | V381D \| I385V |
| | DPAVTLAHELIHAGHRLYGIAINPNRVTKVNTNAY | I433T \| T438D |
| | YEMSGLEVSFEELRTFGGHDAKFIDSLQEKEFREY | |
| | YYNKFKDIASTLNKAKSIVGTTASLQYMKNVFKE | |
| | KYKLDEDTSGKFSVDKDKFDKLYKMLTEIYTEDN | |
| | FAKFFKVQNRKTYLNFDKAVFKINIVPKDNYTVY | |
| | DGFNLRNTNLAANFNGQNTEINNMNFTKLKNFTG | |
| | LFEFYKLLCVRGTITSKDKSLDKGYNK | |
| gg_33_14 | PFVNKQFNYKDPVNGVDIAYIKIPNAGQMEPVKAF | Q30E \| S99E \| L199T \| |
| (SEQ ID | KIHNKIWVIPERDTFTNPEEGDLNPPPEAKQVPVSY | F242T \| L276A \| |
| NO: 108) | YDSTYLSTDNEKDNYLKGVTKLFERIYETDLGRM | L283E \| L321K \| |
| | LLTSIVRGIPFWGGSTIDTELKVIDTNCINVIQPDGS | S323D \| L335D \| |
| | YRSEELNLVIIGPSADIIQFECKSFGHEVLNLTRNGY | L360Q \| V381D \| |
| | GSTQYIRFSPDFTFGFEESTEVDTNPLLGAGKFATD | K416S \| I433T \| |
| | PAVTLAHELIHAGHRLYGIAINPNRVTKVNTNAYY | T438D |
| | EMSGLEVSFEELRTFGGHDAKFIDSAQENEFREYY | |
| | YNKFKDIASTLNKAKSIVGTTASLQYMKNVFKEK | |
| | YKLDEDTSGKFSVDKDKFDKLYKMLTEIYTEDNF | |
| | VKFFKVQNRKTYLNFDKAVFKINIVPKDNYTIYDG | |
| | FNLRNTNLAANFNGQNTEINNMNFTKLSNFTGLFE | |
| | FYKLLCVRGTITSKDKSLDKGYNK | |
| gg_65 | PFVNKQFNYKDPVNGVDIAYIKIPNAGQMEPVKAF | Q30E \| S99E \| L199T \| |
| (SEQ ID | KIHNKIWVIPERDTFTNPEEGDLNPPPEAKQVPVSY | F242T \| L276A \| |
| NO: 109) | YDSTYLSTDNEKDNYLKGVTKLFERIYETDLGRM | L283E \| L321K \| |
| | LLTSIVRGIPFWGGSTIDTELKVIDTNCINVIQPDGS | L335D \| V354A \| |
| | YRSEELNLVIIGPSADIIQFECKSFGHEVLNLTRNGY | L360Q \| F368D \| |
| | GSTQYIRFSPDFTFGFEESTEVDTNPLLGAGKFATD | V381D \| Y386S \| |
| | PAVTLAHELIHAGHRLYGIAINPNRVTKVNTNAYY | K416S \| L421V \| |
| | EMSGLEVSFEELRTFGGHDAKFIDSAQENEFREYY | I433T \| T438D |
| | YNKFKDIASTLNKAKSIVGTTASLQYMKNVFKEK | |
| | YKLSEDTSGKFSVDKDKFDKLYKMLTEIYTEDNFA | |
| | KFFKVQNRKTYLNDDKAVFKINIVPKDNYTISDGF | |
| | NLRNTNLAANFNGQNTEINNMNFTKLSNFTGVFEF | |
| | YKLLCVRGTITSKDKSLDKGYNK | |

Methods of Use with Botulinum Toxin Serotype a Light Chain (BoNT/A-LC) and Deimmunized Variants Thereof

In one aspect, the disclosure provides a method of treating or preventing a disease or disorder in a subject that would benefit from a therapeutically effective amount of a botulinum toxin, comprising administering a therapeutically effective amount of the deimmunized BoNT/A-LC or fragment thereof of the disclosure to the subject. Diseases or disorders that may be treated or prevented with a botulinum toxin include diseases or disorders of inappropriate muscle contraction and/or diseases or disorders of inappropriate neuron signaling.

In certain embodiments, the disease or disorder is selected from the group consisting of acute pain, alopecia, aquagenic keratoderma, atrial fibrillation, blepharospasm, bromhidrosis, cerebral palsy, cervical dystonia, chromhidrosis, chronic anal fissures, chronic pain, constipation, depression, dermatosis, eccrine nevus, eczema, esophageal spasms, essential tremor, facial erythema and flushing, genodermatoses, Hailey-Hailey disease, hand dystonia, hemifacial spasm, hidradenitis suppurativa, hyperhydrosis, hypersialorrhoea, hypertrophic scars, keloids, linear IgA bullous dermatosis, migraine headache, notalgia paresthetica, oily skin, postherpetic neuralgia, psoriasis, overactive bladder, premature ejaculation, Raynaud's Disease, spastic paresis, strabismus, tension headache, voice abnormalities, whiplash.

In another aspect, the disclosure provides a method of treating a subject for a cosmetic purpose, comprising administering a therapeutically effective amount of the deimmunized BoNT/A-LC or fragment thereof of the disclosure to the subject.

In certain embodiments, the cosmetic purpose is the reduction of facial wrinkles. In certain embodiments, the facial wrinkles comprise brow line wrinkles and glabellar frown lines.

In yet another aspect, the disclosure provides method for reducing an antibody response against BoNT/A-LC or fragment thereof in a subject, comprising administering a therapeutically effective amount of the deimmunized BoNT/A-LC or fragment thereof of the disclosure to the subject.

### Botulinum Toxin Serotype B-G Light Chain (BoNT/B-G-LC) and Deimmunized Variants Thereof

Deimmunized variants of the other botulinum toxin serotype light chains are also described herein.

As used herein, the term “botulinum neurotoxin serotype B light chain” or “BoNT/B-LC” or “BLC” refers to the wild type amino acid sequence represented by SEQ ID NO: 2, reproduced below. Specific mutation positions in the BoNT/B-LC amino acid sequence are in reference to SEQ ID NO: 2.

(SEQ ID NO: 2)
PVTINNFNYNDPIDNNNIIMMEPPFARGTGRYYKAFKITDRIWIIPERY
TFGYKPEDFNKSSGIFNRDVCEYYDPDYLNTNDKKNIFLQTMIKLFNRI
KSKPLGEKLLEMIINGIPYLGDRRVPLEEFNTNIASVTVNKLISNPGEV
ERKKGIFANLIIFGPGPVLNENETIDIGIQNHFASREGFGGIMQMKFCP
EYVSVFNNVQENKGASIFNRRGYFSDPALILMHELIHVLHGLYGIKVDD
LPIVPNEKKFFMQSTDAIQAEELYTFGGQDPSIITPSTDKSIYDKVLQN
FRGIVDRLNKVLVCISDPNININIYKNKFKDKYKFVEDSEGKYSIDVES
FDKLYKSLMFGFTETNIAENYKIKTRASYFSDSLPPVKIKNLLDNEIYT
IEEGFNISDKDMEKEYRGQNKAINKQAYEEISKEHLAVYKIQMCKSVKA
PGICID

In certain embodiments, the deimmunized BoNT/B-LC or fragment thereof comprises or consists of a mutation at one or more of N16, R31, D82, S100, L140, L157, Q191, S200, I232, I241, P247, Q264, C308, N317, E342, A361, K367, P379, E389, E394, E421 of SEQ ID NO: 2.

In certain embodiments, the mutation comprises or consists of N16R; R31E; D82A; S100E; L140K; L157V; Q191I; S200N; I232T; I241T; P247E; Q264K; C308T; N317D; E342N; A361S; K367Q; P379G; E389D; E394K; E421D; or a combination thereof, of SEQ ID NO: 2.

In certain embodiments, the deimmunized BoNT/B-LC or fragment thereof comprises or consists of a mutation at one or more of R31, S100, L140, L157, I232, I241, Q264, N317, A361, K367, P379, E389, and E394, of SEQ ID NO: 2.

In certain embodiments, the mutation comprises or consists of R31E; S100E; L140K; L157V; I232T; I241T; Q264K; N317D; A361S; K367Q; P379G; E389D; E394K; or a combination thereof, of SEQ ID NO: 2.

In certain embodiments, the deimmunized BoNT/B-LC or fragment thereof comprises or consists of a mutation at N16 of SEQ ID NO: 2. In certain embodiments, the deimmunized BoNT/B-LC or fragment thereof comprises or consists of a N16R mutation in SEQ ID NO: 2. In certain embodiments, the deimmunized BoNT/B-LC or fragment thereof comprises or consists of a mutation at N16 and R31 of SEQ ID NO: 2. In certain embodiments, the deimmunized BoNT/B-LC or fragment thereof comprises or consists of a N16R and R31E mutation in SEQ ID NO: 2. In certain embodiments, the deimmunized BoNT/B-LC or fragment thereof comprises or consists of a mutation at R31 of SEQ ID NO: 2. In certain embodiments, the deimmunized BoNT/B-LC or fragment thereof comprises or consists of an R31E mutation in SEQ ID NO: 2. In certain embodiments, the deimmunized BoNT/B-LC or fragment thereof comprises or consists of a mutation at D82 of SEQ ID NO: 2. In certain embodiments, the deimmunized BoNT/B-LC or fragment thereof comprises or consists of an D82A mutation in SEQ ID NO: 2. In certain embodiments, the deimmunized BoNT/B-LC or fragment thereof comprises or consists of a mutation at S100 of SEQ ID NO: 2. In certain embodiments, the deimmunized BoNT/B-LC or fragment thereof comprises or consists of an S100E mutation in SEQ ID NO: 2. In certain embodiments, the deimmunized BoNT/B-LC or fragment thereof comprises or consists of a mutation at L140 of SEQ ID NO: 2. In certain embodiments, the deimmunized BoNT/B-LC or fragment thereof comprises or consists of an L140K mutation in SEQ ID NO: 2. In certain embodiments, the deimmunized BoNT/B-LC or fragment thereof comprises or consists of a mutation at L140 and L157 of SEQ ID NO: 2. In certain embodiments, the deimmunized BoNT/B-LC or fragment thereof comprises or consists of an L140K and L157V mutation in SEQ ID NO: 2. In certain embodiments, the deimmunized BoNT/B-LC or fragment thereof comprises or consists of a mutation at L157 of SEQ ID NO: 2. In certain embodiments, the deimmunized BoNT/B-LC or fragment thereof comprises or consists of an L157V mutation in SEQ ID NO: 2. In certain embodiments, the deimmunized BoNT/B-LC or fragment thereof comprises or consists of a mutation at Q191 of SEQ ID NO: 2. In certain embodiments, the deimmunized BoNT/B-LC or fragment thereof comprises or consists of an Q191I mutation in SEQ ID NO: 2. In certain embodiments, the deimmunized BoNT/B-LC or fragment thereof comprises or consists of a mutation at S200 of SEQ ID NO: 2. In certain embodiments, the deimmunized BoNT/B-LC or fragment thereof comprises or consists of an S200N mutation in SEQ ID NO: 2. In certain embodiments, the deimmunized BoNT/B-LC or fragment thereof comprises or consists of a mutation at 1232 of SEQ ID NO: 2. In certain embodiments, the deimmunized BoNT/B-LC or fragment thereof comprises or consists of an I232T mutation in SEQ ID NO: 2. In certain embodiments, the deimmunized BoNT/B-LC or fragment thereof comprises or consists of a mutation at 1232 and 1241 of SEQ ID NO: 2. In certain embodiments, the deimmunized BoNT/B-LC or fragment thereof comprises or consists of an I232T and 1241T mutation in SEQ ID NO: 2. In certain embodiments, the deimmunized BoNT/B-LC or fragment thereof comprises or consists of a mutation at 1241 of SEQ ID NO: 2. In certain embodiments, the deimmunized BoNT/B-LC or fragment thereof comprises or consists of an 1241T mutation in SEQ ID NO: 2. In certain embodiments, the deimmunized BoNT/B-LC or fragment thereof comprises or consists of a mutation at 1241 and P247 of SEQ ID NO: 2. In certain embodiments, the deimmunized BoNT/B-LC or fragment thereof comprises or consists of an 1241T and P247E mutation in SEQ ID NO: 2. In certain embodiments, the deimmunized BoNT/B-LC or fragment thereof comprises or consists of a mutation at P247 of SEQ ID NO: 2. In certain embodiments, the deimmunized BoNT/B-LC or fragment thereof comprises or consists of an P247E mutation in SEQ ID NO: 2. In certain embodiments, the deimmunized BoNT/B-LC or fragment thereof comprises or consists of a mutation at Q264 of SEQ ID NO: 2. In certain embodiments, the deimmunized BoNT/B-LC or fragment thereof comprises or consists of an Q264K mutation in SEQ ID NO: 2. In certain embodiments, the deimmunized BoNT/B-LC or fragment thereof comprises or consists of a mutation at C308 of SEQ ID NO: 2. In certain embodiments, the deimmunized BoNT/B-LC or fragment thereof comprises or consists of an C308T mutation in SEQ ID NO: 2. In certain embodiments, the deimmunized BoNT/B-LC or fragment thereof comprises or consists of a mutation at C308 and N317 of SEQ ID NO: 2. In certain embodiments, the deimmunized BoNT/B-LC or fragment thereof comprises or consists of an C308T and N317D mutation in SEQ ID NO: 2. In certain embodiments, the deimmunized BoNT/B-LC or fragment thereof comprises or consists of a mutation at N317 of SEQ ID NO: 2. In certain embodiments, the deimmunized BoNT/B-LC or fragment thereof comprises or consists of an N317D mutation in SEQ ID NO: 2. In certain embodiments, the deimmunized BoNT/B-LC or fragment thereof comprises or consists of a mutation at E342 of SEQ ID NO: 2. In certain embodiments, the deimmunized BoNT/B-LC or fragment thereof comprises or consists of an E342N mutation in SEQ ID NO: 2. In certain embodiments, the deimmunized BoNT/B-LC or fragment thereof comprises or consists of a mutation at A361 of SEQ ID NO: 2. In certain embodiments, the deimmunized BoNT/B-LC or fragment thereof comprises or consists of an A361S mutation in SEQ ID NO: 2. In certain embodiments, the deimmunized BoNT/B-LC or fragment thereof comprises or consists of a mutation at A361 and K367 of SEQ ID NO: 2. In certain embodiments, the deimmunized BoNT/B-LC or fragment thereof comprises or consists of an A361S and K367Q mutation in SEQ ID NO: 2. In certain embodiments, the deimmunized BoNT/B-LC or fragment thereof comprises or consists of a mutation at K367 of SEQ ID NO: 2. In certain embodiments, the deimmunized BoNT/B-LC or fragment thereof comprises or consists of an K367Q mutation in SEQ ID NO: 2. In certain embodiments, the deimmunized BoNT/B-LC or fragment thereof comprises or consists of a mutation at K367 and P379 of SEQ ID NO: 2. In certain embodiments, the deimmunized BoNT/B-LC or fragment thereof comprises or consists of an K367Q and P379G mutation in SEQ ID NO: 2. In certain embodiments, the deimmunized BoNT/B-LC or fragment thereof comprises or consists of a mutation at P379 of SEQ ID NO: 2. In certain embodiments, the deimmunized BoNT/B-LC or fragment thereof comprises or consists of an P379G mutation in SEQ ID NO: 2. In certain embodiments, the deimmunized BoNT/B-LC or fragment thereof comprises or consists of a mutation at P379 and E389 of SEQ ID NO: 2. In certain embodiments, the deimmunized BoNT/B-LC or fragment thereof comprises or consists of an P379G and E389D mutation in SEQ ID NO: 2. In certain embodiments, the deimmunized BoNT/B-LC or fragment thereof comprises or consists of a mutation at E389 of SEQ ID NO: 2. In certain embodiments, the deimmunized BoNT/B-LC or fragment thereof comprises or consists of an E389D mutation in SEQ ID NO: 2. In certain embodiments, the deimmunized BoNT/B-LC or fragment thereof comprises or consists of a mutation at E389 and E394 of SEQ ID NO: 2. In certain embodiments, the deimmunized BoNT/B-LC or fragment thereof comprises or consists of an E389D and E394K mutation in SEQ ID NO: 2. In certain embodiments, the deimmunized BoNT/B-LC or fragment thereof comprises or consists of a mutation at E394 of SEQ ID NO: 2. In certain embodiments, the deimmunized BoNT/B-LC or fragment thereof comprises or consists of an E394K mutation in SEQ ID NO: 2. In certain embodiments, the deimmunized BoNT/B-LC or fragment thereof comprises or consists of a mutation at E421 of SEQ ID NO: 2. In certain embodiments, the deimmunized BoNT/B-LC or fragment thereof comprises or consists of an E421D mutation in SEQ ID NO: 2.

As used herein, the term “botulinum neurotoxin serotype C light chain” or “BoNT/C-LC” or “CLC” refers to the wild type amino acid sequence represented by SEQ ID NO: 3, reproduced below. Specific mutation positions in the BoNT/C-LC amino acid sequence are in reference to SEQ ID NO: 3.

(SEQ ID NO: 3)
PITINNFNYSDPVDNKNILYLDTHLNTLANEPEKAFRITGNIWVIPDRF
SRNSNPNLNKPPRVTSPKSGYYDPNYLSTDSDKDTFLKEIIKLFKRINS
REIGEELIYRLSTDIPFPGNNNTPINTFDFDVDFNSVDVKTRQGNNWVK
TGSINPSVIITGPRENIIDPETSTFKLTNNTFAAQEGFGALSIISISPR
FMLTYSNATNDVGEGRFSKSEFCMDPILILMHELNHAMHNLYGIAIPND
QTISSVTSNIFYSQYNVKLEYAEIYAFGGPTIDLIPKSARKYFEEKALD
YYRSIAKRLNSITTANPSSFNKYIGEYKQKLIRKYRFVVESSGEVTVNR
NKFVELYNELTQIFTEFNYAKIYNVQNRKIYLSNVYTPVTANILDDNVY
DIQNGFNIPKSNLNVLFMGQNLSRNPALRKVNPENMLYLFTKFCHKAID
GRSLYNKTLDCR

In certain embodiments, the deimmunized BoNT/C-LC or fragment thereof comprises or consists of a mutation at one or more of K16, S80, S98, R161, L199, F218, N231, I240, T247, E265, E290, A309, G319, R33K, A363, P381, N390, Q395, and R421 of SEQ ID NO: 3.

In certain embodiments, the mutation comprises or consists of K16R; S80A; S98E; R161G; L199N; F218Y; N231T; I240T; T247E; E265K; E290D; A309T; G319D; R330K; A363S; P381G; N390D; Q395K; R421D; or a combination thereof, of SEQ ID NO: 3.

In certain embodiments, the deimmunized BoNT/C-LC or fragment thereof comprises or consists of a mutation at one or more of S98, L199, F218, I240, A309, G319, R33K, A363, P381, and R421 of SEQ ID NO: 3.

In certain embodiments, the mutation comprises or consists of S98E; L199N; F218Y; I240T; A309T; G319D; R330K; A363S; P381G; R421D; or a combination thereof, of SEQ ID NO: 3.

In certain embodiments, the deimmunized BoNT/C-LC or fragment thereof comprises or consists of a mutation at K16 of SEQ ID NO: 3. In certain embodiments, the deimmunized BoNT/C-LC or fragment thereof comprises or consists of a K16R mutation in SEQ ID NO: 3. In certain embodiments, the deimmunized BoNT/C-LC or fragment thereof comprises or consists of a mutation at S80 of SEQ ID NO: 3. In certain embodiments, the deimmunized BoNT/C-LC or fragment thereof comprises or consists of a S80A mutation in SEQ ID NO: 3. In certain embodiments, the deimmunized BoNT/C-LC or fragment thereof comprises or consists of a mutation at S98 of SEQ ID NO: 3. In certain embodiments, the deimmunized BoNT/C-LC or fragment thereof comprises or consists of a S98E mutation in SEQ ID NO: 3. In certain embodiments, the deimmunized BoNT/C-LC or fragment thereof comprises or consists of a mutation at R161 of SEQ ID NO: 3. In certain embodiments, the deimmunized BoNT/C-LC or fragment thereof comprises or consists of a R161G mutation in SEQ ID NO: 3. In certain embodiments, the deimmunized BoNT/C-LC or fragment thereof comprises or consists of a mutation at L199 of SEQ ID NO: 3. In certain embodiments, the deimmunized BoNT/C-LC or fragment thereof comprises or consists of a L199N mutation in SEQ ID NO: 3. In certain embodiments, the deimmunized BoNT/C-LC or fragment thereof comprises or consists of a mutation at F218 of SEQ ID NO: 3. In certain embodiments, the deimmunized BoNT/C-LC or fragment thereof comprises or consists of a F218Y mutation in SEQ ID NO: 3. In certain embodiments, the deimmunized BoNT/C-LC or fragment thereof comprises or consists of a mutation at F218 and N231 of SEQ ID NO: 3. In certain embodiments, the deimmunized BoNT/C-LC or fragment thereof comprises or consists of a F218Y and N231T mutation in SEQ ID NO: 3. In certain embodiments, the deimmunized BoNT/C-LC or fragment thereof comprises or consists of a mutation at N231 of SEQ ID NO: 3. In certain embodiments, the deimmunized BoNT/C-LC or fragment thereof comprises or consists of a N231T mutation in SEQ ID NO: 3. In certain embodiments, the deimmunized BoNT/C-LC or fragment thereof comprises or consists of a mutation at N231 and I240 of SEQ ID NO: 3. In certain embodiments, the deimmunized BoNT/C-LC or fragment thereof comprises or consists of a N231T and I240T mutation in SEQ ID NO: 3. In certain embodiments, the deimmunized BoNT/C-LC or fragment thereof comprises or consists of a mutation at I240 of SEQ ID NO: 3. In certain embodiments, the deimmunized BoNT/C-LC or fragment thereof comprises or consists of a I240T mutation in SEQ ID NO: 3. In certain embodiments, the deimmunized BoNT/C-LC or fragment thereof comprises or consists of a mutation at I240 and T247 of SEQ ID NO: 3. In certain embodiments, the deimmunized BoNT/C-LC or fragment thereof comprises or consists of a I240T and T247E mutation in SEQ ID NO: 3. In certain embodiments, the deimmunized BoNT/C-LC or fragment thereof comprises or consists of a mutation at T247 of SEQ ID NO: 3. In certain embodiments, the deimmunized BoNT/C-LC or fragment thereof comprises or consists of a T247E mutation in SEQ ID NO: 3. In certain embodiments, the deimmunized BoNT/C-LC or fragment thereof comprises or consists of a mutation at E265 of SEQ ID NO: 3. In certain embodiments, the deimmunized BoNT/C-LC or fragment thereof comprises or consists of a E265K mutation in SEQ ID NO: 3. In certain embodiments, the deimmunized BoNT/C-LC or fragment thereof comprises or consists of a mutation at A309 of SEQ ID NO: 3. In certain embodiments, the deimmunized BoNT/C-LC or fragment thereof comprises or consists of a A309T mutation in SEQ ID NO: 3. In certain embodiments, the deimmunized BoNT/C-LC or fragment thereof comprises or consists of a mutation at G319 of SEQ ID NO: 3. In certain embodiments, the deimmunized BoNT/C-LC or fragment thereof comprises or consists of a G319D mutation in SEQ ID NO: 3. In certain embodiments, the deimmunized BoNT/C-LC or fragment thereof comprises or consists of a mutation at G319 and R330 of SEQ ID NO: 3. In certain embodiments, the deimmunized BoNT/C-LC or fragment thereof comprises or consists of a G319D and R330K mutation in SEQ ID NO: 3. In certain embodiments, the deimmunized BoNT/C-LC or fragment thereof comprises or consists of a mutation at R330 of SEQ ID NO: 3. In certain embodiments, the deimmunized BoNT/C-LC or fragment thereof comprises or consists of a R330K mutation in SEQ ID NO: 3. In certain embodiments, the deimmunized BoNT/C-LC or fragment thereof comprises or consists of a mutation at P381 of SEQ ID NO: 3. In certain embodiments, the deimmunized BoNT/C-LC or fragment thereof comprises or consists of a P381G mutation in SEQ ID NO: 3. In certain embodiments, the deimmunized BoNT/C-LC or fragment thereof comprises or consists of a mutation at P381 and N390 of SEQ ID NO: 3. In certain embodiments, the deimmunized BoNT/C-LC or fragment thereof comprises or consists of a P381G and N390D mutation in SEQ ID NO: 3. In certain embodiments, the deimmunized BoNT/C-LC or fragment thereof comprises or consists of a mutation at N390 of SEQ ID NO: 3. In certain embodiments, the deimmunized BoNT/C-LC or fragment thereof comprises or consists of a N390D mutation in SEQ ID NO: 3. In certain embodiments, the deimmunized BoNT/C-LC or fragment thereof comprises or consists of a mutation at N390 and Q395 of SEQ ID NO: 3. In certain embodiments, the deimmunized BoNT/C-LC or fragment thereof comprises or consists of a N390D and Q395K mutation in SEQ ID NO: 3. In certain embodiments, the deimmunized BoNT/C-LC or fragment thereof comprises or consists of a mutation at Q395 of SEQ ID NO: 3. In certain embodiments, the deimmunized BoNT/C-LC or fragment thereof comprises or consists of a Q395K mutation in SEQ ID NO: 3. In certain embodiments, the deimmunized BoNT/C-LC or fragment thereof comprises or consists of a mutation at R421 of SEQ ID NO: 3. In certain embodiments, the deimmunized BoNT/C-LC or fragment thereof comprises or consists of a R421D mutation in SEQ ID NO: 3.

As used herein, the term “botulinum neurotoxin serotype D light chain” or “BoNT/D-LC” or “DLC” refers to the wild type amino acid sequence represented by SEQ ID NO: 4, reproduced below. Specific mutation positions in the BoNT/D-LC amino acid sequence are in reference to SEQ ID NO: 4.

(SEQ ID NO: 4)
TWPVKDFNYSDPVNDNDILYLRIPQNKLITTPVKAFMITQNIWVIPERF
SSDTNPSLSKPPRPTSKYQSYYDPSYLSTDEQKDTFLKGIIKLFKRINE
RDIGKKLINYLVVGSPFMGDSSTPEDTFDFTRHTTNIAVEKFENGSWKV
TNIITPSVLIFGPLPNILDYTASLTLQGQQSNPSFEGFGTLSILKVAPE
FLLTFSDVTSNQSSAVLGKSIFCMDPVIALMHELTHSLHQLYGINIPSD
KRIRPQVSEGFFSQDGPNVQFEELYTFGGLDVEIIPQIERSQLREKALG
HYKDIAKRLNNINKTIPSSWISNIDKYKKIFSEKYNFDKDNTGNFVVNI
DKFNSLYSDLTNVMSEVVYSSQYNVKNRTHYFSRHYLPVFANILDDNIY
TIRDGFNLTNKGFNIENSGQNIERNPALQKLSSESVVDLFTKVCLRLTK
NSRDDSTCIK

In certain embodiments, the deimmunized BoNT/D-LC or fragment thereof comprises or consists of a mutation at one or more of N16, T31, E80, E138, L161, L199, F218, 1240, R247, Q265, E290, N330, D344, K369, P381, N390, R395, and Q421 of SEQ ID NO: 4.

In certain embodiments, the mutation comprises or consists of N16R; T31E; E80A; E138K; L161G; L199N; F218Y; 1240T; R247E; Q265K; E290D; N330K; D344N; K369Q; P381G; N390D; R395K; Q421D; or a combination thereof, of SEQ ID NO: 4.

In certain embodiments, the deimmunized BoNT/D-LC or fragment thereof comprises or consists of a mutation at one or more of T31, L161, L199, F218, T231, I240, R247, E290, P381, N390, R395, and Q421 of SEQ ID NO: 4.

In certain embodiments, the mutation comprises or consists of T31E; L161G; L199N; F218Y; T231T; 1240T; R247E; E290D; P381G; N390D; R395K; Q421D; or a combination thereof, of SEQ ID NO: 4.

In certain embodiments, the deimmunized BoNT/D-LC or fragment thereof comprises or consists of a mutation at N16 of SEQ ID NO: 4. In certain embodiments, the deimmunized BoNT/D-LC or fragment thereof comprises or consists of a N16R mutation in SEQ ID NO: 4. In certain embodiments, the deimmunized BoNT/D-LC or fragment thereof comprises or consists of a mutation at T31 of SEQ ID NO: 4. In certain embodiments, the deimmunized BoNT/D-LC or fragment thereof comprises or consists of a T31E mutation in SEQ ID NO: 4. In certain embodiments, the deimmunized BoNT/D-LC or fragment thereof comprises or consists of a mutation at E80 of SEQ ID NO: 4. In certain embodiments, the deimmunized BoNT/D-LC or fragment thereof comprises or consists of a E80A mutation in SEQ ID NO: 4. In certain embodiments, the deimmunized BoNT/D-LC or fragment thereof comprises or consists of a mutation at E80 of SEQ ID NO: 4. In certain embodiments, the deimmunized BoNT/D-LC or fragment thereof comprises or consists of a E80A mutation in SEQ ID NO: 4. In certain embodiments, the deimmunized BoNT/D-LC or fragment thereof comprises or consists of a mutation at E138 of SEQ ID NO: 4. In certain embodiments, the deimmunized BoNT/D-LC or fragment thereof comprises or consists of a E138K mutation in SEQ ID NO: 4. In certain embodiments, the deimmunized BoNT/D-LC or fragment thereof comprises or consists of a mutation at L161 of SEQ ID NO: 4. In certain embodiments, the deimmunized BoNT/D-LC or fragment thereof comprises or consists of a L161G mutation in SEQ ID NO: 4. In certain embodiments, the deimmunized BoNT/D-LC or fragment thereof comprises or consists of a mutation at L199 of SEQ ID NO: 4. In certain embodiments, the deimmunized BoNT/D-LC or fragment thereof comprises or consists of a L199N mutation in SEQ ID NO: 4. In certain embodiments, the deimmunized BoNT/D-LC or fragment thereof comprises or consists of a mutation at F218 of SEQ ID NO: 4. In certain embodiments, the deimmunized BoNT/D-LC or fragment thereof comprises or consists of a F218Y mutation in SEQ ID NO: 4. In certain embodiments, the deimmunized BoNT/D-LC or fragment thereof comprises or consists of a mutation at 1240 of SEQ ID NO: 4. In certain embodiments, the deimmunized BoNT/D-LC or fragment thereof comprises or consists of a 1240T mutation in SEQ ID NO: 4. In certain embodiments, the deimmunized BoNT/D-LC or fragment thereof comprises or consists of a mutation at R247 of SEQ ID NO: 4. In certain embodiments, the deimmunized BoNT/D-LC or fragment thereof comprises or consists of a R247E mutation in SEQ ID NO: 4. In certain embodiments, the deimmunized BoNT/D-LC or fragment thereof comprises or consists of a mutation at Q265 of SEQ ID NO: 4. In certain embodiments, the deimmunized BoNT/D-LC or fragment thereof comprises or consists of a Q265K mutation in SEQ ID NO: 4. In certain embodiments, the deimmunized BoNT/D-LC or fragment thereof comprises or consists of a mutation at E290 of SEQ ID NO: 4. In certain embodiments, the deimmunized BoNT/D-LC or fragment thereof comprises or consists of a E290D mutation in SEQ ID NO: 4. In certain embodiments, the deimmunized BoNT/D-LC or fragment thereof comprises or consists of a mutation at E330 of SEQ ID NO: 4. In certain embodiments, the deimmunized BoNT/D-LC or fragment thereof comprises or consists of a E330K mutation in SEQ ID NO: 4. In certain embodiments, the deimmunized BoNT/D-LC or fragment thereof comprises or consists of a mutation at D344 of SEQ ID NO: 4. In certain embodiments, the deimmunized BoNT/D-LC or fragment thereof comprises or consists of a D344N mutation in SEQ ID NO: 4. In certain embodiments, the deimmunized BoNT/D-LC or fragment thereof comprises or consists of a mutation at K369 of SEQ ID NO: 4. In certain embodiments, the deimmunized BoNT/D-LC or fragment thereof comprises or consists of a K369Q mutation in SEQ ID NO: 4. In certain embodiments, the deimmunized BoNT/D-LC or fragment thereof comprises or consists of a mutation at P381 of SEQ ID NO: 4. In certain embodiments, the deimmunized BoNT/D-LC or fragment thereof comprises or consists of a P381G mutation in SEQ ID NO: 4. In certain embodiments, the deimmunized BoNT/D-LC or fragment thereof comprises or consists of a mutation at N390 of SEQ ID NO: 4. In certain embodiments, the deimmunized BoNT/D-LC or fragment thereof comprises or consists of a N390D mutation in SEQ ID NO: 4. In certain embodiments, the deimmunized BoNT/D-LC or fragment thereof comprises or consists of a mutation at R395 of SEQ ID NO: 4. In certain embodiments, the deimmunized BoNT/D-LC or fragment thereof comprises or consists of a R395K mutation in SEQ ID NO: 4. In certain embodiments, the deimmunized BoNT/D-LC or fragment thereof comprises or consists of a mutation at Q421 of SEQ ID NO: 4. In certain embodiments, the deimmunized BoNT/D-LC or fragment thereof comprises or consists of a Q421D mutation in SEQ ID NO: 4.

As used herein, the term “botulinum neurotoxin serotype E light chain” or “BoNT/E-LC” or “ELC” refers to the wild type amino acid sequence represented by SEQ ID NO: 5, reproduced below. Specific mutation positions in the BoNT/E-LC amino acid sequence are in reference to SEQ ID NO: 5.

(SEQ ID NO: 5)
PKINSFNYNDPVNDRTILYIKPGGCQEFYKSFNIMKNIWIIPERNVIGT
TPQDFHPPTSLKNGDSSYYDPNYLQSDEEKDRFLKIVTKIFNRINNNLS
GGILLEELSKANPYLGNDNTPDNQFHIGDASAVEIKFSNGSQDILLPNV
IIMGAEPDLFETNSSNISLRNNYMPSNHRFGSIAIVTFSPEYSFRFNDN
CMNEFIQDPALTLMHELIHSLHGLYGAKGITTKYTITQKQNPLITNIRG
TNIEEFLTFGGTDLNIITSAQSNDIYTNLLADYKKIASKLSKVQVSNPL
LNPYKDVFEAKYGLDKDASGIYSVNINKFNDIFKKLYSFTEFDLRTKFQ
VKCRQTYIGQYKYFKLSNLLNDSIYNISEGYNINNLKVNFRGQNANLNP
RIITPITGRGLVKKIIRFCKNIVSVKGIRKSICIE

In certain embodiments, the deimmunized BoNT/E-LC or fragment thereof comprises or consists of a mutation at one or more of E77, N95, E153, F191, F201, I214, A223, Y230, N247, T272, N273, S291, N296, G307, R339, K345, Y356, S366, S371, and T396 of SEQ ID NO: 5.

In certain embodiments, the mutation comprises E77A; N95E; E153G; F191N; F201Y; I214T; A223T; Y230E; N247K; T272D; N273K; S291T; N296D; G307K; R339S; K345Q; Y356G; S366D; S371K; T396D; or a combination thereof, of SEQ ID NO: 5.

In certain embodiments, the deimmunized BoNT/E-LC or fragment thereof comprises or consists of a mutation at one or more of N95, E153, F191, F201, I214, A223, Y230, N247, T272, S291, N296, K345, Y356, S366, S371, and T396 of SEQ ID NO: 5.

In certain embodiments, the mutation comprises N95E; E153G; F191N; F201Y; I214T; A223T; Y230E; N247K; T272D; S291T; N296D; K345Q; Y356G; S366D; S371K; T396D; or a combination thereof, of SEQ ID NO: 5.

As used herein, the term “botulinum neurotoxin serotype F light chain” or “BoNT/F-LC” or “FLC” refers to the wild type amino acid sequence represented by SEQ ID NO: 6, reproduced below. Specific mutation positions in the BoNT/F-LC amino acid sequence are in reference to SEQ ID NO: 6.

(SEQ ID NO: 6)
PVAINSFNYNDPVNDDTILYMQIPYEEKSKKYYKAFEIMRNVWIIPERN
TIGTNPSDFDPPASLKNGSSAYYDPNYLTTDAEKDRYLKTTIKLFKRIN
SNPAGKVLLQEISYAKPYLGNDHTPIDEFSPVTRTTSVNIKLSTNVESS
MLLNLLVLGAGPDIFESCCYPVRKLIDPDVVYDPSNYGFGSINIVTFSP
EYEYTFNDISGGHNSSTESFIADPAISLAHELIHALHGLYGARGVTYEE
TIEVKQAPLMIAEKPIRLEEFLTFGGQDLNIITSAMKEKIYNNLLANYE
KIATRLSEVNSAPPEYDINEYKDYFQWKYGLDKNADGSYTVNENKFNEI
YKKLYSFTESDLANKFKVKCRNTYFIKYEFLKVPNLLDDDIYTVSEGFN
IGNLAVNNRGQSIKLNPKIIDSIPDKGLVEKIVKFCKSVIPRKGTKAPP
RLCIR

In certain embodiments, the deimmunized BoNT/F-LC or fragment thereof comprises or consists of a mutation at one or more of D16, K31, S99, L152, Y200, F216, I229, A238, R262, N287, N288, A306, N313, G324, A356, K362, F373, and S388 of SEQ ID NO: 6.

In certain embodiments, the mutation comprises D16R; K31E; S99E; L152V; Y200N; F216Y; 1229T; A238T; R262K; N287D; N288K; A306T; N313D; G324K; A356S; K362Q; F373G; S388K; or a combination thereof, of SEQ ID NO: 6.

In certain embodiments, the deimmunized BoNT/F-LC or fragment thereof comprises or consists of a mutation at one or more of K31, S99, Y200, F216, I229, A238, R262, N287, A306, N313, A356, F373, and S388 of SEQ ID NO: 6.

In certain embodiments, the mutation comprises K31E; S99E; Y200N; F216Y; I229T; A238T; R262K; N287D; A306T; N313D; A356S; F373G; S388K; or a combination thereof, of SEQ ID NO: 6.

As used herein, the term “botulinum neurotoxin serotype G light chain” or “BoNT/G-LC” or “GLC” refers to the wild type amino acid sequence represented by SEQ ID NO: 7, reproduced below. Specific mutation positions in the BoNT/G-LC amino acid sequence are in reference to SEQ ID NO: 7.

(SEQ ID NO: 7)
PVNIKXFNYNDPINNDDIIMMEPFNDPGPGTYYKAFRIIDRIWIVPERF
TYGFQPDQFNASTGVFSKDVYEYYDPTYLKTDAEKDKFLKTMIKLFNRI
NSKPSGQRLLDMIVDAIPYLGNASTPPDKFAANVANVSINKKIIQPGAE
DQIKGLMTNLIIFGPGPVLSDNFTDSMIMNGHSPISEGFGARMMIRFCP
SCLNVFNNVQENKDTSIFSRRAYFADPALTLMHELIHVLHGLYGIKISN
LPITPNTKEFFMQHSDPVQAEELYTFGGHDPSVISPSTDMNIYNKALQN
FQDIANRLNIVSSAQGSGIDISLYKQIYKNKYDFVEDPNGKYSVDKDKF
DKLYKALMFGFTETNLAGEYGIKTRYSYFSEYLPPIKTEKLLDNTIYTQ
NEGFNIASKNLKTEFNGQNKAVNKEAYEEISLEHLVIYRIAMCKPVMYK
NTGKSEQCII

In certain embodiments, the deimmunized BoNT/G-LC or fragment thereof comprises or consists of a mutation at one or more of D16, T31, S100, L157, M191, I232, I241, P247, Q264, N289, A308, S316, D327, D341, A360, K366, P378, T388, N393, and E420 of SEQ ID NO: 7.

In certain embodiments, the mutation comprises D16R; T31E; S100E; L157V; M191I; I232T; I241T; P247E; Q264K; N289D; A308T; S316D; D327K; D341N; A360S; K366Q; P378G; T388D; N393K; E420D; or a combination thereof, of SEQ ID NO: 7.

In certain embodiments, the deimmunized BoNT/G-LC or fragment thereof comprises or consists of a mutation at one or more of D16, T31, S100, L157, I232, I241, P247, Q264, N289, A308, S316, A360, K366, P378, T388, N393, and E420 of SEQ ID NO: 7.

In certain embodiments, the mutation comprises D16R; T31E; S100E; L157V; I232T; I241T; P247E; Q264K; N289D; A308T; S316D; A360S; K366Q; P378G; T388D; N393K; E420D; or a combination thereof, of SEQ ID NO: 7.

In certain embodiments, an N-terminal initiator methionine is present in any of the BoNT/B-LC, BoNT/C-LC, BoNT/D-LC, BoNT/E-LC, BoNT/F-LC, or BoNT/G-LC amino acid sequences. The N-terminal methionine is encoded in the nucleic acids encoding the wild-type and deimmunized BLC, CLC, DLD, ELC, FLC, and GLC variants of the disclosure, however, in some circumstances the N-terminal methionine is removed by the host cell expressing the protein. In certain embodiments, the N-terminal methionine may remain after expression and purification of the deimmunized BLC, CLC, DLD, ELC, FLC, and GLC variants of the disclosure.

As used herein, the term “fragment” in reference to any one of BoNT/B-LC, BoNT/C-LC, BoNT/D-LC, BoNT/E-LC, BoNT/F-LC, or BoNT/G-LC (i.e., BoNT/B-LC, BoNT/C-LC, BoNT/D-LC, BoNT/E-LC, BoNT/F-LC, or BoNT/G-LC fragments), refers to a BoNT/B-LC, BoNT/C-LC, BoNT/D-LC, BoNT/E-LC, BoNT/F-LC, or BoNT/G-LC amino acid sequence that comprises fewer amino acids than the amino acid sequence of SEQ ID NO: 2 (BoNT/B-LC), SEQ ID NO: 3 (BoNT/C-LC), SEQ ID NO: 4 (BoNT/D-LC), SEQ ID NO: 5 (BoNT/E-LC), SEQ ID NO: 6 (BoNT/F-LC), or SEQ ID NO: 7 (BoNT/G-LC). For example, but in no way limiting, a fragment of any one of BoNT/B-LC, BoNT/C-LC, BoNT/D-LC, BoNT/E-LC, BoNT/F-LC, or BoNT/G-LC may have one or more amino acids removed from the N terminus, the C terminus, or internally of SEQ ID NO: 2 (BoNT/B-LC), SEQ ID NO: 3 (BoNT/C-LC), SEQ ID NO: 4 (BoNT/D-LC), SEQ ID NO: 5 (BoNT/E-LC), SEQ ID NO: 6 (BoNT/F-LC), or SEQ ID NO: 7 (BoNT/G-LC). The fragment may have 1, 2, 3, 4, 5, 6, 7, 8, 9, 10, 11, 12, 13, 14, 15, 16, 17, 18, 19, 20, 25, 30, 35, 40, 45, 50, or more amino acids removed from the N terminus, the C terminus, or internally of any one of SEQ ID NO: 2 (BoNT/B-LC), SEQ ID NO: 3 (BoNT/C-LC), SEQ ID NO: 4 (BoNT/D-LC), SEQ ID NO: 5 (BoNT/E-LC), SEQ ID NO: 6 (BoNT/F-LC), or SEQ ID NO: 7 (BoNT/G-LC). The fragment should retain some level of activity of any one of BoNT/B-LC, BoNT/C-LC, BoNT/D-LC, BoNT/E-LC, BoNT/F-LC, or BoNT/G-LC, such as the ability to cleave SNAP-25 and the fragment should contain one or more deimmunizing mutations described herein.

The term “deimmunized” as applied to BoNT/A-LC above, also applies to any one of BoNT/B-LC, BoNT/C-LC, BoNT/D-LC, BoNT/E-LC, BoNT/F-LC, or BoNT/G-LC (e.g., BoNT/B-LC, BoNT/C-LC, BoNT/D-LC, BoNT/E-LC, BoNT/F-LC, or BoNT/G-LC variants, derivatives and/or homologues thereof), wherein the specific removal and/or modification of highly immunogenic regions or residues has occurred. The term “deimmunized” is well-known in the art and, among other things, has been employed for the removal of T-cell epitopes from other therapeutic molecules including antibodies (See, e.g., WO 98/52976 or WO 00/34317).

The following non-limiting examples are provided to further illustrate the present disclosure.

### EXAMPLES

### Example 1—Botulinum Toxin Serotype a Light Chain (BoNT/A-LC) Library Design and General Methods

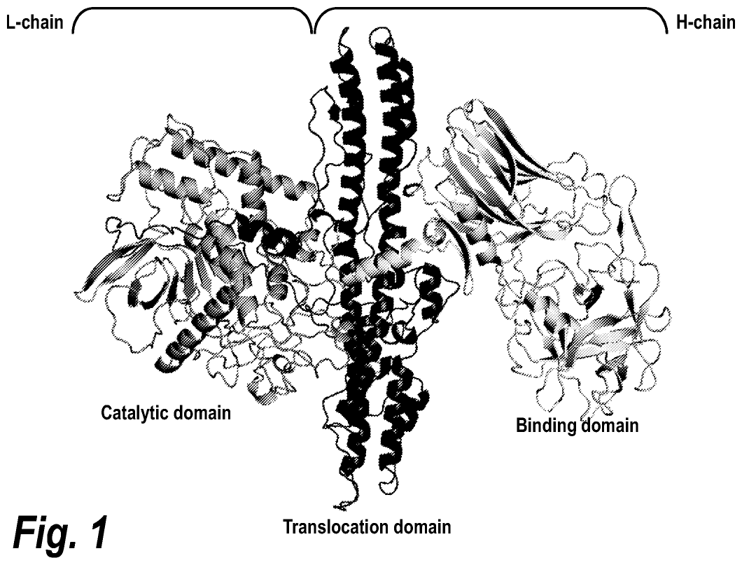

Specific residues that are important to maintain the structural and functional integrity of redesigned BoNT/A-LC molecules were locked down during the library design. To select those specific positions, an x-ray crystal structure of the BoNT/A (PDB id 3 BTA, FIG. 1) was employed to understand the important functional and structural regions of the BoNT/A-LC domain. The BoNT/A-LC and BoNT/A heavy chains (BoNT/A-HC) have a large interacting interface and a peptide belt where the BoNT/A-HC domain wraps around the BoNT/A-LC domain, which also protects the active site. To maintain this structural integrity of the complex, the interface residues of the BoNT/A-LC and the residues interacting with the belt were fixed and not allowed for redesigning. In addition to the locked down residues above, the zinc binding motif residues H222, E223, H226, and E261, and other active site residues of the BoNT/A-LC were also considered non-mutable. Another crystal structure of BoNT/A complexed with the Synaptosomal-associated protein 25 (PDB id 1×TG) was also used for assessing the interactions between the BoNT/A-LC and the synapse peptide, that mainly form hydrophobic interactions. These interacting residues were also not allowed to mutate (Table 4). The residue numbering of Table 4 begins with the N-terminal methionine as residue 1. However, the residue numbering for BoNT/A-LC in all other instances of the disclosure does not include the N-terminal methionine for numbering purposes.

TABLE 4
BoNt/A-LC residues interacting with
other domain of BoNT/A were locked
down during the computational guided
BoNT/A-LC deimmunization design

| Residue | Amino | Active site | Interact with | Interface |
|---|---|---|---|---|
| Number | acid | region | belt/substrate | residues |
| 1 | M | — | yes | yes |
| 22 | I | — | yes | yes |
| 23 | K | — | yes | yes |
| 24 | I | — | yes | yes |
| 25 | P | — | yes | yes |
| 26 | N | — | yes | yes |
| 27 | A | — | yes | yes |
| 28 | G | — | yes | yes |
| 29 | Q | — | yes | yes |
| 36 | F | — | yes | yes |
| 38 | I | — | yes | yes |
| 39 | H | — | yes | yes |
| 40 | N | — | yes | yes |
| 51 | F | — | yes | yes |
| 52 | T | — | yes | yes |
| 53 | N | — | yes | yes |
| 63 | P | — | yes | yes |
| 64 | E | — | yes | yes |
| 65 | A | — | yes | yes |
| 66 | K | — | yes | yes |
| 67 | Q | — | — | yes |
| 68 | V | — | — | yes |
| 98 | I | — | yes | yes |
| 101 | T | — | yes | yes |
| 102 | D | — | yes | yes |
| 104 | G | — | yes | yes |
| 105 | R | — | yes | yes |
| 108 | L | — | yes | yes |
| 109 | T | — | yes | yes |
| 111 | I | — | yes | yes |
| 112 | V | — | yes | yes |
| 114 | G | — | yes | yes |
| 117 | F | — | yes | yes |
| 128 | K | — | yes | yes |
| 130 | I | — | yes | yes |
| 131 | D | — | yes | yes |
| 133 | N | — | yes | yes |
| 134 | C | — | yes | yes |
| 135 | I | — | yes | yes |
| 143 | S | — | yes | yes |
| 144 | Y | — | yes | yes |
| 145 | R | — | yes | yes |
| 146 | S | — | yes | yes |
| 147 | E | — | yes | yes |
| 162 | Q | yes | — | — |
| 163 | F | yes | yes | yes |
| 164 | E | yes | yes | yes |
| 165 | C | — | yes | yes |
| 166 | K | — | yes | yes |
| 167 | S | — | yes | yes |
| 168 | F | — | yes | yes |
| 172 | V | — | yes | yes |
| 173 | L | — | yes | yes |
| 174 | N | — | yes | yes |
| 175 | L | — | yes | yes |
| 176 | T | — | yes | yes |
| 181 | G | — | yes | yes |
| 185 | Y | yes | — | — |
| 186 | I | — | yes | yes |
| 187 | R | yes | — | — |
| 196 | F | — | — | yes |
| 197 | E | — | — | yes |
| 198 | E | — | — | yes |
| 199 | S | — | — | yes |
| 201 | E | — | — | yes |
| 202 | V | — | — | yes |
| 205 | N | — | — | yes |
| 206 | P | — | — | yes |
| 207 | L | — | — | yes |
| 208 | L | — | — | yes |
| 209 | G | — | — | yes |
| 210 | A | — | — | yes |
| 218 | A | yes | — | — |
| 219 | V | yes | — | — |
| 220 | T | yes | — | — |
| 221 | L | yes | — | — |
| 222 | A | yes | — | — |
| 223 | H | yes | — | — |
| 224 | E | yes | — | — |
| 225 | L | yes | — | — |
| 226 | I | yes | — | — |
| 227 | H | yes | — | — |
| 228 | A | yes | — | — |
| 229 | G | yes | — | — |
| 230 | H | yes | yes | yes |
| 238 | N | — | yes | yes |
| 239 | P | — | yes | yes |
| 241 | R | — | — | yes |
| 242 | V | — | — | yes |
| 243 | F | — | yes | yes |
| 244 | K | — | — | yes |
| 245 | V | — | — | yes |
| 246 | N | — | yes | yes |
| 247 | T | — | yes | yes |
| 248 | N | — | yes | yes |
| 249 | A | — | yes | yes |
| 250 | Y | — | — | yes |
| 251 | Y | — | — | yes |
| 252 | E | — | — | yes |
| 253 | M | — | yes | yes |
| 254 | S | — | yes | yes |
| 255 | G | — | yes | yes |
| 256 | L | — | yes | yes |
| 257 | E | — | — | yes |
| 258 | V | — | — | — |
| 259 | S | — | — | yes |
| 260 | F | yes | — | — |
| 261 | E | yes | — | — |
| 262 | E | yes | — | yes |
| 263 | L | yes | — | — |
| 264 | R | yes | — | — |
| 265 | T | yes | — | — |
| 268 | G | — | — | yes |
| 271 | A | — | yes | yes |
| 272 | K | — | — | yes |
| 273 | F | — | — | yes |
| 274 | I | — | — | yes |
| 275 | D | — | — | yes |
| 276 | S | — | — | yes |
| 277 | L | — | — | yes |
| 278 | Q | — | — | yes |
| 280 | N | — | — | yes |
| 304 | V | — | yes | yes |
| 305 | G | — | yes | yes |
| 306 | T | — | yes | yes |
| 307 | T | — | yes | yes |
| 311 | Q | — | yes | yes |
| 312 | Y | — | yes | yes |
| 315 | N | — | yes | yes |
| 318 | K | — | yes | yes |
| 334 | D | — | — | yes |
| 339 | D | — | yes | yes |
| 342 | Y | — | yes | yes |
| 343 | K | — | yes | yes |
| 346 | T | — | yes | yes |
| 347 | E | — | yes | yes |
| 348 | I | — | yes | yes |
| 350 | T | yes | — | — |
| 352 | D | — | yes | yes |
| 355 | V | — | yes | yes |
| 356 | K | — | yes | yes |
| 362 | N | yes | — | — |
| 364 | K | — | — | yes |
| 365 | T | yes | — | — |
| 366 | Y | — | — | yes |
| 367 | L | — | — | yes |
| 368 | N | — | — | yes |
| 370 | D | — | — | yes |
| 419 | F | — | — | yes |
| 420 | T | — | — | — |
| 421 | G | — | — | yes |
| 422 | L | — | — | yes |
| 423 | F | — | yes | yes |
| 424 | E | — | yes | yes |
| 425 | F | — | yes | yes |
| 426 | Y | — | yes | yes |
| 427 | K | — | yes | yes |
| 428 | L | — | yes | yes |
| 429 | L | — | yes | yes |
| 430 | C | — | yes | yes |
| 431 | V | — | yes | yes |
| 432 | R | — | yes | yes |
| 433 | G | — | — | yes |
| 434 | I | — | — | yes |
| 435 | I | — | — | yes |
| 436 | T | — | — | yes |
| 437 | S | — | — | yes |
| 438 | K | — | — | yes |
| 439 | T | — | yes | yes |
| 440 | K | — | — | yes |
| 441 | S | — | — | yes |
| 442 | L | — | — | yes |
| 443 | D | — | — | yes |
| 446 | Y | — | — | yes |
| 447 | N | — | — | yes |
| 448 | K | — | — | yes |

To preprocess mutation choices, a BLAST search was performed using the PG-1T, BoNT/A-LC protein sequence to collect closely related homolog sequences. A multiple sequence alignment was constructed and processed to identify sequences that contained gaps not beyond 25% of their total length, were sufficiently similar to BoNT/A-LC (at least 350%) and sufficiently different from other sequences in the homolog dataset (at most 90% identical). For each position in the multiple sequence alignment, amino acid choices were extracted and assessed for in silico structure stability and peptide immunogenicity.

Since it is infeasible to assess each variant in a billion-member library, a previously developed combinatorial library designing protocol called SOCoM (Zhao et al. Chemistry & biology. 22, 629-639. 2015; Salvat et al. PNAS. 114, e5085-e5093. 2017; Choi et al. Methods in molecular biology. 1529, 375-398. 2017; Griswold et al. Current opinion in structural biology. 39, 79-88. 2016), which employs a Cluster Expansion (CE) technique to enable mapping of aglobal protein property of interest as a function of its amino acid sequence, was used to precompute structure-based sequence potential. To enable CE model training, 18000 random BoNT/A-LC variants were designed in silico using the filtered mutation choices as explained above. The stability of the variants was assessed by Rosetta (Rohl et al. Methods in enzymology. 383, 66-93. 2004) and referred to as a potential score hereon. A statistically trained CE model employs these potential scores and breaks them into position-specific amino acid components. The potential of the native BoNT/A-LC is referenced at 0, i.e., the native amino acids do not contribute to any relative change in the overall stability of the structure. Any change due to a mutation in the native sequence can either be more stable (referenced by a negative potential score), less stable (referenced by a positive potential score) or no change.

Since the amino acid potential contributions are position-specific and treated as independent of each other, they can be used for an BoNT/A-LC variant stability assessment directly from its sequence. This breakdown also makes the stability assessment of libraries in SOCoM very fast, which processes the potentials using averages in a library setting. The energy Ψ for a possible BoNT/A-LC variant S can be expressed via a sum over position-specific one- and two-body sequence potentials:

Ψ(S)=Σᵢψᵢ(aᵢ)+Σ_{i,j}ψ_{i,j}(aᵢ,aⱼ) (1)

where the sums involve amino acid aᵢ at position i and aⱼ at position j. The trained CE model gave more than 90% accuracy in predicting potential scores on a randomly generated variant set.

To precompute peptide immunogenicity, a list of 15-mer peptides was generated using the amino acid sequence of BoNT/A-LC. Peptide variants were also generated using the available mutation choices (all combinatorial mutations were considered when generating the variant peptides). These peptides were then assessed for immunogenicity against 26 MHC-II HLA alleles using a standalone version of NetMHCII (Karosiene et al. Immunogenetics. 65, 711-724. 2013). NetMHCII is a neural net MHC-II-peptide binding affinity prediction method that was developed using experimentally assessed MHC-peptide binding affinity data obtained from the Immune Epitope Database, which covers HLA-DR, HLA-DQ and HLA-DP molecules. The list of alleles targeted in silico in this study at 5% threshold are: HLA-DRB1 MHC alleles (DRB1*0101, 0301, 0401, 0405, 0701, 0802, 0901, 1101, 1201, 1302, and 1501), HLA-DRB3 MHC alleles (DRB3*0101 and 0202), HLA-DRB4 MHC allele (DRB4*0101), HLA-DRB5 MHC allele (DRB5*0101), HLA-DQA1*0501/DQB1*0201, HLA-DQA1*0501/DQB1*0301, HLA-DQA1*0301/DQB1*0302, HLA-DQA1*0401/DQB1*0402, HLA-DQA1*0101/DQB1*0501, HLA-DQA1*0102/DQB1*0602, HLA-DPA1*0201/DPB1*0101, HLA-DPA1*0103/DPB1*0201, HLA-DPA1*01/DPB1*0401, HLA-DPA1*0301/DPB1*0402, and HLA-DPA1*0201/DPB1*0501.

The precomputed epitope scores of each variant peptide along with stability contribution of amino acid mutations in the peptide were fed into structure-based deimmunized combinatorial library designing (EpiSOCoM) (Salvat, supra; Zhao 2015, supra; Choi, supra) to generate optimized deimmunized libraries. EpiSOCoM is a combinatorial library designing method that uses optimization techniques to enrich a library with stable and deimmunized variants. EpiSOCoM is based on SOCoM, which is a structure-based library design approach coupled with epitope analysis. It employs a sweep-based Pareto optimization algorithm (Parker et al. Journal of computational biology: a journal of computational molecular cell biology. 20, 152-165. 2013) to simultaneously optimize both the structure stability and epitope content in a protein.

Given a set of possible positions at which to mutate and possible amino acids to incorporate at those positions (as described above in the preprocessing step) along with a desired library size, EpiSOCoM selects a subset of the positions and subsets of the substitutions at those positions. It thereby specifies the construction of a library comprised of all combinations of the substitutions and corresponding wild-type residues. EpiSOCoM optimizes a library for the average energy score and the average epitope score over its constituent variants. To calculate the averaged potential (or energy) scores, EpiSOCoM precomputes ψ̅ᵢ, the average energetic contributions of possible subsets of amino acids that could be chosen at a position i, and ψ̅_{i,j}, the average for pair of positions i and j, based on the allowed mutations. It then evaluates the average energy Ψ̅ over a whole library T with an equation analogous to that for a single variant:

Ψ̅(T)=Σᵢψ̅ᵢ(Tᵢ)+Σ_{i,j}ψ̅_{i,j}(Tᵢ,Tⱼ) (2)

where the sums now involve sets of amino acids Tᵢ at position i and Tⱼ at position j. Thus, assessment of a library within the optimization is as efficient as assessment of a single variant.

The average epitope scores were calculated analogous to EpiSOCoM's concept of average energy scores. If amino acids (Tᵢ, Tᵢ₊₁, . . . , Tᵢ₊₁₄) are to be incorporated at the fifteen contiguous positions (in a peptide) starting at i, then the average epitope score contribution ēᵢ from the various 15 mer combinations of amino acids at position i is calculated as:

$${\overline{e}}_{i} = \frac{{Σ}_{{a}_{i} ∈ {T}_{i} , {a}_{i + 1} ∈ {T}_{i + 1 ,} … , {a}_{i + 8} ∈ {T}_{i + 14}} e ( {a}_{i} {a}_{i + 1} … {a}_{i + 14} )}{\vert {T}_{i} \vert \cdot \vert {T}_{i + 1} \vert … \vert {T}_{i + 14} \vert} \qquad ( 3 )$$

where the sum is over each combination of amino acids, one from each set, and the function e(·) (e(aᵢaᵢ₊₁ . . . aᵢ₊₁₄)) gives the epitope score of the 15 mer. Then the average epitope score, Ξ, of the library is simply the sum over all 15 mers:

Ξ=Σᵢ₌₁ⁿ⁻¹⁴e̅ᵢ (4)

A Pareto optimization algorithm in EpiSOCoM identified all library designs (positions and substitutions) making undominated trade-offs between the epitope scores and the energy scores, in that no other design is better for both. EpiSOCoM used an integer linear programming formulation to choose an optimal set of positions and sets of amino acids so as to optimize Eq. 2 subject to library size constraints. Since there is no a priori means to determine the best balance between these incommensurate properties, EpiSOCoM generates all Pareto optimal designs representing the best balance, enabling subsequent characterization of the trade-offs and selection of suitable designs.

Molecular Cloning

Unless noted otherwise, genes were inserted into vectors through Gibson Assembly® and transformed into chemically competent Escherichia coli strain Top10 by heat shock. The gene encoding the wild-type BoNT/A-LC was amplified from the template synthesized by Synbio Technologies, Inc. using primers

(SEQ ID NO: 110)
5′-TTAACTTTAATAAGGAGATATACCATGGCATTCGTGAACAAGCAGT
TTAACTA-3′
and
(SEQ ID NO: 111)
5′-CTGTTCGACTTAAGCATTATGCGGCCGCAAGCTTACTTGTTGTAGC
CTTTGTCCAG-3′

and cloned into pRSF-Duet vector. Splice overlap extension PCR was used to inactive BoNT/A-LC (IALC) by introducing E224Q and Y366F mutations using primers

(SEQ ID NO: 112)
5′ACGATGACGATAAGGATCCGAGCTCGACCGAATTCATGCCATTCGTG
AACAAGCAG-3′,
(SEQ ID NO: 113)
5′-GATCAGTTGGTGGGCCAGGGTCACGGCGGGGTCGGTGGCG-3′,
(SEQ ID NO: 114)
5′-CGCCACCGACCCCGCCGTGACCCTGGCCCACCAACTGATC-3′,
(SEQ ID NO: 115)
5′-GCTTTGTCGAAGTTCAGAAATGTTTTCCGGTTGAGCACTT-3′,
(SEQ ID NO: 116)
5′-AAGTGCTCAACCGGAAAACATTTCTGAACTTCGACAAAGC-3′,
and
(SEQ ID NO: 117)
5′ATCTTCTCTCATCCGCCAAAACAGCCAAGCTGCAGATCTTTAGAACT
CGAACAGCC-3′,

and cloned into pRSF-Duet vector. The gene encoding the Clover-SNAP-25-mRuby2 FRET sensor (Clover and mRuby2 fluorescent proteins flanking SNAP-25 residues 141-206) was amplified from a construct provided by Prof. Min Dong's lab at Boston Children's hospital using forward primer

(SEQ ID NO: 118)
5′ATTAGTTAAGTATAAGAAGGAGATATACATATGGTGAGCAAGGGCGA
GGAGCTGT-3′,
reverse primer
(SEQ ID NO: 119)
5′CAGCAGCGGTTTCTTTACCAGACTCGAGGTTATTACTTGTACAGCTC
GTCCATCC-3′,
forward primer
(SEQ ID NO: 120)
5′-TTTAACTTTAAGAAGGAGATATACATATGATCAAGGAAAATATGCG
TATGAAG-3′,
reverse primer
(SEQ ID NO: 121)
5′-CAGTGGTGGTGGTGGTGGTGCTCGAGGGCGGCGGTCACGAACTCCA
GCAG-3′,

and cloned into pRSF-Duet vector and pET26b vector, respectively. Genes encoding deimmunized BoNT/A-LC variants were amplified with 5′-TTTAACTTTAAGAAGGAGATATACATATGGCATTCGTCAACAAACAGTTCAA-3′ (SEQ ID NO: 122) and 5′-CAGTGGTGGTGGTGGTGGTGCTCGAGTTATTTGTTGTAGCCTTTGTCCAGGCT-3′ (SEQ ID NO: 123), and cloned into pET26b vector. Truncated versions of the BoNT/A-LC genes (residues 1-424) were amplified with forwarded primer 5′-TTTAACTTTAAGAAGGAGATATACATATGGCATTCGTCAACAAACAGTTCAA-3′ (SEQ ID NO: 124) and reverse primers 5′-CAGTGGTGGTGGTGGTGGTGCTCGAGGAACTCGAACAGGCCGGTGAAGT-3′ (SEQ ID NO: 125) for wild-type BoNT/A-LC or deimmunized BoNT/A-LC variants without the F418G mutation and 5′-CAGTGGTGGTGGTGGTGGTGCTCGAGGAACTCGAACAGGCCGGTGCCGT-3′ (SEQ ID NO: 126) for deimmunized BoNT/A-LC variants incorporating an F418G mutation.

Protein Expression and Purification

Truncated BoNT/A-LC proteins and Clover-SNAP-25-mRuby2 FRET sensor in pET26b vectors were transformed into chemically competent Escherichia coli BL21(DE3) ((F-ompT gal dcm lon hsdS_{B}(r_{B}-m_{B}-)λ(DE3 [lacI lacUV5-T7p07 ind1 sam7 nin5]) [malB⁺]K-12 (λS)) cells via heat shock for expression. Cells were cultured in Luria-Bertani medium containing 50 μg/ml kanamycin (LB-Kana) at 30° C. overnight to saturation, diluted 1:100 into fresh LB-Kana, grown at 30° C. to an OD600 of 0.4-0.8, and induced with 0.1 mM IPTG at 20° C. for 20 hours (truncated BoNT/A-LC proteins) or 30° C. for 14 hours (FRET sensors), respectively. Induced cell pellets were resuspended in phosphate buffered saline (PBS, 137 mM NaCl, 2.7 mM KCl, 10 mM Na2HPO4, 1.8 mM KH2PO4, pH 7.4) and lysed by sonication (Fisher Scientific). After centrifugation at 16000 rpm for 20 minutes, soluble cell lysates were separated, filtered by Millex Filter Unit, 0.22 μm (Millipore Sigma), and incubated with Ni-NTA agarose resin (Qiagen) in rotation at 4° C. for 2 hours. The resin was subsequently loaded to a Pierce™ Disposable Column, 5 mL (ThermoFisher Scientific), and washed with 20 column volumes of wash buffer A (PBS with 25 mM imidazole, pH 7.5) and 10 column volumes of wash buffer B (PBS with 50 mM imidazole, pH 7.5). The BoNT/A-LC proteins or FRET sensor were eluted with elution buffer (PBS with 250 mM imidazole, pH 7.5), buffer exchanged into PBS using Amicon Ultra-15 Centrifugal Filter Units, 30 kDa (Millipore Sigma). Protein purity was evaluated by SDS-PAGE (NuPAGE™ 4-12% Bis-Tris Protein Gels, Thermo Fisher).

Library Screen

The synthesized library ST1250-2 was cloned into a pRSF-Duet vector which co-expressed the Clover-SNAP-25-mRuby2 FRET sensor protein and then transformed into E. coli BL21 (DE3). Cells were grown in LB-Kana at 30° C. overnight to saturation, diluted 1:100 into fresh LB-Kana, grown at 30° C. to an OD600 of 0.4-0.8, and induced with 0.1 mM IPTG at 30° C. for 14-16 hours. Induced cells were pelleted down by centrifugation, washed with PBS twice, resuspended in PBS, and sorted on an iCYT Synergy flow cytometer equipped with a 70 μm nozzle. For sorting, excitation 1, 488 nm; emission 1, 525 nm; excitation 2, 561 nm; emission 2, 585 nm were set for monitoring the fluorescent intensity of Clover and mRuby2, respectively. Sorted cells were washed with PBS twice, re-cultured in LB-Kana at 30° C. overnight to saturation, and stored in glycerol at −80° C. In addition, sorted cells were washed with LB media twice and placed on “indicating” plates (LB-Kana supplemented with 0.1 mM IPTG) and grown at 30° C. for 2 days. Green colonies under blue LED light were selected for sequencing.

Initial Analysis of the Catalytic Activities of Deimmunized BoNT/A-LC Variants

Colonies from “indicating” plates, which exhibited strong green color under a blue LED light, were isolated, individually grown in LB-Kana at 30° C. overnight to saturation, diluted 1:100 into fresh LB-Kana, grown at 30° C. to an OD600 of 0.4-0.8, and induced with 0.1 mM IPTG at 30° C. for 14-16 hours. Induced cells were pelleted down by centrifugation, washed with PBS twice, resuspended in PBS, and analyzed on a SpectroMax Gemini plate reader (Molecular Devices, Sunnyvale, Calif.) using endpoint reading, auto cutoff, auto PMT, and excitation 1, 488 nm; emission 1, 525 nm (cutoff 515 nm); excitation 2, 561 nm; emission 2, 585 nm (cutoff 570 nm).

Genes encoding the deimmunized BoNT/A-LC variants were amplified, cloned into pET26b vectors, and transformed and into E. coli BL21(DE3) for expression. Transformants were grown in 500 μL LB-Kana in deep-well 2 ml 96-well Polypropylene sterile plates at 30° C. overnight to saturation, diluted 1:50 into 500 μL fresh LB-Kana in deep-well 2 ml 96-well polypropylene sterile plates, grown at 30° C. for 3 hours, and induced with 0.5 mM IPTG at 20° C. for 14-16 hours. After centrifugation, cell pellets were resuspended in 100 μL of BugBuster® HT Protein Extraction Reagent (Millipore Sigma) and incubated at room temperature for two hours with gentle shaking. Cell debris was pelleted down by centrifugation and the soluble whole cell lysates were collected. The catalytic activities of the deimmunized BoNT/A-LC variants were measured by adding 5 μL cell lysates into 235 μL BoNT/A-LC reaction buffer (50 mM Tris-HCl, 10 μM ZnCl2, pH 7.2) with 10 μL FRET sensor at a concentration of 0.1 mg/mL. The reaction mixtures were incubated at 37° C. for 2 hours and analyzed on a SpectraMax® Paradigm® Multi-Mode Microplate Detection Platform (Molecular Devices LLC) using endpoint reading, PMT and optics with 140 ms integration time and 1.00 mm read height, and an excitation of 488 nm, emission 1 of 525 nm, and emission 2 of 600 nm.

Characterization of Deimmunized BoNT/A-LC Variants

Purified Clover-SNAP-25-mRuby2 FRET sensor protein were diluted to 67 nM in 240 μL of reaction buffer (50 mM Tris-HCl, 10 μM ZnCl2, pH 7.2) added to a Corning® 96 Well Black Polystyrene Microplate 96-well black-sided, black-bottom plate. All at once, 10 μL of deimmunized truncated BoNT/A-LC (tALC) variants, along with wild-type BoNT/A-LC and truncated inactive BoNT/A-LC (tIALC), were added to each well at a final concentration of 8 nM and the plate was immediately read on a SpectraMax® Paradigm® Multi-Mode Microplate Detection Platform (Molecular Devices LLC) using kinetic mode (time: 1 hour, interval: 30 seconds, and reads: 121), PMT and optics with 140 ms integration time and 1.00 mm read height, and an excitation of 488 nm, emission 1 of 525 nm, and emission 2 of 600 nm. The ratio between the fluorescent signal of Clover (emission1:525 nm) and FRET signal of mRuby2 (emission2:600 nm) was used to indicate the catalytic activity of tALCs. The specific activity of each variant was defined as the rate of change for the Clover:FRET emission ratio (Em 525:Em 600), which was calculated from the slope of the linear portion of the time course. The relative activity of each protein was calculated by normalizing the specific activity of each variant to the wild-type tALC.

The melting temperatures (Tₘ) of the selected tALC variants, along with wild-type tALC, were measured by differential scanning fluorimetry (DSF) following a previously reported procedure (Niesen et al. Nature protocols. 2, 2212-2221, 2007; Choi et al. Bioinformatics. 34, i245-i253. 2018). In brief, 5 μM of purified tALC variants were mixed with 5× SYPRO© Orange dye. Sextuplicate samples of each protein were measure on a CFX96 Touch™ Real-Time PCR Detection System (Bio-Rad) using a temperature gradient from 25° C. to 99° C. with a 1° C. per minute. Tₘ values were determined by the Bio-Rad CFX Manager 3.0 software.

Production of Full-Length Botulinum Neurotoxins

Full-length botulinum neurotoxins were generated using a previously reported sortase ligation method (Zhang et al. Nature communications. 8, 14130. 2017; Garland et al. ACS chemical biology. 14, 76-87. 2019; Tao et al. Nature communications. 8, 53. 2017). In brief, the thrombin site was introduced between the light chain and the heavy chain N terminal domain (Hn/A) for activation of botulinum toxin. The sortase tag (sort) LPETGG (SEQ ID NO: X) was introduced at the C-terminus of Hn/A. The His6 tagged heavy chain C terminal domain (Hc/A) was cleaved by thrombin to expose the free Glycine residue at the N-terminus of He/A. The ligation mixture was set up in 25 μL Tris-buffer with 5 μM LCH_{N}-sort, 40 μM He/A, 0.5 μM sortase, and 10 mM CaCl₂ for 40 minutes at room temperature. The ligated full-length botulinum neurotoxins were activated by incubating with thrombin at room temperature for 30 minutes.

Activity of Full-Length Botulinum Neurotoxins on Rat Cortical Cultured Neuron

Primary rat cortical neurons were prepared from E19 embryos using the papain dissociation kit (Worthington Biochemical). Neurons were cultured in neurobasal medium (Thermo Fisher Scientific) containing B27 (Thermo Fisher Scientific) and 0.5% fetal bovine serum. Cultured neurons were exposed to full-length botulinum neurotoxins (sortase ligation mixtures) for 12 hours and lysed with RIPA buffer. Lysates were centrifuged at 4° C. for 10 minutes to collect supernatants, which were then analyzed by SDS-PAGE and immunoblotting.

Digit Abduction Score (DAS) Assay

The DAS assay was carried out following the previously described procedure. In brief, mice (CD-1 strain, male) were purchased from Charles River and activated full-length botulinum neurotoxins (10 μL) were injected into mice right hind limb muscle using a 30-gauge needle attached to a Hamilton syringe. The muscle paralysis was scored by counting the spread of toes after 24 hours (Aoki et al. Toxicon: official journal of the International Society on Toxinology. 39, 1815-1820. 2001).

### Example 2—Structure-Based Design of Combinatorial Deimmunized Libraries

Pareto optimal deimmunized BoNT/A-LC libraries were generated using EpiSOCoM with the goal of isolating BoNT/A-LC variants exhibiting reduced immunogenicity yet high functionability (e.g., catalytic activity, thermostability, and biologic activity as a full-length toxin). NetMHCII analysis (Karosiene, supra; Nielsen et al. BMC bioinformatics. 8, 238. 2007; Jensen et al. Immunology. 154, 394-406. 2018), which predicts binding affinities between peptides and MHC Class II molecules, at a 5% threshold was used to assess epitope content, and mutations were designed to deimmunize BoNT/A-LC against a set of 27 Class II HLA supertypes known to be representative of HLA peptide binding specificity for the global population (Greenbaum et al. Immunogenetics. 63, 325-335. 2011; Ahmad et al. Applied microbiology and biotechnology. 98, 5301-5317. 2014; Sidney et al. Journal of immunology. 185, 4189-4198. 2010). Subsequently, a repertoire of 508 deimmunized library designs were generated on the Pareto curve, wherein the libraries traded off predicted immunogenicity and predicted functionality to varying degrees. Wild type BoNT/A-LC (WT BoNT/A-LC), which serves as the reference, has a potential score of 0 and an epitope score of 1222. Library plan ID 444 was designed based on the following constraints: fixed number of 28 mutable positions allowed anywhere on the protein, and a library size ranging from 1×10⁶-1.2×10⁹. Table 5 below lists the complete library plan of Library 444 with all the proposed mutations, their corresponding epitope deletion power, and potential score contribution. Table 5 also lists the positions that are likely to be interacting. Relative solvent accessibility was calculated by obtaining raw values from Dictionary of Protein Secondary Structure (DSSP) (Kabsch et al. Biopolymers. 22, 2577-2637. 1983) and the values were normalized with ASAView algorithm (Ahmad, supra). Coupled positions were those mutated pairs of positions with non-zero two-body terms in the sequence potential. Potential score is the average, over the set of mutations at the position, of their one-body terms in the sequence potential. Likewise, Epitope deletion score is the average over the mutations of the change each induces in the NetMHCII score.

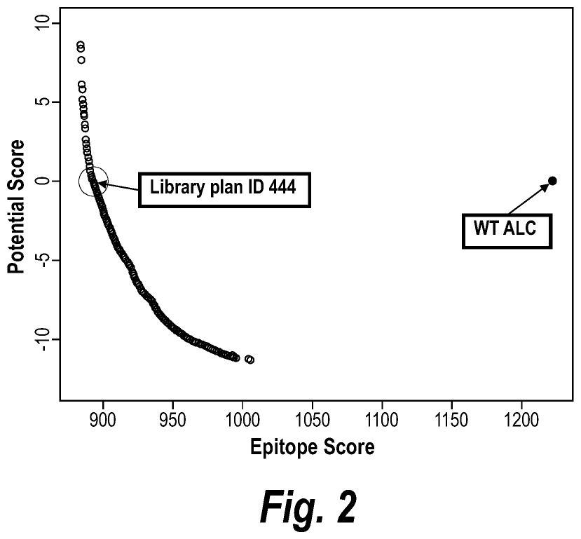

Library 444 had a theoretical size of 2.68×10⁸ protein members, an average epitope score of 892, and an average potential score of −0.05. Because Library 444 possessed a dramatically reduced epitope score while having a potential score close to that of WT BoNT/A-LC (FIG. 2), it was chosen for experimental evaluation.

TABLE 5
Mutation choices for Library design 444.

| | | | | | | Epitope score |
|---|---|---|---|---|---|---|
| | WT- | Solvent | Proposed | Coupled | Potential score | deletion |
| Position | Residue | accessibility | mutations | positions | contribution | contribution |
| 16 | V | 66% | RV | | −2.84 | 2.5 |
| 30 | Q | 100% | EQ | | 0.04 | 7 |
| 81 | N | 81% | AN | | 0.04 | 4.5 |
| 99 | S | 38% | ES | (99, 386) | 0.35 | 18.5 |
| 137 | I | 27% | IK | | 0.25 | 9.5 |
| 150 | L | 0% | LV | | −0.38 | 6.5 |
| 156 | S | 1% | GS | | −0.69 | 7.5 |
| 184 | Y | 0% | IY | | 0.08 | 5 |
| 193 | F | 9% | FN | (193, 371) | 0.32 | 19.5 |
| 212 | F | 1% | FY | (212, 360) | −0.23 | 4.5 |
| 225 | I | 1% | IT | | 1 | 7.5 |
| 234 | I | 0% | IT | (234, 344) | 0.94 | 16 |
| 240 | R | 31% | ER | (240, 258) | 0.31 | 10.5 |
| 258 | S | 4% | KS | (240, 258) | 1.28 | 14 |
| 283 | L | 74% | DL | (283, 284) | 0.12 | 20.5 |
| 284 | Y | 42% | KY | (283, 284) | 0.38 | 19 |
| 302 | I | 15% | IT | | −0.51 | 8 |
| 310 | Q | 38% | DQ | | −0.36 | 12.5 |
| 321 | L | 51% | KL | | −1.84 | 14 |
| 335 | L | 100% | LN | | −0.19 | 12 |
| 344 | L | 1% | KL | (234, 344) | 2.12 | 16 |
| 354 | V | 19% | SV | | −0.54 | 19 |
| 360 | L | 3% | LQ | (212, 360) | 0.45 | 16.5 |
| 371 | A | 9% | AG | (193, 371) | −0.93 | 11 |
| 381 | V | 98% | DV | | −0.37 | 21.5 |
| 386 | Y | 84% | KY | (99, 386) | −0.19 | 7 |
| 413 | T | 76% | DT | | 0.22 | 10 |
| 418 | F | 37% | FG | | 1.32 | 23.5 |

### Example 3—Design and Validation of a FRET Sensor System for the Detection and Quantitative Analysis of BoNT/A-LC Catalytic Activity

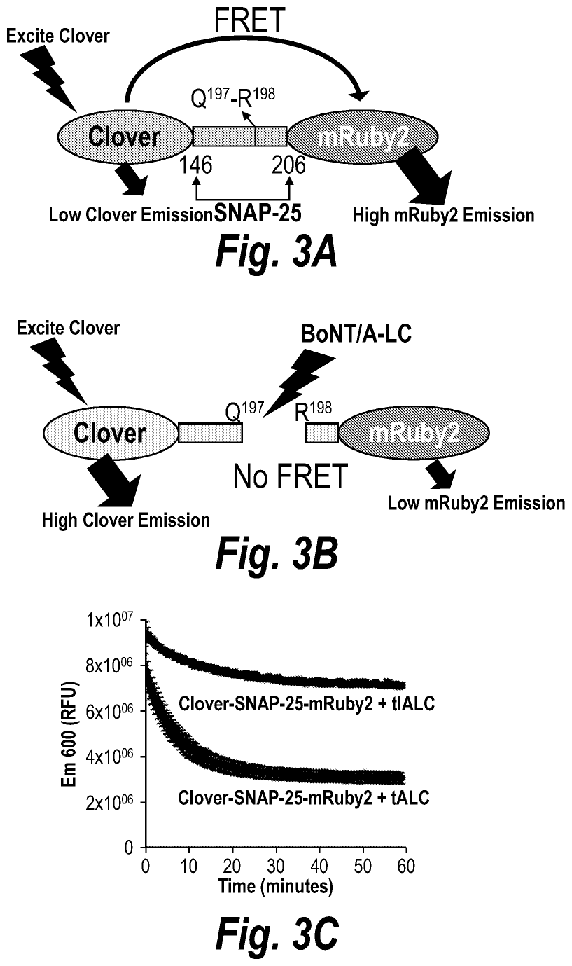

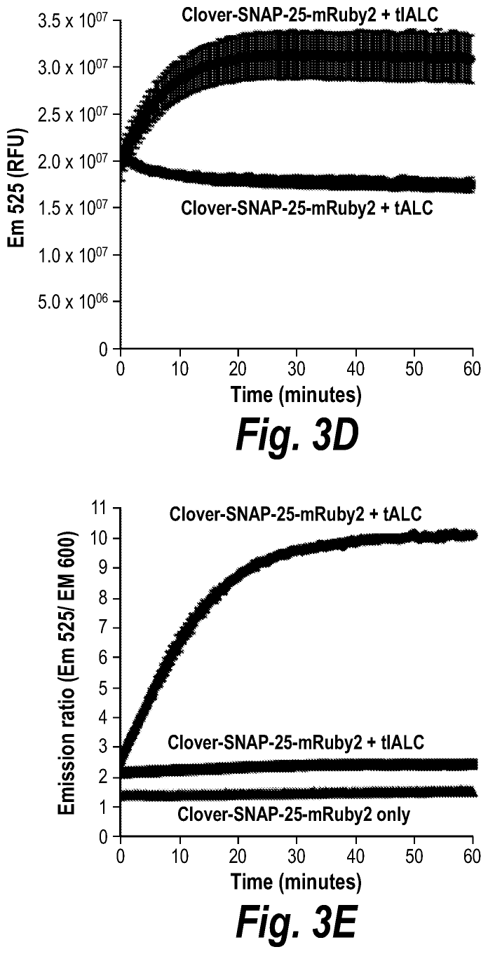

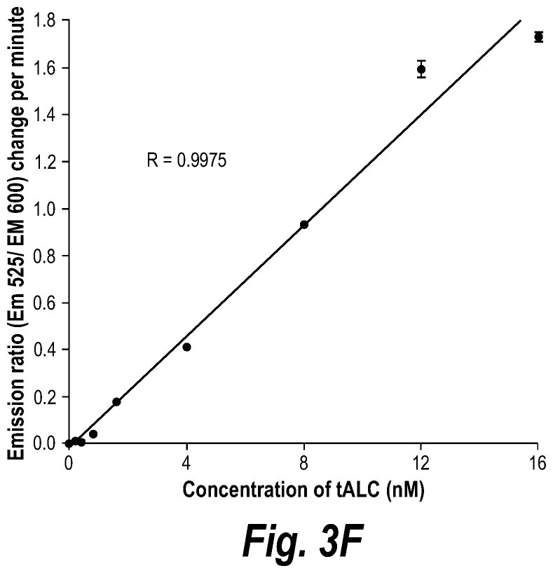

To facilitate the detection and quantitative analysis of BoNT/A-LC catalytic activity, a novel FRET sensor was designed based on a previously published BoNT reporter, in which the native substrate of BoNT/A-LC, SNAP-25, was fused between a cyan fluorescent protein (CFP) and yellow fluorescent protein (YFP) FRET pair (Dong et al. PNAS.101, 14701-14706. 2004). When SNAP-25 is cleaved by BoNT/A-LC in vitro, the acceptor and donor fluorescent proteins diffuse beyond their Förster radius, thus abolishing the FRET signal. Here, a modified sensor system was constructed by replacing the original CFP-YFP FRET pair with the alternative Clover and mRuby2 fluorescent protein pair (FIG. 3A). This change led to reduced phototoxicity and higher FRET dynamic range than the CFP-YFP pair (Masuyer et al. Scientific reports. 8, 4518. 2018; Lam et al. Nature methods. 9, 1005-1012. 2012; Bajar et al. Scientific reports. 6, 20889. 2016).

To test the performance of the modified FRET sensor in an in vitro enzymatic assay, a C-terminal (residues 1-424, truncated BoNT/A-LC (tALC)) (Feltrup et al. Scientific reports. 8, 8884. 2018; Gul et al. PloS one. 5, e12872. 2010; Silvaggi et al. Chemistry & Biology. 14, 533-542. 2007; Roxas-Duncan et al. Antimicrobial agents and chemotherapy. 53, 3478-3486. 2009) was cloned, expressed, and purified as a positive control. As a negative control, two mutations (E224Q and Y366F), which are known to reduce the enzymatic activity of BoNT/A-LC to baseline levels (Gu et al. Current topics in microbiology and immunology 364, 21-44. 2013; Binz et al. Biochemistry 41, 1717-1723. 2002; Breidenbach et al. Nature 432, 925-929. 2004; Fu et al. Biochemistry 45, 8903-8911. 2006; Li et al. Biochemistry 39, 2399-2405. 2000), were introduced to create an inactivated variant of the truncated light chain (tIALC). Purified tALC and tIALC enzymes were each incubated with a fixed concentration of purified sensor protein, and the reduction in FRET signal (excitation, 488 nm; emission 1, 525 nm; emission 2, 600 nm) was monitored at 37° C. in 96-well plates. The active tALC efficiently cleaved the FRET sensor (FIG. 3B), resulting in a decrease in mRuby2 emission at 600 nm (FIG. 3C) and a concurrent increase in the Clover emission at 525 nm (FIG. 3D). As an integrated measure that captures both fluorescent signals, the change of Clover:mRuby2 emission ratio (Em 525:Em 600) was plotted versus time (FIG. 3E). In contrast to tALC, the tIALC resulted in little change in FRET signal, indicating that the variant was largely inactive as expected. In addition, the rate of change of Clover:mRuby2 emission ratio was proportional to the concentration of tALC over at least a one order of magnitude range in enzyme concentration (FIG. 3F). This observation suggests that the assay can be used to quantitatively differentiate BoNT/A-LC variants based on specific activity. Compared with the original CFP-SNAP-25-YFP sensor (1.4-fold emission ratio in 80 minutes) (Dong et al., supra), the modified Clover-SNAP-25-mRuby2 sensor system produces a larger dynamic range in FRET (6-fold emission ratio in 30 minutes) upon SNAP-25 cleavage, yielding a more sensitive enzymatic reporter for BoNT/A-LC. Additionally, Clover and mRuby2 are compatible with common laser lines and emission filters on fluorescence activated cell sorters (FACS), which enabled the subsequent development of a high-throughput FACS screening methodology.

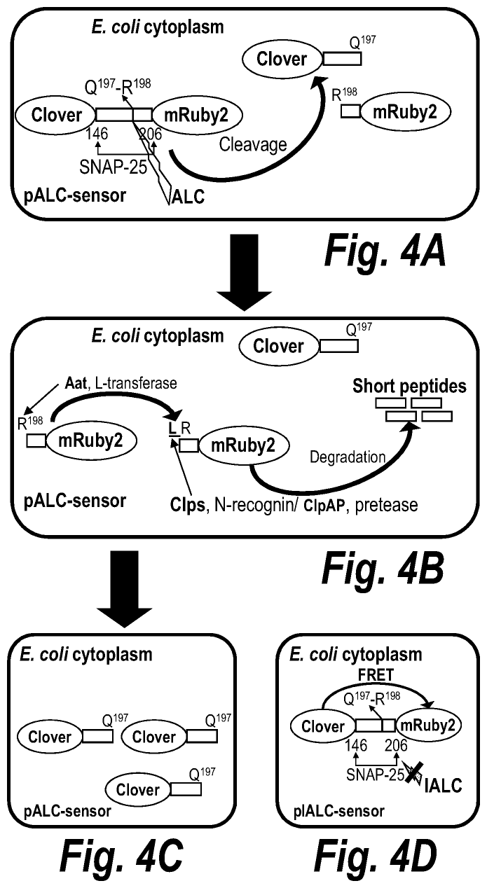

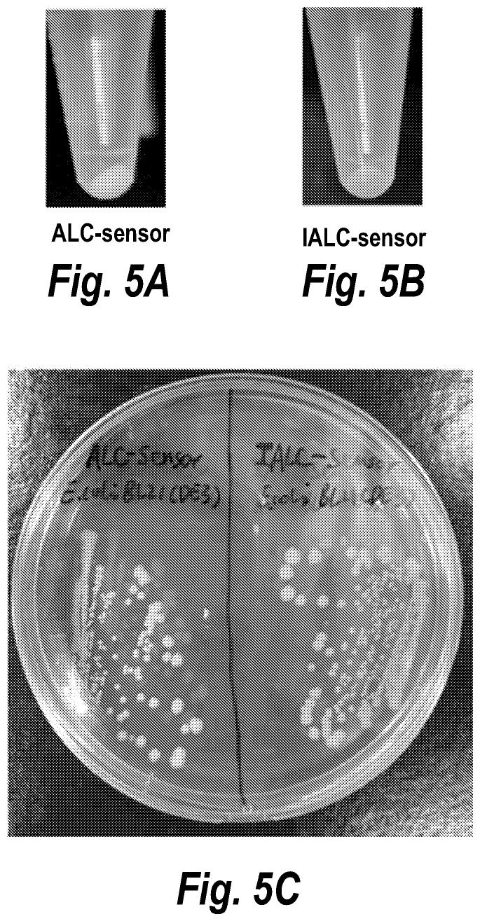

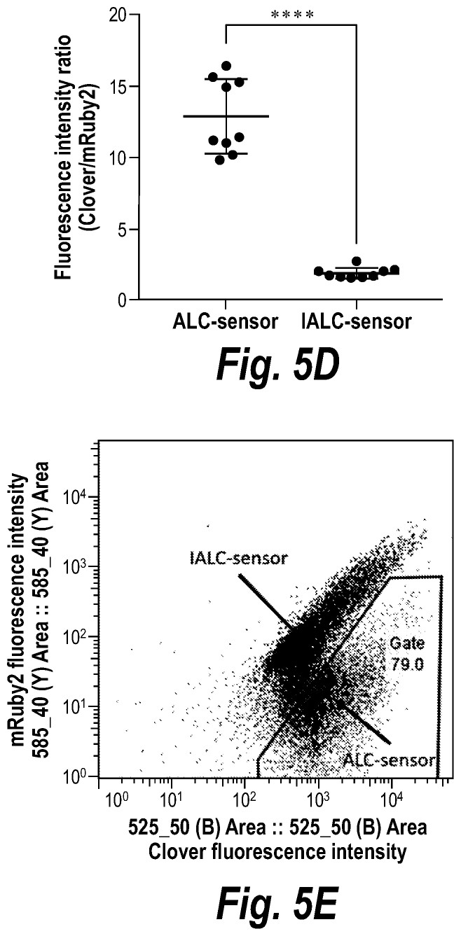

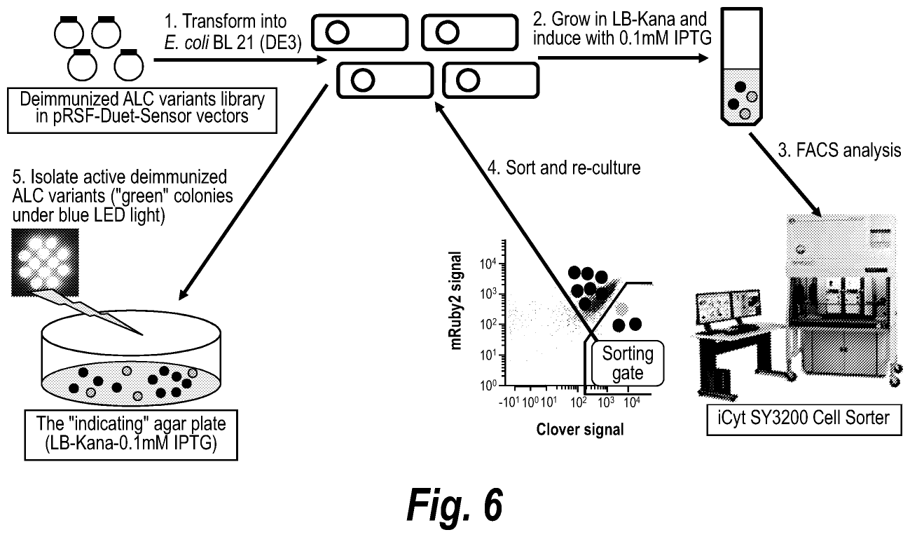

The FRET sensor worked well in a 96-well plate format using purified sensor and BoNT/A-LCs, enabling facile characterization and quantitative analysis of individual BoNT/A-LC variants. However, the throughput of this methodology is limited by the need for purified BoNT/A-LCs, such that only small libraries (10³-10⁵) of variants could be reasonably screened, even with automated liquid-handling robotics. Given the size of the computationally optimized deimmunization libraries described above (Table 5, Library 444, ˜3×10⁸ variants), there was a need to develop a higher throughput screening strategy. As noted above, the Clover-mRuby2 FRET system is compatible with standard FACS lasers and filters, and FACS based screening has the capacity to analyze and sort 10⁷-10⁸ cells per hour (Salvat, supra). To evaluate the feasibility of a FACS based BoNT/A-LC screen, a “pRSF-Duet” based vector system was constructed enabling co-expression of BoNT/A-LC or inactivated BoNT/A-LC with the FRET sensor protein in the E. coli cytoplasm. For E. coli co-expressing the active enzyme and sensor (BoNT/A-LC-sensor), putative cleavage of the SNAP-25 linker would liberate the mRuby2 fragment with an N-terminal arginine, R¹⁹⁸ (FIG. 4A). R¹⁹⁸-mRuby2 is expected to be modified by the E. coli enzyme Aat, which appends an N-terminal leucine or phenylalanine onto proteins possessing N-terminal arginine. Any such N-terminally modified mRuby2 represents an “N-degron” that is subject to ClpS-mediated targeting to, and degradation by, the E. coli ClpAP proteasome (FIG. 4B), a process which is commonly referred to as the “N-end rule” in E. coli (Dougan et al. Molecular microbiology. 76, 545-558. 2010; Sekar et al. PloS one. 11, e0149746. 2016; Tobias et al. Science. 254, 1374-1377. 1991; Humbard et al. The Journal of biological chemistry. 288, 28913-28924. 2013). Thus, cleavage of the FRET sensor by BoNT/A-LC should decrease the FRET signal (excitation, 488 nm; emission, 585 nm), increase the Clover signal (excitation, 488 nm; emission, 525 nm), and also decrease the liberated mRuby2 signal (excitation, 561 nm; emission, 585 nm) as a result of the N-end rule (FIG. 4B). In practice, it was found that ALC-sensor cells grown in liquid culture appeared green under ambient light (FIG. 5A). In contrast, when co-expressed with IALC (IALC-sensor), the sensor is expected to remain intact and maintain FRET (FIG. 4D). Indeed, IALC-sensor cells grown in liquid culture appeared reddish under ambient light (FIG. 5B). Similarly, when the cells were grown on indicating agar plates supplemented with 0.1 mM IPTG, colonies of ALC-sensor and IALC-sensor appeared green and reddish, respectively, under ambient light (FIG. 5C). To further evaluate this cell-based FRET system, induced ALC-sensor and IALC-sensor cells, cultured in replicate on eight different days, were measured by 96-well fluorescence microplate reader, and the fluorescence intensity ratio between Clover (excitation, 488 nm; emission, 525 nm) and mRuby2 (excitation, 561 nm; emission, 585 nm) was used as an indicator of catalytic activity. As shown in FIG. 5D, the system provides reasonable day-to-day reproducibility, and the difference between positive (13±3) and negative controls (1.9±0.4) are readily distinguished (2-tailed T test, p<0.0001). Next, ALC-sensor and IALC-sensor cells were analyzed by flow cytometry, revealing two populations on a Clover versus mRuby2 dot plot (FIG. 5E). Importantly, a gate could be drawn such that approximately 80% of the positive ALC-sensor population were captured while less than 0.10% of the negative IALC-sensor population fell in the same gate. This separation was deemed sufficient to enable FACS screening of an ALC library.

### Example 4—FACS Isolation of Active BoNT/A-LC Variants from the Deimmunized BoNT/A-LC Library

The combinatorial library ST1250-2 was synthesized (Synbio Technologies, Inc., Monmouth Junction, N.J., USA) based on the design of library 444. The gene library was cloned into a pRSF-Duet vector engineered to co-express the FRET sensor protein, and was then transformed into E. coli BL21 (DE3) yielding approximately 2×10⁸ transformants (referred to hereafter as ST1250-2 lib3.0). The library population was iteratively grown, induced, and sorted by FACS, with the goal of isolating cells expressing highly active enzyme variants (FIG. 6).

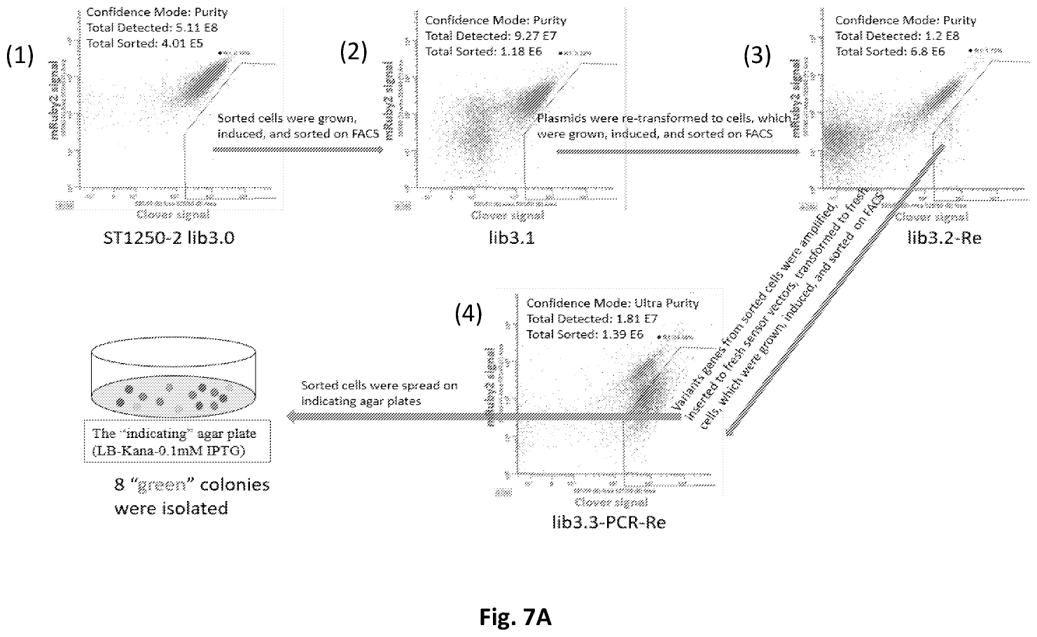

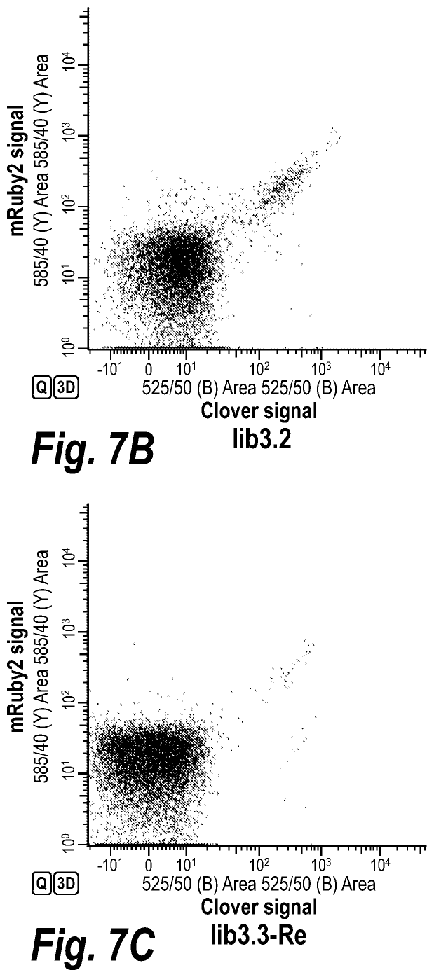

An initial sort gate, based on higher Clover signal and lower mRuby2 signal, excluded 99.9% of IALC-sensor cells (negative control) while retaining approximately 80% of ALC-sensor cells (positive control). For the naive ST1250-2 lib3.0 library, only 0.2% of the population was captured by this initial sort gate, indicating most of the population at this stage was comprised of inactive variants (FIG. 7A). A total of 5.1×10⁸ cells were screened (2-fold of the theoretical library size), and 4×10⁵ positive events were sorted. The sorted cells were grown overnight, sub-cultured, and induced at mid-log growth phase. The induced cells (lib3.1) were sorted using the same gate as above, capturing approximately 0.3% of the population (FIG. 7A). A total of 9.3×10⁷ cells were screened and 1.2×10⁶ events were sorted during the second round of FACS screening, and the sorted cells were grown to saturation overnight (lib3.2). It was noted during preliminary experiments that cells bearing either pALC-sensor or pIALC-sensor manifested a non-fluorescent subpopulation during serial passage (data not shown). This phenomenon is speculated to arise from fluorescent protein toxicity, resulting in a selective pressure to eliminate Clover and mRuby2 (Ansari et al. Stem cell reviews. 12, 553-559. 2016; Jensen. Anatomical record. 295, 2031-2036. 2012). Unfortunately, ST1250-2 lib3.1 suffered the same problem, as around 10% of the population exhibited low fluorescence after overnight outgrowth, subculture, and induction (FIG. 7A). The lib3.2 cells exhibited a greater loss of function, as >90% of the population lacked fluorescence upon outgrowth, induction, and reanalysis (FIG. 7B). To solve this issue, plasmid was isolated from lib3.2 and re-transformed into fresh E. coli BL21 (DE3), resulting in population lib3.2-Re. The freshly transformed lib3.2-Re cells were grown, induced, and screened by FACS. Around 2% of the population was captured by the original sorting gate (FIG. 7A), indicating a 10-fold enrichment was achieved relative to the naïve library. Using the same gate, 1.2×10⁸ cells were screened and 6.8×10⁶ events were sorted during FACS round 3 (lib3.3). Interestingly, isolation of lib3.3 plasmid and retransformation into fresh E. coli BL21 (DE3) did not eliminate loss of function mutants, as >90% of the re-transformed cells (lib3.3-Re) exhibited low fluorescence (FIG. 7C). To recover the fluorescent signal, genes encoding the lib3.3 ALC variants were PCR amplified from the sorted cells, subcloned into fresh pRSF-sensor vector, and transformed into fresh E. coli BL21 (DE3), yielding library lib3.3-PCR. Following induction, 25% of this population fell within the original sort gate, indicating a 125-fold enrichment relative to the naïve library (FIG. 7A). A fourth round of sorting screened 1.9×10⁷ cells and collected 1.4×10⁶ events (the top 7% of lib3.3-PCR). Sorted cells were washed with LB medium twice before plating on agar medium containing 0.1 mM IPTG inducer (lib3.4).

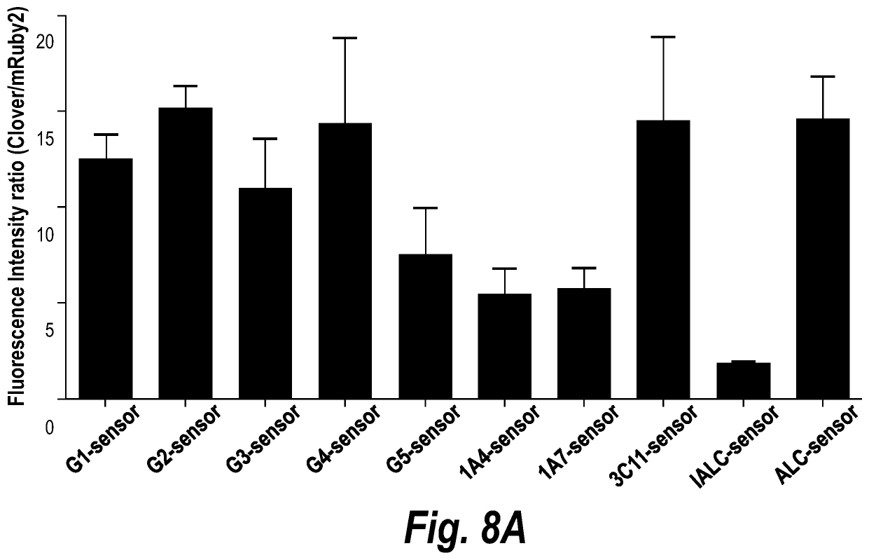

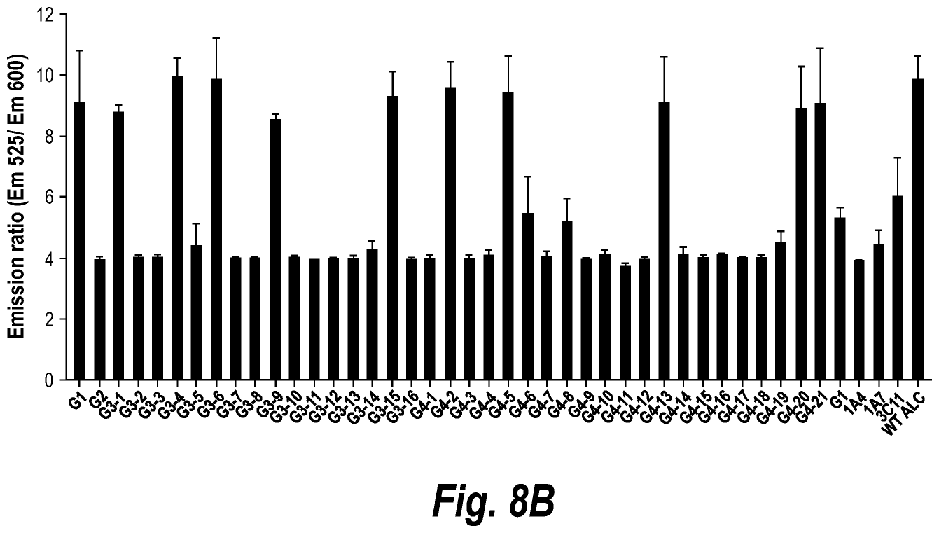

After incubation at 30° C. for 96 hours, eight colonies exhibiting strong green color under a blue LED light were isolated (inactive clones appeared red under the LED light). As a quick analysis to confirm their catalytic activity, the eight “green” colonies (labeled as G1, G2, G3, G4, G5, 1A4, 1A7, and 3C11) were individually grown in LB-Kan along with ALC-sensor and IALC-sensor, induced, and measured by 96-well fluorescence microplate reader (Ex1, 488 nm; Em1, 525 nm; Ex2, 561 nm; Em2, 585 nm). As shown in FIG. 8A, all 8 isolated variants exhibited fluorescence intensity ratio (Clover:mRuby2) higher than 6, suggesting moderate to high levels of proteolytic activity. Each clone was archived as a glycerol stock, and the mutant genes were amplified, sub-cloned into pET26b, sequenced, and employed in further in vitro characterization of the variants.

### Example 5—In Vitro Characterization of Deimmunized ALC Variants

An unexpected result was discovered when G3 and G4 were sub-cloned into pET26b. Unlike the other 6 mutants, which were all monoclonal, sequencing revealed that G3 and G4 were polyclonal populations composed of multiple alc gene sequences. A total of 40 individual colonies from the pET26b sub-cloning step were sequenced from the G3 (16 colonies) and G4 (24 colonies) populations and only two (G4-5 and G4-24) had the same sequences. Along with G1, G2, G5, 1A4, 1A7, and 3C11, the sequences of the 45 unique variants covered 27 of the 28 target mutation sites as designed, and only 2 of the 45 variants encoded off-target mutations, one each: V16L for G4-13 and G119S for 1A4.

Before purifying each individual protein for analysis, a medium throughput in vitro enzymatic assay was performed to select variants with high catalytic activity and/or expression titers. In brief, all variants were expressed as full-length ALCs (residues 1-448) in deep-well microplate format, and soluble cell lysates were incubated with a fixed concentration of purified FRET sensor molecule. The Clover:FRET fluorescence ratio (Ex=488 nm; Em1=525 nm:Em2=600 nm) was then determined using a fluorescence microplate reader. Variants G1, G3-1, G3-4, G3-6, G3-9, G3-15, G4-2, G4-5, G4-13, G4-21, G4-22, and 3C11 exhibited relatively high emission ratio (ranging from 6 to 10) in this assay format, which was comparable to cells expressing WT ALC (FIG. 8B). The high activity/expression variants were selected for further characterization, along with several additional variants of interest:
G4-5-F418G, which encodes an additional F418G deimmunization mutation relative to G4-5
G2 and G5, which were both positive on indicating agar plates and monoclonal
G4-8, which had a Clover:FRET ratio of only 5, but possessed the lowest predicated epitope score
G4-20, which was the only isolated variant encoding the F193N mutation
G4-23, which possessed the second lowest predicated epitope score
1A7, the isolated variant bearing the highest mutational load, including the Y184I mutation
Note: G4-13 was excluded, as it only differs from G4-5 by 1 off-target mutation

In total, 18 variants were advanced to more detailed analytical studies.

Full-length ALC has an extra cysteine at position 430. When screening intact cells, the C430 residue remains reduced in the E. coli cytoplasm and does not pose a problem. In contrast, following cell lysis and purification of ALC, the C430 residue is subject to oxidation, leading to gradual dimerization, destabilization, and aggregation of the protein (data not shown). To obtain stable and pure deimmunized ALC variants, pET26b containing truncated versions (residues 1-424) (Feltrup et al. Scientific reports 8, 8884. 2018; Gul et al. PloS one. 5, e12872. 2010; Silvaggi et al. Chemistry & biology. 14, 533-542. 2007; Roxas-Duncan et al. Antimicrobial agents and chemotherapy. 53, 3478-3486. 2009) of the chosen variants were constructed, expressed, and purified.

The purified truncated variants, as well as wild type tALC control, were incubated with a fixed concentration of purified FRET sensor, and the specific activity of each protein was defined as the rate of change for the Clover:FRET emission ratio (Em 525:Em 600), which was calculated from the slope of the linear portion of the time course. Variants were compared to the WT tALC to obtain relative activities. Additionally, the melting temperature (Tₘ) of each variant was measured by differential scanning fluorimetry (DSF) (Niesen, supra).

Each protein's relative activity, Tₘ, predicted epitope score, and predicted potential score are summarized in Table 6. The most active deimmunized ALC variant in vitro is G4-22, which has the same specific activity as WT. There were eight deimmunization mutations that appeared to be well tolerated (Q30E, N81A, S156G, L283D, Q310D, V354S, A371G, T413D), as they were encoded by a large proportion of variants having >50% wild type specific activity (7, 8, 7, 11, 8, 10, 9, and 7 of the 11 high activity variants, respectively). The majority of tested variants exhibited only small losses of thermostability (Tₘ within 3° C. of wild type), with only four variants having greater losses of 4 to 7° C.

TABLE 6
Performance parameters of tested deimmunized ALC variants.
N.D. means not determined due to low relative activities.

| | | | Activity on | | | |
|---|---|---|---|---|---|---|
| | | | neuron cells | | | |
| | | | as full- | | | |
| | | % activity | length toxin | | Predicted | Predicted |
| Protein | Mutation | relative to | (% SNAP-25 | Tm | epitope | potential |
| ID | load | WT tALC | cleavage) | value | score | score |
| WT tALC | 0 | 100 | 80% | 42° C. | 1222 | 0 |
| tG1 | 10 | 52 ± 3 | 0% | 35° C. | 982 | 0.178513 |
| tG2 | 11 | 42 ± 1 | 80% | 36° C. | 983 | −2.730256 |
| tG3-1 | 9 | 83 ± 3 | 30% | 40° C. | 1017 | −1.864675 |
| tG3-4 | 9 | 93 ± 4 | 70% | 39° C. | 999 | −6.307599 |
| tG3-6 | 8 | 94 ± 7 | 80% | 38° C. | 1041 | −0.093753 |
| tG3-9 | 11 | 75 ± 7 | N.A. | 39° C. | 943 | 0.246034 |
| tG3-15 | 10 | 88 ± 5 | 40% | 41° C. | 1003 | −7.241558 |
| tG4-2 | 10 | 90 ± 4 | N.A. | 42° C. | 990 | −4.720785 |
| tG4-5 | 14 | 78 ± 5 | 70% | 40° C. | 914 | −11.448634 |
| tG4-5-F418G | 15 | 53 ± 3 | 80% | 41° C. | 867 | −8.808578 |
| tG4-8 | 15 | 34 ± 4 | 0% | N.D. | 872 | −6.740511 |
| tG4-20 | 13 | 5 ± 1 | 0% | N.D. | 919 | 3.121067 |
| tG4-21 | 12 | 76 ± 4 | 50% | 39° C. | 968 | −6.673434 |
| tG4-22 | 10 | 102 ± 3 | 80% | 40° C. | 990 | −9.134224 |
| tG4-23 | 15 | 12 ± 1 | 0% | N.D. | 873 | −2.009122 |
| tG5 | 12 | 23 ± 1 | 0% | 38° C. | 966 | 1.906439 |
| t1A7 | 15 | 14 ± 5 | 0% | N.D. | 891 | −4.59076 |
| t3C11 | 10 | 37 ± 5 | 89% | 41° C. | 982 | 2.458698 |

### Example 6—Biological Activity of Full-Length BoNT/a with the Deimmunized Light Chain

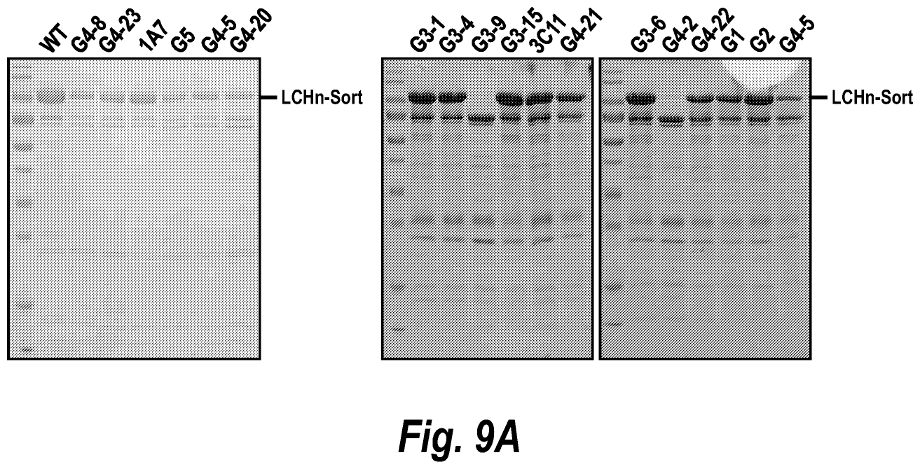

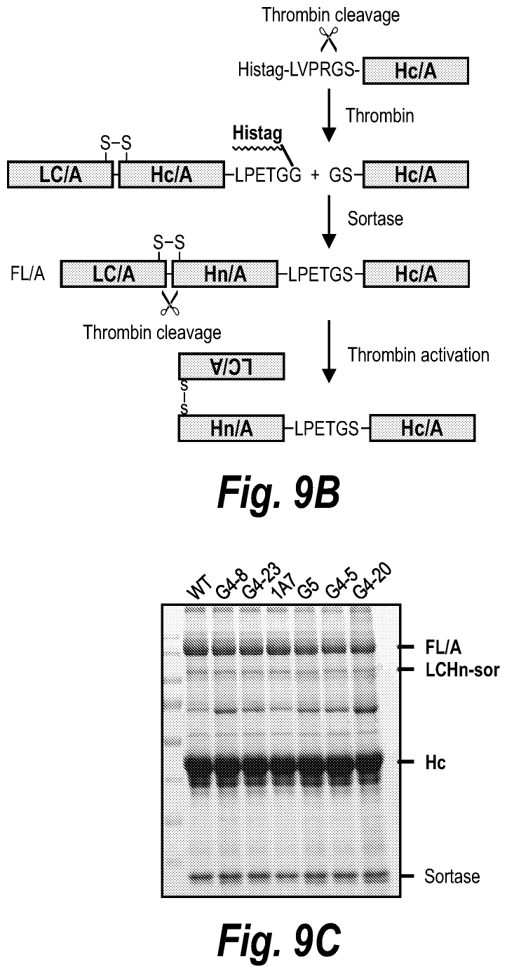

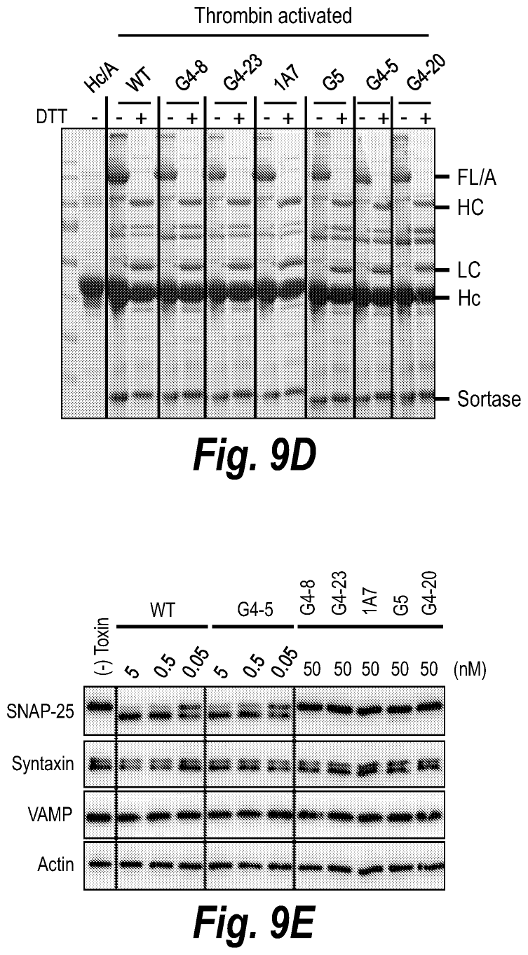

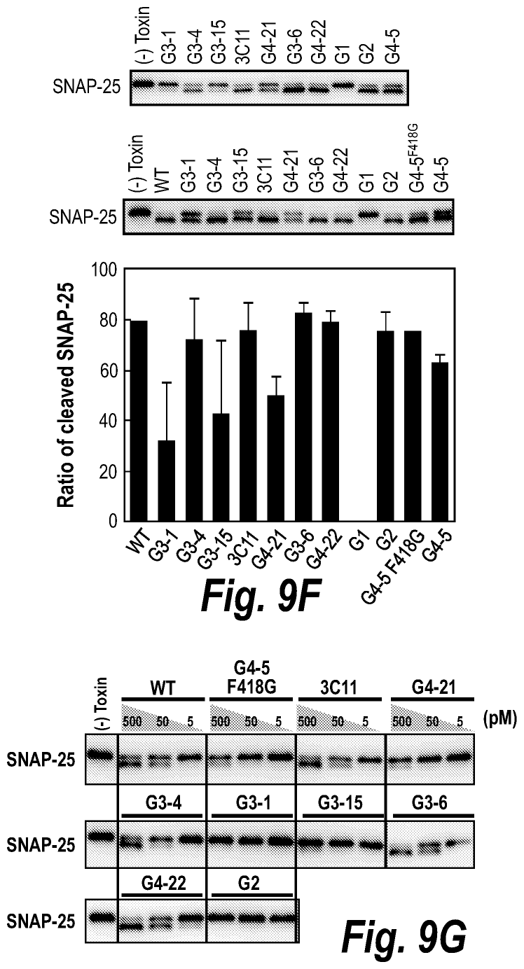

To determine whether the deimmunized ALC variants could assemble with the BoNT/A-HC to form full-length functional neurotoxins, a previously developed sortase-mediated protein ligation method was applied (Zhang 2017, supra). Full-length WT and deimmunized BoNT/A were generated in vitro. Briefly, ALC was genetically fused with the translocation domain (HN) of BoNT/A through a thrombin cleavage site (LVPR/GS), and this gene fusion was appended with coding sequences for a C-terminal sortase substrate motif (LPETGG) and a His₆-tag, yielding the construct LCH_{N}-sort. Separately, a gene for the receptor binding domain of BoNT/A-HC was appended with an N-terminal His₆-tag separated with a thrombin cleavage site spacer, yielding Hc. WT and deimmunized LCH_{N}-sort were expressed in E. coli BL21 (DE3) using an auto-induction medium and purified by Ni-NTA agarose beads (FIG. 9A). Except for G3-9 and G4-2, all of the other 16 deimmunized ALC variants showed reasonable expression levels as LCH_{N}-sort. Hc was expressed in E. coli BL21 (DE3) using an auto-induction medium and purified using Ni-NTA agarose beads. A schematic drawing of the full-length toxin assembly via sortase ligation is illustrated FIG. 9B. Hc is cleaved by thrombin exposing a free N-terminal glycine. Cleaved Hc is then incubated with LC-H_{N} in the presence of sortase, resulting in sortase-mediated fusion of the Hc and LC-H_{N} polypeptides. Finally, full-length toxin is activated by incubation with thrombin, which cleaves the LC-HN fusion protein to yield a disulfide bonded, full length, activated neurotoxin (FIG. 9B), designated FL/A. SDS-PAGE analysis demonstrated that variants G4-8, G4-23, 1A7, G5, G4-5 and G4-20 exhibited assembly and activation efficiency similar to WT toxin (FIG. 9C and FIG. 9D).

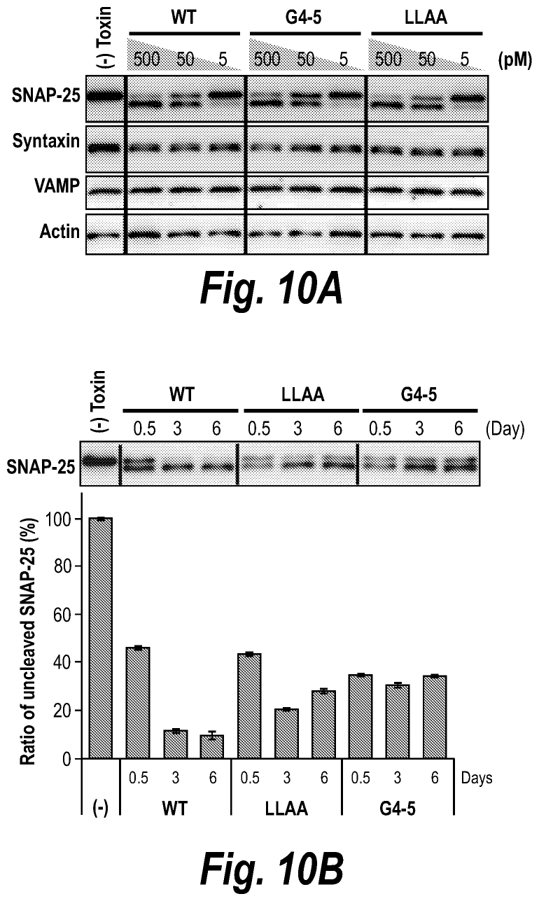

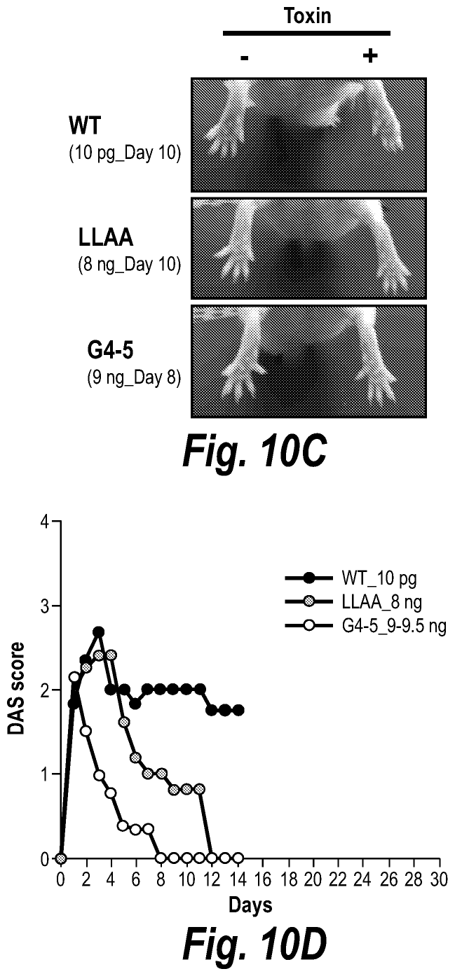

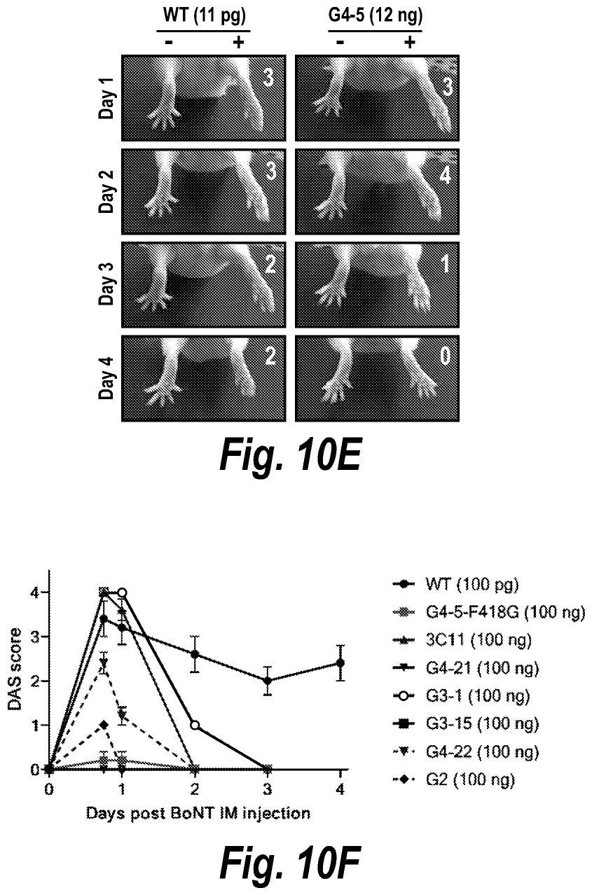

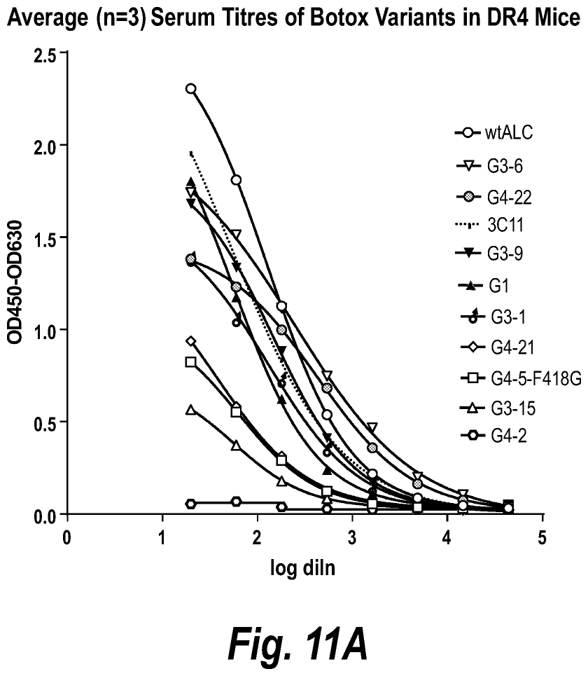

As an initial test of neurotoxin activity, cultured rat cortical neurons were exposed to sortase ligation mixture of WT or six deimmunized FL/A toxins for 12 hours, and subsequently the cells were lysed and analyzed by immunoblotting to identify BoNT/A-mediated SNAP25 degradation. Variant G4-5 showed qualitatively similar cleavage activity of SNAP-25 relative to WT. G4-8 and G5 exhibited weak activity at a high concentration (50 nM), while no detectable activity was observed for 1A7, G5, and G4-20 (FIG. 9E). Following a similar procedure, activity of another eleven deimmunized FL/A toxins, WT, and G4-5 were tested on cultured rat cortical neurons at 5 nM. Variants G2, G3-4, G3-6, G4-22, G4-5-F418G, and 3C11 showed comparable activity with WT while G1 did not exhibit any detectable activity. Variants G3-1, G3-15, and G4-21 showed 30%, 40%, and 50% activity of WT, respectively (FIG. 9F). A subsequent dose-response study suggested that proteolytic activities of G3-4, G3-6, G4-22, and 3C11 on neurons were qualitatively equivalent to WT while G2 and G4-5-F418G exhibited lower activity at a lower concentration of 50 μM (FIG. 9G). To further confirm the biological activity of G4-5, additional rat cortical neuron studies were conducted at lower neurotoxin concentrations, and an additional ALC variant, L428A/L429A or LLAA, engineered for a shorter in vivo half-life (Wang et al. The Journal of biological chemistry. 286, 6375-6385. 2011), was included in the analysis. As shown in FIG. 10A, G4-5, LLAA, and WT exhibited qualitatively similar activity on neuronal cells at concentrations from 5-500 μM. Next, neuronal cells were exposed to 50 μM of ligated FL/A (WT, G4-5 and LLAA) for 0.5, 3 or 6 days. As shown in FIG. 10B, cells treated with G4-5 and LLAA have a similar ratio of cleaved SNAP-25 throughout the 6-day incubation, suggesting that G4-5 and LLAA have similar stability and longitudinal activity on rat cortical neurons in vitro (FIG. 10B).

Based on the results of the neuronal cell assays, G4-5 was selected for in vivo activity analysis in a murine model of muscle paralysis. The in vivo potencies of the BoNT/A toxins were measured using a Digit Abduction Score (DAS), a standard non-lethal murine assay in which local muscle paralysis is quantified after injecting BoNT/A into the mouse hind limb muscles. In a first study, 10 μg of FL/A-WT, 8 ng of FL/A-LLAA, and 9 ng of FL/A-G4-5 were injected into the gastrocnemius muscles of the right hind limb in mice. An active neurotoxin will induce typical flaccid paralysis, evidenced by the failure of toes to spread (FIG. 10A). The G4-5 and LLAA variants produced similar DAS results on day 1 following injection, indicating that both toxin variants are active and able to cause flaccid paralysis in vivo. However, both G4-5 and LLAA were approximately 1,000 times less potent than WT BoNT/A based on the difference in dosing. Moreover, longitudinal tracking of DAS over a two-week period indicated that both LLAA and G4-5 had substantially reduced in vivo duration of action (FIG. 10D), consistent with prior reports on LLAA activity (Wang 2011, supra). LLAA-treated mice fully recovered after day 11 and G4-5-treated mice fully recovered after day 7 (FIG. 10C). In a separate study, 11 μg of FL/A-WT and 12 ng of FL/A-G4-5 were injected to mouse hind limb muscle and the injected limb developed typical flaccid paralysis. The paralysis was also scored by the spread of toes (DAS score; 0-4). On day 1, 11 μg of FL/A-WT and 12 ng of FL/A-G4-5 induced the same level paralysis. While the DAS score of mice injected with WT dropped to 2 in day 3 and 4, the DAS score of G4-5 injected mice dropped to 1 in day 3 and 0 in day 4, indicating the paralysis by G4-5 was recovered 4 days after injection (FIG. 10E). This again confirmed that full-length BoNT/A containing the deimmunized light chain (G4-5) has a shorter duration of action than WT BoNT/A in mice. The above described DAS assay was repeated with additional variants. Specifically, variants G2, G3-1, G3-4, G3-6, G4-21, G4-22, 3C11, and G4-5-F418G were tested (FIG. 10F). In each of the tested variants displayed a degree of in vivo potency relative to wild-type.

### Example 7—Immunogenicity of BoNT/a Deimmunized Light Chain Variants

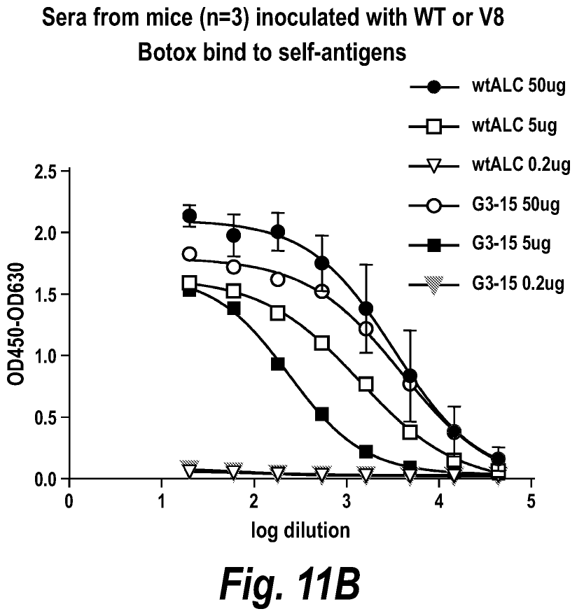

To determine whether the deimmunized ALC variants possess reduced immunogenicity, two different transgenic humanized HLA mice were immunized with the variants and wild-type ALC. DR4 mice encoding the functional variant of human HLA DRB1*0401 were immunized once per a week for 4 weeks with 50 μg of purified light chain variant in PBS. Serum was collected 1 week after the final immunization and anti-drug IgG antibodies were quantified by direct ELISA against the protein immunogen. FIG. 11A and Table 7 below demonstrate that the tested variants all possess reduced immunogenicity compared to a wild-type light chain.

TABLE 7
BoNT/A-LC Immunogenicity in DR4 Mice

| Best-fit values | | | | | |
|---|---|---|---|---|---|
| ALC | wtALC | G4-5-F418G | G1 | G3-9 | G4-21 |
| Bottom | 0.0183 | 0.02772 | 0.02709 | 0.02113 | 0.02517 |
| Top | 2.676 | 1.058 | 2.453 | 1.902 | 1.434 |
| LogIC50 | 2.104 | 1.799 | 1.739 | 2.163 | 1.585 |
| HillSlope | −0.9796 | −1.054 | −0.9877 | −0.9985 | −0.9197 |
| IC50 | 126.9 | 63.01 | 54.78 | 145.7 | 38.45 |
| Span | 2.658 | 1.031 | 2.426 | 1.881 | 1.409 |
| ALC | G4-22 | G3-15 | G3-6 | G4-2 | G3-1 |
| Bottom | 0.009832 | 0.0309 | −0.01703 | 0.02479 | 0.006821 |
| Top | 1.47 | 0.7321 | 2.07 | 0.06052 | 1.62 |
| LogIC50 | 2.632 | 1.753 | 2.389 | ~2.250 | 2.111 |
| HillSlope | −0.8599 | −1.125 | −0.6804 | ~39.29 | −0.9037 |
| IC50 | 428.7 | 56.68 | 244.9 | ~177.7 | 129 |
| Span | 1.46 | 0.7013 | 2.087 | 0.03572 | 1.613 |

DR2 mice encoding the functional variant of human HLA DRB1*1501 were immunized with 50 μg, 5 μg, or 0.2 μg of variant G3-15 in PBS. FIG. 11B and Table 8 below demonstrate that the tested variant possesses reduced immunogenicity compared to a wild-type light chain.

TABLE 8
BoNT/A-LC Immunogenicity in DR2 Mice

| Best-fit values | | | | | | |
|---|---|---|---|---|---|---|
| | wtALC | wtALC | wtALC | G3-15 | G3-15 | G3-15 |
| | 50 ug | 5 ug | 0.2 ug | 50 ug | 5 ug | 0.2 ug |
| Bottom | −0.009036 | −0.04147 | 0.02313 | −0.06094 | 0.03238 | 0.0261 |
| Top | 2.102 | 1.634 | 0.05931 | 1.796 | 1.656 | 0.0964 |
| LogIC50 | 3.501 | 3.137 | 2.09 | 3.586 | 2.376 | 1.722 |
| HillSlope | −0.9461 | −0.8253 | −1.198 | −0.8554 | −1.068 | −0.887 |
| IC50 | 3168 | 1370 | 123 | 3854 | 237.8 | 52.77 |
| Span | 2.111 | 1.676 | 0.03618 | 1.857 | 1.623 | 0.07031 |

### Example 8—Botulinum Toxin Serotype a Light Chain (BoNT/A-LC) Individual Optimized Design

The combinatorial library design approach for identifying deimmunized BoNT/A-LC variants is described above. An alternative optimized design approach was also employed to identify deimmunized BoNT/A-LC variants. An initial set of multi-mutation individual variants predicted to have low immunogenicity were designed. Single point mutations that were common among the best scoring (predictive scoring) initial set of designs were then identified. BoNT/A-LC variant genes for each point mutation were produced, expressed in E. coli, and preliminary activity analysis was conducted to choose functionally validated point mutations. A second round of design using only the functionally validated point mutations was performed, thereby generating a new set of multi-mutation designs with good predicted scores. BoNT/A-LC variant genes for the new multi-mutation designs were produced, expressed in E. coli, and preliminary activity analysis was performed using crude preparations. Variants with good activity in the preliminary analysis were purified and characterized in more detail including: 1) In vitro kinetic data; 2) Thermostability; 3) Activity on neurons following assembly as full-length toxin; 4) For variants active on neurons, activity in a murine paralysis model; and 5) Immunogenicity in two different humanized mouse strains. Table 7 below describes the activity of the point mutants to identify the functionally validated point mutations. Activities were measured on three different days and the average activity was determined.

TABLE 7
BoNT/A-LC Point Mutant Activity

| Variant ID | Mutation | Average Activity | Category |
|---|---|---|---|
| A1-1 | Q30T | 7.66 | med |
| A1-2 | Q30T | 7.72 | med |
| A1-4 | Q30E | 9.81 | high |
| B1-1 | I41V | 7.82 | med |
| C1-1 | V43I | 5.33 | low |
| D1-2 | D80N | 5.80 | low |
| E1-1 | S99E | 8.29 | med |
| F1-4 | F193S | 8.55 | high |
| G1-2 | L199T | 7.16 | med |
| G1-5 | L199Q | 6.60 | med |
| H1-1 | I236G | 6.12 | med |
| A2-1 | F242T | 7.84 | med |
| A2-2 | F242S | 7.89 | med |
| B2-1 | M252Q | 4.21 | low |
| C2-1 | L276A | 7.75 | med |
| D2-1 | E278K | 7.23 | med |
| E2-1 | N279K | 6.86 | med |
| F2-2 | L283N | 8.58 | high |
| F2-3 | L283T | 7.48 | med |
| F2-5 | L283D | 6.08 | med |
| F2-14 | L283E | 8.34 | med |
| G2-2 | Y284K | 9.63 | high |
| H2-1 | Y285A | 6.32 | med |
| A3-1 | F289Y | 5.94 | low |
| B3-4 | S294K | 6.84 | med |
| C3-4 | K298E | 6.95 | med |
| D3-2 | Q310D | 6.60 | med |
| E3-3 | L321G | 6.46 | med |
| E3-4 | L321K | 7.12 | med |
| F3-1 | S323D | 7.49 | med |
| G3-1 | F330Y | 6.26 | med |
| H3-2 Old | L335N | 7.42 | med |
| H3-1 New | L335D | 8.79 | high |
| H3-2 New | L335E | 8.82 | high |
| A4-3 | V354S | 6.23 | med |
| A4-4 | V354A | 8.75 | high |
| B4-1 | K358N | 5.67 | low |
| C4-5 | L360Q | 7.97 | med |
| C4-6 | L360I | 6.92 | med |
| D4-1 | K363Q | 8.34 | med |
| E4-1 | T364S | 7.31 | med |
| F4-1 | N367G | 8.09 | med |
| G4-1 | F368Q | 6.51 | med |
| G4-4 | F368D | 8.38 | med |
| H4-1 | F373K | 1.27 | poor |
| A5-2 | V381D | 8.19 | med |
| A5-3 | V381E | 7.27 | med |
| B5-1 | I385V | 7.66 | med |
| C5-2 | Y386S | 8.45 | med |
| D5-4 | T413D | 6.79 | med |
| D5-6 | T413E | 3.59 | poor |
| E5-3 | K416S | 8.94 | high |
| F5-2 | F418K | 5.26 | low |
| F5-4 | F418E | 6.48 | med |
| G5-2 | L421V | 7.73 | med |
| H5-2 | F422V | 8.07 | med |
| A6-2 | I433T | 8.96 | high |
| B6-3 | I434K | 3.94 | poor |
| C6-1 | T435N | 6.83 | med |
| D6-1 | T438D | 8.56 | high |
| LCN silent | Wild-type | 1.42 | poor |
| LCN sensor | Neg Cont | 11.30 | high |

Based on the results of the preliminary point mutant screen, groups of mutations were made to individual light chains. Table 8 below describes several multi-mutation designs with good predicted scores.

TABLE 8
BoNT/A-LC Full Designs Mutations

| S011426 | Design ID | Mutation |
|---|---|---|
| 1 | Yj_6_5 | S99E \| F193S \| L283E \| V354A \| V381D \| I433T |
| | (N1) | |
| 2 | Yj_7_6 | S99E \| F193S \| L283E \| L335D \| V354A \| V381D |
| | (N2) | \| I433T |
| 3 | Yj_8_14 | Q30E \| S99E \| F193S \| L283E \| L335D \| V354A |
| | (N3) | \| V381D \| T438D |
| 4 | Yj_9_1 | Q30E \| S99E \| L283E \| L335D \| V354A \| V381D |
| | (N4) | \| K416S \| I433T \| T438D |
| 5 | GG_1_10 | S99E \| L199T \| N279K \| L283E \| L321K \| S323D |
| | (N5) | \| L335E \| V381D \| K416S \| T438D |
| 6 | GG_1_10r2 | S99E \| N279K \| L283E \| L321K \| S323D \| |
| | | V381D \| K416S \| T438D |
| 7 | Yj_10_2 | Q30E \| S99E \| F193S \| L283E \| L335D \| V354A |
| | (N6) | \| V381D \| K416S \| I433T \| T438D |
| 8 | gg_8_11 | S99E \| L199T \| N279K \| L283E \| L321K \| S323D |
| | (N7) | \| L335E \| L360Q \| V381D \| L421V \| T438D |
| 9 | yj_11_3 | Q30E \| S99E \| L283E \| Y285K \| L335D \| V354A \| |
| | (N8) | V381D \| Y386S \| K416S \| I433T \| T438D |
| 10 | gg_6_12 | Q30E \| S99E \| L199T \| N279K \| L283E \| L321K \| |
| | (N9) | S323D \| L335D \| V381D \| L421V \| I433T \| |
| | | T438D |
| 11 | Yj_12_1 | Q30E \| S99E \| F193S \| L283E \| Y285K \| L335D \| |
| | (N10) | V354A \| V381D \| Y386S \| K416S \| I433T \| |
| | | T438D |
| 12 | gg_7_13 | Q30E \| S99E \| L199T \| L276A \| N279K \| L283E \| |
| | (N11) | S323D \| L335D \| V381D \| K416S \| L421V \| |
| | | I433T \| T438D |
| 13 | gg_7_13r2 | Q30E \| S99E \| L276A \| N279K \| L283E \| S323D |
| | | \| V381D \| K416S \| L421V \| I433T \| T438D |
| 14 | gg_25_13 | S99E \| L199T \| F242T \| N279K \| L283E \| S323D |
| | (N12) | \| L335D \| L360Q \| V381D \| K416S \| L421V \| |
| | | I433T \| T438D |
| 15 | gg_37_14 | S99E \| L199T \| F242T \| N279K \| L283E \| L321K |
| | (N13) | \| S323D \| L335D \| V354A \| L360Q \| V381D \| |
| | | I385V \| I433T \| T438D |
| 16 | gg_33_14 | Q30E \| S99E \| L199T \| F242T \| L276A \| L283E \| |
| | (N14) | L321K \| S323D \| L335D \| L360Q \| V381D \| |
| | | K416S \| I433T \| T438D |
| 17 | gg_45_16 | Q30E \| S99E \| L199T \| F242T \| L283E \| L321K \| |
| | | S323D \| L335D \| V354A \| L360Q \| N367G \| |
| | | V381D \| I385V \| K416S \| I433T \| T438D |
| 18 | gg_62 | Q30E \| S99E \| F193S \| L199T \| F242T \| L283E \| |
| | | L321K \| L335D \| V354A \| L360Q \| N367G \| |
| | | V381D \| I385V \| K416S \| L421V \| I433T \| |
| | | T438D |
| 19 | gg_62r2 | Q30E \| S99E \| F193S \| F242T \| L283E \| L321K \| |
| | | V354A \| L360Q \| N367G \| V381D \| I385V \| |
| | | K416S \| L421V \| I433T \| T438D |
| 20 | gg_65 | Q30E \| S99E \| L199T \| F242T \| L276A \| L283E \| |
| | (N15) | L321K \| L335D \| V354A \| L360Q \| F368D \| |
| | | V381D \| Y386S \| K416S \| L421V \| I433T \| |
| | | T438D |
| 21 | Gg_78 | Q30E \| S99E \| F193S \| L199T \| F242S \| L276A \| |
| | | L283E \| L321K \| L335D \| V354A \| L360Q \| |
| | | K363Q \| F368D \| V381D \| Y386S \| K416S \| |
| | | L421V \| I433T \| T438D |
| 22 | Gg_83 | Q30E \| S99E \| F193S \| L199T \| F242T \| E278K \| |
| | | L283E \| L321K \| L335E \| V354A \| L360Q \| |
| | | K363Q \| N367G \| F368D \| V381D \| Y386S \| |
| | | K416S \| L421V \| I433T \| T438D |
| 23 | Gg_83r1 | Q30E \| S99E \| F193S \| F242T \| E278K \| L283E \| |
| | | L321K \| V354A \| L360Q \| K363Q \| N367G \| |
| | | F368D \| V381D \| Y386S \| K416S \| L421V \| |
| | | I433T \| T438D |
| 24 | Gg_83r2 | F193S \| F242T \| E278K \| L321K \| V354A \| |
| | | L360Q \| K363Q \| N367G \| Y386S \| K416S \| |
| | | L421V \| I433T |
| | WT-ALC | wild-type |
| | ALC-silent | poorly active negative control |

The BoNT/A-LC variants described above were produced, expressed in E. coli, and preliminary activity analysis was performed using crude preparations. The FRET sensor described in Example 3 was employed to measure BoNT/A-LC variant activity, which is recited below in Table 9.

TABLE 9
BoNT/A-LC Full Designs Activity

| | Activity 1 | Activity 2 | Activity 3 | Activity 4 | Activity 5 | | |
|---|---|---|---|---|---|---|---|
| | (Clover/FRET) | (Clover/FRET) | (Clover/FRET) | (Clover/FRET) | (Clover/FRET) | Avg | |
| Design | Timepoint 1 | Timepoint 2 | Timepoint 3 | Timepoint 4 | Timepoint 5 | Activity | Category |
| Yj_6_5 | 9.40 | 9.83 | 9.93 | 8.42 | 8.67 | 9.25 | high |
| Yj_7_6 | 9.43 | 9.50 | 9.76 | 8.71 | 8.89 | 9.26 | high |
| Yj_8_14 | 9.54 | 10.20 | 9.93 | 8.79 | 9.36 | 9.56 | high |
| Yj_9_1 | 9.60 | 9.86 | 9.44 | 7.97 | 8.11 | 9.00 | high |
| GG_1_10 | 5.22 | 7.45 | 6.56 | 5.07 | 5.35 | 5.93 | medium |
| GG_1_10r2 | 5.91 | 7.70 | 7.43 | 5.31 | 5.57 | 6.38 | medium |
| Yj_10_2 | 4.47 | 5.80 | 4.91 | 4.74 | 4.68 | 4.92 | low |
| gg_8_11 | 8.12 | 10.86 | 10.37 | 8.59 | 8.70 | 9.33 | high |
| yj_11_3 | 8.07 | 10.06 | 10.08 | 8.76 | 8.73 | 9.14 | high |
| gg_6_12 | 7.73 | 10.25 | 9.92 | 8.86 | 8.87 | 9.12 | high |
| Yj_12_1 | 5.77 | 8.81 | 8.39 | 6.66 | 6.98 | 7.32 | medium |
| gg_7_13 | 7.52 | 10.51 | 9.63 | 7.77 | 7.59 | 8.61 | high |
| gg_7_13r2 | 6.63 | 9.92 | 9.70 | 7.28 | 6.96 | 8.10 | medium |
| gg_25_13 | 6.70 | 5.82 | 4.71 | 4.91 | 4.54 | 5.34 | low |
| gg_37_14 | 9.48 | 9.94 | 9.73 | 8.73 | 9.13 | 9.40 | high |
| gg_33_14 | 5.49 | 6.89 | 6.69 | 5.69 | 5.07 | 5.97 | medium |
| gg_45_16 | 4.68 | 4.69 | 4.76 | 4.62 | 4.39 | 4.63 | low |
| gg_62 | 3.89 | 3.99 | 4.02 | 4.48 | 4.26 | 4.13 | poor |
| gg_62r2 | 4.52 | 4.18 | 4.31 | 4.45 | 4.42 | 4.38 | poor |
| gg_65 | 5.22 | 5.99 | 6.18 | 4.99 | 4.85 | 5.45 | low |
| Gg_78 | 4.45 | 4.29 | 4.17 | 4.43 | 4.25 | 4.32 | poor |
| Gg_83 | 4.39 | 4.43 | 4.35 | 4.55 | 4.40 | 4.42 | poor |
| Gg_83r1 | 4.38 | 4.40 | 4.22 | 4.31 | 4.36 | 4.33 | poor |
| Gg_83r2 | 4.32 | 4.47 | 4.35 | 4.45 | 4.37 | 4.39 | poor |
| WT-ALC | 10.19 | 11.94 | 10.59 | 6.98 | 9.19 | 9.78 | high |
| ALC-silent | 4.31 | 3.91 | 4.07 | 4.37 | 4.27 | 4.19 | poor |

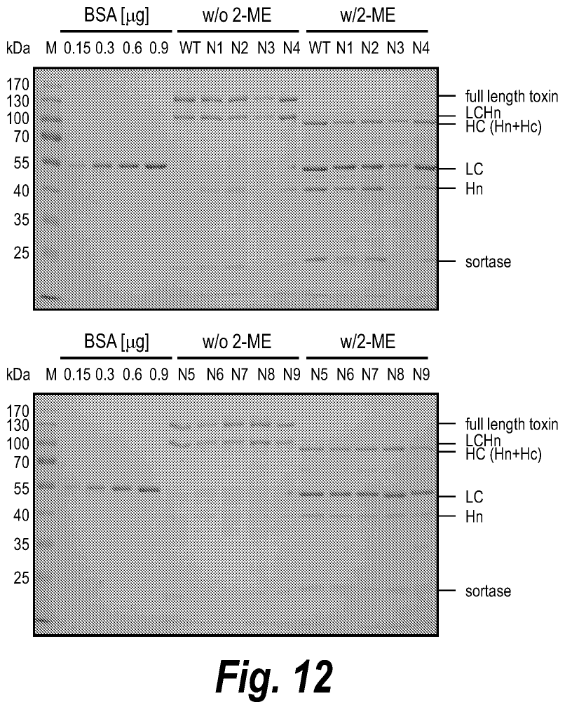

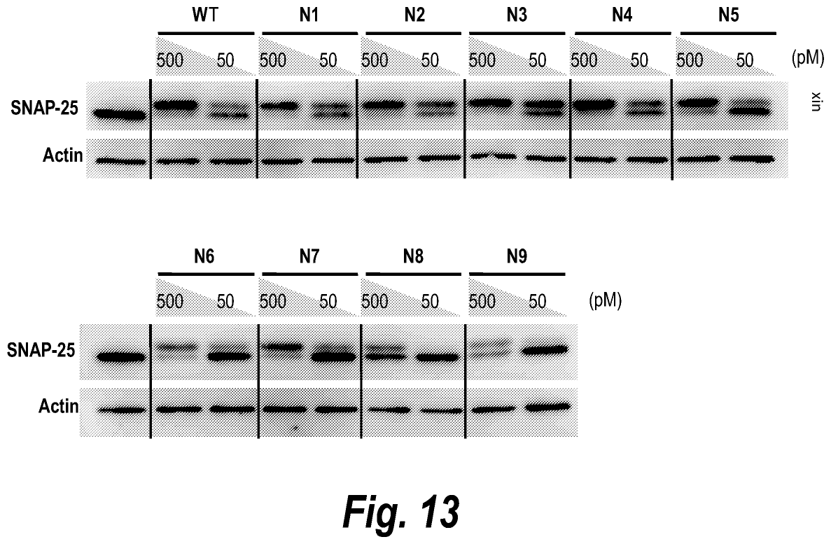

Following the results of the preliminary analysis, full length BoNT/A with select variants were produced with a sortase ligation method. FIG. 12 depicts an electrophoresis gel demonstrating successful generation of full length variants. The full length variants were then used in the SNAP-25 cleavage assay as described above in Example 6. As depicted in FIG. 13, all of the tested variants displayed similar SNAP-25 cleavage activity relative to wild type.

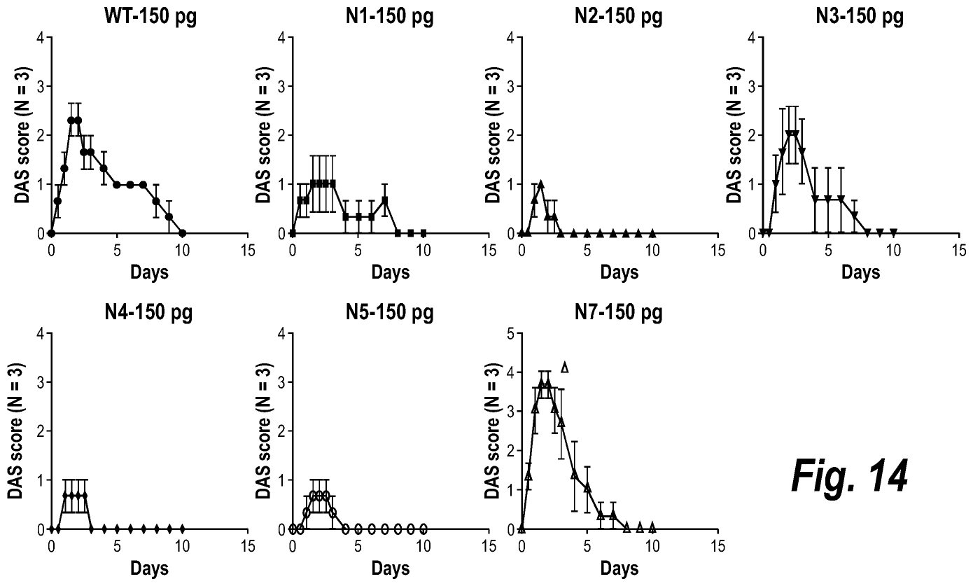

The in vivo potency of the variants was tested next in a DAS assay, as described above in Example 6. The average DAS score was measured from three mice. As depicted in FIG. 14, each of the tested variants displayed in vivo activity. Among the variants tested, variant N7 displayed greater in vivo potency than even wild type.

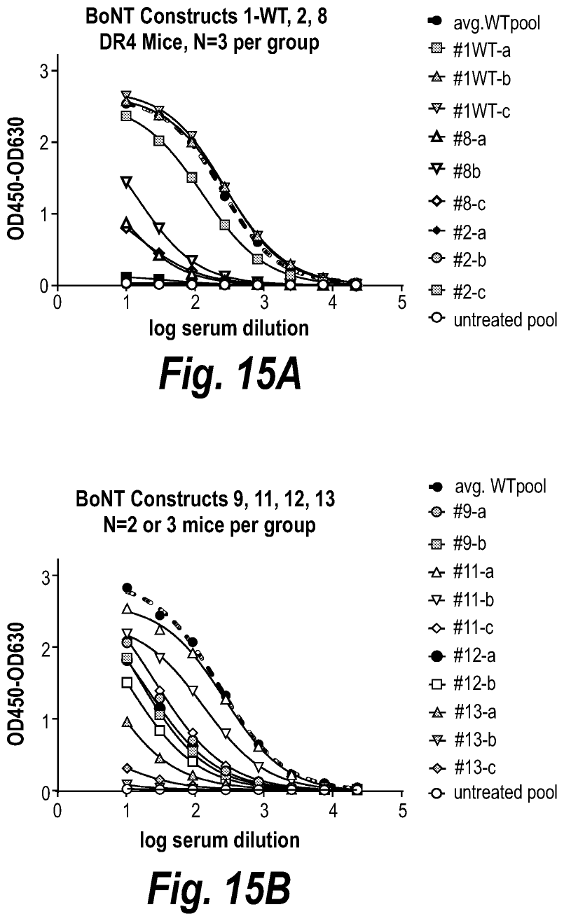

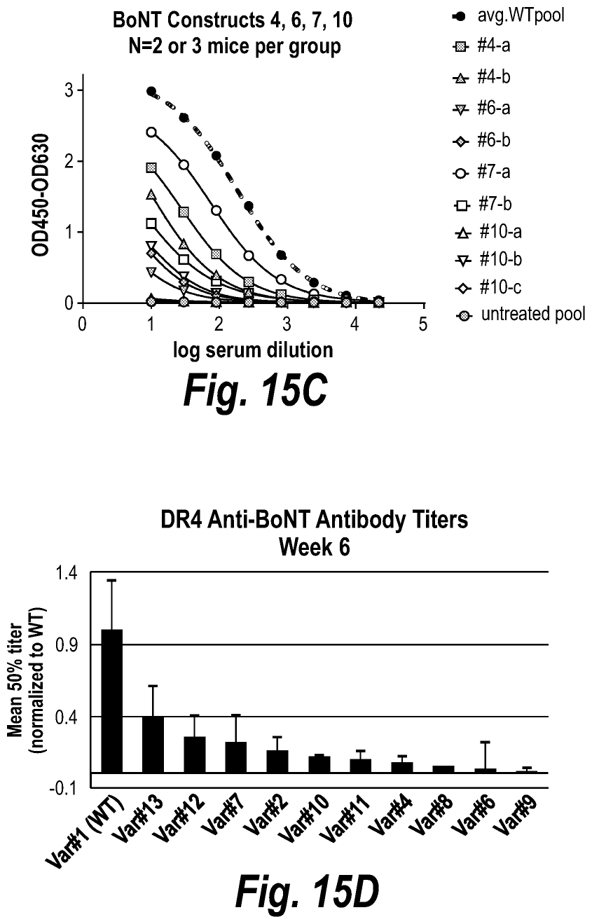

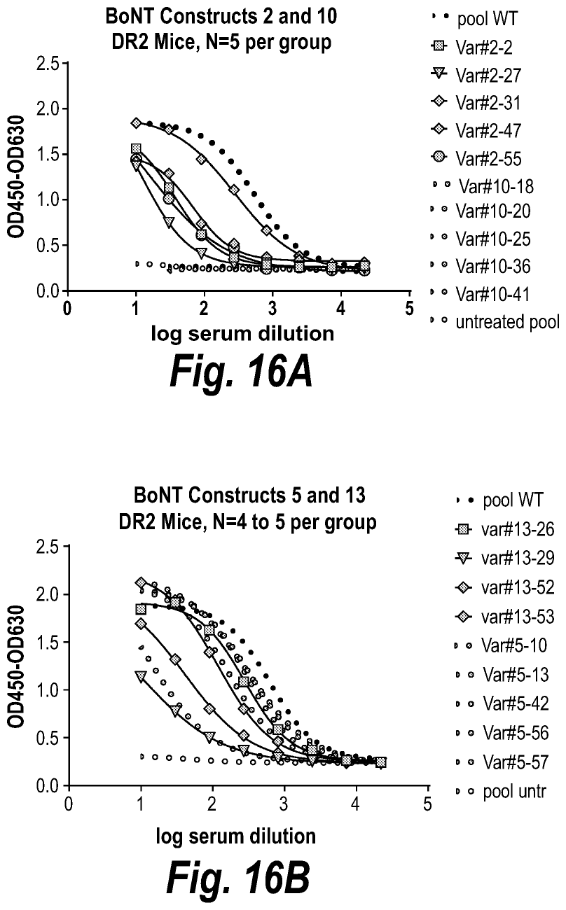

Additional immunogenicity studies were performed with several of the BoNT/A-LC variants. Several of the library-based design variants were tested in DR4 and DR2 mice, as described in Example 7. As shown in FIG. 15A-15C, all of the tested variants displayed reduced immunogenicity compared to WT BoNT/A-LC. FIG. 15D shows a significant reduction in anti-BoNT/A-LC antibody titers up to 6 weeks after administration compared to WT BoNT/A-LC. These effects were observed in the DR2 mouse model as well, as shown in FIG. 16A and FIG. 16B.

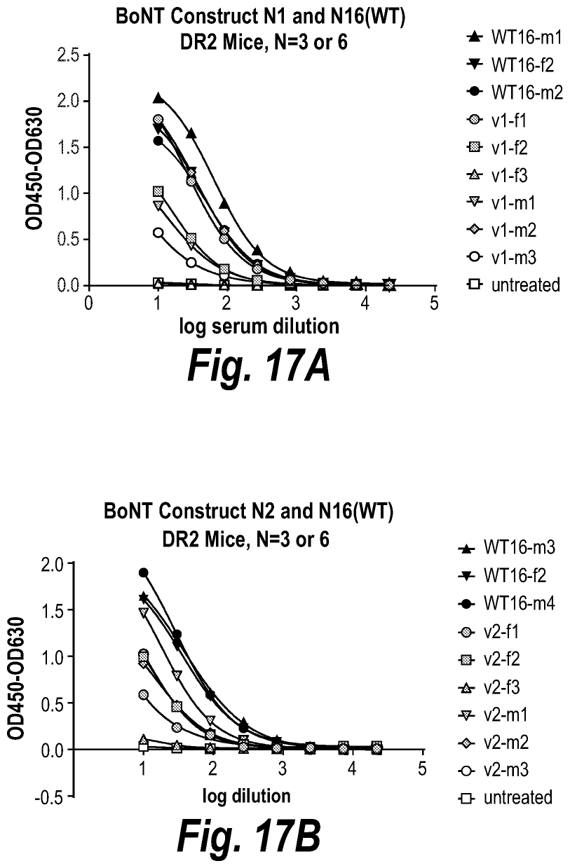

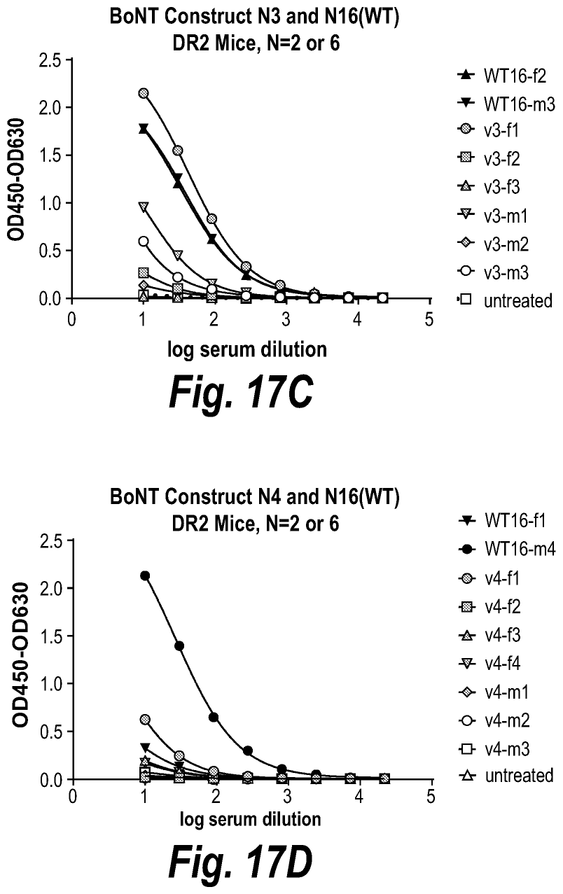

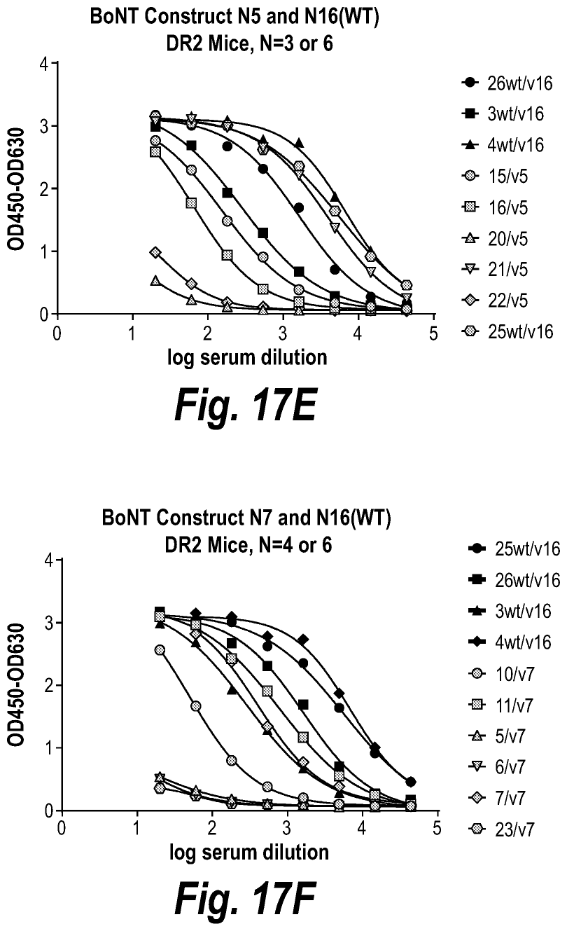

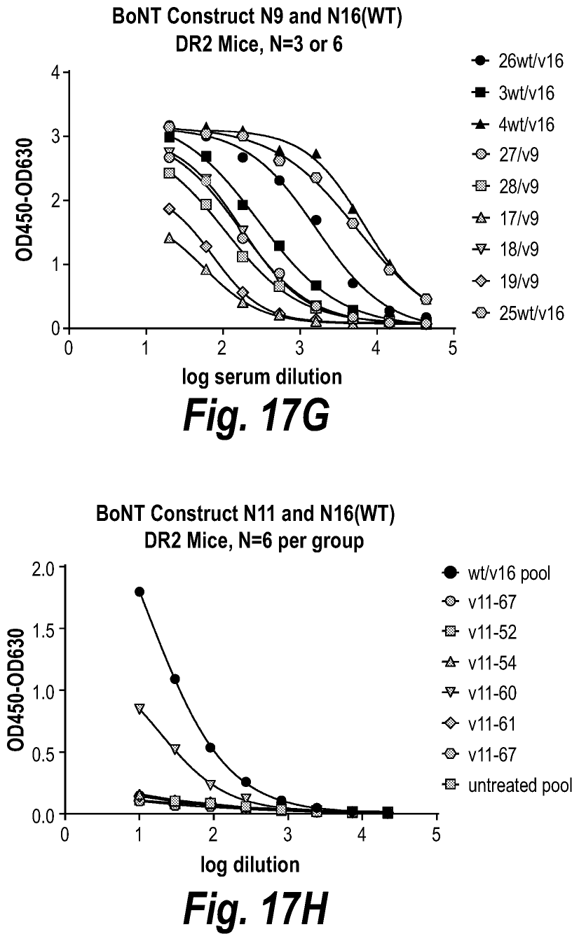

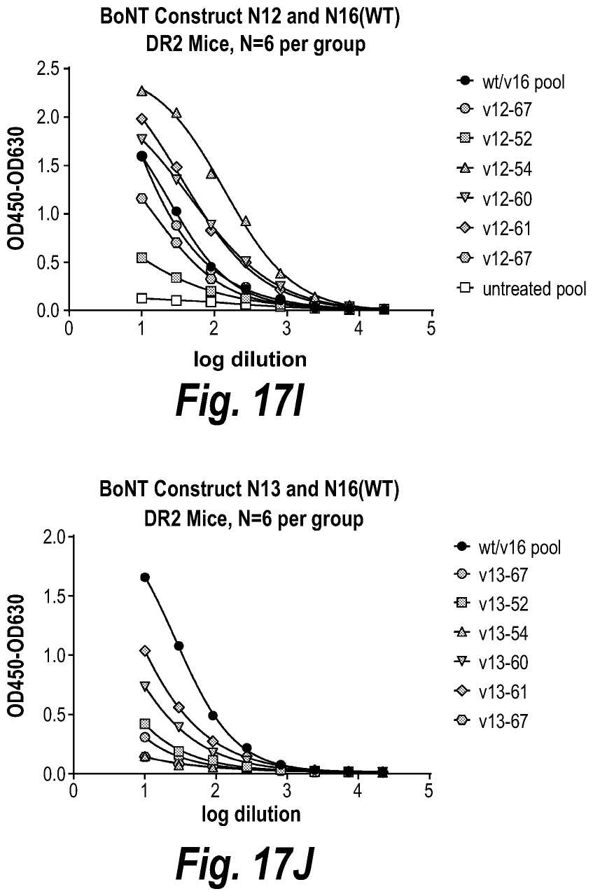

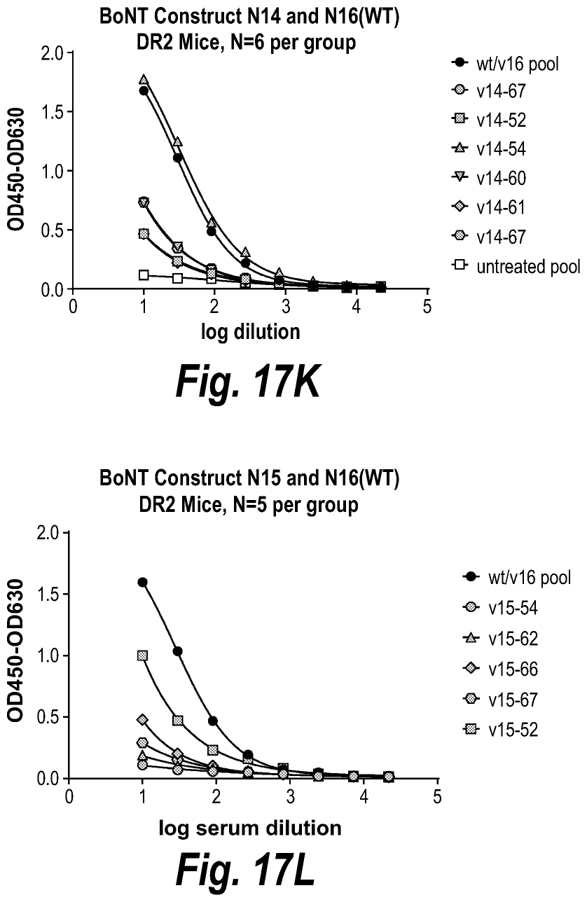

Several of the individual optimal design variants were also tested in DR2 mice, as described in Example 7. As shown in FIG. 17A-FIG. 17L, numerous BoNT/A-LC variants displayed reduced immunogenicity relative to WT BoNT/A-LC.

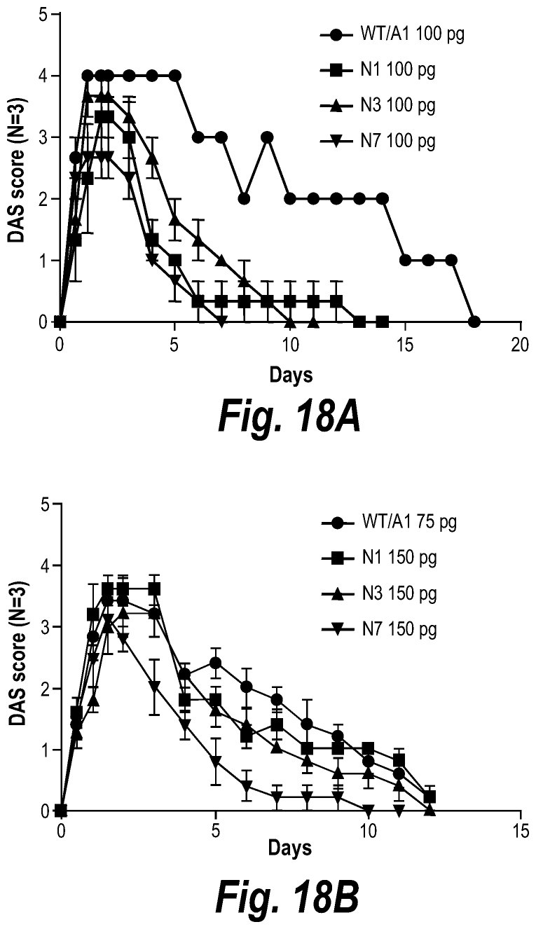

In addition to testing the immunogenicity of the library-based design and individual optimal design variants, the in vivo potency was also tested. Variants N1, N3, and N7 were tested in the DAS assay described in Example 6. As shown in FIG. 18A and FIG. 18B, each of these variants displayed activity at both 100 μg and 150 μg of the variant.

A computational analysis was performed to identify and rank numerous deimmunizing point mutations BoNT/A-LC. Each mutation recited below is ranked according to the frequency with which said mutation appears in a structure-based analysis with a Cluster Expansion (CE) technique as described above. The mutations are recited below in Table 10. The delta episcore is the reduction in predicted T cell epitope content, relative to wild-type, achieved by each individual mutation. In this scoring scheme, each predicted peptide-MHC II binding event is scored equally (i.e., given a value of 1), and the sum of all predicted epitopes for each variant is compared to the sum for wild-type. The delta episcore is therefore the number of predicted peptide-MHC II binding events that are deleted via the cited mutation.

TABLE 10
BoNT/A-LC Point Mutations

| Mutation | Frequency | delta_episcore | priority |
|---|---|---|---|
| V381E | 6520 | 41 | 1 |
| L335D | 6095 | 25 | 2 |
| T413D | 4818 | 20 | 3 |
| Y386H | 4609 | 15 | 4 |
| L360K | 4309 | 36 | 5 |
| S294K | 3961 | 22 | 6 |
| S99E | 3924 | 37 | 7 |
| I433T | 3892 | 38 | 8 |
| L321G | 3884 | 32 | 9 |
| F422V | 2975 | 24 | 10 |
| I41V | 2771 | 5 | 11 |
| L360Q | 2096 | 33 | 12 |
| L199T | 1966 | 35 | 13 |
| F242T | 1942 | 53 | 14 |
| L321N | 1481 | 30 | 15 |
| V43I | 1294 | 5 | 16 |
| Y284K | 1277 | 38 | 17 |
| F418E | 1252 | 29 | 18 |
| K363Q | 1053 | 16 | 19 |
| L283E | 1017 | 39 | 20 |
| I434K | 947 | 16 | 21 |
| F289Y | 923 | 23 | 22 |
| T435N | 864 | 12 | 23 |
| F330Y | 481 | 5 | 24 |
| L360I | 391 | 22 | 25 |
| S323D | 334 | 7 | 26 |
| Y285A | 329 | 28 | 27 |
| T438D | 303 | 23 | 28 |
| L283D | 296 | 41 | 29 |
| K298E | 279 | 15 | 30 |
| V381D | 174 | 43 | 31 |
| M252Q | 140 | 16 | 32 |
| Q30T | 132 | 11 | 33 |
| F418K | 119 | 31 | 34 |
| F242S | 117 | 49 | 35 |
| L335E | 100 | 24 | 36 |
| T364S | 98 | 10 | 37 |
| N367G | 91 | 14 | 38 |
| K358N | 82 | 15 | 39 |
| F373K | 70 | 46 | 40 |
| I236G | 60 | 37 | 41 |
| L421V | 60 | 14 | 42 |
| Q310D | 51 | 25 | 43 |
| F368D | 46 | 29 | 44 |
| N279K | 41 | 5 | 45 |
| I385V | 39 | 10 | 46 |
| L199Q | 37 | 26 | 47 |
| S294D | 34 | 21 | 48 |
| E278K | 32 | 7 | 49 |
| L283T | 30 | 34 | 50 |
| L335N | 22 | 24 | 51 |
| L321K | 20 | 28 | 52 |
| V354S | 19 | 38 | 53 |
| D80N | 14 | 5 | 54 |
| F193S | 8 | 34 | 55 |
| L283N | 8 | 37 | 56 |
| Y386S | 7 | 10 | 57 |
| K416S | 6 | 13 | 58 |
| F368Q | 4 | 16 | 59 |
| Q30E | 3 | 14 | 60 |
| V354A | 3 | 34 | 61 |
| T413E | 2 | 23 | 62 |
| L276A | 1 | 11 | 63 |
| I137K | 0 | 19 | 100 |
| I137N | 0 | 19 | 100 |
| A157E | 0 | 16 | 100 |
| I225N | 0 | 16 | 100 |
| S156G | 0 | 15 | 100 |
| I225T | 0 | 15 | 100 |
| L150V | 0 | 13 | 100 |
| I153F | 0 | 13 | 100 |
| V69Q | 0 | 9 | 100 |
| V69S | 0 | 9 | 100 |
| I160F | 0 | 9 | 100 |
| I160N | 0 | 9 | 100 |
| F212Y | 0 | 9 | 100 |
| I152V | 0 | 8 | 100 |
| Q183N | 0 | 7 | 100 |
| I129T | 0 | 6 | 100 |
| V16K | 0 | 5 | 100 |
| V16N | 0 | 5 | 100 |
| V16R | 0 | 5 | 100 |
| I159L | 0 | 5 | 100 |
| K211E | 0 | 5 | 100 |
| F400N | 0 | 5 | 100 |
| I234A | 0 | 26 | 1000 |
| F281I | 0 | 26 | 1000 |
| I375A | 0 | 23 | 1000 |
| I375S | 0 | 21 | 1000 |
| K336S | 0 | 20 | 1000 |
| N417D | 0 | 20 | 1000 |
| V32E | 0 | 19 | 1000 |
| K374T | 0 | 19 | 1000 |
| F412L | 0 | 19 | 1000 |
| Y184Q | 0 | 18 | 1000 |
| V303S | 0 | 18 | 1000 |
| K317E | 0 | 18 | 1000 |
| I302A | 0 | 16 | 1000 |
| I302T | 0 | 16 | 1000 |
| K414E | 0 | 15 | 1000 |
| V303N | 0 | 13 | 1000 |
| F412I | 0 | 13 | 1000 |
| N417G | 0 | 13 | 1000 |
| A235N | 0 | 12 | 1000 |
| F357Y | 0 | 12 | 1000 |
| Y184M | 0 | 11 | 1000 |
| S327D | 0 | 11 | 1000 |
| L415V | 0 | 11 | 1000 |
| I21L | 0 | 10 | 1000 |
| Y184I | 0 | 10 | 1000 |
| N81A | 0 | 9 | 1000 |
| N81D | 0 | 9 | 1000 |
| N81E | 0 | 9 | 1000 |
| F412Y | 0 | 9 | 1000 |
| I44M | 0 | 7 | 1000 |
| N81S | 0 | 7 | 1000 |
| T384N | 0 | 7 | 1000 |
| A227V | 0 | 6 | 1000 |
| S258N | 0 | 6 | 1000 |
| K329N | 0 | 6 | 1000 |
| I377L | 0 | 6 | 1000 |
| R392G | 0 | 6 | 1000 |
| N395G | 0 | 6 | 1000 |
| L93I | 0 | 5 | 1000 |
| T100K | 0 | 5 | 1000 |
| A227S | 0 | 5 | 1000 |
| N297S | 0 | 5 | 1000 |
| D387N | 0 | 5 | 1000 |
| T405A | 0 | 5 | 1000 |
| T405I | 0 | 5 | 1000 |
| T405K | 0 | 5 | 1000 |
| N411A | 0 | 5 | 1000 |
| F193H | 0 | 29 | 10000 |
| V381N | 0 | 28 | 10000 |
| F373V | 0 | 27 | 10000 |
| L321R | 0 | 23 | 10000 |
| K416N | 0 | 18 | 10000 |
| Y386Q | 0 | 15 | 10000 |
| Y386K | 0 | 14 | 10000 |
| S294T | 0 | 13 | 10000 |
| F330V | 0 | 13 | 10000 |
| Q310N | 0 | 12 | 10000 |
| K416T | 0 | 11 | 10000 |
| V381S | 0 | 10 | 10000 |
| Y386E | 0 | 9 | 10000 |
| Y386N | 0 | 9 | 10000 |
| K298S | 0 | 6 | 10000 |
| S99N | 0 | 5 | 10000 |

### Example 9—Botulinum Toxin Serotypes B-G Light Chain (BoNT/B-G-LC) Deimmunization

Deimmunizing mutations were identified for each of BoNT/B-LC, BoNT/C-LC, BoNT/D-LC, BoNT/E-LC, BoNT/F-LC, and BoNT/G-LC, using the methods recited above. The mutations were selected, in part, on an alignment against BoNT/A-LC, making the same or similar point mutations at the corresponding position in BoNT/B-LC, BoNT/C-LC, BoNT/D-LC, BoNT/E-LC, BoNT/F-LC, or BoNT/G-LC. Each mutation or mutation combination was given a delta episcore, as described above. The mutations are recited below in Tables 11-16.

TABLE 11
BoNT/B-LC Mutations Relative to SEQ ID NO: 2

| Mutations relative to | |
|---|---|
| SEQ ID NO: 2 | Delta episcore |
| N16R | 7 |
| N16R/R31E | −23 |
| R31E | −30 |
| D82A | 5 |
| S100E | −58 |
| L140K | −21 |
| L140K/L157V | −26 |
| L157V | −5 |
| L157V/G163G | −5 |
| G163G | 0 |
| Q191I | 41 |
| Q191I/S200N | 32 |
| S200N | 0 |
| Y199Y | 0 |
| Y199Y/I232T | −19 |
| I232T | −19 |
| I232T/I241T | −43 |
| I241T | −24 |
| I241T/P247E | −15 |
| P247E | 8 |
| Q264K | −4 |
| D289D | 0 |
| D289D/K290K | 0 |
| K290K | 0 |
| C308T | 3 |
| C308T/N317D | 3 |
| N317D | −2 |
| N317D/K328K | −2 |
| K328K | 0 |
| K328K/E342N | 3 |
| E342N | 3 |
| A361S | −9 |
| A361S/K367Q | −12 |
| K367Q | −3 |
| K367Q/P379G | −19 |
| P379G | −16 |
| P379G/E389D | −17 |
| E389D | −1 |
| E389D/E394K | −14 |
| E394K | −21 |
| E421D | 0 |

Any one or more of the above recited mutations in Table 11 can be introduced into the WT BoNT/B-LC of SEQ ID NO: 2 to produce a deimmunized BoNT/B-LC variant.

TABLE 12
BoNT/C-LC Mutations Relative to SEQ ID NO: 3

| Mutations relative to | |
|---|---|
| SEQ ID NO: 3 | Delta episcore |
| K16R | 1 |
| K16R/E31E | 1 |
| E31E | 0 |
| S80A | 0 |
| S98E | −58 |
| K138K | 0 |
| K138K/V155V | 0 |
| V155V | 0 |
| V155V/R161G | 0 |
| R161G | 0 |
| I190I | 0 |
| I190I/L199N | −54 |
| L199N | −54 |
| F218Y | −38 |
| F218Y/N231T | −36 |
| N231T | 2 |
| N231T/I240T | 0 |
| I240T | −1 |
| I240T/T247E | 1 |
| T247E | 7 |
| E265K | 21 |
| E290D | 5 |
| E290D/K291K | 5 |
| K291K | 0 |
| A309T | −21 |
| A309T/G319D | −30 |
| G319D | −9 |
| G319D/R330K | −16 |
| R330K | −8 |
| R330K/N344N | −8 |
| N344N | 0 |
| A363S | −32 |
| A363S/Q369Q | −32 |
| Q369Q | 0 |
| Q369Q/P381G | −18 |
| P381G | −18 |
| P381G/N390D | −22 |
| N390D | 0 |
| N390D/Q395K | 3 |
| Q395K | 0 |
| R421D | −15 |

Any one or more of the above recited mutations in Table 12 can be introduced into the WT BoNT/C-LC of SEQ ID NO: 3 to produce a deimmunized BoNT/C-LC variant.

TABLE 13
BoNT/D-LC Mutations Relative to SEQ ID NO: 4

| Mutations relative to | |
|---|---|
| SEQ ID NO: 4 | Delta episcore |
| N16R | 5 |
| N16R/T31E | −13 |
| T31E | −18 |
| E80A | 0 |
| E98E | 0 |
| E138K | 9 |
| E138K/V155V | 9 |
| V155V | 0 |
| V155V/L161G | −11 |
| L161G | −11 |
| I190I | 0 |
| I190I/L199N | −53 |
| L199N | −53 |
| F218Y | −23 |
| F218Y/T231T | −23 |
| T231T | 0 |
| T231T/I240T | −10 |
| I240T | −10 |
| I240T/R247E | −10 |
| R247E | −4 |
| Q265K | 0 |
| E290D | −1 |
| E290D/K291K | −1 |
| K291K | 0 |
| T309T | 0 |
| T309T/D319D | 0 |
| D319D | 0 |
| D319D/N330K | 4 |
| N330K | 4 |
| N330K/D344N | 29 |
| D344N | 25 |
| S363S | 0 |
| S363S/K369Q | 4 |
| K369Q | 4 |
| K369Q/P381G | −22 |
| P381G | −26 |
| P381G/N390D | −29 |
| N390D | −3 |
| N390D/R395K | −4 |
| R395K | −1 |
| Q421D | −9 |

Any one or more of the above recited mutations in Table 13 can be introduced into the WT BoNT/D-LC of SEQ ID NO: 4 to produce a deimmunized BoNT/D-LC variant.

TABLE 14
BoNT/E-LC Mutations Relative to SEQ ID NO: 5

| Mutations relative to | |
|---|---|
| SEQ ID NO: 5 | Delta episcore |
| R15R | 0 |
| R15R/E27E | 0 |
| E27E | 0 |
| E77A | 0 |
| N95E | −29 |
| K134K | 0 |
| K134K/V147V | 0 |
| V147V | 0 |
| V147V/E153G | −11 |
| E153G | −11 |
| I182I | 0 |
| I182I/F191N | −28 |
| F191N | −28 |
| F201Y | −25 |
| F201Y/I214T | −69 |
| I214T | −46 |
| I214T/A223T | −57 |
| A223T | −22 |
| A223T/Y230E | −22 |
| Y230E | −4 |
| N247K | −1 |
| T272D | −11 |
| T272D/N273K | −10 |
| N273K | 6 |
| S291T | −13 |
| S291T/N296D | −16 |
| N296D | −10 |
| N296D/G307K | −5 |
| G307K | 5 |
| G307K/N321N | 5 |
| N321N | 0 |
| R339S | 13 |
| R339S/K345Q | 13 |
| K345Q | −1 |
| K345Q/Y356G | −48 |
| Y356G | −47 |
| Y356G/S366D | −60 |
| S366D | −14 |
| S366D/S371K | −25 |
| S371K | −10 |
| T396D | −20 |

Any one or more of the above recited mutations in Table 14 can be introduced into the WT BoNT/E-LC of SEQ ID NO: 5 to produce a deimmunized BoNT/E-LC variant.

TABLE 15
BoNT/F-LC Mutations Relative to SEQ ID NO: 6

| Mutations relative to | |
|---|---|
| SEQ ID NO: 6 | Delta episcore |
| D16R | 19 |
| D16R/K31E | 7 |
| K31E | −12 |
| A81A | 0 |
| S99E | −63 |
| K139K | 0 |
| K139K/L152V | 5 |
| L152V | 5 |
| L152V/G158G | 5 |
| G158G | 0 |
| I191I | 0 |
| I191I/Y200N | −5 |
| Y200N | −5 |
| F216Y | −31 |
| F216Y/I229T | −73 |
| I229T | −42 |
| I229T/A238T | −49 |
| A238T | −14 |
| A238T/E245E | −14 |
| E245E | 0 |
| R262K | −10 |
| N287D | −19 |
| N287D/N288K | −8 |
| N288K | 27 |
| A306T | −7 |
| A306T/N313D | −8 |
| N313D | −1 |
| N313D/G324K | 5 |
| G324K | 7 |
| G324K/N338N | 7 |
| N338N | 0 |
| A356S | −16 |
| A356S/K362Q | −16 |
| K362Q | 0 |
| K362Q/F373G | −18 |
| F373G | −17 |
| F373G/D383D | −17 |
| D383D | 0 |
| D383D/S388K | −15 |
| S388K | −15 |
| D413D | 0 |

Any one or more of the above recited mutations in Table 15 can be introduced into the WT BoNT/F-LC of SEQ ID NO: 6 to produce a deimmunized BoNT/F-LC variant.

TABLE 16
BoNT/G-LC Mutations Relative to SEQ ID NO: 7

| Mutations relative to | |
|---|---|
| SEQ ID NO: 7 | Delta episcore |
| D16R | −15 |
| D16R/T31E | −17 |
| T31E | −2 |
| A82A | 0 |
| S100E | −35 |
| K140K | 0 |
| K140K/L157V | −8 |
| L157V | −8 |
| L157V/G163G | −8 |
| G163G | 0 |
| M191I | 8 |
| M191I/N200N | 8 |
| N200N | 0 |
| Y219Y | 0 |
| Y219Y/I232T | −23 |
| I232T | −23 |
| I232T/I241T | −62 |
| I241T | −31 |
| I241T/P247E | −33 |
| P247E | −25 |
| Q264K | −13 |
| N289D | −1 |
| N289D/K290K | −1 |
| K290K | 0 |
| A308T | −16 |
| A308T/S316D | −25 |
| S316D | −9 |
| S316D/D327K | 7 |
| D327K | 14 |
| D327K/D341N | 21 |
| D341N | 7 |
| A360S | −4 |
| A360S/K366Q | −10 |
| K366Q | −6 |
| K366Q/P378G | −18 |
| P378G | −13 |
| P378G/T388D | −15 |
| T388D | −2 |
| T388D/N393K | −7 |
| N393K | −7 |
| E420D | −18 |

Any one or more of the above recited mutations in Table 16 can be introduced into the WT BoNT/G-LC of SEQ ID NO: 7 to produce a deimmunized BoNT/G-LC variant.

The above recited mutations in Tables 11-16 are expected to confer reduced immunogenicity to the botulinum toxin light chain serotypes (i.e., yield deimmunized botulinum toxin light chains). The in vitro characterization assays of Example 5 or the in vivo assays (e.g., DAS assay) of Example 8 can be used to validate the efficacy of each mutation, either alone or in combination.

What is claimed:

1. A deimmunized botulinum toxin light chain or fragment thereof comprising at least one mutation in a botulinum toxin light chain amino acid sequence selected from the group consisting of:
a) a botulinum toxin serotype A light chain (BoNT/A-LC) or fragment thereof of SEQ ID NO: 1;
b) a botulinum toxin serotype B light chain (BoNT/B-LC) or fragment thereof of SEQ ID NO: 2;
c) a botulinum toxin serotype C light chain (BoNT/C-LC) or fragment thereof of SEQ ID NO: 3;
d) a botulinum toxin serotype D light chain (BoNT/D-LC) or fragment thereof of SEQ ID NO: 4;
e) a botulinum toxin serotype E light chain (BoNT/E-LC) or fragment thereof of SEQ ID NO: 5;
f) a botulinum toxin serotype F light chain (BoNT/F-LC) or fragment thereof of SEQ ID NO: 6; or
g) a botulinum toxin serotype G light chain (BoNT/G-LC) or fragment thereof of SEQ ID NO: 7,
wherein the at least one mutation reduces the immunogenicity of the botulinum toxin light chain or fragment thereof.

2. The deimmunized BoNT/A-LC or fragment thereof of claim 1, comprising a mutation at one or more of V16, Q30, I41, V43, D80, N81, S99, G119, I137, L150, S156, Y184, F193, L199, F212, I225, I234, I236, R240, F242, M252, S258, L276, E278, N279, L283, Y284, Y285, F289, S294, K298, I302, Q310, L321, S323, F330, L335, V354, K358, L360, K363, T364, N367, F368, A371, F373, V381, I385, Y386, T413, K416, F418, L421, F422, I433, I434, T435, and T438 of SEQ ID NO: 1.

3. The deimmunized BoNT/A-LC or fragment thereof of claims 1 or 2, wherein the mutation comprises V16R or V16L; Q30E or Q30T; I41V; V43I; D80N; N81A; S99E; G119S; I137K; L150V; S156G; Y184I; F193S or F193N; L199T or L199Q; F212Y; I225T; I234T; I236G; R240E; F242T or F242S; M252Q; S258K; L276A; E278K; N279K; L283D, L283N, L283E, or L283T; Y284K; Y285A; F289Y; S294K or S249D; K298E; I302T; Q310D; L321K, L321G, or L321N; S323D; F330Y; L335D, L335E, or L335N; V354S or V354A; K358N; L360Q, L360I, or L360K; K363Q; T364S; N367G; F368Q or F368D; A371G; F373K; V381D or V381E; I385V; Y386K, Y386S, or Y386H; T413D or T413E; K416S; F418G, F418K, or F418E; L421V; F422V; I433T; I434K; T435N; T438D; or a combination thereof, of SEQ ID NO: 1.

4. The deimmunized BoNT/B-LC or fragment thereof of claim 1, comprising a mutation at one or more of N16, R31, D82, S100, L140, L157, Q191, S200, I232, I241, P247, Q264, C308, N317, E342, A361, K367, P379, E389, E394, and E421 of SEQ ID NO: 2.

5. The deimmunized BoNT/B-LC or fragment thereof of claim 1 or claim 4, wherein the mutation comprises N16R; R31E; D82A; S100E; L140K; L157V; Q191I; S200N; I232T; I241T; P247E; Q264K; C308T; N317D; E342N; A361S; K367Q; P379G; E389D; E394K; E421D; or a combination thereof, of SEQ ID NO: 2.

6. The deimmunized BoNT/C-LC or fragment thereof of claim 1, comprising a mutation at one or more of K16, S80, S98, R161, L199, F218, N231, I240, T247, E265, E290, A309, G319, R33K, A363, P381, N390, Q395, and R421 of SEQ ID NO: 3.

7. The deimmunized BoNT/C-LC or fragment thereof of claim 1 or claim 6, wherein the mutation comprises K16R; S80A; S98E; R161G; L199N; F218Y; N231T; I240T; T247E; E265K; E290D; A309T; G319D; R330K; A363S; P381G; N390D; Q395K; R421D; or a combination thereof, of SEQ ID NO: 3.

8. The deimmunized BoNT/D-LC or fragment thereof of claim 1, comprising a mutation at one or more of N16, T31, E80, E138, L161, L199, F218, I240, R247, Q265, E290, N330, D344, K369, P381, N390, R395, and Q421 of SEQ ID NO: 4.

9. The deimmunized BoNT/D-LC or fragment thereof of claim 1 or claim 8, wherein the mutation comprises N16R; T31E; E80A; E138K; L161G; L199N; F218Y; I240T; R247E; Q265K; E290D; N330K; D344N; K369Q; P381G; N390D; R395K; Q421D; or a combination thereof, of SEQ ID NO: 4.

10. The deimmunized BoNT/E-LC or fragment thereof of claim 1, comprising a mutation at one or more of E77, N95, E153, F191, F201, I214, A223, Y230, N247, T272, N273, S291, N296, G307, R339, K345, Y356, S366, S371, and T396 of SEQ ID NO: 5.

11. The deimmunized BoNT/E-LC or fragment thereof of claim 1 or claim 8, wherein the mutation comprises E77A; N95E; E153G; F191N; F201Y; I214T; A223T; Y230E; N247K; T272D; N273K; S291T; N296D; G307K; R339S; K345Q; Y356G; S366D; S371K; T396D; or a combination thereof, of SEQ ID NO: 5.

12. The deimmunized BoNT/F-LC or fragment thereof of claim 1, comprising a mutation at one or more of D16, K31, S99, L152, Y200, F216, I229, A238, R262, N287, N288, A306, N313, G324, A356, K362, F373, and S388 of SEQ ID NO: 6.

13. The deimmunized BoNT/F-LC or fragment thereof of claim 1 or claim 12, wherein the mutation comprises D16R; K31E; S99E; L152V; Y200N; F216Y; I229T; A238T; R262K; N287D; N288K; A306T; N313D; G324K; A356S; K362Q; F373G; S388K; or a combination thereof, of SEQ ID NO: 6.

14. The deimmunized BoNT/G-LC or fragment thereof of claim 1, comprising a mutation at one or more of D16, T31, S100, L157, M191, I232, I241, P247, Q264, N289, A308, S316, D327, D341, A360, K366, P378, T388, N393, and E420 of SEQ ID NO: 7.

15. The deimmunized BoNT/G-LC or fragment thereof of claim 1 or claim 14, wherein the mutation comprises D16R; T31E; S100E; L157V; M191I; I232T; I241T; P247E; Q264K; N289D; A308T; S316D; D327K; D341N; A360S; K366Q; P378G; T388D; N393K; E420D; or a combination thereof, of SEQ ID NO: 7.

16. A deimmunized botulinum toxin serotype A light chain (BoNT/A-LC) or fragment thereof comprising a mutation at one or more of V16, Q30, I41, V43, D80, N81, S99, G119, I137, L150, S156, Y184, F193, L199, F212, I225, I234, I236, R240, F242, M252, S258, L276, E278, N279, L283, Y284, Y285, F289, S294, K298, I302, Q310, L321, S323, F330, L335, V354, K358, L360, K363, T364, N367, F368, A371, F373, V381, I385, Y386, T413, K416, F418, L421, F422, I433, I434, T435, and T438 of SEQ ID NO: 1.

17. A deimmunized botulinum toxin serotype A light chain (BoNT/A-LC) or fragment thereof comprising a mutation at one or more of V16, Q30, N81, S99, G119, I137, L150, 5156, Y184, F193, F212, I225, I234, R240, S258, L283, Y284, I302, Q310, L321, L335, V354, L360, A371, V381, Y386, T413, and F418 of SEQ ID NO: 1.

18. A deimmunized botulinum toxin serotype A light chain (BoNT/A-LC) or fragment thereof comprising a mutation at one or more of Q30, I41, V43, D80, S99, F193, L199, I236, F242, M252, L276, E278, N279, L283, Y284, Y285, F289, S294, K298, Q310, L321, S323, F330, L335, V354, K358, L360, K363, T364, N367, F368, F373, V381, I385, Y386, T413, K416, F418, L421, F422, I433, I434, T435, and T438 of SEQ ID NO: 1.

19. The deimmunized BoNT/A-LC or fragment thereof of claim 16, wherein the mutation comprises V16R or V16L; Q30E or Q30T; I41V; V43I; D80N; N81A; S99E; G119S; I137K; L150V; S156G; Y184I; F193S or F193N; L199T or L199Q; F212Y; I225T; I234T; I236G; R240E; F242T or F242S; M252Q; S258K; L276A; E278K; N279K; L283D, L283N, L283E, or L283T; Y284K; Y285A; F289Y; S294K or S249D; K298E; I302T; Q310D; L321K, L321G, or L321N; S323D; F330Y; L335D, L335E, or L335N; V354S or V354A; K358N; L360Q, L360I, or L360K; K363Q; T364S; N367G; F368Q or F368D; A371G; F373K; V381D or V381E; I385V; Y386K, Y386S, or Y386H; T413D or T413E; K416S; F418G, F418K, or F418E; L421V; F422V; I433T; I434K; T435N; T438D; or a combination thereof.

20. The deimmunized BoNT/A-LC or fragment thereof of claim 17, wherein the mutation comprises V16R or V16L; Q30E or Q30T; N81A; S99E; G119S; I137K; L150V; S156G; Y184I; F193S or F193N; F212Y; I225T; I234T; R240E; S258K; L283D, L283N, L283E, or L283T; Y284K; I302T; Q310D; L321K, L321G, or L321N; L335D, L335E, or L335N; V354S or V354A; L360Q, L360I, or L360K; A371G; V381D or V381E; Y386K, Y386S, or Y386H; T413D or T413E; F418G, F418K, or F418E; or a combination thereof.

21. The deimmunized BoNT/A-LC or fragment thereof of claim 18, wherein the mutation comprises Q30E or Q30T; I41V; V43I; D80N; S99E; F193S or F193N; L199T or L199Q; I236G; F242T or F242S; M252Q; L276A; E278K; N279K; L283D, L283N, L283E, or L283T; Y284K; Y285A; F289Y; S294K or S249D; K298E; Q310D; L321K, L321G, or L321N; S323D; F330Y; L335D, L335E, or L335N; V354S or V354A; K358N; L360Q, L360I, or L360K; K363Q; T364S; N367G; F368Q or F368D; F373K; V381D or V381E; I385V; Y386K, Y386S, or Y386H; T413D or T413E; K416S; F418G, F418K, or F418E; L421V; F422V; I433T; I434K; T435N; T438D; or a combination thereof.

22. The deimmunized BoNT/A-LC or fragment thereof of claim 17, comprising a mutation at one or more of V16, Q30, N81, S99, I137, L150, S156, F212, R240, S258, L283, Y284, I302, Q310, L335, V354, L360, A371, V381, T413, and F418 of SEQ ID NO: 1.

23. The deimmunized BoNT/A-LC or fragment thereof of claim 22, wherein the mutation comprises V16R; Q30E; N81A; S99E; I137K; L150V; S156G; F212Y; R240E; S258K; L283D, L283N, L283E, or L283T; Y284K; I302T; Q310D; L335D, L335E, or L335N; V354S or V354A; L360Q, L360I, or L360K; A371G; V381D or V381E; T413D or T413E; F418G, F418K, or F418E; or a combination thereof.

24. The deimmunized BoNT/A-LC or fragment thereof of claim 18, comprising a mutation at one or more of Q30, I41, S99, F193, L199, I236, F242, L276, E278, N279, L283, Y284, Y285, S294, K298, Q310, L321, S323, F330, L335, V354, L360, K363, T364, N367, F368, V381, I385, Y386, T413, K416, F418, L421, F422, I433, T435, and T438 of SEQ ID NO: 1.

25. The deimmunized BoNT/A-LC or fragment thereof of claim 24, wherein the mutation comprises Q30E or Q30T; I41V; S99E; F193S or F193N; L199T or L199Q; I236G; F242T or F242S; L276A; E278K; N279K; L283D, L283N, L283E, or L283T; Y284K; Y285A; S294K or S249D; K298E; Q310D; L321K, L321G, or L321N; S323D; F330Y; L335D, L335E, or L335N; V354S or V354A; L360Q, L360I, or L360K; K363Q; T364S; N367G; F368Q or F368D; V381D or V381E; I385V; Y386K, Y386S, or Y386H; T413D; K416S; F418G or F418E; L421V; F422V; I434K; T435N; T438D; or a combination thereof.

26. The deimmunized BoNT/A-LC or fragment thereof of claim 18, comprising a mutation at one or more of Q30, S99, F193, L199, F242, L276, N279, L283, Y285, L321, S323, L335, V354, L360, V381, I385, Y386, K416, L421, I433, and T438 of SEQ ID NO: 1.

27. The deimmunized BoNT/A-LC or fragment thereof of claim 26, wherein the mutation comprises Q30E or Q30T; S99E; F193S or F193N; L199T or L199Q; F242T or F242S; L276A; N279K; L283D, L283N, L283E, or L283T; Y285A; L321K, L321G, or L321N; S323D; L335D, L335E, or L335N; V354S or V354A; L360Q, L360I, or L360K; V381D or V381E; I385V; Y386K, Y386S, or Y386H; K416S; L421V; T438D; or a combination thereof.

28. The deimmunized BoNT/A-LC or fragment thereof of claim 16, comprising a mutation at one or more of Q30, S99, Y184, F212, L283, I302, A371, V381, T413, and F418 of SEQ ID NO: 1.

29. The deimmunized BoNT/A-LC or fragment thereof of claims 16 or 28, wherein the mutation comprises Q30E; S99E; Y184I; F212Y; L283D; I302T; A371G; V381D; T413D; F418G; or a combination thereof, of SEQ ID NO: 1.

30. The deimmunized BoNT/A-LC or fragment thereof of claims 16 or 28, comprising an amino acid sequence set forth in SEQ ID NO: 8, or an amino acid sequence having at least 90% identity to the amino acid sequence set forth in SEQ ID NO: 8.

31. The deimmunized BoNT/A-LC or fragment thereof of claim 16, comprising a mutation at one or more of V16, Q30, N81, F212, S258, I302, V354, L360, V381, T413, and F418 of SEQ ID NO: 1.

32. The deimmunized BoNT/A-LC or fragment thereof of claims 16 or 31, wherein the mutation comprises V16R; Q30E; N81A; F212Y; S258K; I302T; V354S; L360Q; V381D; T413D; F418G; or a combination thereof, of SEQ ID NO: 1.

33. The deimmunized BoNT/A-LC or fragment thereof of claims 16 or 31, comprising an amino acid sequence set forth in SEQ ID NO: 9, or an amino acid sequence having at least 90% identity to the amino acid sequence set forth in SEQ ID NO: 9.

34. The deimmunized BoNT/A-LC or fragment thereof of claim 16, comprising a mutation at one or more of Q30, N81, S156, L283, I302, V354, A371, T413, and F418 of SEQ ID NO: 1.

35. The deimmunized BoNT/A-LC or fragment thereof of claims 16 or 34, wherein the mutation comprises Q30E; N81A; S156G; L283D; I302T; V354S; A371G; T413D; F418G; or a combination thereof, of SEQ ID NO: 1.

36. The deimmunized BoNT/A-LC or fragment thereof of claims 16 or 34, comprising an amino acid sequence set forth in SEQ ID NO: 10, or an amino acid sequence having at least 90% identity to the amino acid sequence set forth in SEQ ID NO: 10.

37. The deimmunized BoNT/A-LC or fragment thereof of claim 16, comprising a mutation at one or more of V16, Q30, S99, L150, L283, Y284, Q310, V354, and T413 of SEQ ID NO: 1.

38. The deimmunized BoNT/A-LC or fragment thereof of claims 16 or 37, wherein the mutation comprises V16R; Q30E; S99E; L150V; L283D; Y284K; Q310D; V354S; T413D; or a combination thereof, of SEQ ID NO: 1.

39. The deimmunized BoNT/A-LC or fragment thereof of claims 16 or 37, comprising an amino acid sequence set forth in SEQ ID NO: 13, or an amino acid sequence having at least 90% identity to the amino acid sequence set forth in SEQ ID NO: 13.

40. The deimmunized BoNT/A-LC or fragment thereof of claim 16, comprising a mutation at one or more of Q30, N81, S156, S258, L283, I302, V354, and T413 of SEQ ID NO: 1.

41. The deimmunized BoNT/A-LC or fragment thereof of claims 16 or 40, wherein the mutation comprises Q30E; N81A; S156G; S258K; L283D; I302T; V354S; T413D; or a combination thereof, of SEQ ID NO: 1.

42. The deimmunized BoNT/A-LC or fragment thereof of claims 16 or 40, comprising an amino acid sequence set forth in SEQ ID NO: 15, or an amino acid sequence having at least 90% identity to the amino acid sequence set forth in SEQ ID NO: 15.

43. The deimmunized BoNT/A-LC or fragment thereof of claim 16, comprising a mutation at one or more of Q30, N81, S99, L150, S258, L283, Y284, Q310, V354, A371, T413, and F418 of SEQ ID NO: 1.

44. The deimmunized BoNT/A-LC or fragment thereof of claims 16 or 43, wherein the mutation comprises Q30E; N81A; S99E; L150V; S258K; L283D; Y284K; Q310D; V354S; A371G; T413D; F418G; or a combination thereof, of SEQ ID NO: 1.

45. The deimmunized BoNT/A-LC or fragment thereof of claims 16 or 43, comprising an amino acid sequence set forth in SEQ ID NO: 18, or an amino acid sequence having at least 90% identity to the amino acid sequence set forth in SEQ ID NO: 18.

46. The deimmunized BoNT/A-LC or fragment thereof of claim 16, comprising a mutation at one or more of V16, Q30, N81, S156, L283, Q310, V354, A371, T413, and F418 of SEQ ID NO: 1.

47. The deimmunized BoNT/A-LC or fragment thereof of claims 16 or 46, wherein the mutation comprises V16R; Q30E; N81A; S156G; L283D; Q310D; V354S; A371G; T413D; F418G; or a combination thereof, of SEQ ID NO: 1.

48. The deimmunized BoNT/A-LC or fragment thereof of claims 16 or 46, comprising an amino acid sequence set forth in SEQ ID NO: 24, or an amino acid sequence having at least 90% identity to the amino acid sequence set forth in SEQ ID NO: 24.

49. The deimmunized BoNT/A-LC or fragment thereof of claim 16, comprising a mutation at one or more of Q30, N81, I137, L150, F212, L283, Q310, V354, A371, and V381 of SEQ ID NO: 1.

50. The deimmunized BoNT/A-LC or fragment thereof of claims 16 or 49, wherein the mutation comprises Q30E; N81A; I137K; L150V; F212Y; L283D; Q310D; V354S; A371G; V381D; or a combination thereof, of SEQ ID NO: 1.

51. The deimmunized BoNT/A-LC or fragment thereof of claims 16 or 49, comprising an amino acid sequence set forth in SEQ ID NO: 27, or an amino acid sequence having at least 90% identity to the amino acid sequence set forth in SEQ ID NO: 27.

52. The deimmunized BoNT/A-LC or fragment thereof of claim 16, comprising a mutation at one or more of V16, N81, I137, S156, F212, R240, L283, I302, Q310, L335, V354, L360, A371, and V381 of SEQ ID NO: 1.

53. The deimmunized BoNT/A-LC or fragment thereof of claims 16 or 52, wherein the mutation comprises V16R; N81A; I137K; S156G; F212Y; R240E; L283D; I302T; Q310D; L335N; V354S; L360Q; A371G; V381D; or a combination thereof, of SEQ ID NO: 1.

54. The deimmunized BoNT/A-LC or fragment thereof of claims 16 or 52, comprising an amino acid sequence set forth in SEQ ID NO: 30, or an amino acid sequence having at least 90% identity to the amino acid sequence set forth in SEQ ID NO: 30.

55. The deimmunized BoNT/A-LC or fragment thereof of any one of claims 52-54, further comprising a F418G mutation.

56. The deimmunized BoNT/A-LC or fragment thereof of claim 16, comprising a mutation at one or more of V16, N81, I137, S156, F212, R240, L283, Q310, V354, A371, T413, and F418 of SEQ ID NO: 1.

57. The deimmunized BoNT/A-LC or fragment thereof of claims 16 or 56, wherein the mutation comprises V16R; N81A; I137K; S156G; F212Y; R240E; L283D; Q310D; V354S; A371G; T413D; F418G; or a combination thereof, of SEQ ID NO: 1.

58. The deimmunized BoNT/A-LC or fragment thereof of claims 16 or 56, comprising an amino acid sequence set forth in SEQ ID NO: 41, or an amino acid sequence having at least 90% identity to the amino acid sequence set forth in SEQ ID NO: 41.

59. The deimmunized BoNT/A-LC or fragment thereof of claim 16, comprising a mutation at one or more of V16, S99, I137, S156, F212, R240, L283, Q310, V354, and A371 of SEQ ID NO: 1.

60. The deimmunized BoNT/A-LC or fragment thereof of claims 16 or 59, wherein the mutation comprises V16R; S99E; I137K; S156G; F212Y; R240E; L283D; Q310D; V354S; A371G; or a combination thereof, of SEQ ID NO: 1.

61. The deimmunized BoNT/A-LC or fragment thereof of claims 16 or 59, comprising an amino acid sequence set forth in SEQ ID NO: 42, or an amino acid sequence having at least 90% identity to the amino acid sequence set forth in SEQ ID NO: 42.

62. The deimmunized BoNT/A-LC or fragment thereof of claim 16, comprising a mutation at one or more of Q30, N81, I137, L150, R240, L283, I302, L360, V381, T413, and F418 of SEQ ID NO: 1.

63. The deimmunized BoNT/A-LC or fragment thereof of claims 16 or 62, wherein the mutation comprises Q30E; N81A; I137K; L150V; R240E; L283D; I302T; L360Q; V381D; T413D; F418G; or a combination thereof, of SEQ ID NO: 1.

64. The deimmunized BoNT/A-LC or fragment thereof of claims 16 or 62, comprising an amino acid sequence set forth in SEQ ID NO: 46, or an amino acid sequence having at least 90% identity to the amino acid sequence set forth in SEQ ID NO: 46.

65. The deimmunized BoNT/A-LC or fragment thereof of claim 16, comprising a mutation at one or more of S99, F193, L283, V354, V381, and I433 of SEQ ID NO: 1.

66. The deimmunized BoNT/A-LC or fragment thereof of claims 16 or 65, wherein the mutation comprises S99E; F193S; L283E; V354A; V381D; I433T; or a combination thereof, of SEQ ID NO: 1.

67. The deimmunized BoNT/A-LC or fragment thereof of claims 16 or 65, comprising an amino acid sequence set forth in SEQ ID NO: 94, or an amino acid sequence having at least 90% identity to the amino acid sequence set forth in SEQ ID NO: 94.

68. The deimmunized BoNT/A-LC or fragment thereof of claim 16, comprising a mutation at one or more of S99, F193, L283, L335, V354, V381, and I433 of SEQ ID NO: 1.

69. The deimmunized BoNT/A-LC or fragment thereof of claims 16 or 68, wherein the mutation comprises S99E; F193S; L283E; L335D; V354A; V381D; I433T; or a combination thereof, of SEQ ID NO: 1.

70. The deimmunized BoNT/A-LC or fragment thereof of claims 16 or 68, comprising an amino acid sequence set forth in SEQ ID NO: 95, or an amino acid sequence having at least 90% identity to the amino acid sequence set forth in SEQ ID NO: 95.

71. The deimmunized BoNT/A-LC or fragment thereof of claim 16, comprising a mutation at one or more of Q30, S99, F193, L283, L335, V354, V381, and T438 of SEQ ID NO: 1.

72. The deimmunized BoNT/A-LC or fragment thereof of claims 16 or 71, wherein the mutation comprises Q30E; S99E; F193S; L283E; L335D; V354A; V381D; T438D; or a combination thereof, of SEQ ID NO: 1.

73. The deimmunized BoNT/A-LC or fragment thereof of claims 16 or 71, comprising an amino acid sequence set forth in SEQ ID NO: 96, or an amino acid sequence having at least 90% identity to the amino acid sequence set forth in SEQ ID NO: 96.

74. The deimmunized BoNT/A-LC or fragment thereof of claim 16, comprising a mutation at one or more of Q30, S99, L283, L335, V354, V381, K416, I433, and T438 of SEQ ID NO: 1.

75. The deimmunized BoNT/A-LC or fragment thereof of claims 16 or 74, wherein the mutation comprises Q30E; S99E; L283E; L335D; V354A; V381D; K416S; I433T; T438D; or a combination thereof, of SEQ ID NO: 1.

76. The deimmunized BoNT/A-LC or fragment thereof of claims 16 or 74, comprising an amino acid sequence set forth in SEQ ID NO: 97, or an amino acid sequence having at least 90% identity to the amino acid sequence set forth in SEQ ID NO: 97.

77. The deimmunized BoNT/A-LC or fragment thereof of claim 16, comprising a mutation at one or more of S99, L199, N279, L283, L321, S323, L335, V381, K416, and T438 of SEQ ID NO: 1.

78. The deimmunized BoNT/A-LC or fragment thereof of claims 16 or 77, wherein the mutation comprises S99E; L199T; N279K; L283E; L321K; S323D; L335E; V381D; K416S; T438D; or a combination thereof, of SEQ ID NO: 1.

79. The deimmunized BoNT/A-LC or fragment thereof of claims 16 or 77, comprising an amino acid sequence set forth in SEQ ID NO: 98, or an amino acid sequence having at least 90% identity to the amino acid sequence set forth in SEQ ID NO: 98.

80. The deimmunized BoNT/A-LC or fragment thereof of claim 16, comprising a mutation at one or more of S99, L199, N279, L283, L321, S323, L335, L360, V381, L421, and T438 of SEQ ID NO: 1.

81. The deimmunized BoNT/A-LC or fragment thereof of claims 16 or 80, wherein the mutation comprises S99E; L199T; N279K; L283E; L321K; S323D; L335E; L360Q; V381D; L421V; T438D; or a combination thereof, of SEQ ID NO: 1.

82. The deimmunized BoNT/A-LC or fragment thereof of claims 16 or 77, comprising an amino acid sequence set forth in SEQ ID NO: 100, or an amino acid sequence having at least 90% identity to the amino acid sequence set forth in SEQ ID NO: 100.

83. The deimmunized BoNT/A-LC or fragment thereof of any one of claims 1-82, comprising an amino acid sequence set forth in any one of SEQ ID NOs: 8-109, or an amino acid sequence having at least 90% identity to the amino acid sequence set forth in any one of SEQ ID NO: NOs: 8-109.

84. The deimmunized botulinum toxin light chain or fragment thereof of any one of claims 1-83, comprising 2, 3, 4, 5, 6, 7, 8, 9, 10, 11, 12, 13, 14, 15, 16, 17, 18, 19, 20, or more mutations.

85. The deimmunized botulinum toxin light chain or fragment thereof of any one of claims 1-84, comprising about 0.10% activity or greater relative to a wildtype botulinum toxin light chain or fragment thereof.

86. The deimmunized botulinum toxin light chain or fragment thereof of any one of claims 1-84, comprising about 0.1%, about 1%, about 5%, about 10%, about 20%, about 30%, about 40%, about 50%, about 60%, about 70%, about 80%, about 90%, or about 100% activity relative to a wildtype botulinum toxin light chain or fragment thereof.

87. The deimmunized botulinum toxin light chain or fragment thereof of any one of claims 1-86, comprising thermostability within about 10° C. of a wildtype botulinum toxin light chain or fragment thereof.

88. The deimmunized botulinum toxin light chain or fragment thereof of any one of claims 1-86, comprising thermostability within about 3° C. to about 8° C. of a wildtype botulinum toxin light chain or fragment thereof.

89. The deimmunized botulinum toxin light chain or fragment thereof of any one of claims 1-86, comprising thermostability about equal to a wildtype botulinum toxin light chain or fragment thereof.

90. The deimmunized botulinum toxin light chain or fragment thereof of any one of claims 1-18, fused to a functional moiety.

91. The deimmunized botulinum toxin light chain or fragment thereof of claim 90, wherein the functional moiety comprises a targeting activity and/or binding activity.

92. The deimmunized botulinum toxin light chain or fragment thereof of claim 90, wherein the functional moiety is selected from the group consisting of an antigen binding protein or fragment thereof, an imaging molecule, an oligonucleotide, a targeting peptide, and polyethylene glycol (PEG).

93. The deimmunized botulinum toxin light chain or fragment thereof of claim 92, wherein the antigen binding protein fragment comprises an Fc domain, a Fab domain, an scFv, or a single domain antibody.

94. The deimmunized BoNT/A-LC or fragment thereof of any one of claims 1-93, further comprising one or both of an L427A mutation and an L428A mutation.

95. The deimmunized BoNT/A-LC or fragment thereof of any one of claims 1-93, further comprising an L427A mutation and an L428A mutation.

96. The deimmunized BoNT/A-LC or fragment thereof of any one of claims 1-95, further comprising a P1A mutation.

97. The deimmunized BoNT/A-LC or fragment thereof of any one of claims 1-96, further comprising a A26V mutation.

98. The deimmunized botulinum toxin light chain or fragment thereof of any one of claims 1-98, further comprising an N terminal methionine.

99. The deimmunized botulinum toxin light chain or fragment thereof of any one of claims 1-98, further comprising a botulinum toxin heavy chain (BoNT-HC) or fragment thereof.

100. The deimmunized botulinum toxin light chain or fragment thereof of any one of claims 1-99, further comprising a botulinum toxin serotype A heavy chain (BoNT/A-HC) or fragment thereof.

101. The deimmunized botulinum toxin light chain or fragment thereof of claim 99, wherein the BoNT-HC or fragment thereof is a serotype other than serotype A.

102. The deimmunized botulinum toxin light chain or fragment thereof of claim 101, wherein the BoNT-HC serotype is selected from the group consisting of serotype B, serotype C, serotype D, serotype E, serotype F, and serotype G.

103. A pharmaceutical composition comprising the deimmunized botulinum toxin light chain or fragment thereof of any one of claims 1-102 and a pharmaceutically acceptable carrier.

104. A vector encoding the deimmunized botulinum toxin light chain or fragment thereof of any one of claims 1-102.

105. A host cell comprising the vector of claim 104.

106. The host cell of claim 105, comprising prokaryotic host cell or a eukaryotic host cell.

107. The host cell of claim 105, comprising an E. coli host cell, a Clostridium genus host cell, a yeast host cell, an insect host cell, or a mammalian host cell.

108. The host cell of claim 107, wherein the clostridium genus host cell comprises C. botulinum, C. baratii, C. butyricum, or C. argentinense.

109. A method of treating or preventing a disease or disorder in a subject that would benefit from a therapeutically effective amount of a botulinum toxin, comprising administering a therapeutically effective amount of the deimmunized botulinum toxin light chain or fragment thereof of any one of claims 1-102 to the subject.

110. A method of treating or preventing a disease or disorder of inappropriate muscle contraction in a subject, comprising administering a therapeutically effective amount of the deimmunized botulinum toxin light chain or fragment thereof of any one of claims 1-102 to the subject.

111. A method of treating or preventing a disease or disorder of inappropriate neuron signaling in a subject, comprising administering a therapeutically effective amount of the deimmunized botulinum toxin light chain or fragment thereof of any one of claims 1-102 to the subject.

112. The method of any one of claims 109-111, wherein the disease or disorder is selected from the group consisting of acute pain, alopecia, aquagenic keratoderma, atrial fibrillation, blepharospasm, bromhidrosis, cerebral palsy, cervical dystonia, chromhidrosis, chronic anal fissures, chronic pain, constipation, depression, dermatosis, eccrine nevus, eczema, esophageal spasms, essential tremor, facial erythema and flushing, genodermatoses, Hailey-Hailey disease, hand dystonia, hemifacial spasm, hidradenitis suppurativa, hyperhydrosis, hypersialorrhoea, hypertrophic scars, keloids, linear IgA bullous dermatosis, migraine headache, notalgia paresthetica, oily skin, postherpetic neuralgia, psoriasis, overactive bladder, premature ejaculation, Raynaud's Disease, spastic paresis, strabismus, tension headache, voice abnormalities, whiplash.

113. A method of treating a subject for a cosmetic purpose, comprising administering a therapeutically effective amount of the deimmunized botulinum toxin light chain or fragment thereof of any one of claims 1-102 to the subject.

114. The method of claim 113 wherein the cosmetic purpose is the reduction of facial wrinkles.

115. The method of claim 114, wherein the facial wrinkles comprise brow line wrinkles and glabellar frown lines.

116. A method for reducing an antibody response against botulinum toxin light chain or fragment thereof in a subject, comprising administering a therapeutically effective amount of the deimmunized BoNT/A-LC or fragment thereof of any one of claims 1-102 to the subject.

117. A method for producing a deimmunized botulinum toxin light chain or fragment thereof in a host cell, comprising:
a) introducing a vector encoding the deimmunized botulinum toxin light chain or fragment thereof of any one of claims 1-102, into a host cell to produce a deimmunized botulinum toxin light chain-expressing host cell;
b) culturing the host cell in a culture system; and
c) isolating the deimmunized botulinum toxin light chain or fragment thereof from the culture system.
